(19) United States
(12) Patent Application Publication
(10) Pub. No.: US 2016/0213742 A1
(43) Pub. Date: Jul. 28, 2016

(54) COMPOSITIONS AND METHODS FOR THE TREATMENT OF FUNGAL INFECTIONS

(71) Applicant: CIDARA THERAPEUTICS, INC, San Diego, CA (US)

(72) Inventors: Kevin Michael FORREST, Del Mar, CA (US); James Kevin JUDICE, El Granada, CA (US); H. Shaw WARREN, Cambridge, MA (US); James Michael BALKOVEC, Martinsville, NJ (US); Thomas Patrick BRADY, San Diego, CA (US)

(73) Assignees: Cidara Therapeutics, Inc., San Diego, CA (US); Cidara Therapeutics, Inc., San Diego, CA (US)

(21) Appl. No.: 14/916,414

(22) PCT Filed: Sep. 4, 2014
(86) PCT No.: PCT/US2014/054164

§ 371 Date: Mar. 3, 2016

Related U.S. Application Data
(60) Provisional application No. 61/873,761, filed on Sep. 4, 2013.
(60) Provisional application No. 61/932,150, filed on Jan. 27, 2014.

(51) Int. Cl. A61K 38/12; A61K 47/48; C07K 7/64

(52) U.S. Cl. CPC A61K 38/12; C07K 7/64; A61K 47/48246

(57) ABSTRACT

Compositions and methods for the treatment of fungal infections including compounds containing a pathogen pattern recognition receptor ligand and a β 1,3-glucan synthase inhibitor are disclosed. In particular, compounds containing a lipopeptide moiety and a formyl peptide receptor ligand can be used in the treatment of fungal infections caused by a fungus of the genus Aspergillus or Candida.

### BACKGROUND

The need for novel antifungal treatments is significant and is especially critical in the medical field. Immunocompromised patients provide perhaps the greatest challenge to modern health care delivery. The most common pathogens associated with invasive fungal infections are the opportunistic yeast, Candida albicans, and the filamentous fungus, Aspergillus fumigatus.

The development of antifungal treatment regimens has been a continuing challenge. When the echinocandin, caspofungin, was approved for sale in 2001, it represented the first new class of antifungal agents to be approved in over a decade. Since that time, two other echinocandin antifungals, anidulafungin and micafungin, have been approved in various markets. Each agent in this class of compound acts by inhibition of β-1,3-glucan synthase, which is a key enzyme in the synthesis of glucan in the cell wall of many fungi. All three of these drugs are made semisynthetically, starting with natural products obtained through fermentation.

The echinocandins are a broad group of antifungal agents that typically comprise a cyclic hexapeptide and a lipophilic tail, the latter of which is attached to the hexapeptide core through an amide linkage. Although many echinocandins are natural products, the clinically relevant members of this class have all been semisynthetic derivatives.

### SUMMARY

The disclosure relates to compositions, methods for inhibiting fungal growth, and methods for the treatment of fungal infections. In particular, such compositions include bifunctional molecules comprising a moiety that inhibits β-1,3-glucan synthase and a moiety that interacts with a pathogen pattern recognition receptor (e.g., a chemotaxis receptor). Such compositions are useful in methods for the inhibition of fungal growth and in methods for the treatment of fungal infections, such as those caused by a fungus of the genus Aspergillus or Candida.

Accordingly, in a first aspect, the invention features a compound (e.g., a synthetic bifunctional non-antibody compound), or a pharmaceutically acceptable salt thereof, comprising, consisting essentially of, or consisting of a pathogen pattern recognition receptor ligand conjugated to an inhibitor of β-1,3-glucan synthase. Compounds including a pathogen pattern recognition receptor (e.g., a chemotaxis receptor, a formyl peptide receptor such as FPR1, FPR2, or FPR3, or a member of the formyl peptide receptor family such as, FPRL1, FPRL2) ligand (e.g., a chemotatic peptide) conjugated (e.g., by an amide bond, for example, between the C-terminus of a chemotactic peptide and a terminal primary or secondary amine of the inhibitor of β-1,3-glucan synthase or a linker) to an inhibitor of β-1,3-glucan synthase (e.g., a naturally occurring, semi-synthetic, or total synthetic cyclic lipopeptide, such as a member of the aculeacin, echinocandin, pneumocandin, cyclopeptamine, mulundocandin, sporiofungin, or WF11899A class of antifungal drugs, which include caspofungin, echinocandin B, cilofungin, pneumocandin A₀, pneumocandin B₀, L-705589, L-731373, L-733560, A-174591, A-172013, A-175800, micafungin, and anidulafungin) are disclosed herein. An inhibitor of β-1,3-glucan synthase also includes amino-biafungin, amino-AF-053, and amino-AF-033, described herein.

In some embodiments, the inhibitor of β-1,3-glucan synthase can comprise, consist essentially of, or consist of a compound having the structure:

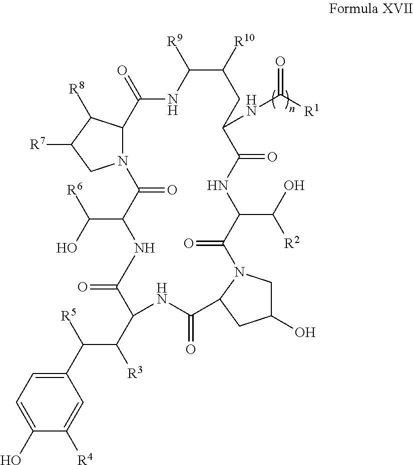

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; R¹¹, R¹², and R¹³ are independently hydrogen or C₁-C₃ alkyl; n is 0 or 1; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, R⁷, R⁹, or R¹⁰ contains —NR¹¹R¹².

In other embodiments, the inhibitor of β-1,3-glucan synthase can comprise, consist essentially of, or consist of a compound having the structure:

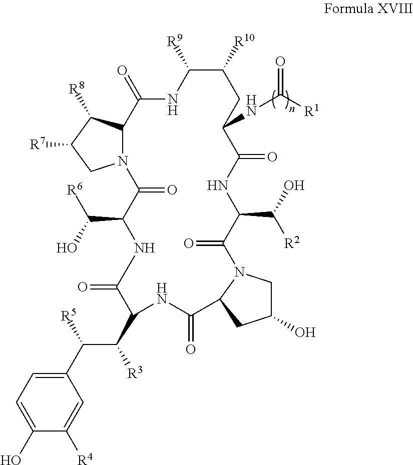

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R¹², NH(CH₂)ₐN⁺R¹¹R¹², or —NH(CH₂)ₐN⁺R¹¹R¹²R¹³; R¹¹, R¹², and R¹³ are independently hydrogen or C₁-C₃ alkyl; n is 0 or 1; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, R⁷, R⁹, or R¹⁰ contains an —NR¹¹R¹².

In some embodiments, the inhibitor of β-1,3-glucan synthase can comprise, consist essentially of, or consist of a compound having the structure:

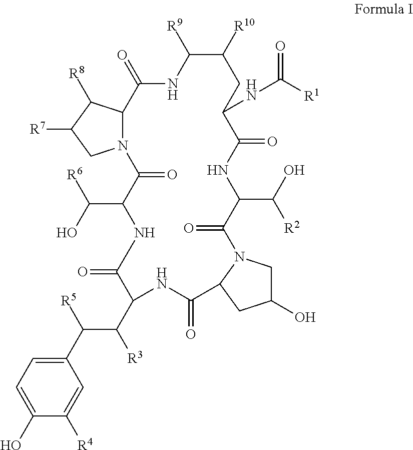

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R¹², NH(CH₂)ₐN⁺R¹¹R¹², or —NH(CH₂)ₐN⁺R¹¹R¹²R¹³; R¹¹, R¹², and R¹³ are independently hydrogen or C₁-C₃ alkyl; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, or, R⁹ contains —NR¹¹R¹².

In other embodiments, the inhibitor of β-1,3-glucan synthase can comprise, consist essentially of, or consist of a compound having the structure:

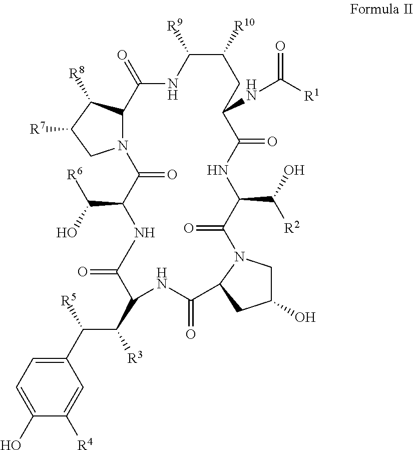

where R¹ is a C₁₀-C₃₆ moiety containing 0-8 heteroatoms selected from O, S, and N, optionally substituted with halo (preferably chloro or fluoro); R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R₁₂, NH(CH₂)ₐN⁺R¹¹R¹², or —NH(CH₂)ₐN⁺R¹¹R¹²R¹³; R¹¹, R¹², and R¹³ are independently hydrogen or C₁-C₃ alkyl; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, or R⁹ contains an —NR¹¹R¹².

In certain embodiments, the inhibitor of β-1,3-glucan synthase can comprise, consist essentially of, or consist of a compound having the structure of Formula I, II, XVII, or XVIII, where R¹ is optionally substituted C₁₃-C₁₇ alkyl, optionally substituted C₁₃-C₁₇ alkenyl, optionally substituted aryl, or optionally substituted heteroaryl; R² is methyl; R³ and R⁵ are hydroxyl; R⁴ is hydrogen, hydroxyl, —OSO₃H or —NR¹¹R¹², preferably hydrogen; R⁶ is methyl, —CH₂(C═O)NR¹¹R¹², —CH₂CH₂NR¹¹R¹², or —CH₂CH₂N⁺R¹¹R¹²R¹³, preferably —CH₂CH₂NR¹¹R¹²; R⁷ is hydrogen, methyl, —NR¹¹R¹², or —NH(C═NH)NR¹¹R¹²; R⁸ is hydroxyl; R⁹ is hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐN⁺R¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³; preferably —O(CH₂)ₐN⁺R¹¹R¹² or —NH(CH₂)ₐN⁺R¹¹R¹²; R¹⁰ is hydrogen, hydroxyl, oxo, —NR¹¹R¹², NH(CH₂)ₐN⁺R¹¹R¹², or —NH(CH₂)ₐN⁺R¹¹R¹²R¹³; R¹¹, R¹², and R¹³ are independently hydrogen or methyl; n is 0 or 1; and a is 2 to 4; where at least one of R⁴, R⁶, R⁷, R⁹, or R¹⁰ contains an —NR¹¹R¹².

In some embodiments, R⁴ of Formula I, II, XVII or XVIII contains —NR¹¹R¹².

In some embodiments, R⁶ of Formula I, II, XVII or XVIII contains —NR¹¹R¹².

In some embodiments, R⁷ of Formula I, II, XVII or XVIII contains —NR¹¹R¹².

In some embodiments, R⁹ of Formula I, II, XVII or XVIII contains —NR¹¹R¹².

In some embodiments, R¹⁰ of Formula I, II, XVII or XVIII contains —NR¹¹R¹².

In some embodiments, R¹ of Formula I, II, XVII or XVIII contains a C₁-C₁₇ alkyl or heteroalkyl, C₂-C₁₇ alkenyl or heteroalkenyl, aryl or heteroaryl, cyclic, polycyclic, heterocyclic or heteropolycyclic moiety, or a combination thereof to form a C₁₀-C₃₆ moiety.

In some embodiments, R¹ of Formula I, II, XVII or XVIII contains optionally substituted C₁₃-C₁₇ alkyl, optionally substituted C₁₃-C₁₇ alkenyl, optionally substituted aryl, or optionally substituted heteroaryl.

In some embodiments, R⁹ of Formula I, II, XVII or XVIII contains hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐN⁺R¹¹R¹²R¹³, —S(CH₂)ₐN⁺R¹¹R¹², —S(CH₂)ₐN⁺R¹¹R¹²R¹³, —CH₂NR¹¹R¹², or —CH₂N⁺R¹¹R¹²R¹³.

In some embodiments, R⁹ of Formula I, II, XVII or XVIII contains hydrogen, hydroxyl, —NH₂, —O(CH₂)₂NH₂, —O(CH₂)₂N⁺(CH₃)₃, —O(CH₂)₂O(CH₂)₂NH₂, —NH(CH₂)₂NH₂, —NH(CH₂)₂N⁺(CH₃)₃, —S(CH₂)₂NH₂, or —CH₂NH₂.

In some embodiments, R¹ of Formula I, II, XVII or XVIII is

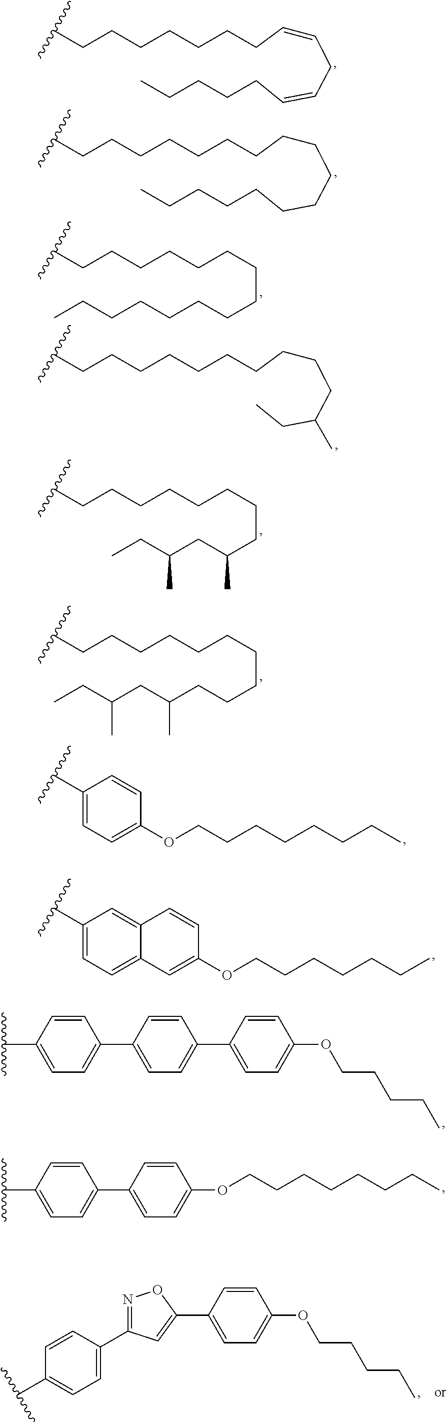

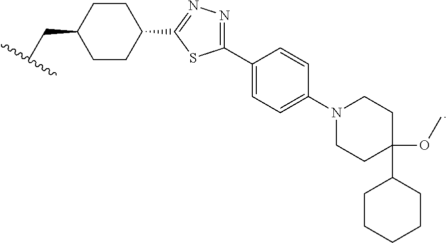 [SMILES: *C[C@H]1CC[C@H](c2nnc(-c3ccc(N4CCC(OC)(C5CCCCC5)CC4)cc3)s2)CC1]

In some embodiments, the inhibitor of β-1,3-glucan synthase can comprise, consist essentially of, or consist of a compound having the structure:

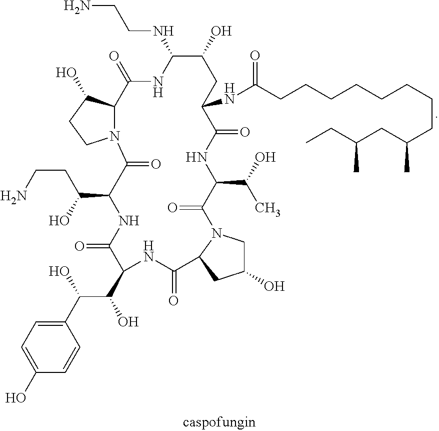 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

In other embodiments, the inhibitor of β-1,3-glucan synthase can comprise, consist essentially of, or consist of a compound having the structure:

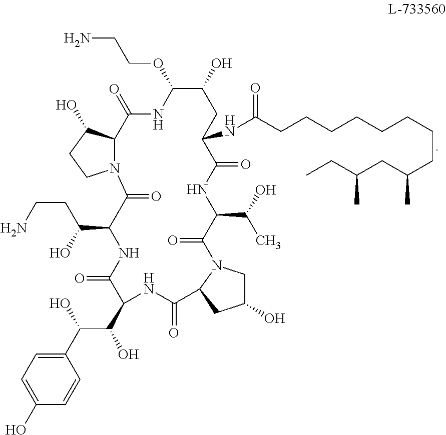 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

In further embodiments, the inhibitor of β-1,3-glucan synthase can comprise, consist essentially of, or consist of a compound having the structure:

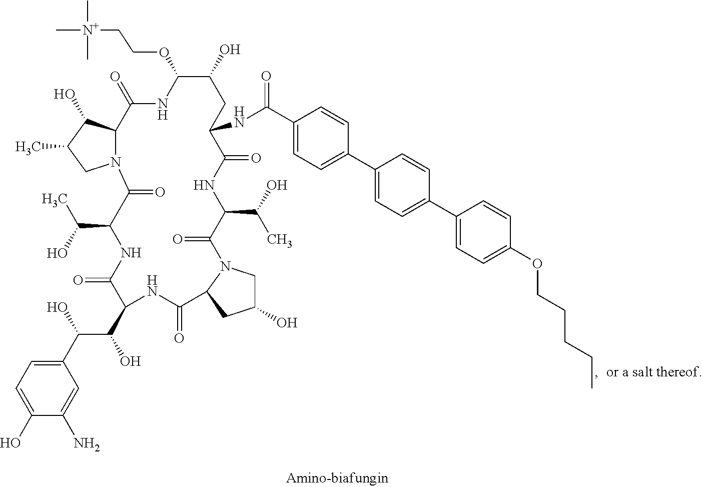 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

In further embodiments, the inhibitor of β-1,3-glucan synthase can comprise, consist essentially of, or consist of a compound having the structure:

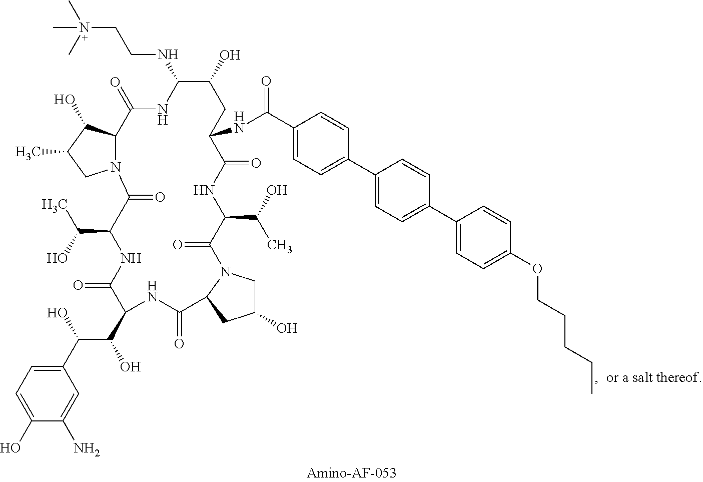

In further embodiments, the inhibitor of β-1,3-glucan synthase can comprise, consist essentially of, or consist of a compound having the structure:

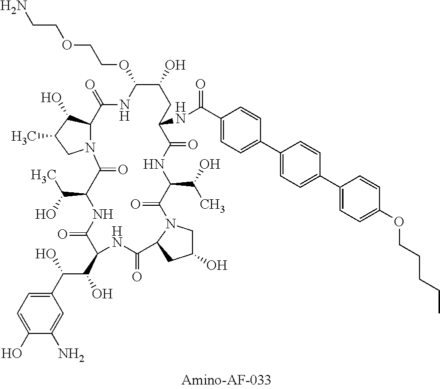 [SMILES: CCCCCOc1ccc(-c2ccc(-c3ccc(C(=O)N[C@H]4C[C@@H](O)[C@@H](OCCOCCN)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

In other embodiments, the inhibitor of β-1,3-glucan synthase can comprise, consist essentially of, or consist of a compound having the structure:

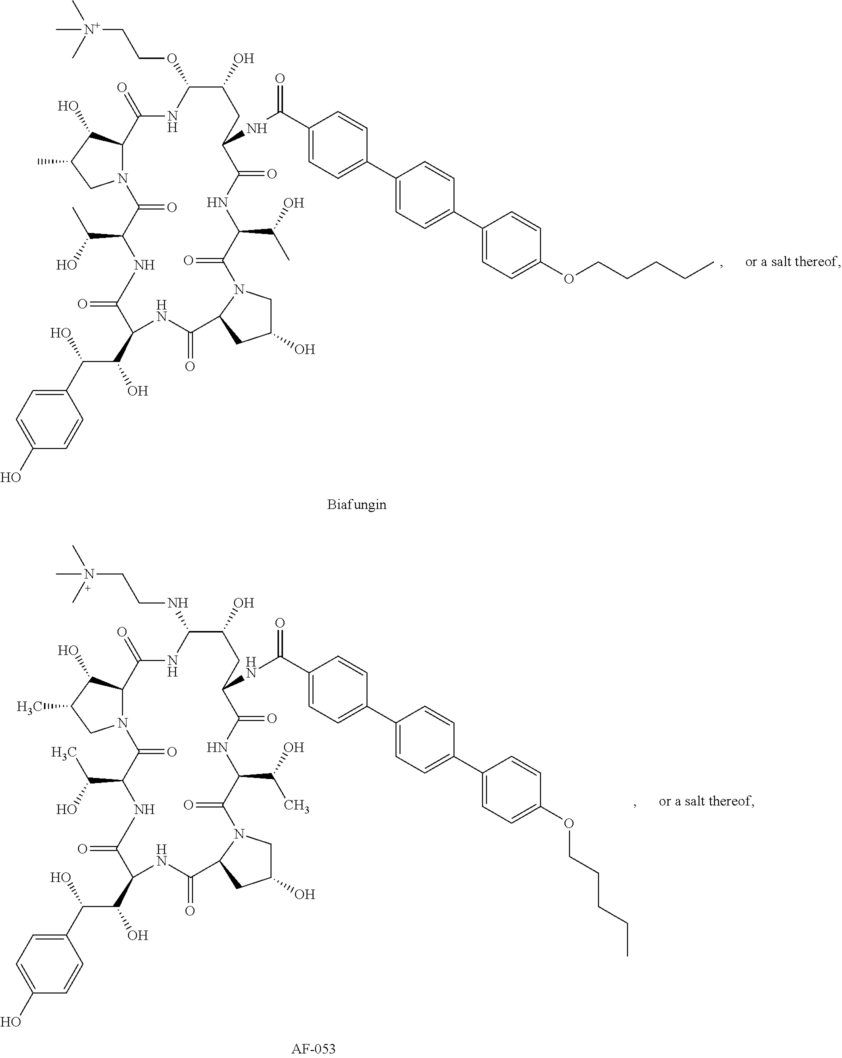

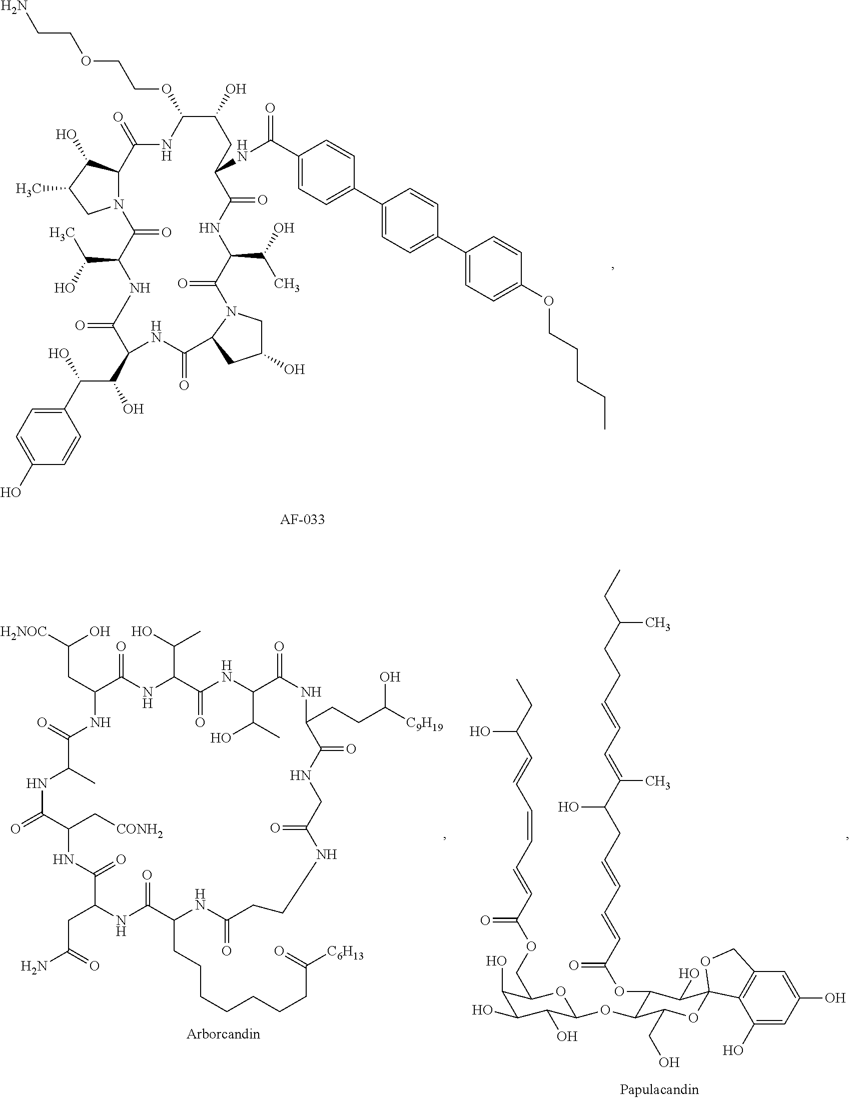

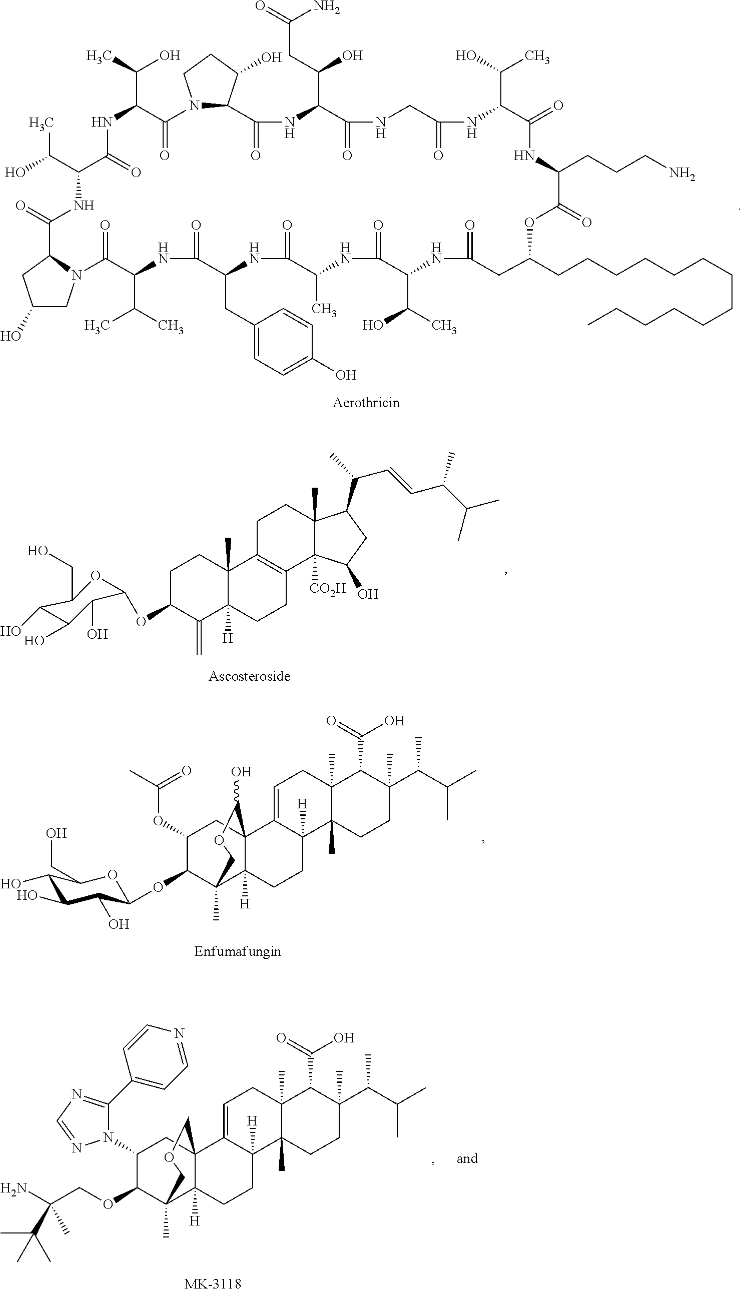

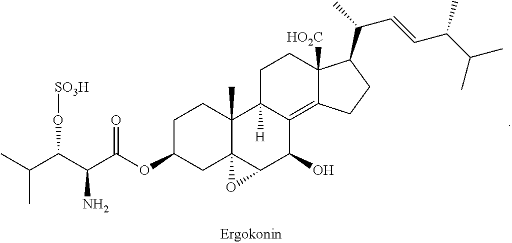

or an amine-containing derivative thereof. Derivitization of glucan synthase inhibitors that do not already possess an amino group may be accomplished by any one of several standard synthetic methods. For example, for the arborcandins, dehydration and reduction of a primary carboxamide would yield a primary amine. For the papulacandins, protection of the hydroxyl groups, followed by nitration of the phenolic group, reduction of the nitro to an amine and deprotection would yield an aniline group. For ascosteroside or enfumafungin derivitization of the carboxylic acid by alkylation with allyl bromide followed by ozonolysis of the allyl group and reductive amination would introduce a 2-aminoethyl ester group.

In certain embodiments, the pathogen pattern recognition receptor ligand is a chemotactic peptide comprising, consisting essentially of, or consisting of an amino acid residue having the formula:

R¹⁴—X1-X2-X3-X4-X5-X6-X7-X8-X9 Formula III

where X9 is any amino acid; X1-X8 are any amino acid or absent; R¹⁴ is hydrogen or

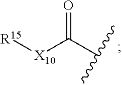

where X₁₀ is a bond, NH, or O; and R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic; where if R¹⁴ is hydrogen X1 is not absent (e.g., X1 and X2 are not absent).

In particular embodiments, the chemotactic peptide can comprise, consist essentially of, or consist of an amino acid residue having the formula:

R¹⁴—X1-X2-X9 Formula IV

where X1 is any amino acid; X2 is leucine or isoleucine, or is absent; X9 is any amino acid; R¹⁴ is hydrogen or

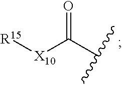

where X₁₀ is a bond, NH, or O; and R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic.

Amino acids that can be included in the chemotactic peptide can be selected from naturally occurring amino acids (e.g., Ala, Arg, Asn, Asp, Cys, Gin, Glu, Gly, His, Ile, Leu, Lys, Met, Phe, Pro, Ser, Thr, Trp, Tyr, or Val), or non-naturally occurring amino acids. A “non-naturally occurring amino acid” is an amino acid which is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; diaminopropionic acid; and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methylaminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methylhomophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-β-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine.

In certain embodiments, X1 is selected from methionine, oxymethionine, or norleucine, or is absent. In certain embodiments, X2 is selected from leucine and isoleucine, or is absent. In certain embodiments, X9 is selected from phenylalanine, 1-amino-2-phenylcyclopropane-1-carboxylic acid, methionine, and serine, or is absent. In certain embodiments, R¹⁴ is —C(O)H. In certain embodiments, R¹⁴ is —C(O)CH₃. In other embodiments, R¹⁴ is —C(O)OCH₂CH(CH₃)₂. In various embodiments, each of the aforementioned selections for X1, X2, X9, and R¹⁴ can each be considered independently and in various combinations with each other.

In some embodiments, the pathogen pattern recognition receptor ligand comprises, consists essentially of, or consists of a compound having the structure:

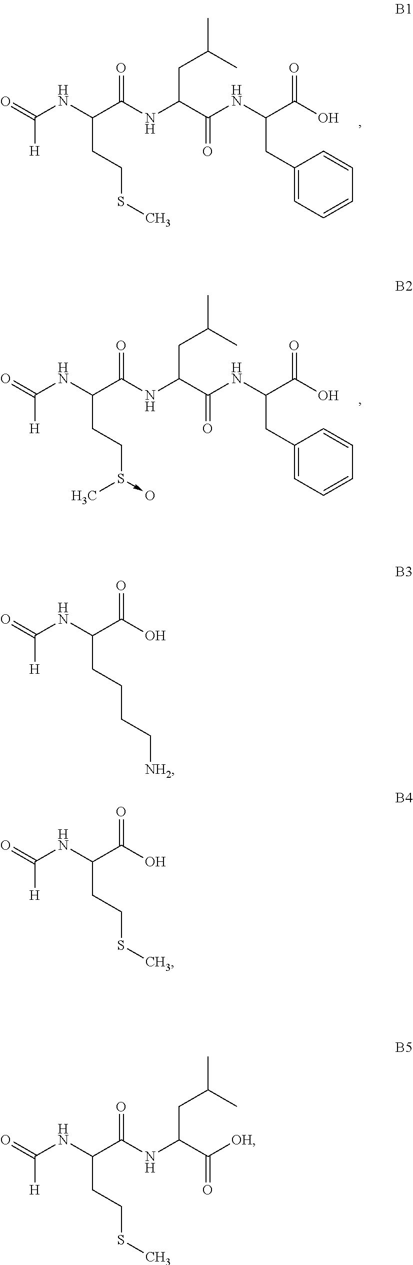 [SMILES: CSCCC(NC=O)C(=O)NC(CC(C)C)C(=O)NC(Cc1ccccc1)C(=O)O ; CC(C)CC(NC(=O)C(CCS(C)=O)NC=O)C(=O)NC(Cc1ccccc1)C(=O)O ; NCCCCC(NC=O)C(=O)O ; CSCCC(NC=O)C(=O)O ; CSCCC(NC=O)C(=O)NC(CC(C)C)C(=O)O]

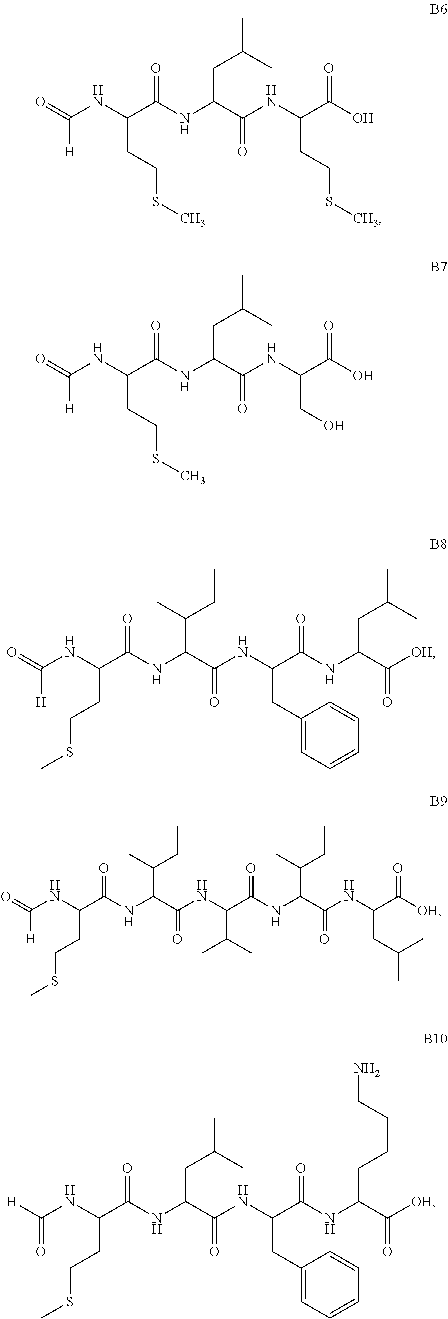 [SMILES: CSCCC(NC(=O)C(CC(C)C)NC(=O)C(CCSC)NC=O)C(=O)O ; CSCCC(NC=O)C(=O)NC(CC(C)C)C(=O)NC(CO)C(=O)O ; CCC(C)C(NC(=O)C(CCSC)NC=O)C(=O)NC(Cc1ccccc1)C(=O)NC(CC(C)C)C(=O)O ; CCC(C)C(NC(=O)C(CCSC)NC=O)C(=O)NC(C(=O)NC(C(=O)NC(CC(C)C)C(=O)O)C(C)CC)C(C)C ; CSCCC(NC=O)C(=O)NC(CC(C)C)C(=O)NC(Cc1ccccc1)C(=O)NC(CCCCN)C(=O)O]

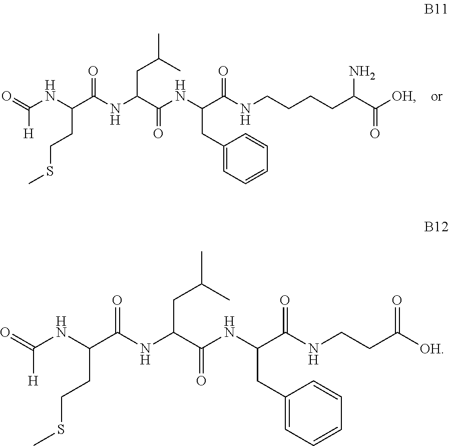 [SMILES: CSCCC(NC=O)C(=O)NC(CC(C)C)C(=O)NC(Cc1ccccc1)C(=O)NCCCCC(N)C(=O)O ; "or" ; CSCCC(NC=O)C(=O)NC(CC(C)C)C(=O)NC(Cc1ccccc1)C(=O)NCCC(=O)O]

In other embodiments, the pathogen pattern recognition receptor ligand comprises, consists essentially of, or consists of a compound having the structure:

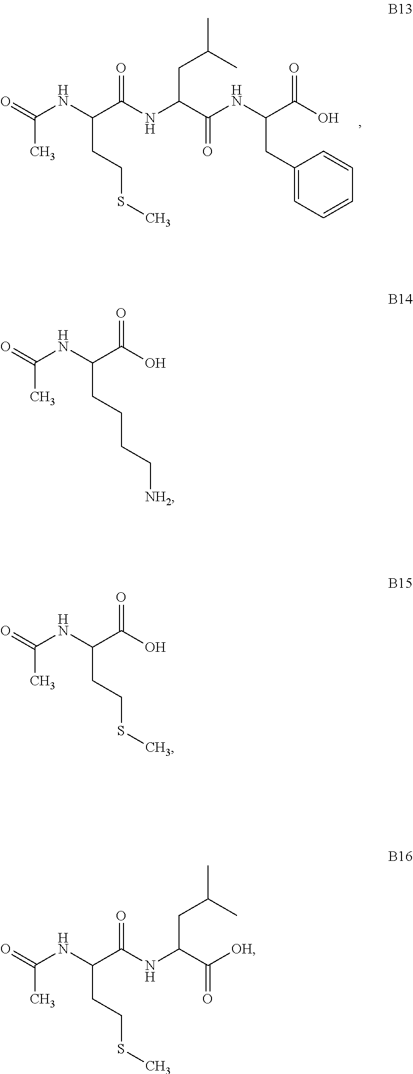 [SMILES: CSCCC(NC(C)=O)C(=O)NC(CC(C)C)C(=O)NC(Cc1ccccc1)C(=O)O ; CC(=O)NC(CCCCN)C(=O)O ; CSCCC(NC(C)=O)C(=O)O ; CSCCC(NC(C)=O)C(=O)NC(CC(C)C)C(=O)O]

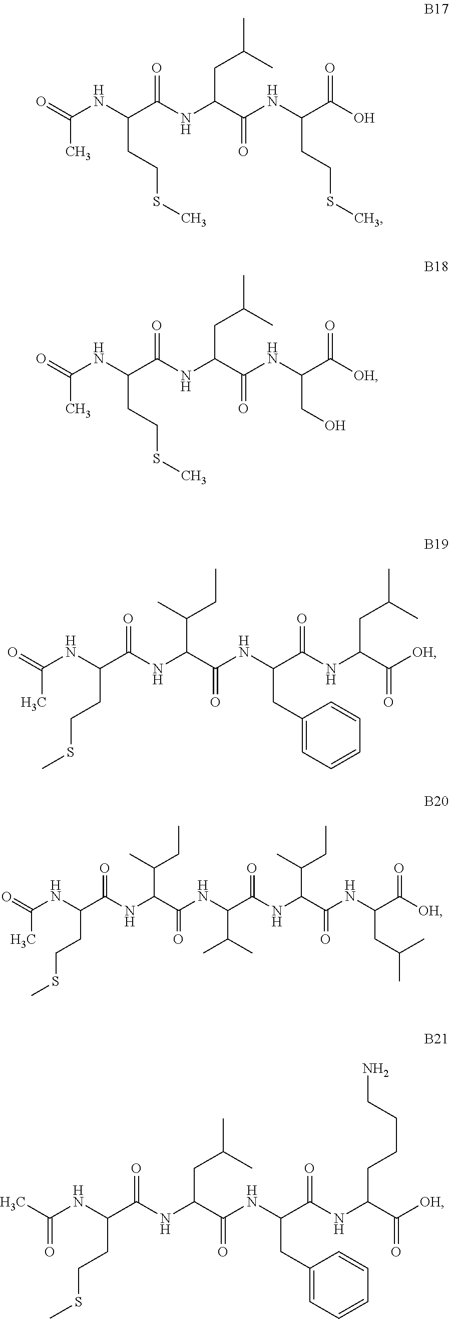

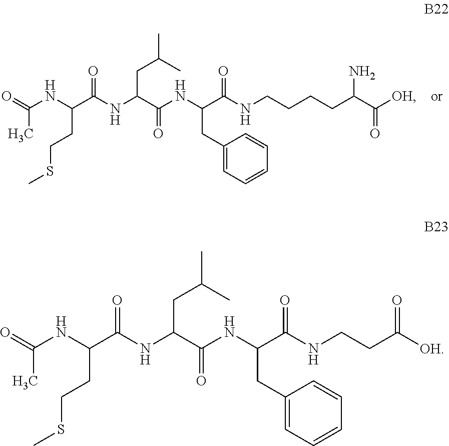 [SMILES: CSCCC(NC(C)=O)C(=O)NC(CC(C)C)C(=O)NC(Cc1ccccc1)C(=O)NCCCCC(N)C(=O)O ; "or" ; CSCCC(NC(C)=O)C(=O)NC(CC(C)C)C(=O)NC(Cc1ccccc1)C(=O)NCCC(=O)O]

In certain embodiments, the pathogen pattern recognition receptor ligand comprises, consists essentially of, or consists of a compound having the structure:

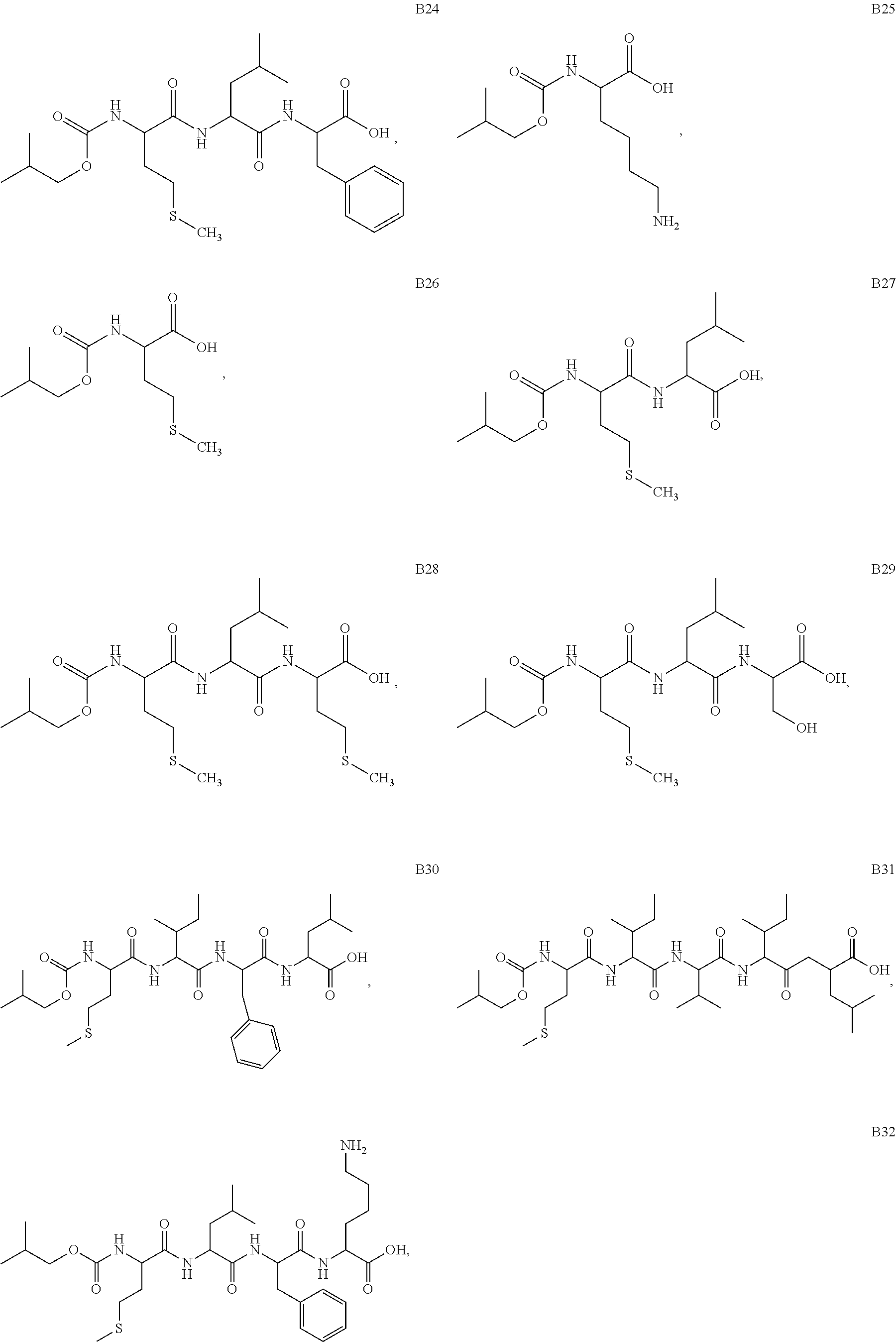

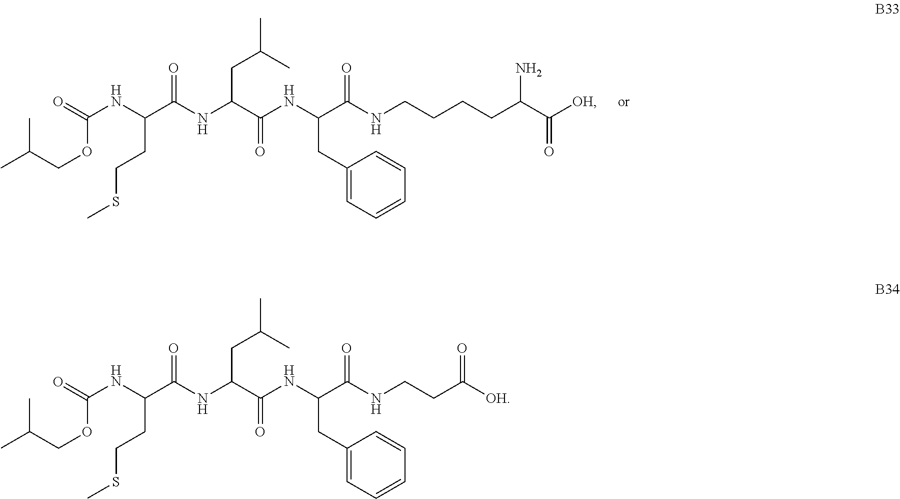 [SMILES: CSCCC(NC(=O)OCC(C)C)C(=O)NC(CC(C)C)C(=O)NC(Cc1ccccc1)C(=O)NCCCCC(N)C(=O)O ; "or" ; CSCCC(NC(=O)OCC(C)C)C(=O)NC(CC(C)C)C(=O)NC(Cc1ccccc1)C(=O)NCCC(=O)O]

Generally, the pathogen pattern recognition receptor ligand and inhibitor of β-1,3-glucan synthase are conjugated by a linker (i.e., a linking moiety), for example, by an amide bond at X9 of the pathogen pattern recognition receptor ligand and by an amide bond at an R¹¹ of the inhibitor of β-1,3-glucan synthase, e.g., at R⁴, R⁶, R⁷, R⁹, or R¹⁰ of any of Formulas I, II, XVII, and XVIII. For the avoidance of any doubt, when the pathogen pattern recognition receptor ligand and inhibitor of β-1,3-glucan synthase are conjugated by a bond, there is a single amide bond between the carbonyl carbon of X9 of the pathogen pattern recognition receptor ligand and the nitrogen of —NR¹¹R¹² of the inhibitor of β-1,3-glucan synthase.

The pathogen pattern recognition receptor ligand and inhibitor of β-1,3-glucan synthase can be conjugated by a linker that is a bond or that includes a non-reactive linking moiety of 1-100 atoms in length. The pathogen pattern recognition receptor ligand may be directly conjugated to the inhibitor of β-1,3-glucan synthase when the linker is a bond.

In some embodiments, the linker has the structure:

G¹-(Z¹)_{b}—(Y¹)_{c}—(Z²)_{d}—(R¹⁶)—(Z³)ₑ—(Y²)_{f}—(Z⁴)_{g}-G² Formula V

where G¹ is a bond between linker and inhibitor of β-1,3-glucan synthase; G² is a bond between pathogen pattern recognition receptor ligand and linker; each of Z¹, Z², Z³, and Z⁴ is independently selected from optionally substituted C₁-C₂ alkylene, optionally substituted C₁-C₃ heteroalkylene, 0, S, and NR¹⁷; R¹⁷ is hydrogen, optionally substituted C₁₋₄ alkyl, optionally substituted C₃₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; Y¹ and Y² are, independently, selected from carbonyl, thiocarbonyl, sulphonyl, and phosphoryl; b, c, d, e, f, and g are, independently, 0 or 1; and R¹⁶ is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₆₋₁₂ arylene, optionally substituted C₂-C₁₀₀ polyethylene glycolene, or optionally substituted C₁₋₁₀ heteroalkylene, or a bond linking G¹-(Z¹)_{b}—(Y¹)_{c}—(Z²)_{d}— to —(Z³)ₑ—(Y²)_{f}—(Z⁴)_{g}-G².

In some embodiments, the linker has the structure of Formula V, wherein Z⁴ is NH, g is 1, Y¹ is carbonyl, c is 1, and b is 0.

In other embodiments, the linker is selected from:

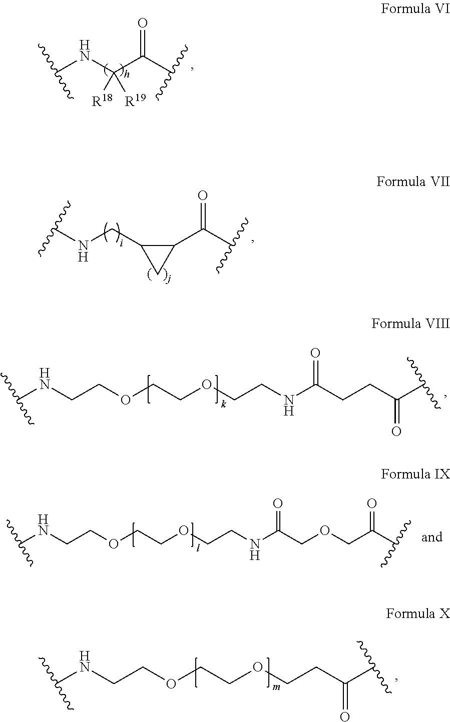

where h, i, j, k, l, and m are independently 1 to 12; R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocycyl, or R¹⁸ and R¹⁹ taken together form a 3 to 6-membered cycloalkyl or heterocycle, and where the NH group on the linker forms an amide bond with the carbonyl carbon of the terminal amino acid of the pathogen pattern recognition receptor and the carbonyl carbon of the linker forms an amide bond with an NR¹¹R¹² group of the inhibitor of β-1,3-glucan synthase (e.g., present in R⁴, R⁶, R⁷, R⁹, or R¹⁰ of Formulas I, II, XVII, or XVII).

In certain embodiments, the linker is selected from:

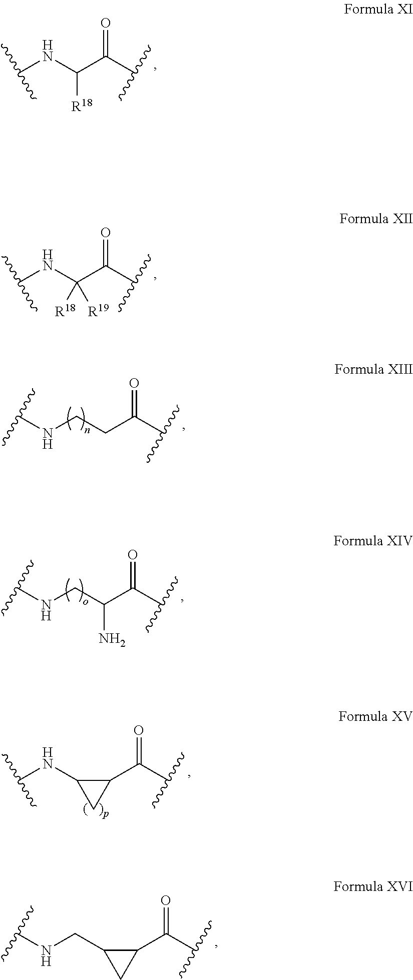

where n, o, and p are 1 to 4; and R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocyclic, or R¹⁸ and R¹⁹ taken together form a 3 to 6 membered cycloalkyl or heterocycle, and where the NH group on the linker forms an amide bond with the carbonyl carbon of the terminal amino acid of the pathogen pattern recognition receptor and the carbonyl carbon of the linker forms an amide bond with an NR¹¹R¹² group of the inhibitor of β-1,3-glucan synthase (e.g., present in R⁴, R⁶, R⁷, R⁹, or R¹⁰ of Formulas I, II, XVII, or XVIII).

In another aspect, the invention features a compound comprising, consisting essentially of, or consisting of:

(a) a moiety having the structure:

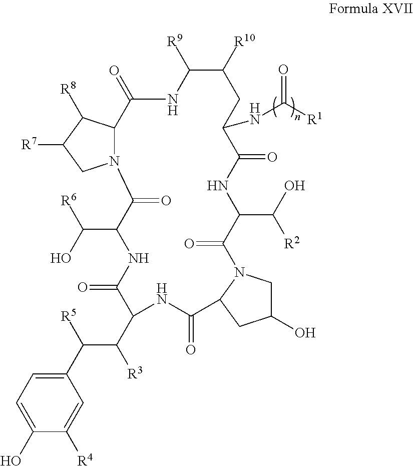

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; R¹¹, R¹², and R¹³ are independently hydrogen or C₁-C₃ alkyl; n is 0 or 1; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, R⁷, R⁹, or R¹⁰ contains —NR¹¹R¹², wherein at least one R¹¹ is a bond; conjugated (e.g., by an amide bond (i.e., R¹¹ of Formula I), for example, between the C-terminus of a peptide and a —NR¹¹R¹² of a compound of Formula I such as R⁴, R⁶, R⁷, R⁹, or R¹⁰, or by a linker) and

(b) a chemotactic peptide comprising an amino acid residue having the formula:

R¹⁴—X1-X2-X3-X4-X5-X6-X7-X8-X9- Formula III

where X9 is any amino acid; X1-X8 are any amino acid or absent; R¹⁴ is hydrogen or

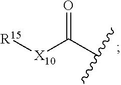

where X₁₀ is a bond, NH, or O; and R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic; where if R¹⁴ is hydrogen X1 is not absent (e.g., X1 and X2 are not absent); or a pharmaceutically acceptable salt thereof.

In some embodiments, (a) has the structure:

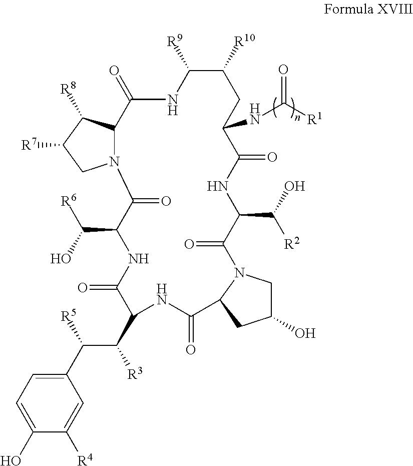

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, OR²⁰, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; R¹¹, R¹², and R¹³ are independently hydrogen or C₁-C₃ alkyl; n is 0 or 1; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, R⁷, R⁹, or R¹⁰ contains —NR¹¹R¹², wherein at least one R¹¹ is a bond conjugating (a) to (b) or to the linker.

In some embodiments, (a) has the structure:

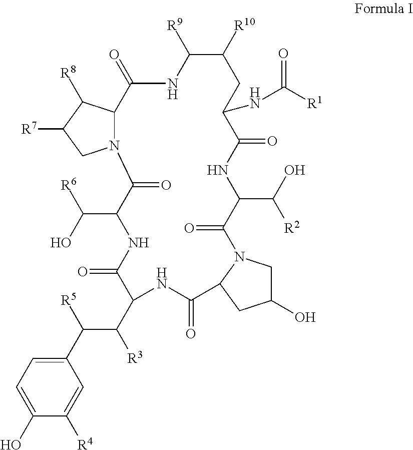

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, hydroxyl, —OSO₃H or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, hydrogen, or hydroxyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; R¹¹, R¹², and R¹³ are independently hydrogen or C₁-C₃ alkyl; a is 2 to 4; Q₁ is S, O or NH; b is 2-6 (preferably 2-4); c is 1-8 (preferably 1-3); Q₂ is NR¹¹R¹², N⁺R¹¹R¹²R¹³, OR²⁰; and R²⁰ is CH₃, CH₂CH₃, CH₂CH₂CH₃, CH(CH₃)₂, or H; where at least one of R⁴, R⁶, or R⁹ contains —NR¹¹R¹², wherein at least one R¹¹ is a bond conjugating (a) to (b) or to the linker.

In some embodiments, (a) has the structure:

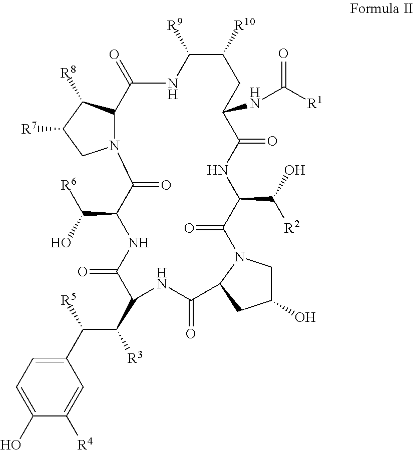

where R¹ is a C₁₀-C₃₆ moiety containing 0-8 heteroatoms selected from O, S, and N; R² is hydrogen or methyl; R³ and R⁵ are independently hydrogen or hydroxyl; R⁴ is hydrogen, hydroxyl, —OSO₃H or —NR¹¹R¹²; R⁶ is hydrogen, methyl, —CH₂(C═O)NR¹¹R¹², —CH₂CH₂NR¹¹R¹², or —CH₂CH₂N⁺R¹¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, hydrogen, or hydroxyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; R¹¹, R¹², and R¹³ are independently hydrogen or C₁-C₃ alkyl; a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, or, R⁹ contains —NR¹¹R¹², wherein at least one R¹¹ is a bond conjugating (a) to (b) or to the linker.

In other embodiments, (a) has the structure of Formula I, II, XVII, or XVIII, where R¹ is optionally substituted C₁₃-C₁₇ alkyl, optionally substituted C₁₃-C₁₇ alkenyl, optionally substituted aryl, or optionally substituted heteroaryl; R² is methyl; R³ and R⁵ are hydroxyl; R⁴ is hydrogen, hydroxyl, —OSO₃H or —NR¹¹R¹², preferably hydrogen; R⁶ is methyl, —CH₂(C═O)NR¹¹R¹², —CH₂CH₂NR¹¹R¹², or —CH₂CH₂N⁺R¹¹R¹²R¹³, preferably —CH₂CH₂NR¹¹R¹²; R⁷ is hydrogen, methyl, —NR¹¹R¹², or —NH(C═NH)NR¹¹R¹²; R⁸ is hydroxyl; R⁹ is hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³, preferably —O(CH₂)ₐNR¹¹R¹² or —NH(CH₂)ₐNR¹¹R¹²; R¹¹, R¹², and R¹³ are independently hydrogen or methyl; R¹⁰ is hydrogen, hydroxyl, oxo, —NR¹¹R¹², NH(CH₂)ₐNR¹¹R¹², or —NH(CH₂)ₐN⁺R¹¹R¹²R¹³; n is 0 or 1; a is 2 to 4; X₁ is O or NH; b is 2-6 (preferably 2-4); c is 1-8 (preferably 1-3); X₂ is NR¹¹R¹², N⁺R¹¹R¹²R¹³, or OR²⁰; and R²⁰ is CH₃, CH₂CH₃, CH₂CH₂CH₃, CH(CH₃)₂, or H; where at least one of R⁴, R⁶, R⁷, R⁹, or R¹⁰ contains an —NR¹¹R¹², wherein at least one R¹¹ is a bond conjugating (a) to (b) or to the linker.

In some embodiments, R⁴ of Formula I, II, XVII, or XVIII contains —NR¹¹R¹², wherein R¹¹ is a bond joining the inhibitor of β-1,3-glucan synthase to the pathogen pattern recognition receptor ligand or to the linker.

In some embodiments, R⁶ of Formula I, II, XVII, or XVIII contains —NR¹¹R¹², wherein R¹¹ is a bond joining the inhibitor of β-1,3-glucan synthase to the pathogen pattern recognition receptor ligand or to the linker.

In some embodiments, R⁷ of Formula I, II, XVII, or XVIII contains —NR¹¹R¹², wherein R¹¹ is a bond joining the inhibitor of β-1,3-glucan synthase to the pathogen pattern recognition receptor ligand or to the linker.

In some embodiments, R⁹ of Formula I, II, XVII, or XVIII contains —NR¹¹R¹², wherein R¹¹ is a bond joining the inhibitor of β-1,3-glucan synthase to the pathogen pattern recognition receptor ligand or to the linker.

In some embodiments, R¹⁰ of Formula I, II, XVII, or XVIII contains —NR¹¹R¹², wherein R¹¹ is a bond joining the inhibitor of β-1,3-glucan synthase to the pathogen pattern recognition receptor ligand or to the linker.

In some embodiments, R¹ of Formula I, II, XVII, or XVIII contains a C₁-C₁₇ alkyl or heteroalkyl, C₂-C₁₇ alkenyl or heteroalkenyl, aryl or heteroaryl, cyclic, polycyclic, heterocyclic or heteropolycyclic moiety, or a combination thereof to form a C₁₀-C₃₆ moiety.

In some embodiments, R¹ of Formula I, II, XVII, or XVIII contains optionally substituted C₁₃-C₁₇ alkyl, optionally substituted C₁₃-C₁₇ alkenyl, optionally substituted aryl, or optionally substituted heteroaryl.

In some embodiments, R⁹ of Formula I, II, XVII, or XVIII contains hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐNR¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³.

In some embodiments, R⁹ of Formula I, II, XVII, or XVIII contains hydrogen, hydroxyl, —NH₂, —O(CH₂)₂NH₂, —O(CH₂)₂N⁺(CH₃)₃, —O(CH₂)₂O(CH₂)₂NH₂, —NH(CH₂)₂NH₂, —NH(CH₂)₂N+(CH₃)₃, —S(CH₂)₂NH₂, or —CH₂NH₂.

In some embodiments, R¹ of Formula I, II, XVII, or XVIII contains a C₁₀-C₃₆ moiety containing 0-8 heteroatoms selected from O, S, and N.

In some embodiments, R¹ of Formula I, II, XVII, or XVIII is:

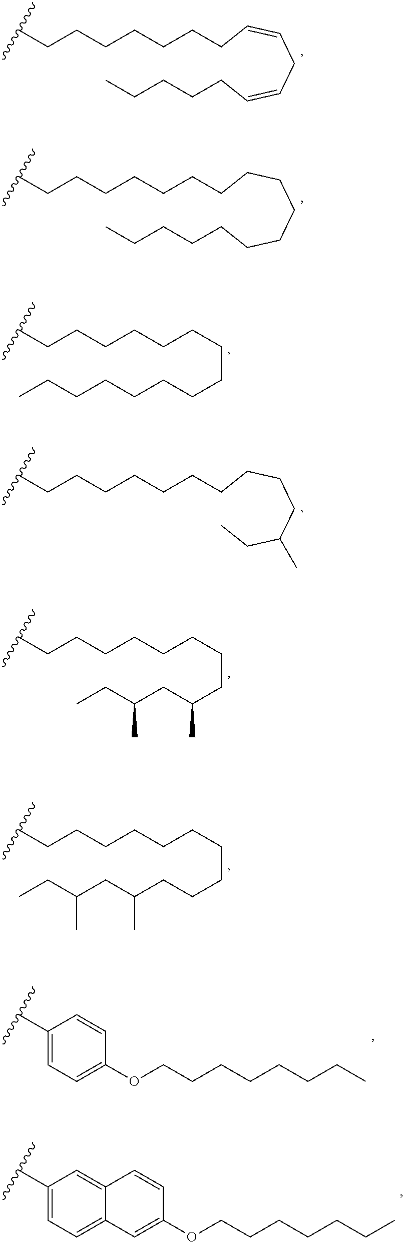

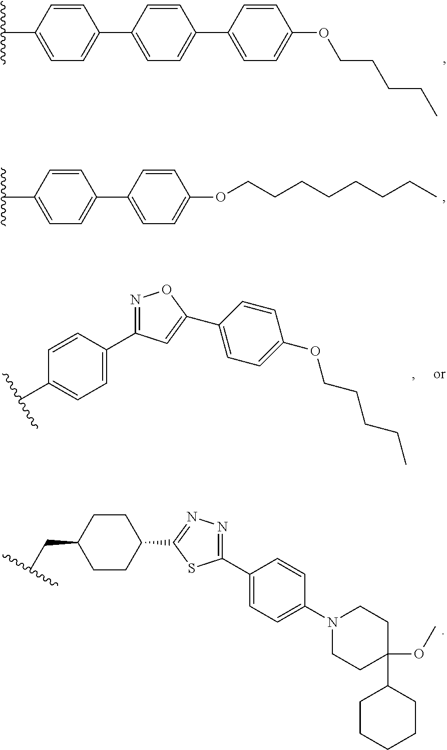 [SMILES: *c1ccc(-c2ccc(-c3ccc(OCCCCC)cc3)cc2)cc1 ; *c1ccc(-c2ccc(OCCCCCCCC)cc2)cc1 ; *c1ccc(-c2cc(-c3ccc(OCCCCC)cc3)on2)cc1 ; *C[C@H]1CC[C@H](c2nnc(-c3ccc(N4CCC(OC)(C5CCCCC5)CC4)cc3)s2)CC1]

In certain embodiments, (a) has the structure:

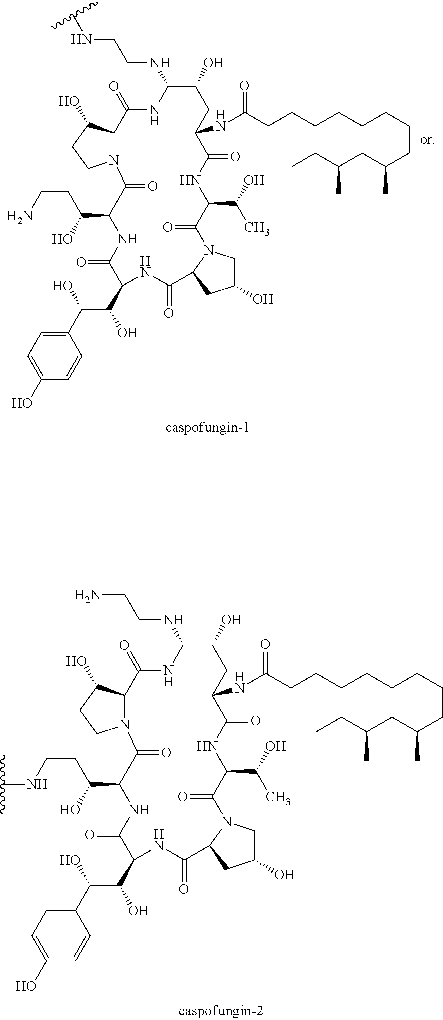 [SMILES: *NCCN[C@H]1NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@H]1O ; *NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O]

In other embodiments, (a) has the structure:

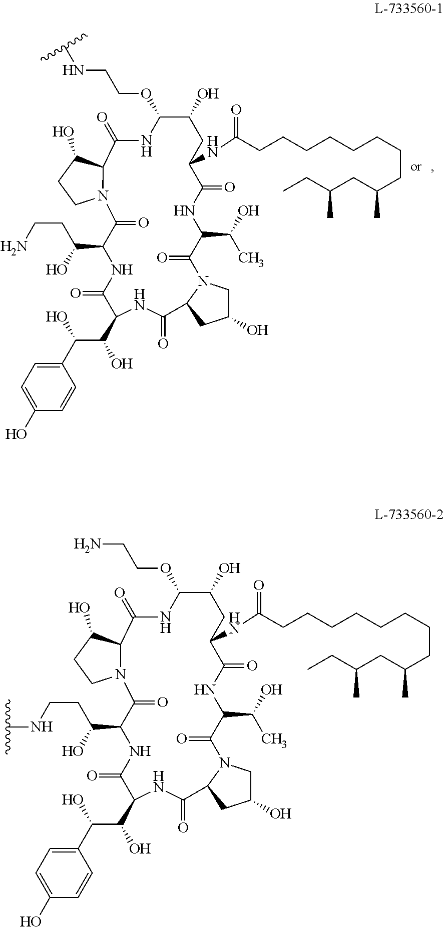

In further embodiments, (a) has the structure:

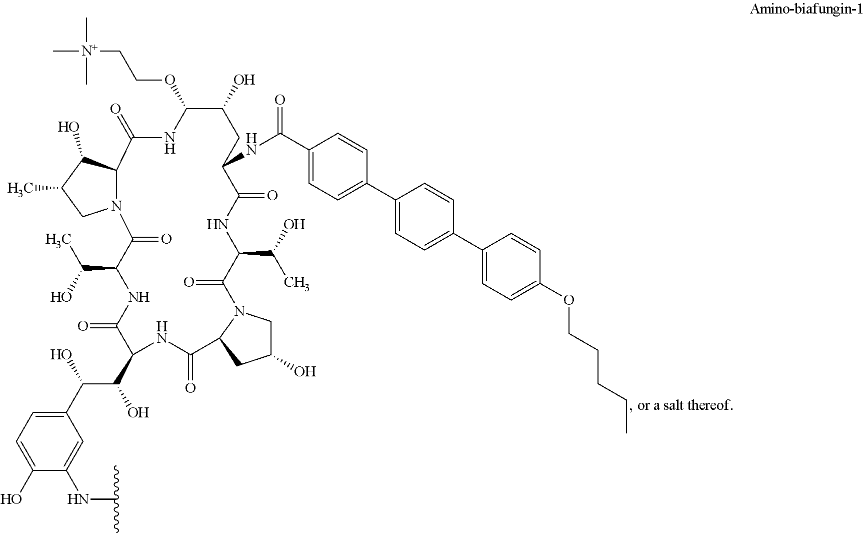 [SMILES: *Nc1cc([C@H](O)[C@@H](O)[C@@H]2NC(=O)[C@@H]3C[C@@H](O)CN3C(=O)[C@H]([C@@H](C)O)NC(=O)[C@@H](NC(=O)c3ccc(-c4ccc(-c5ccc(OCCCCC)cc5)cc4)cc3)C[C@@H](O)[C@@H](OCC[N+](C)(C)C)NC(=O)[C@@H]3[C@@H](O)[C@@H](C)CN3C(=O)[C@H]([C@@H](C)O)NC2=O)ccc1O]

In further embodiments, the inhibitor of β-1,3-glucan synthase can comprise, consist essentially of, or consist of a compound having the structure:

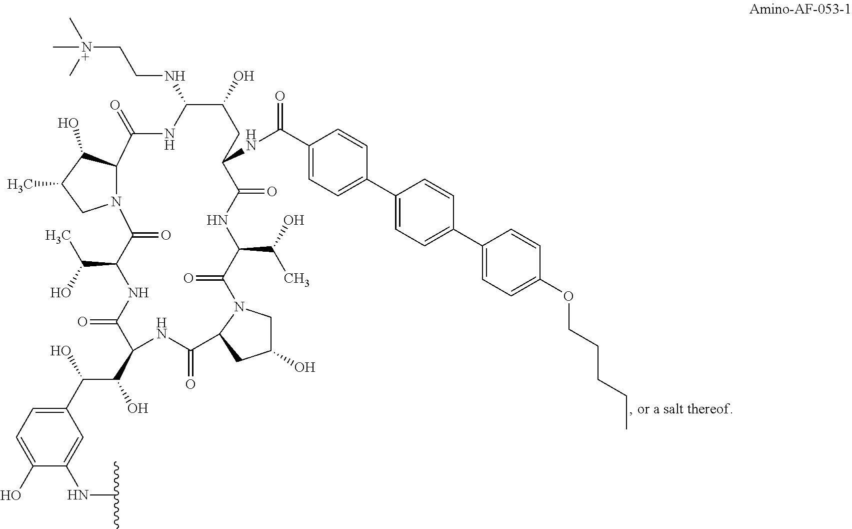 [SMILES: *Nc1cc([C@H](O)[C@@H](O)[C@@H]2NC(=O)[C@@H]3C[C@@H](O)CN3C(=O)[C@H]([C@@H](C)O)NC(=O)[C@@H](NC(=O)c3ccc(-c4ccc(-c5ccc(OCCCCC)cc5)cc4)cc3)C[C@@H](O)[C@@H](NCC[N+](C)(C)C)NC(=O)[C@@H]3[C@@H](O)[C@@H](C)CN3C(=O)[C@H]([C@@H](C)O)NC2=O)ccc1O]

In further embodiments, the inhibitor of β-1,3-glucan synthase can comprise, consist essentially of, or consist of a compound having the structure:

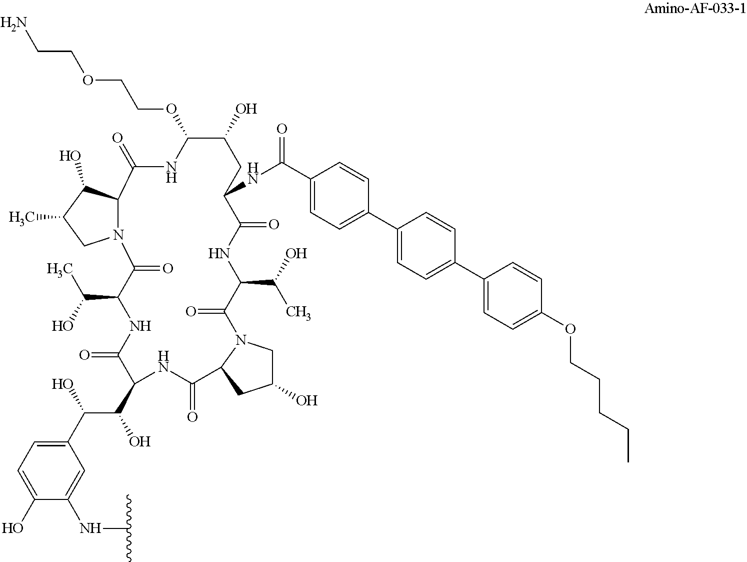 [SMILES: *Nc1cc([C@H](O)[C@@H](O)[C@@H]2NC(=O)[C@@H]3C[C@@H](O)CN3C(=O)[C@H]([C@@H](C)O)NC(=O)[C@@H](NC(=O)c3ccc(-c4ccc(-c5ccc(OCCCCC)cc5)cc4)cc3)C[C@@H](O)[C@@H](OCCOCCN)NC(=O)[C@@H]3[C@@H](O)[C@@H](C)CN3C(=O)[C@H]([C@@H](C)O)NC2=O)ccc1O]

In some embodiments, (b) includes an amino acid residue having the formula:

R¹⁴—X1-X2-X9- Formula IV

where X1 is any amino acid; X2 is leucine or isoleucine, or is absent; X9 is any amino acid; R¹⁴ is hydrogen or

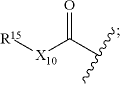

where X₁₀ is a bond, NH, or O; and R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic.

Amino acids that can be included in the chemotactic peptide (b) can be selected from naturally occurring amino acids (e.g., Ala, Arg, Asn, Asp, Cys, Gin, Glu, Gly, His, Ile, Leu, Lys, Met, Phe, Pro, Ser, Thr, Trp, Tyr, or Val), or non-naturally occurring amino acids, as are described above.

In certain embodiments, (b) has the structure:

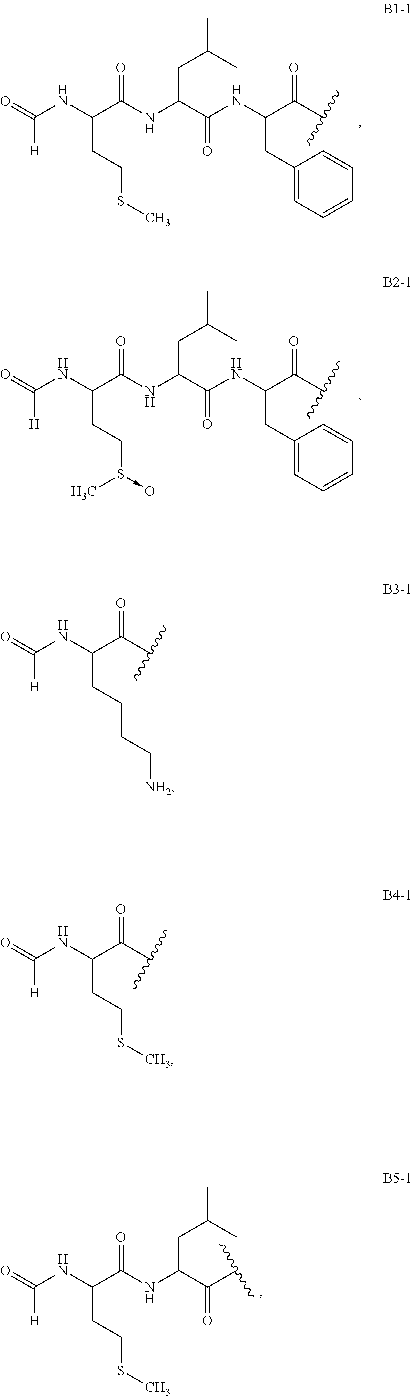 [SMILES: *C(=O)C(Cc1ccccc1)NC(=O)C(CC(C)C)NC(=O)C(CCSC)NC=O ; *C(=O)C(Cc1ccccc1)NC(=O)C(CC(C)C)NC(=O)C(CCS(C)=O)NC=O ; *C(=O)C(CCCCN)NC=O ; *C(=O)C(CCSC)NC=O ; *C(=O)C(CC(C)C)NC(=O)C(CCSC)NC=O]

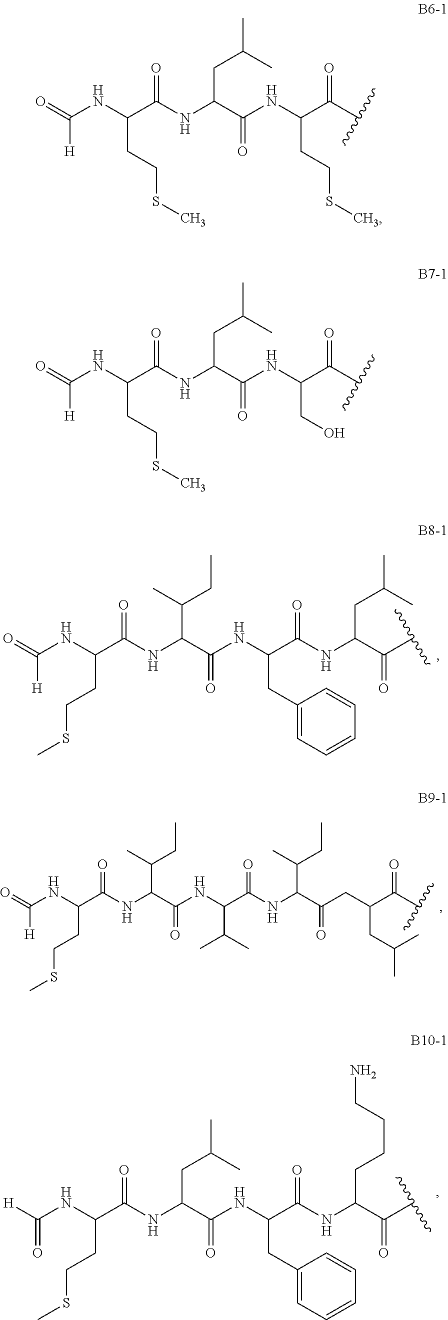 [SMILES: *C(=O)C(CCSC)NC(=O)C(CC(C)C)NC(=O)C(CCSC)NC=O ; *C(=O)C(CO)NC(=O)C(CC(C)C)NC(=O)C(CCSC)NC=O ; *C(=O)C(CC(C)C)NC(=O)C(Cc1ccccc1)NC(=O)C(NC(=O)C(CCSC)NC=O)C(C)CC ; *C(=O)C(CC(=O)C(NC(=O)C(NC(=O)C(NC(=O)C(CCSC)NC=O)C(C)CC)C(C)C)C(C)CC)CC(C)C ; *C(=O)C(CCCCN)NC(=O)C(Cc1ccccc1)NC(=O)C(CC(C)C)NC(=O)C(CCSC)NC=O]

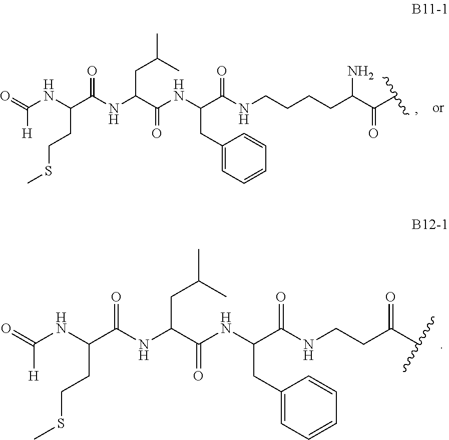 [SMILES: *C(=O)C(N)CCCCNC(=O)C(Cc1ccccc1)NC(=O)C(CC(C)C)NC(=O)C(CCSC)NC=O ; "or" ; *C(=O)CCNC(=O)C(Cc1ccccc1)NC(=O)C(CC(C)C)NC(=O)C(CCSC)NC=O]

In other embodiments, (b) has the structure:

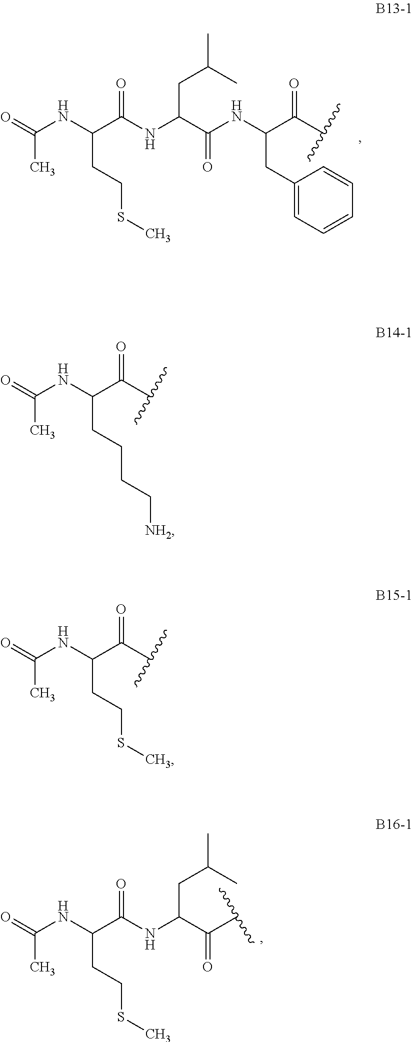 [SMILES: *C(=O)C(Cc1ccccc1)NC(=O)C(CC(C)C)NC(=O)C(CCSC)NC(C)=O ; *C(=O)C(CCCCN)NC(C)=O ; *C(=O)C(CCSC)NC(C)=O ; *C(=O)C(CC(C)C)NC(=O)C(CCSC)NC(C)=O]

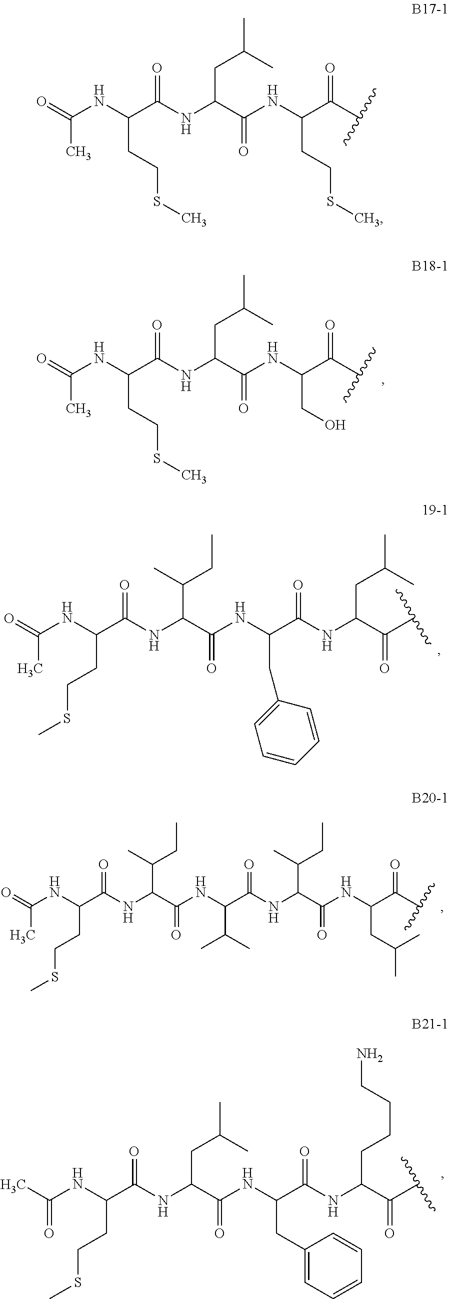 [SMILES: *C(=O)C(CCSC)NC(=O)C(CC(C)C)NC(=O)C(CCSC)NC(C)=O ; *C(=O)C(CO)NC(=O)C(CC(C)C)NC(=O)C(CCSC)NC(C)=O ; *C(=O)C(CC(C)C)NC(=O)C(Cc1ccccc1)NC(=O)C(NC(=O)C(CCSC)NC(C)=O)C(C)CC ; *C(=O)C(CC(C)C)NC(=O)C(NC(=O)C(NC(=O)C(NC(=O)C(CCSC)NC(C)=O)C(C)CC)C(C)C)C(C)CC ; *C(=O)C(CCCCN)NC(=O)C(Cc1ccccc1)NC(=O)C(CC(C)C)NC(=O)C(CCSC)NC(C)=O]

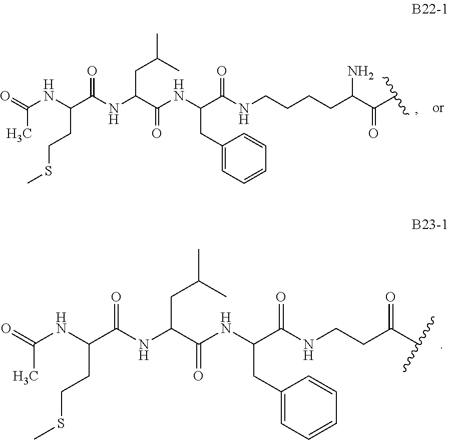 [SMILES: *C(=O)C(N)CCCCNC(=O)C(Cc1ccccc1)NC(=O)C(CC(C)C)NC(=O)C(CCSC)NC(C)=O ; "or" ; *C(=O)CCNC(=O)C(Cc1ccccc1)NC(=O)C(CC(C)C)NC(=O)C(CCSC)NC(C)=O]

In still other embodiments, (b) has the structure:

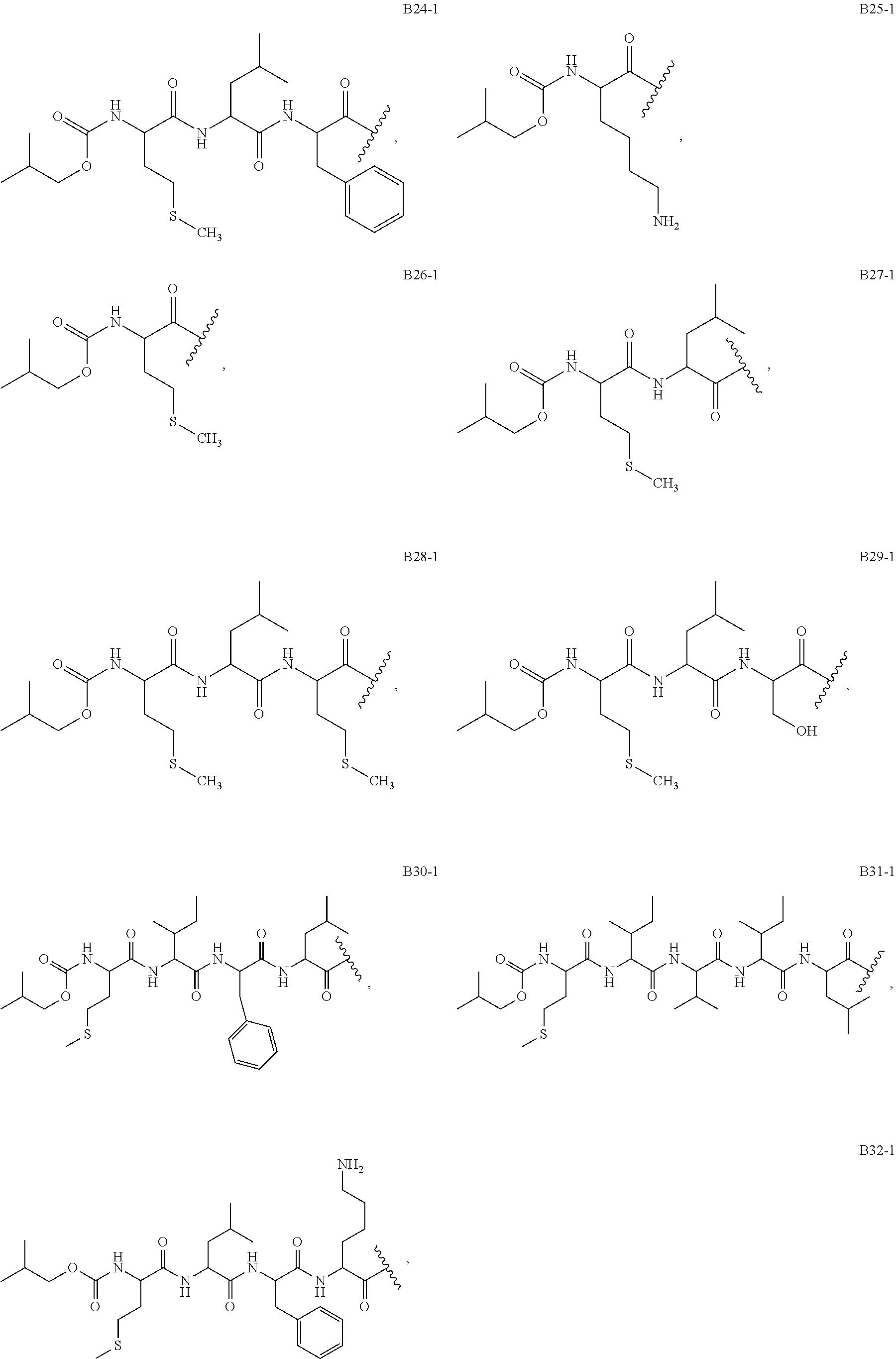

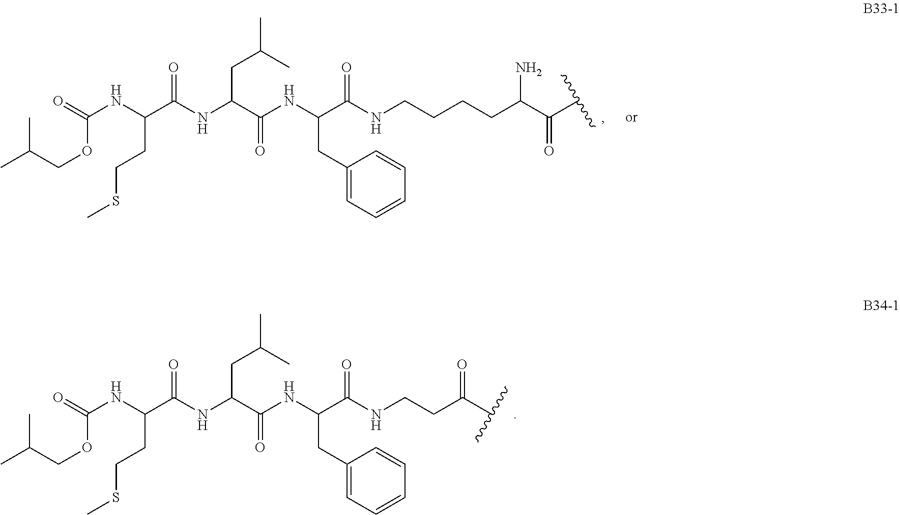 [SMILES: *C(=O)C(N)CCCCNC(=O)C(Cc1ccccc1)NC(=O)C(CC(C)C)NC(=O)C(CCSC)NC(=O)OCC(C)C ; "or" ; *C(=O)CCNC(=O)C(Cc1ccccc1)NC(=O)C(CC(C)C)NC(=O)C(CCSC)NC(=O)OCC(C)C]

Generally, the pathogen pattern recognition receptor ligand and inhibitor of β-1,3-glucan synthase are conjugated by a linker (i.e., a linking moiety), for example, by an amide bond at X9 of the pathogen pattern recognition receptor and by an amide bond at an R¹¹ of the inhibitor of β-1,3-glucan synthase, e.g., at R⁴, R⁶, R⁷, R⁹, or R¹⁰ of any of Formulas I, II, XVII, and XVIII. For the avoidance of any doubt, when the pathogen pattern recognition receptor ligand and inhibitor of β-1,3-glucan synthase are conjugated by a bond, there is a single amide bond between the carbonyl carbon of X9 of the pathogen pattern recognition receptor ligand and the nitrogen of —NR¹¹R¹² of the inhibitor of β-1,3-glucan synthase.

The pathogen pattern recognition receptor ligand and inhibitor of β-1,3-glucan synthase can be conjugated by a linker that is a bond or that includes a non-reactive linking moiety of 1-100 atoms in length.

In some embodiments, the linker has the structure:

G¹-(Z¹)_{b}—(Y¹)_{c}—(Z²)_{d}—(R¹⁶)—(Z³)ₑ—(Y²)_{f}—(Z⁴)⁹-G² Formula V

where G¹ is a bond between linker and inhibitor of β-1,3-glucan synthase; G² is a bond between pathogen pattern recognition receptor ligand and linker; Z¹, Z², Z³, and Z⁴ each, is independently, selected from optionally substituted C₁-C₂ alkylene, optionally substituted C₁-C₃ heteroalkylene, O, S, and NR¹⁷; R¹⁷ is hydrogen, optionally substituted C₁₋₄ alkyl, optionally substituted C₃₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; Y¹ and Y² are, independently, selected from carbonyl, thiocarbonyl, sulphonyl, and phosphoryl; b, c, d, e, f, and g are, independently, 0 or 1; and R¹⁶ is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₆₋₁₂ arylene, optionally substituted C₂-C₁₀₀ polyethylene glycolene, or optionally substituted C₁₋₁₀ heteroalkylene, or a chemical bond linking G¹-(Z¹)_{b}—(Y¹)_{c}—(Z²)_{d}— to —(Z³)ₑ—(Y²)_{f}—(Z⁴)_{g}-G².

In some embodiments, the linker has the structure of Formula V, wherein Z⁴ is NH, g is 1, Y¹ is carbonyl, c is 1, and b is 0.

In other embodiments, the linker is selected from:

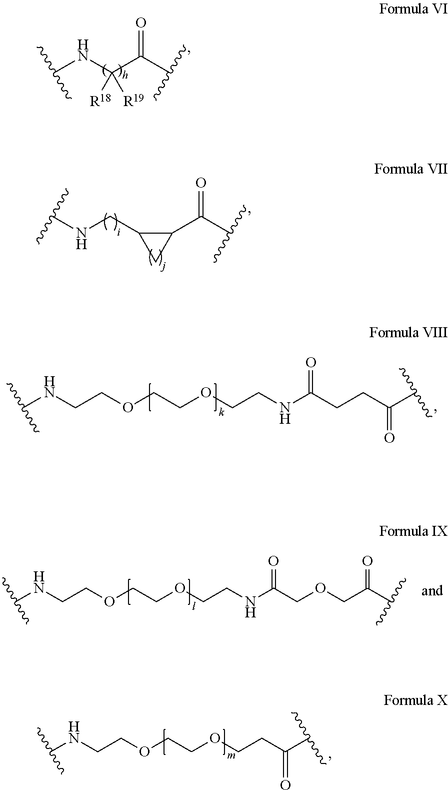

where h, i, j, k, l, and m are independently 1 to 12; R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocycyl, or R¹⁸ and R¹⁹ taken together form a 3 to 6-membered cycloalkyl or heterocycle, and where the NH group on the linker forms an amide bond with the carbonyl carbon of the terminal amino acid of the pathogen pattern recognition receptor and the carbonyl carbon of the linker forms an amide bond with an NR¹¹R¹² group of the inhibitor of β-1,3-glucan synthase (e.g., present in R⁴, R⁶, R⁷, R⁹, or R¹⁰ of Formulas I, II, XVII, and XVIII).

In certain embodiments, the linker is selected from:

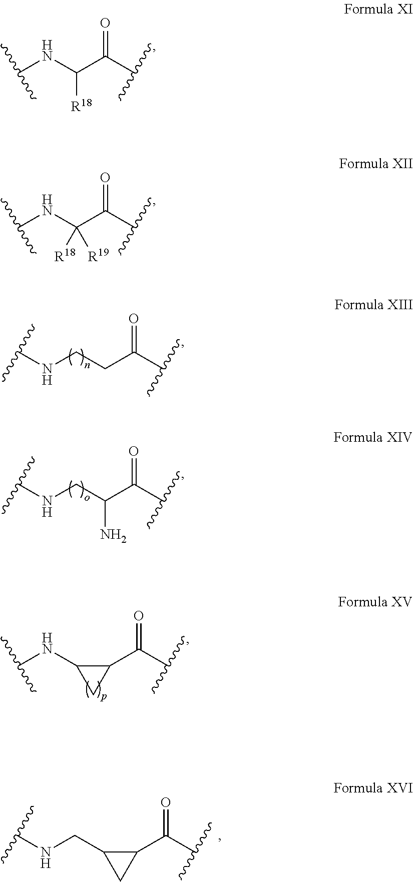

where n, o, and p are 1 to 4; and R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocyclic, or R¹⁸ and R¹⁹ taken together form a 3 to 6 membered cycloalkyl or heterocycle, and where the NH group on the linker forms an amide bond with the carbonyl carbon of the terminal amino acid of the pathogen pattern recognition receptor and the carbonyl carbon of the linker forms an amide bond with an NR¹¹R¹² group of the inhibitor of β-1,3-glucan synthase (e.g., present in R⁴, R⁶, R⁷, R⁹, or R¹⁰ of Formulas I, II, XVII, or XVIII).

In certain embodiments, (a) is attached to the C-terminus of the linker via an amide bond (e.g., at a —NR¹¹R¹² contained in R⁴, R⁶, R⁷, R⁹, or R¹⁰ of Formula I, II, XVII, or XVIII, wherein R¹¹ is the amide bond) and (b) is attached to the N-terminus of the linker via an amide bond (e.g., at the C-terminus of (b)).

In some embodiments, the compound is selected from:

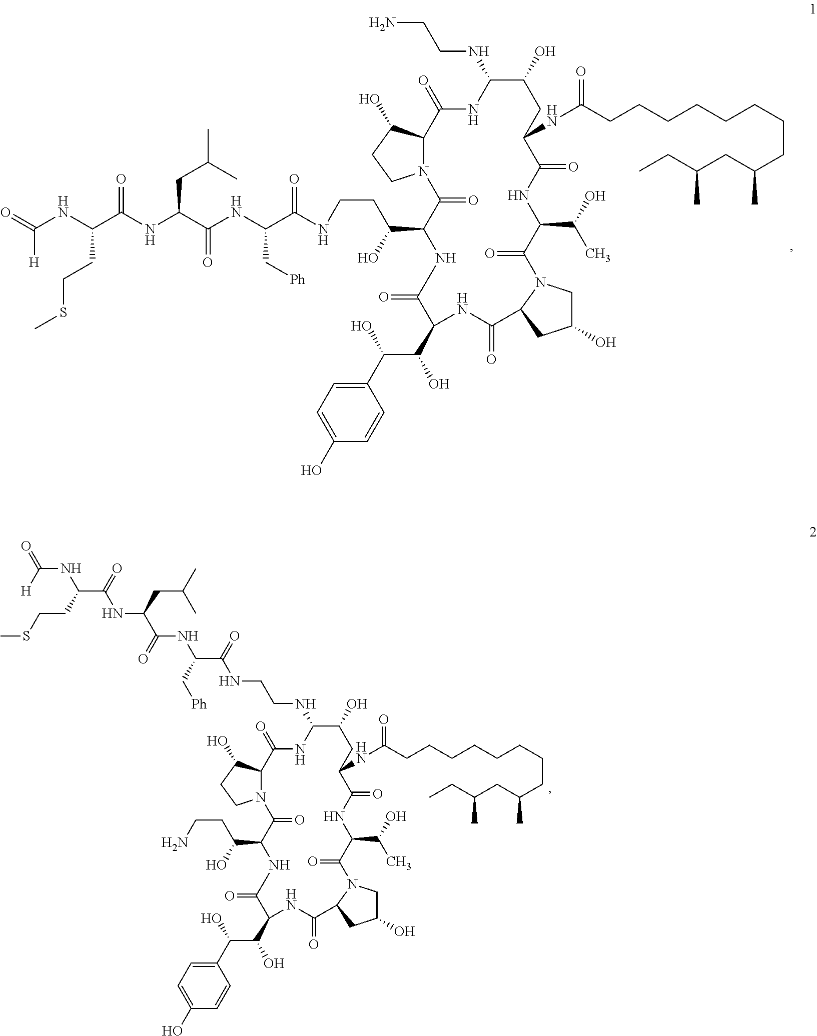

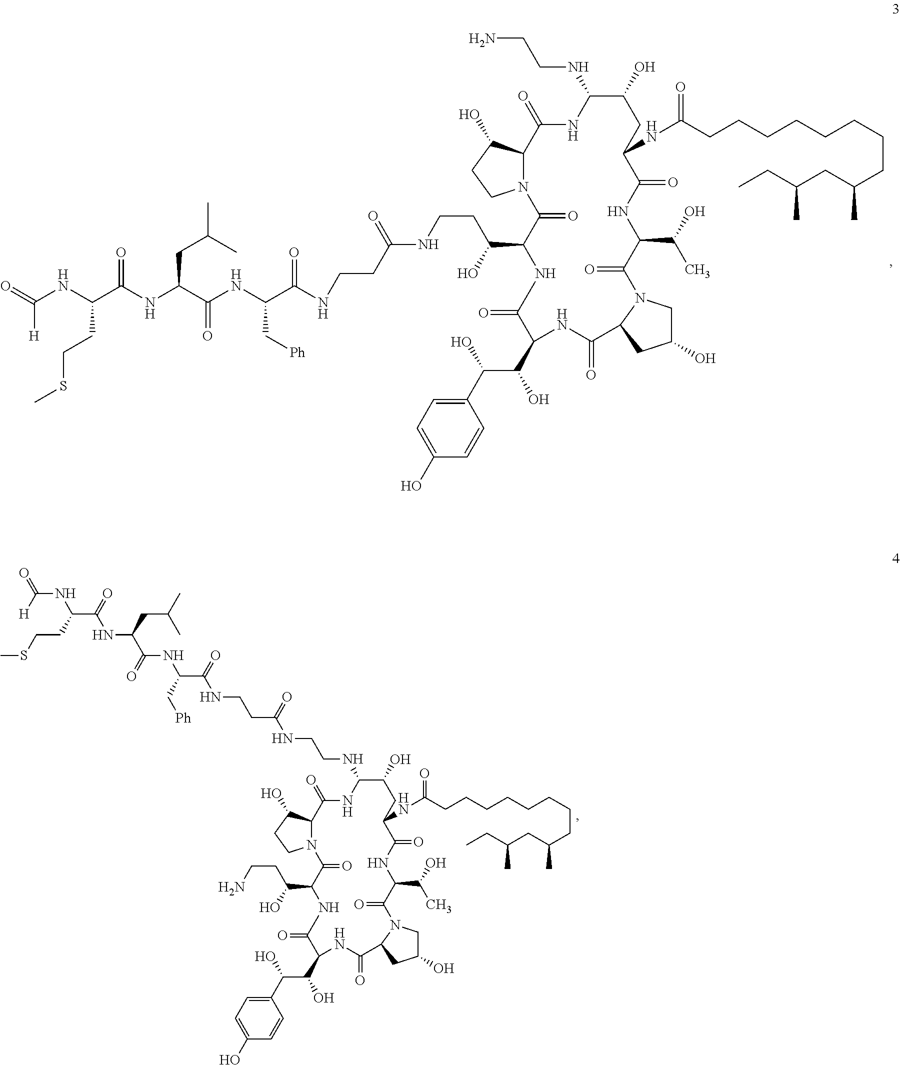

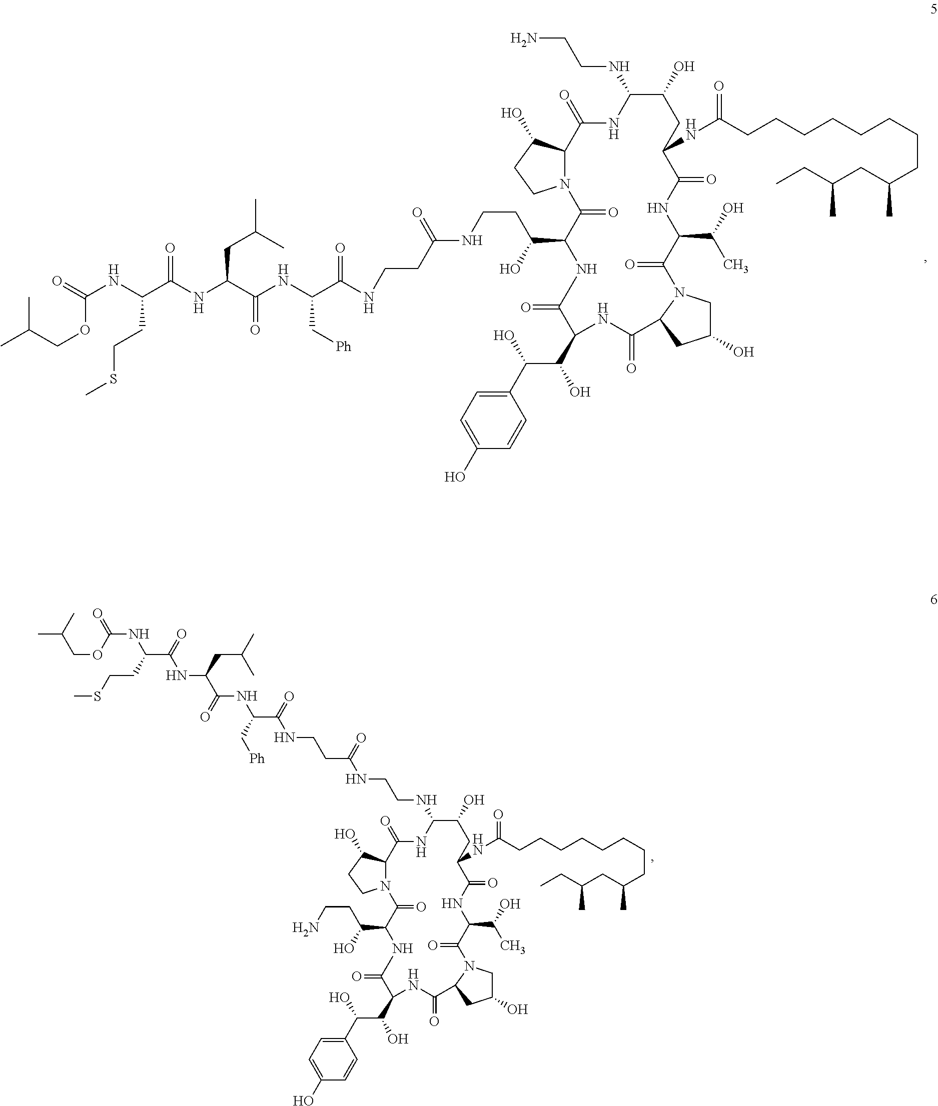

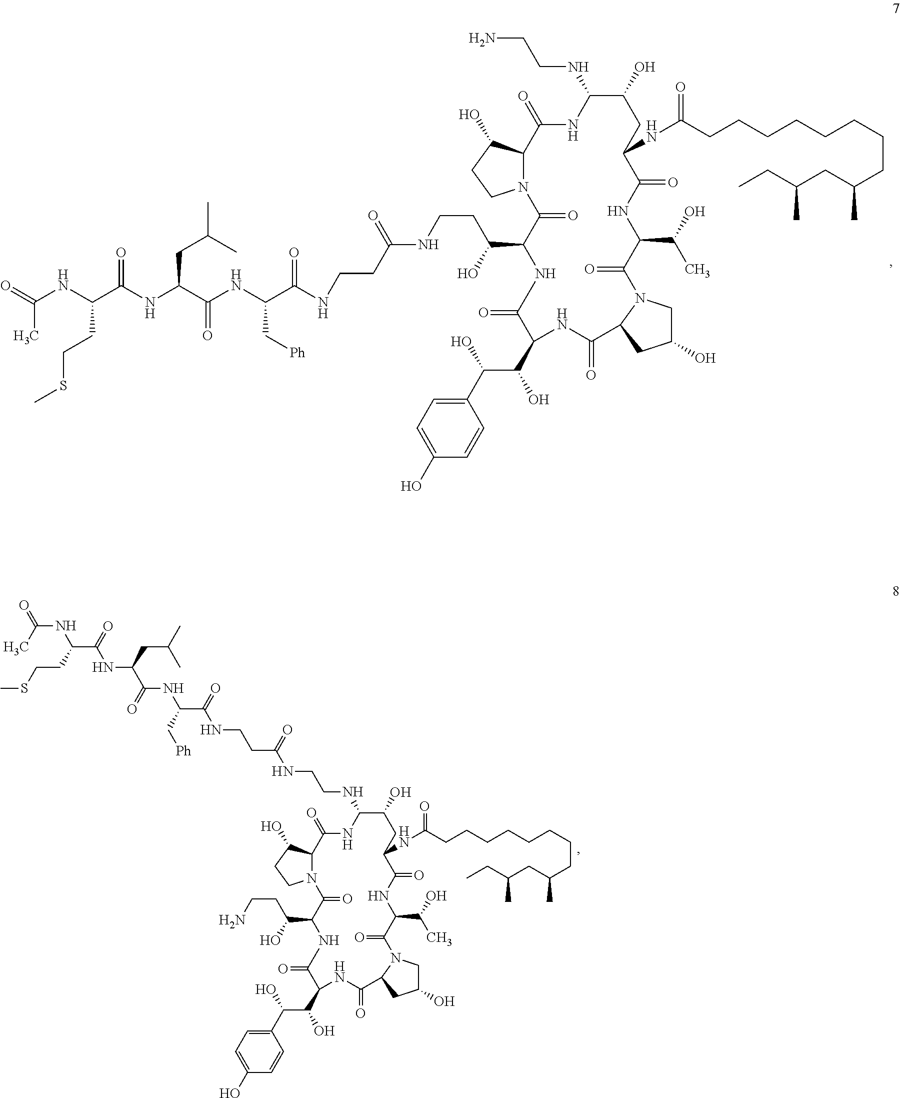

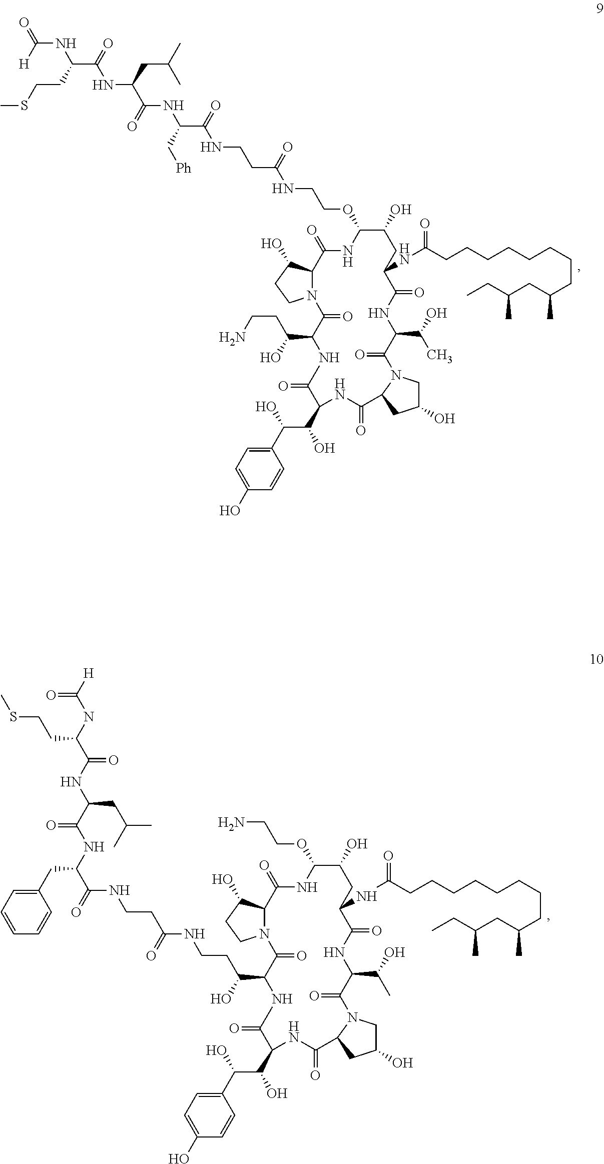

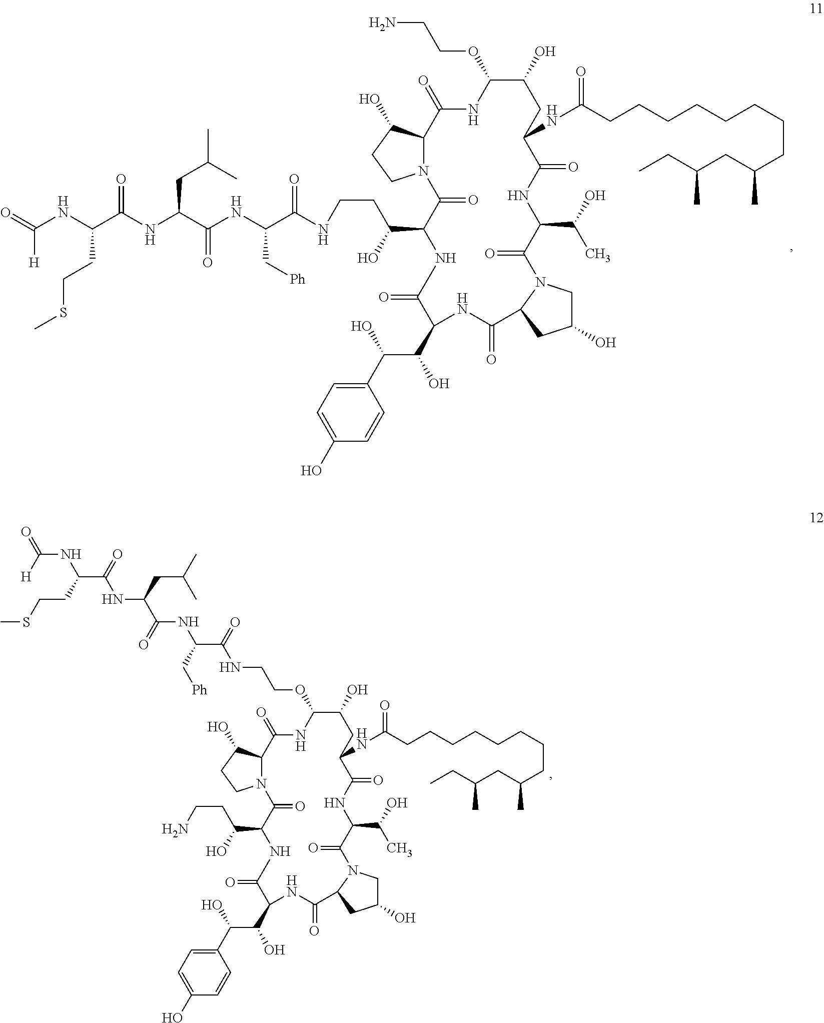

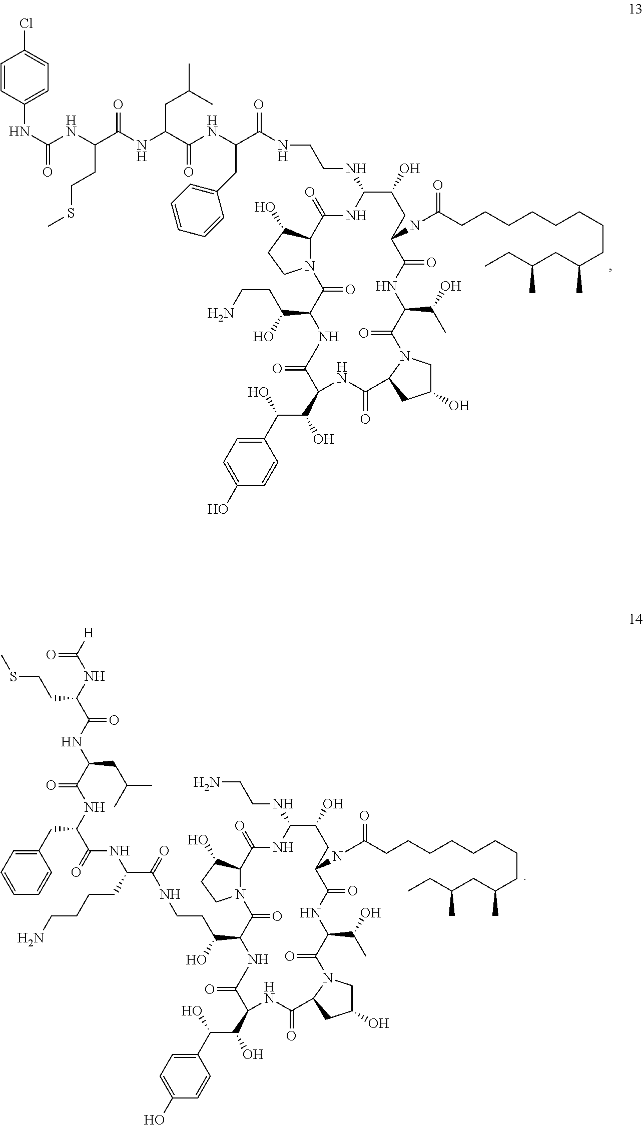

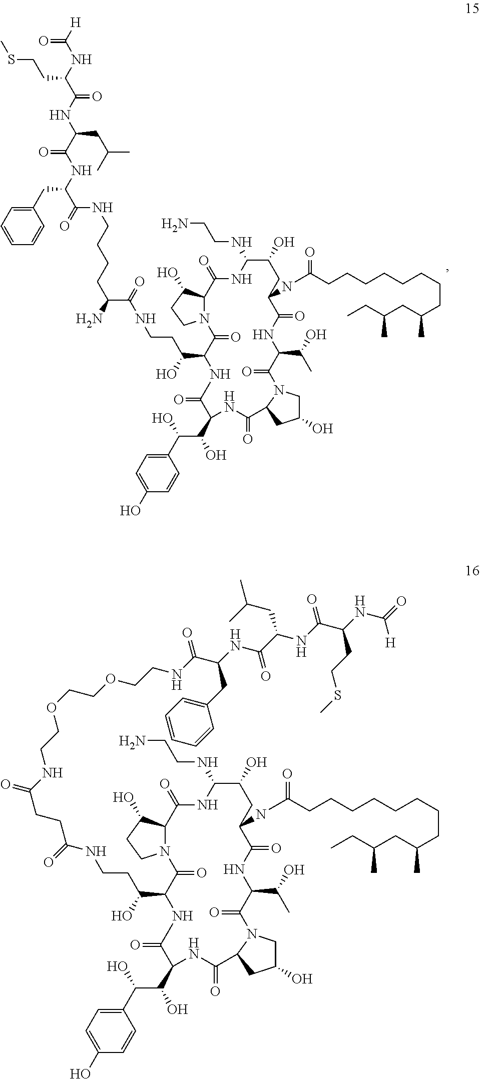

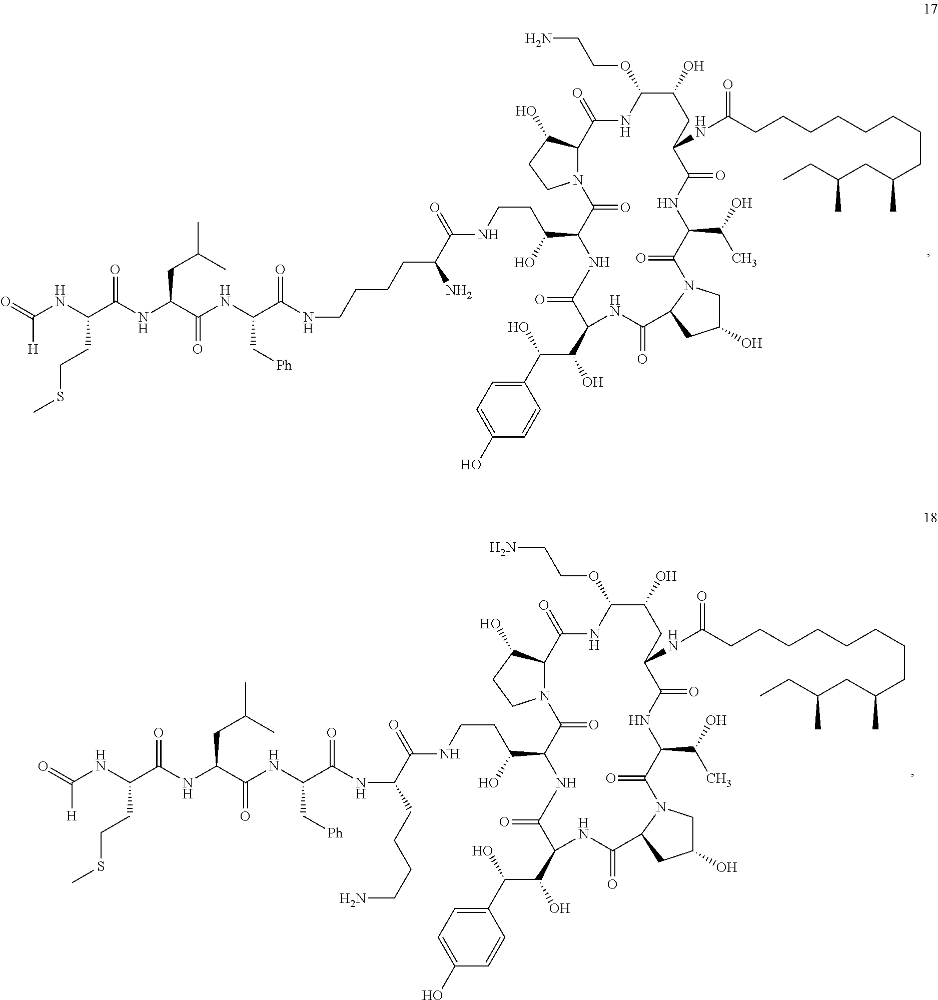

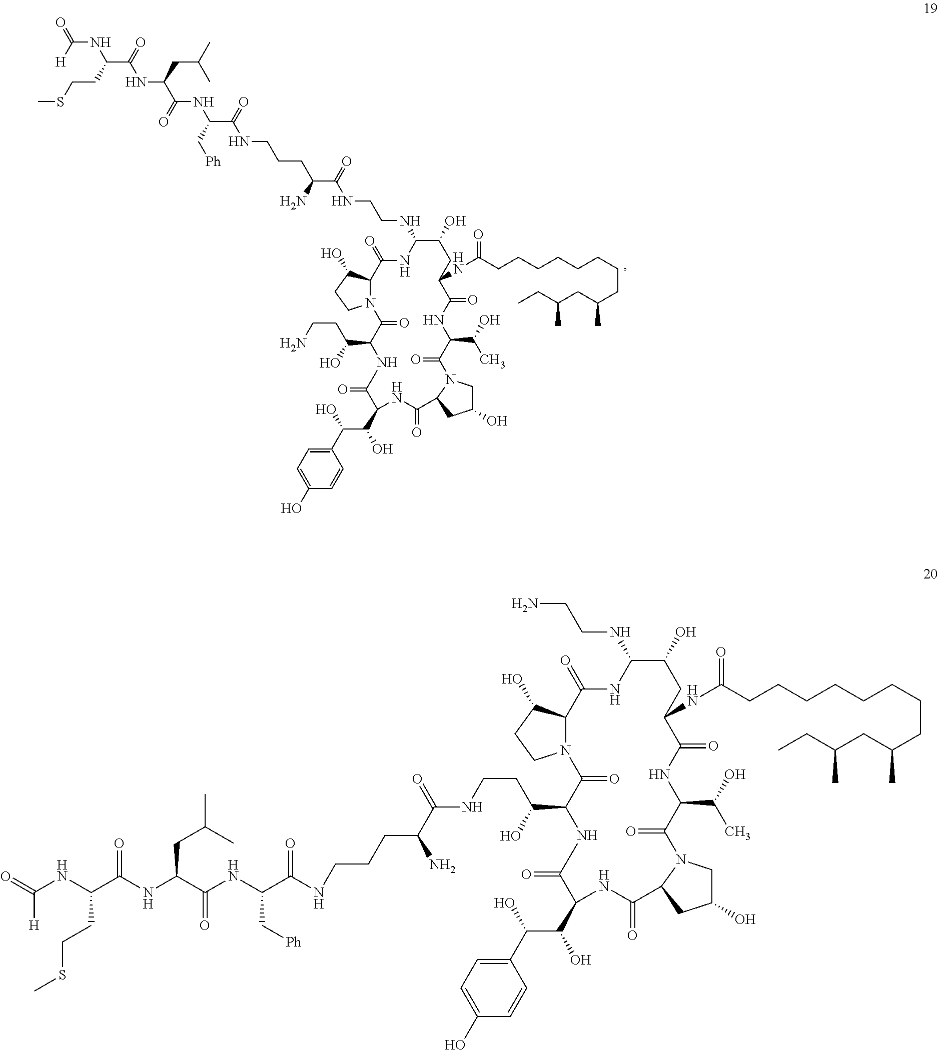

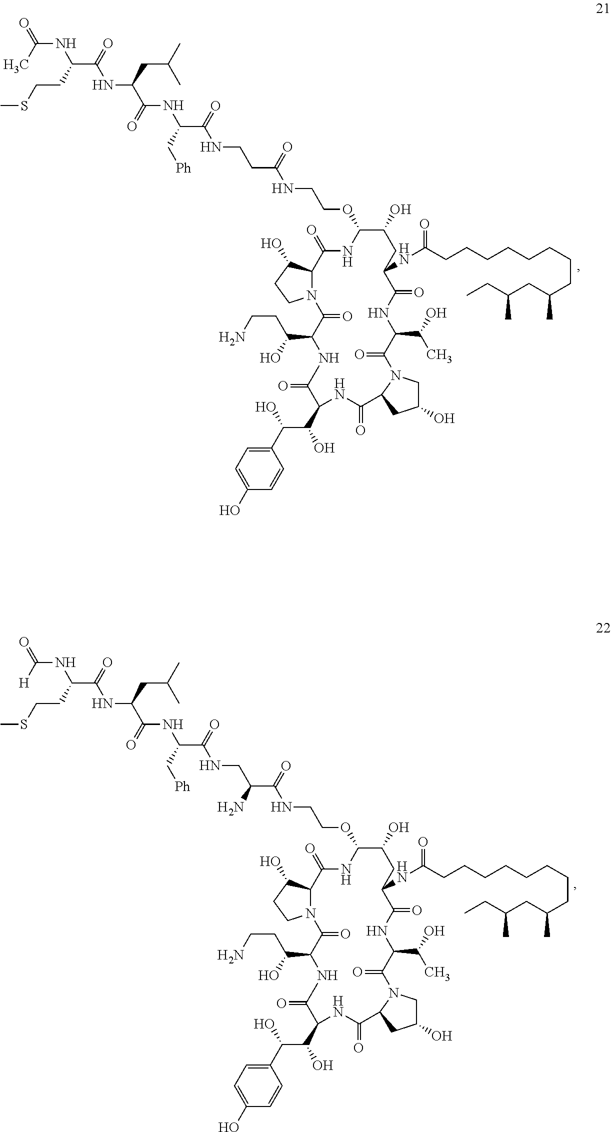

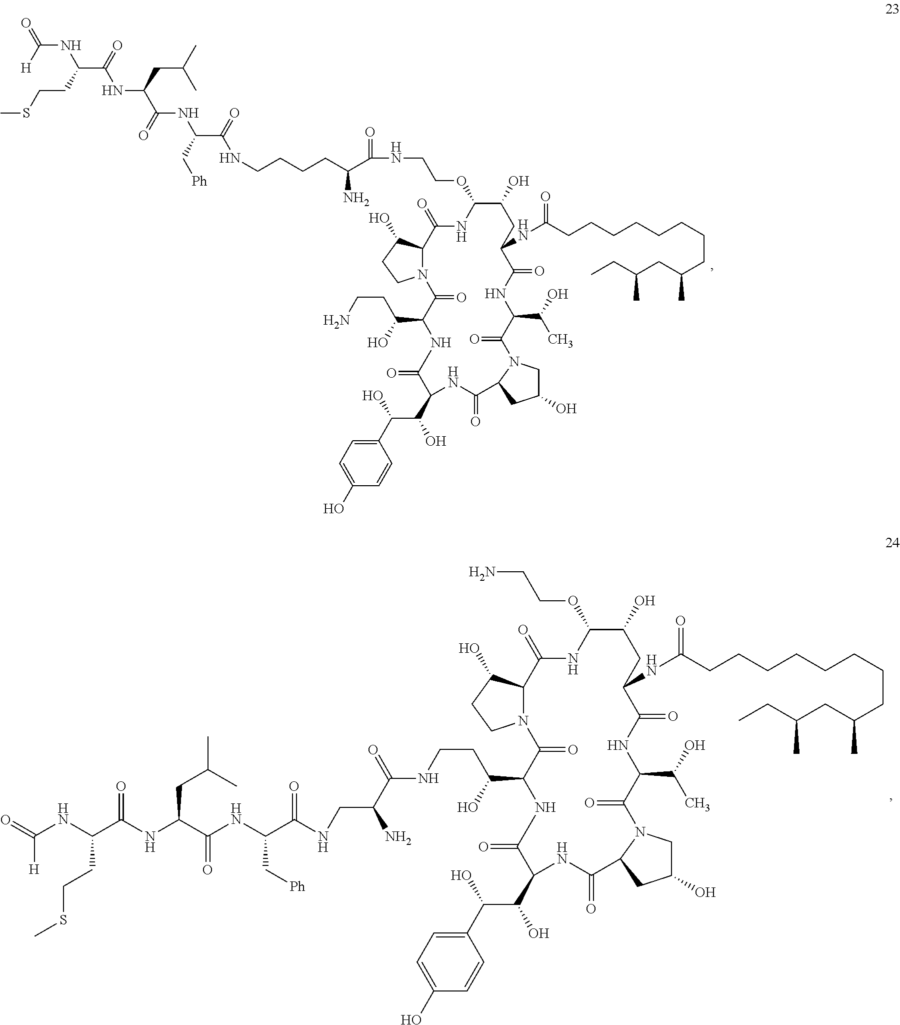

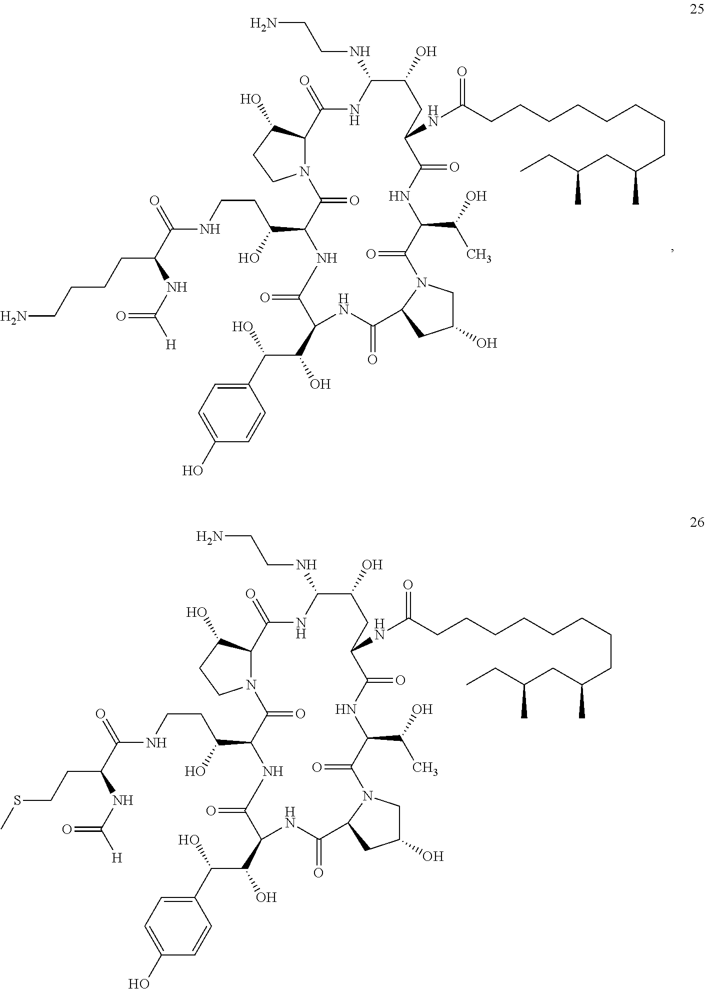 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCCCN)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

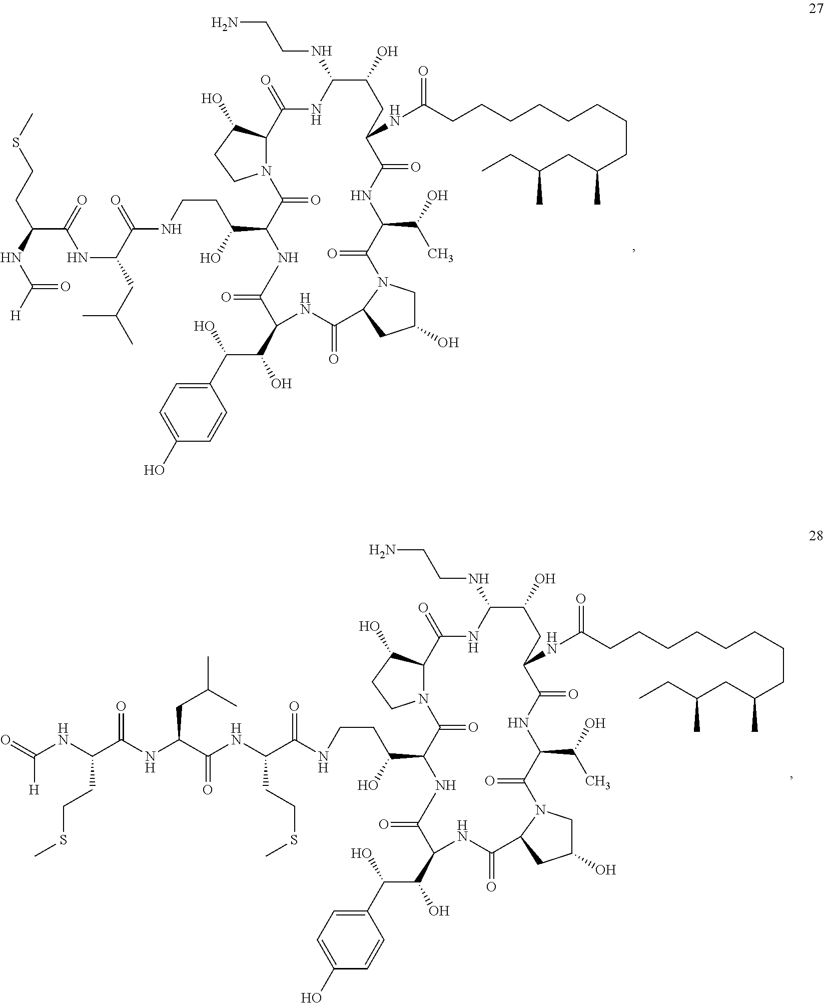

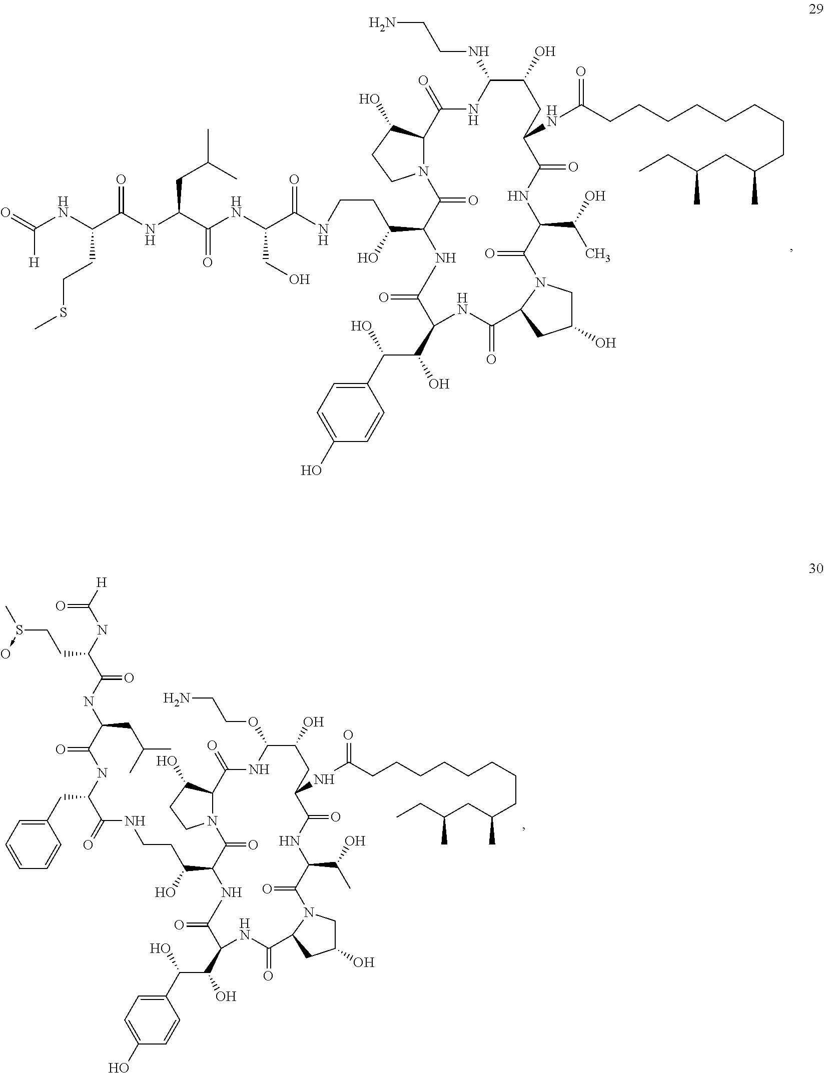

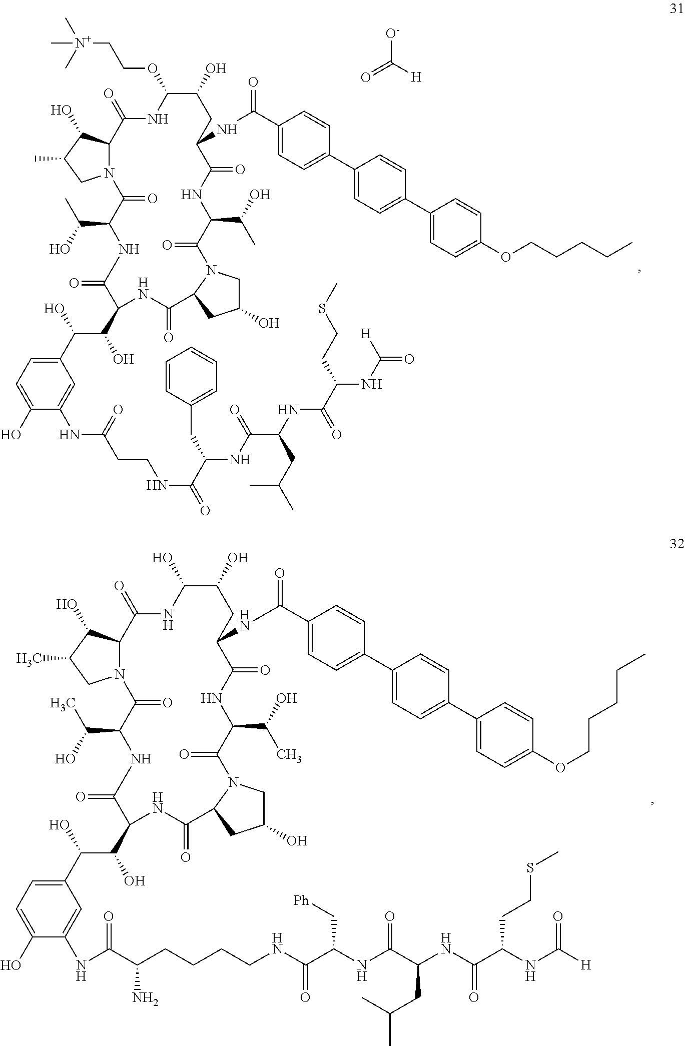

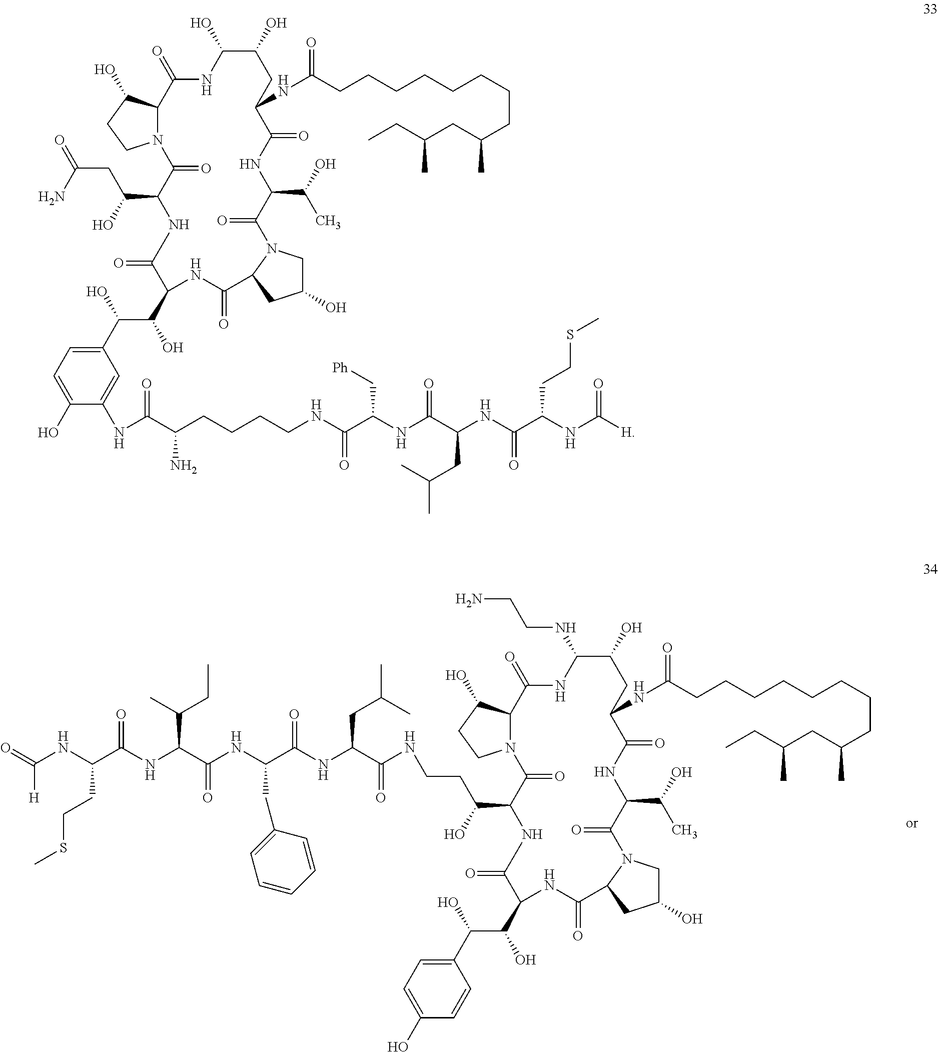

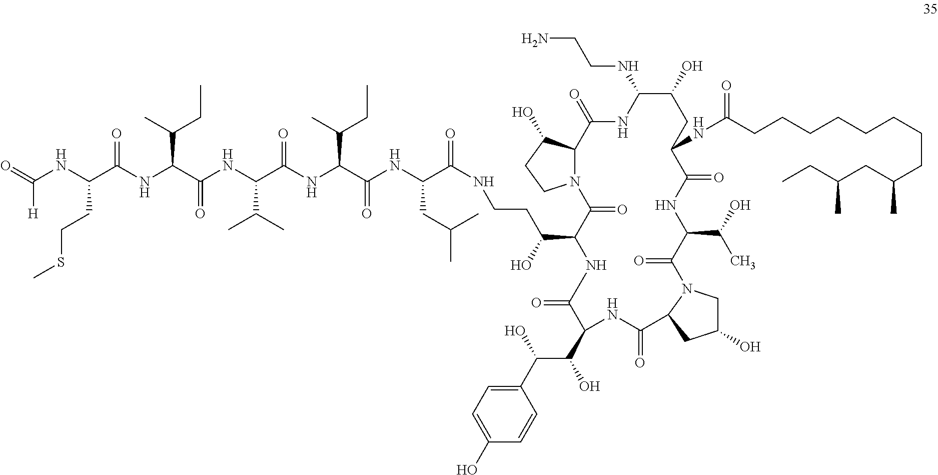 [SMILES: CCC(C)[C@H](NC(=O)[C@H](CCSC)NC=O)C(=O)N[C@H](C(=O)N[C@H](C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O)C(C)CC)C(C)C]

In other embodiments, the compound is selected from compounds 1-35.

In another aspect, the invention features a pharmaceutical composition including any of the foregoing compounds and a pharmaceutically acceptable excipient.

In another aspect, the invention features a method for the inhibition of fungal growth (e.g., a fungus of the genus Aspergillus or Candida). This method includes contacting a fungus with an effective amount of any of the foregoing compounds or compositions.

In another aspect, the invention features a method for the treatment of a fungal infection (e.g., an infection caused by a fungus of the genus Aspergillus or Candida) or a presumed fungal infection. This method includes administering to a subject in need thereof an effective amount of any of the foregoing compounds or compositions.

In another aspect, the invention features a method for the prophylactic treatment of a fungal infection (e.g., an infection caused by a fungus of the genus Aspergillus or Candida). This method includes administering to a subject in need thereof (e.g., a subject at risk of a fungal infection) an effective amount of any of the foregoing compounds or compositions.

In some embodiments, the fungal infection is aspergillosis (e.g., invasive aspergillosis, pulmonary aspergillosis) such as an infection caused by Aspergillus fumigatus.

In other embodiments, the fungal infection is candidiasis (e.g., an intra-abdominal abscess, peritonitis, a pleural cavity infection, esophagitis, candidemia, or invasive candidiasis) such as an infection caused by Candida albicans.

In some embodiments of any of the foregoing methods, the subject is immunocompromised (e.g., the subject has been diagnosed with humoral immune deficiency, T cell deficiency, neutropenia, asplenia, or complement deficiency, the subject is being treated or is about to be treated with immunosuppresive drugs, the subject has been diagnosed with a disease which causes immunosuppression such as, cancer for example, leukemia, lymphoma, multiple myeloma, human immunodeficiency virus infection, or acquired immunodeficiency syndrome). In other embodiments, the subject has undergone or is about to undergo hematopoietic stem cell transplantation, or an organ transplant.

In particular embodiments of any of the foregoing methods, administering comprises administration of any of the foregoing compounds or compositions intramuscularly, intravenously (e.g., as a sterile solution free of particulate emboli and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions).

In another aspect is a compound having the structure:

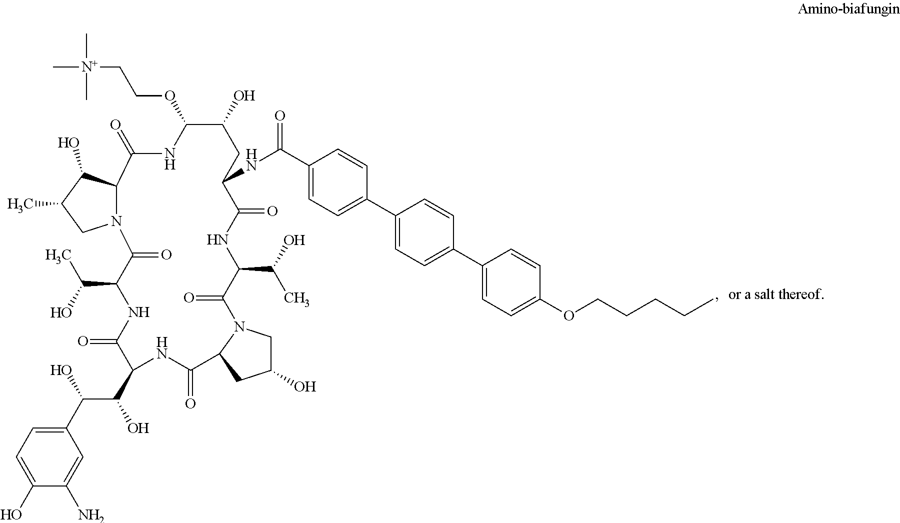 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

In yet another aspect is a compound having the structure:

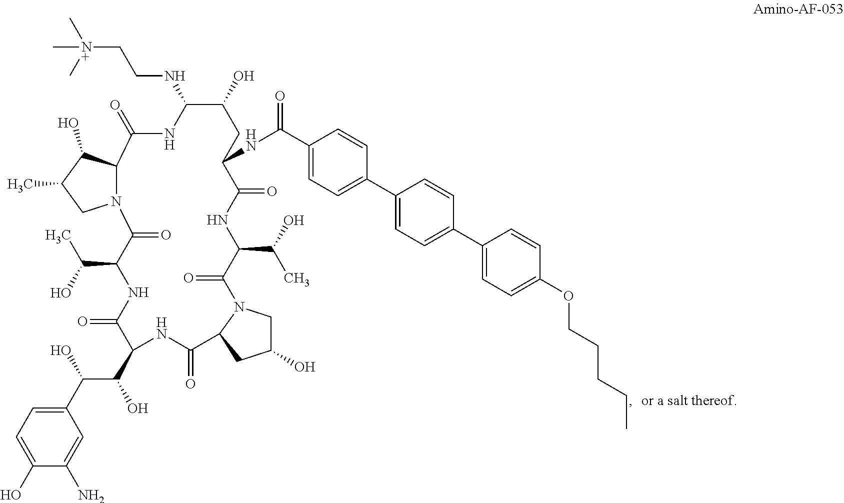 [SMILES: CCCCCOc1ccc(-c2ccc(-c3ccc(C(=O)N[C@H]4C[C@@H](O)[C@@H](NCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

In still another aspect is a compound having the structure:

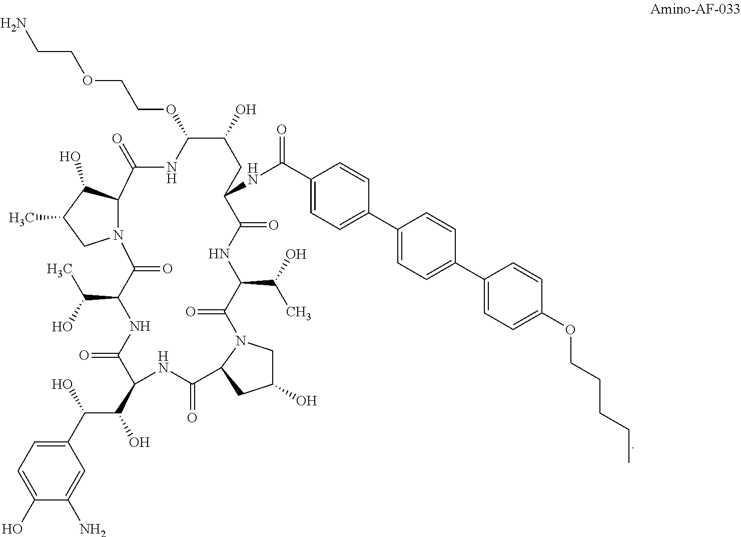 [SMILES: CCCCCOc1ccc(-c2ccc(-c3ccc(C(=O)N[C@H]4C[C@@H](O)[C@@H](OCCOCCN)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

As used herein, the term “alkyl,” “alkenyl,” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. The term “cycloalkyl,” as used herein, represents a monovalent saturated or unsaturated non-aromatic cyclic alkyl group having between three to nine carbons (e.g., a C3-C9 cycloalkyl), unless otherwise specified, and is exemplified by cyclopropyl, cyclobutyl, cyclopentyl, cyclohexyl, cycloheptyl, bicyclo[2.2.1.]heptyl, and the like. When the cycloalkyl group includes at least one carbon-carbon double bond, the cycloalkyl group can be referred to as a “cycloalkenyl” group. Exemplary cycloalkenyl groups include cyclopentenyl, cyclohexenyl, and the like. When the cycloalkyl group includes at least one carbon-carbon triple bond, the cycloalkyl group can be referred to as a “cycloalkynyl” group.

Typically, the alkyl, alkenyl, and alkynyl groups contain 1-12 carbons (e.g., C1-C12 alkyl) or 2-12 carbons (e.g., C2-C12 alkenyl or C2-C12 alkynyl). In some embodiments, the groups are C1-C8, C1-C6, C1-C4, C1-C3, or C1-C2 alkyl groups; or C2-C8, C2-C6, C2-C4, or C2-C3 alkenyl or alkynyl groups. Further, any hydrogen atom on one of these groups can be replaced with a substituent as described herein. For example, the term “aminoalkyl” refers to an alkyl group, as defined herein, comprising an optionally substituted amino group (e.g., NH₂).

Heteroalkyl, heteroalkenyl, and heteroalkynyl are similarly defined and contain at least one carbon atom but also contain one or more O, S, or N heteroatoms or combinations thereof within the backbone residue whereby each heteroatom in the heteroalkyl, heteroalkenyl, or heteroalkynyl group replaces one carbon atom of the alkyl, alkenyl or alkynyl group to which the heteroform corresponds. In some embodiments, the heteroalkyl, heteroalkenyl, and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N. The term “heterocyclyl,” as used herein represents cyclic heteroalkyl or heteroalkenyl that is, e.g., a 3-, 4-, 5-, 6-, or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like.

Heteroalkyl, heteroalkenyl, or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙOR, (CH₂)ₙ COR, (CH₂)ₙCOOR, (CH₂)ₙSR, (CH₂), SOR, (CH₂)ₙSO₂R, (CH₂)ₙCONR₂, NRCOR, NRCOOR, OCONR₂, OCOR, and the like where the R group contains at least one C and the size of the substituent is consistent with the definition of e.g., alkyl, alkenyl, and alkynyl, as described herein (e.g., n is 1, 2, 3, 4, 5, 6, 7, 8, 9, 10, 11, or 12).

As used herein, the terms “alkylene,” “alkenylene,” and “alkynylene,” or the prefix “alk” refer to divalent or trivalent groups having a specified size, typically C1-C2, C1-C3, C1-C4, C1-C6, or C1-C8 for the saturated groups (e.g., alkylene or alk) and C2-C3, C2-C4, C2-C6, or C2-C8 for the unsaturated groups (e.g., alkenylene or alkynylene). They include straight-chain, branched-chain, and cyclic forms as well as combinations of these, containing only C and H when unsubstituted. Because they are divalent, they can link together two parts of a molecule, as exemplified by X in the compounds described herein. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example. For example, the term “alkaryl,” as used herein, represents an aryl group, as defined herein, attached to the parent molecular group through an alkylene group, as defined herein, and the term “alkheteroaryl” refers to a heteroaryl group, as defined herein, attached to the parent molecular group through an alkylene group, as defined herein. The alkylene and the aryl or heteroaryl group are each optionally substituted as described herein.

Heteroalkylene, heteroalkenylene, and heteroalkynylene are similarly defined as divalent groups having a specified size, typically C1-C3, C1-C4, C1-C6, or C1-C8 for the saturated groups and C2-C3, C2-C4, C2-C6, or C2-C8 for the unsaturated groups. They include straight chain, branched chain and cyclic groups as well as combinations of these, and they further contain at least one carbon atom but also contain one or more O, S, or N heteroatoms or combinations thereof within the backbone residue, whereby each heteroatom in the heteroalkylene, heteroalkenylene or heteroalkynylene group replaces one carbon atom of the alkylene, alkenylene, or alkynylene group to which the heteroform corresponds. As is understood in the art, these heteroforms do not contain more than three contiguous heteroatoms.

The term “alkoxy” represents a chemical substituent of formula —OR, where R is an optionally substituted alkyl group (e.g., C1-C6 alkyl group), unless otherwise specified. In some embodiments, the alkyl group can be substituted, e.g., the alkoxy group can have 1, 2, 3, 4, 5, or 6 substituent groups as defined herein.

The term “alkoxyalkyl” represents a heteroalkyl group, as defined herein, that is described as an alkyl group that is substituted with an alkoxy group. Exemplary unsubstituted alkoxyalkyl groups include between 2 to 12 carbons. In some embodiments, the alkyl and the alkoxy each can be further substituted with 1, 2, 3, or 4 substituent groups as defined herein for the respective group.

The term “amino,” as used herein, represents —N(R^{N1})₂, where each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, heterocyclyl (e.g., heteroaryl), alkheterocyclyl (e.g., alkheteroaryl), or two R^{N1} combine to form a heterocyclyl or an N-protecting group, and where each R^{N2} is, independently, H, alkyl, or aryl. In a preferred embodiment, amino is —NH₂, or —NR^{N1}, where R^{N1} is, independently, OH, NO₂, NH₂, NR^{N2}₂, SO₂OR^{N2}, SO₂R^{N2}, SOR^{N2}, alkyl, or aryl, and each R^{N2} can be H, alkyl, or aryl. The term “aminoalkyl,” as used herein, represents a heteroalkyl group, as defined herein, that is described as an alkyl group, as defined herein, substituted by an amino group, as defined herein. The alkyl and amino each can be further substituted with 1, 2, 3, or 4 substituent groups as described herein for the respective group. For example, the alkyl moiety may comprise an oxo(═O) substituent.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl, benzimidazolyl, pyrazolyl, imidazolyl, isoxazolyl, thiazolyl, benzothiazolyl, indolyl, or imidazopyridinyl. Even more particularly, such moiety is phenyl, pyridyl, thiazolyl, imidazopyridinyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of C1-C8, C1-C6, or more particularly C1-C4 or C1-C3 when saturated or C2-C8, C2-C6, C2-C4, or C2-C3 when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(C6-C12)alkyl(C1-C8), aryl(C6-C12)alkenyl(C2-C8), or aryl(C6-C12)alkynyl(C2-C8), plus the heteroforms. A typical example is phenylmethyl, commonly referred to as benzyl.

The term “pathogen pattern recognition receptor ligand,” as used herein refers to a molecule, such as a peptide, that can be recognized by and interact with a pathogen pattern recognition receptor, such as a chemotaxis receptor. The pathogen pattern recognition receptor ligand may be derived from prokaryotic or eukaryotic organisms, such as fungi, bacteria, or mammals. In some embodiments, the pathogen pattern recognition receptor ligand includes host immune stimulating factors. For example, once the pathogen pattern recognition receptor of a host organism interacts with the pathogen pattern recognition receptor ligand, the pathogen pattern recognition receptor ligand may stimulate immune responses in the host organism.

The term “chemotactic peptide,” as used herein refers to peptides that interact with chemotaxis receptors and induce chemotaxis.

The term “chemotaxis receptor,” as used herein refers to receptors that are used by eukaryotic cells to sense the presence of chemotactic stimuli and include formyl peptide receptors (FRP), chemokine receptors (e.g., CCR, CXCR), and leukotriene receptors (BLT).

The term “cyclic lipopeptide,” as used herein refers to compounds consisting of a lipid (e.g., a C10-C20 alkyl or C10-C20 alkenyl, optionally substituted aryl or heteroaryl) connected to a cyclic peptide of four to twenty amino acids (e.g., a member of the aculeacin, echinocandin, pneumocandin, cyclopeptamine, mulundocandin, sporiofungin or WF11899A class of antifungal drugs).

By “fungal infection” is meant the pathogenic growth of fungus in a host organism (e.g., a human subject). For example, the infection may include the excessive growth of fungi that are normally present in or on the body of a subject or growth of fungi that are not normally present in or on a subject. More generally, a fungal infection can be any situation in which the presence of a fungal population(s) is damaging to a host body. Thus, a subject is “suffering” from a fungal infection when an excessive amount of a fungal population is present in or on the subject's body, or when the presence of a fungal population(s) is damaging the cells or other tissue of the subject.

“Halo” may be any halogen atom, especially F, Cl, Br, or I, and more particularly it is fluoro or chloro.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls. In some embodiments, the haloalkyl group can be further substituted with 1, 2, 3, or 4 substituent groups as described herein for alkyl groups.

The term “hydrazone” as used herein, represents the group having the structure:

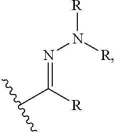

where each R is independently hydrogen, alkyl, or aryl.

The term “hydroxyl,” as used herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like.

The term “C₁₀-C₃₆ moiety” refers to a C₁₀-C₃₆ residue which contains predominantly (i.e., at least 75% of the atoms) carbon and hydrogen. The moiety may be aliphatic or aromatic, straight-chain, cyclic, branched, saturated or unsaturated, or combinations thereof. The moiety may contain aliphatic, aryl, and polycyclic moieties. The moiety may, however, contain heteroatoms over and above the carbon and hydrogen members. Thus, when specifically noted as containing such heteroatoms, the C₁₀-C₃₆ moiety may also contain heteroatoms within the “backbone” of C₁₀-C₃₆ moiety. In some embodiments, the heteroatoms may be contained within the aliphatic moiety. In other embodiments, the heteroatoms may be contained within the aryl and/or polycyclic moieties. In some aspects, a C₁₀-C₃₆ moiety may contain 0-8 heteroatoms selected from O, S and N, such as 0, 1, 2, 3, 4, or 5 heteroatoms, such as 1 or 2 O heteroatoms, 1, 2, or 3 N heteroatoms, 1 or 2 S heteroatoms, or combinations thereof. The β-1,3-glucan synthase inhibitor affects the lipid bilayer of fungal membranes, and therefore, in some aspects, the optional substituents are not highly polar or are non-polar. In some embodiments, optional substituents on R¹ (e.g., a C₁₀-C₃₆ moiety) may include halo, such as F or Cl, and/or contain an alkoxy group, which do not impart significant polarity.

In some embodiments, the C₁₀-C₃₆ moiety may contain 1-6, such as 1-5, optionally substituted, fused or unfused, cyclic or polycyclic moieties, where carbon atoms in the moiety may be located inside or outside of the ring system(s). Optionally substituted, fused or unfused, cyclic or polycyclic moieties include aromatic, heteroaromatic or cycloaliphatic, or cycloheteroaliphatic moieties, or combinations thereof. Typical examples of optionally substituted, fused or unfused, cyclic or polycyclic moieties include optionally substituted phenyl, naphthyl, biphenyl, terphenyl, such as p-terphenyl, oxazolyl, isooxazolyl, cyclohexyl/cyclohexylenyl, piperidinyl, piperizinyl, and thiadiazolyl such as 1,3,4-thiadiazolyl, and bivalent forms thereof, and combinations thereof. Hydrophobic moieties containing optionally substituted, fused or unfused, cyclic or polycyclic moieties may also include a C₁-C₃ alkylenyl moiety, such as methylenyl, between the attachment point of R¹ of any of Formulas I, II, XVII, and XVIII to the amine or amide moiety and the cyclic or polycyclic moieties.

Additional examples of the C₁₀-C₃₆ moiety include a C₁₃-C₁₇, straight or branched alkyl or alkenyl moiety having 0, 1, or 2 unsaturated bonds, such as a C₁₃, C₁₄, C₁₅, C₁₆, or C₁₇ straight or branched alkyl or alkenyl moiety. The moiety may also include a phenyl, naphthyl, or terphenyl moiety substituted with a C₁-C₁₀ alkoxy group, such as a C₅, C₆, C₇, or C₈ alkoxy group. The substituted phenyl, naphthyl, or terphenyl moiety may be attached directly to the amine or amide moiety, such as the amide moiety.

A polycyclic moiety may include 1 or 2 phenyl moieties and a 5 membered heteroaryl moiety, such as an isoxazolyl or a thiadiazolyl group, in which one or more cyclic moieties may be substituted with one or two substituents such as a C₁-C₆ alkyl or C₁-C₆ alkoxy, such as methoxy or C₅-alkoxy, or cyclohexyl. In some aspects, a polycyclic moiety may have 5 cyclic moieties including one or more of cyclohexyl/cyclohexenyl, thiadiazolyl, piperidinyl, phenyl or bivalent forms thereof. In some aspects, a moiety may contain a C₁-C₈ alkylenyl such as a methylenyl moiety attached on one end directly to the amine or amide moiety (such as an amine moiety) attached to R¹ of Formula I, II, XVII, or XVIII, and on the other end attached to a polycyclic moiety containing up to 5 cyclic moieties that may include thiadiazolyl, phenyl/phenlenyl, piperdinyl, and cyclohexyl or bivalent forms thereof.

The term “immunocompromised,” as used herein refers to an immune response that has been weakened by a condition or an immunosuppressive agent.

The term “inhibitor of β-1,3-glucan synthase,” as used herein refers to compounds that inhibit that action of the enzyme β-1,3-glucan synthase derived from a fungus that causes infection at less than 5 μM (e.g., less than 4 μM, 3 μM, 2 μM, 1 μM, 500 nM, or 100 nM) or displays an MIC less than 32 μg/mL (e.g., less than 30 μg/mL, 20 μg/mL, 10 μg/mL, 5 μg/mL, 1 μg/mL).

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

An “oxo” group is a substituent having the structure ═O, where there is a double bond between a carbon and an oxygen atom.

The term “phosphoryl” as used herein, represents the group having the structure:

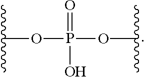 [SMILES: *OP(=O)(O)O*]

The term “sulphonyl” as used herein, represents the group having the structure:

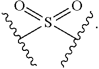 [SMILES: *S(*)(=O)=O]

Typical optional substituents on aromatic or heteroaromatic groups include independently halo, such as chloro, fluoro, CN, NO₂, CF₃, OCF₃, COOR′, CONR′₂, OR′, SR′, SOR′, SO₂R′, NR′₂, NR′(CO)R′, NR′C(O)OR′, NR′C(O)NR′₂, NR′SO₂NR′₂, or NR′SO₂R′, where each R′ is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl. For example, compound 13a and 13b contain a halo substituted aryl such as chlorophenyl at the R¹⁵ position. Optional substituents leave the ability of the compounds herein to be used for their intended purposes, such as treating a fungal infection, qualitatively intact

Optional substituents on a non-aromatic group (e.g., alkyl, alkenyl, and alkynyl groups), are typically selected from the same list of substituents suitable for aromatic or heteroaromatic groups, except as noted otherwise herein. A non-aromatic group may also include an optional substituent selected from ═O and ═NOR′ where R′ is H or an optionally substituted group selected from alkyl, alkenyl, alkynyl, heteroalkyl, heteroalkenyl, heteralkynyl, heteroaryl, and aryl (all as defined above).

In general, a substituent group (e.g., alkyl, alkenyl, alkynyl, or aryl (including all heteroforms defined above)) may itself optionally be substituted by additional substituents. The nature of these substituents is similar to those recited with regard to the substituents on the basic structures above.

Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo (preferably chloro or fluoro) and the like would be included. For example, where a group is substituted, the group may be substituted with 1, 2, 3, 4, 5, or 6 substituents. Optional substituents include, but are not limited to: C1-C6 alkyl or heteroalkyl, C2-C6 alkenyl or heteroalkenyl, C2-C6 alkynyl or heteroalkynyl, halogen; aryl, heteroaryl, azido(-N₃), nitro (—NO₂), cyano (—CN), acyloxy(—OC(═O)R′), acyl (—C(═O)R′), alkoxy (—OR′), amido (—NR′C(═O)R″, or —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, C1-C6 alkyl or heteroalkyl, C2-C6 alkenyl, or heteroalkenyl, 2C-6C alkynyl, or heteroalkynyl, aryl, or heteroaryl. A substituted group may have, for example, 1, 2, 3, 4, 5, 6, 7, 8, or 9 substituents.

In some embodiments, the moieties are amino acid residues. The amino acid residue may be of a naturally occurring amino acid (e.g., Ala, Arg, Asn, Asp, Cys, Gin, Glu, Gly, His, Ile, Leu, Lys, Met, Phe, Pro, Ser, Thr, Trp, Tyr, or Val), or the amino acid residue may be of a non-naturally occurring amino acid. A “non-naturally occurring amino acid” is an amino acid which is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; and hydroxyproline.

The term an “effective amount” of an agent, as used herein, is that amount sufficient to effect beneficial or desired results, such as clinical results, and, as such, an “effective amount” depends upon the context in which it is being applied. For example, in the context of administering an agent that is used for the treatment of a fungal infection, an effective amount of an agent is, for example, an amount sufficient to slow down or reverse the progression of the infection as compared to the response obtained without administration of the agent.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions of the compounds herein may be formulated to be administered intramuscularly, intravenously (e.g., as a sterile solution free of particulate emboli and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, injection, infusion, continuous infusion, localized perfusion bathing target cells directly, catheter, lavage, in cremes, or lipid compositions.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as used herein, represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds herein may have ionizable groups so as to be capable of preparation as pharmaceutically acceptable salts. These salts may be acid addition salts involving inorganic or organic acids or the salts may, in the case of acidic forms of the compounds herein be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “prophylactic treatment,” as used herein, refers to treatment initiated, for example, prior to (“pre-exposure prophylaxis”) or following (“post-exposure prophylaxis”) an event that precedes the onset of the disease, disorder, or conditions (e.g., when a subject is being treated or is about to be treated with immunosuppresive drugs, a subject has been diagnosed with a disease which causes immunosuppression (e.g., cancer such as, leukemia, lymphoma, multiple myeloma, or acquired immunodeficiency syndrome), a subject has undergone or is about to undergo hematopoietic stem cell transplantation, or a subject has undergone or is about to undergo an organ transplant). Prophylactic treatment that includes administration of a compound described herein, or a pharmaceutically acceptable salt thereof, or a pharmaceutical composition thereof, can be acute, short-term, or chronic. The doses administered may be varied during the course of prophylactic treatment.

As used herein, the term “subject” can be a human, non-human primate, or other mammal, such as but not limited to dog, cat, horse, cow, pig, turkey, goat, fish, monkey, chicken, rat, mouse, and sheep.

As used herein, and as well understood in the art, “to treat” a condition or “treatment” of a fungal infection is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “unit dosage form” refers to a physically discrete unit suitable as a unitary dosage for human subjects and other mammals, each unit containing a predetermined quantity of active material calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gelcap, and syrup.

In some cases, the compounds herein contain one or more chiral centers. The compounds include each of the isolated stereoisomeric forms as well as mixtures of stereoisomers in varying degrees of chiral purity, including racemic mixtures. It also encompasses the various diastereomers, enantiomers, and tautomers that can be formed.

Compounds may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically labeled compounds can be prepared by synthesizing a compound using a readily available isotopically labeled reagent in place of a non-isotopically labeled reagent. In some embodiments, the compound, or a composition that includes the compound, has the natural abundance of each element present in the compound.

The compounds described herein are also useful for the manufacture of a medicament useful to treat fungal infections.

Other features and advantages of the invention will be apparent from the following detailed description and the claims.

### DESCRIPTION OF THE DRAWINGS

FIG. 1 shows data generated from movies of neutrophil (NF) migration counts at the 4 hour timepoint after addition of NFs to the cell loading chamber (CLC). The six bars indicate neutrophil (NF) migration in the presence of Aspergillus fumigatus (Af) alone (Af+NF), Af and 10 nM Compound 1, Af and 10 nM Compound 11, Af and 10 nM caspofungin acetate (caspo), Af and 100 nM fMLP, and 100 nM fMLP alone, respectively.

FIG. 2 shows the percentage of hyphal growth versus conidia under different conditions: Control Growth with no additives, added neutrophils alone (NF), added NFs and 10 nM Compound 1, added NFs and 10 nM Compound 11, added NFs and 10 nM caspofungin acetate (caspo), and added NFs and 100 nM fMLP.

FIG. 3 shows representative single frames captured from movies used to generate data in FIGS. 1 and 2.

FIGS. 4a-h show the timecourse of A. fumigatus (Af) growth and neutrophil (PMNs) migration in FCCs using three individual healthy blood donors at various timepoints (t=0, 8 and 16 hours). Elongated cells (i.e., filament-like) are Aspergillus fumigatus while circular cells are PMNs. FIGS. 4a and b show Af growth in the absence of PMNs or drug. FIGS. 4c and d show Af growth in the presence of PMNs alone. FIGS. 4e and f show inhibition of growth of Af and migration of PMNs in the presence of fMLP and PMNs. FIG. 4g shows inhibition of growth of Af and migration of PMNs in presence of Compound 11 and PMNs. FIG. 4h shows growth of Af in presence of caspofungin acetate (caspo) and PMNs.

### DETAILED DESCRIPTION

Provided are synthetic bifunctional compounds (e.g., non-antibody compounds) useful in the treatment of fungal infections including a pathogen pattern recognition receptor ligand and an inhibitor of β-1,3-glucan synthase. The inventors have found that compounds of this type can have increased antifungal activity due to their ability to bind to the fungal cell wall through inhibition of β-1,3-glucan synthase, thereby driving a concentration gradient near the locus of infection, whereby the pathogen pattern recognition receptor moiety then serves as a gradient against which neutrophils chemotax to the site. Suitable compounds herein include, without limitation, compounds 1-29.

β-1,3-glucan synthase Inhibitors

β-1,3-glucan synthase is a glucosyltransferase enzyme involved in the generation of p-glucan in fungi. Inhibition of this enzyme results in disrupting the integrity of the fungal cell well and serves as a pharmacological target for antifungal drugs. Several classes of antifungal drugs inhibit β-1,3-glucan synthase, including but not limited to the aculeacin, echinocandin, pneumocandin, cyclopeptamine, mulundocandin, sporiofungin and WF11899A class of antifungal drugs.

Exemplary β-1,3-glucan synthase inhibitors include, without limitation, compounds having the structure:

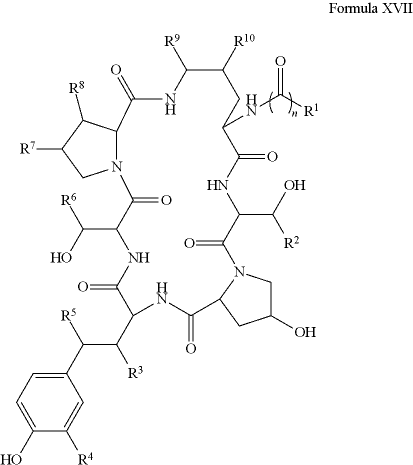

where R¹ is a C₁₀-C₃₆ moiety containing 0-8 heteroatoms selected from O, S, and N, optionally substituted with halo (preferably chloro or fluoro); R² is hydrogen or methyl; R³ and R⁵ are independently hydrogen or hydroxyl; R⁴ is hydrogen, hydroxyl, —OSO₃H, or —NR¹¹R¹²; R⁶ is hydrogen, methyl, —CH₂(C═O)NR¹¹R¹², —CH₂CH₂NR¹¹R¹², or —CH₂CH₂N⁺R¹¹R¹²R¹³; R⁷ is hydrogen, methyl, hydroxyl, —NR¹¹R¹², or —NH(C═NH)NR¹¹R¹²; R⁸ is hydrogen or hydroxyl; R⁹ is -(Q₁(CH₂)_{b})_{c}Q₂, hydrogen, or hydroxyl; R¹¹, R¹², and R¹³ are independently hydrogen or C₁-C₃ alkyl; R¹⁰ is hydrogen, hydroxyl, oxo, —NR¹¹R¹², NH(CH₂)ₐNR¹¹R¹², or —NH(CH₂)ₐN⁺R¹¹R¹³; n is 0 or 1; and a is 2 to 4; Q₁ is S, O or NH; b is 2-6 (preferably 2-4); c is 1-8 (preferably 1-3); Q₂ is NR¹¹R¹², N⁺R¹¹R¹²R¹³, or OR²⁰; and R²⁰ is CH₃, CH₂CH₃, CH₂CH₂CH₃, CH(CH₃)₂, or H; wherein at least one of R⁴, R⁶, R⁷, R⁹, or R¹⁰ contains —NR¹¹R¹², wherein at least one R¹¹ is a bond joining the inhibitor of β-1,3-glucan synthase to the pathogen pattern recognition receptor ligand or to the linker.

In some embodiments, R⁴ of Formula XVII contains —NR¹¹R¹², wherein R¹¹ is a bond joining the inhibitor of β-1,3-glucan synthase to the pathogen pattern recognition receptor ligand or to the linker.

In some embodiments, R⁶ of Formula XVII contains —NR¹¹R¹², wherein R¹¹ is a bond joining the inhibitor of β-1,3-glucan synthase to the pathogen pattern recognition receptor ligand or to the linker.

In some embodiments, R⁷ of Formula XVII contains —NR¹¹R¹², wherein R¹¹ is a bond joining the inhibitor of β-1,3-glucan synthase to the pathogen pattern recognition receptor ligand or to the linker.

In some embodiments, R⁹ of Formula XVII contains —NR¹¹R¹², wherein R¹¹ is a bond joining the inhibitor of β-1,3-glucan synthase to the pathogen pattern recognition receptor ligand or to the linker.

In some embodiments, R¹⁰ of Formula XVII contains —NR¹¹R¹², wherein R¹¹ is a bond joining the inhibitor of β-1,3-glucan synthase to the pathogen pattern recognition receptor ligand or to the linker.

In some embodiments, R¹ of Formula XVII contains a C₁-C₁₇ alkyl or heteroalkyl, C₂-C₁₇ alkenyl or heteroalkenyl, aryl or heteroaryl, cyclic, polycyclic, heterocyclic or heteropolycyclic moiety, or a combination thereof to form a C₁₀-C₃₆ moiety.

In some embodiments, R¹ of Formula XVII contains optionally substituted C₁₃-C₁₇ alkyl, optionally substituted C₁₃-C₁₇ alkenyl, optionally substituted aryl, or optionally substituted heteroaryl.

In some embodiments, R⁹ of Formula XVII contains hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐN⁺R¹¹R¹²R¹³, —S(CH₂)ₐN⁺R¹¹R¹², —S(CH₂)ₐN⁺R¹¹R¹²R¹³, —CH₂NR¹¹R¹², or —CH₂N⁺R¹¹R¹²R¹³.

In some embodiments, R⁹ of Formula XVII contains hydrogen, hydroxyl, —NH₂, —O(CH₂)₂NH₂, —O(CH₂)₂N⁺(CH₃)₃, —O(CH₂)₂O(CH₂)₂NH₂, —NH(CH₂)₂NH₂, —NH(CH₂)₂N⁺(CH₃)₃, —S(CH₂)₂NH₂, or —CH₂NH₂.

In some embodiments, R¹ of Formula I, II, XVII, or XVIII is:

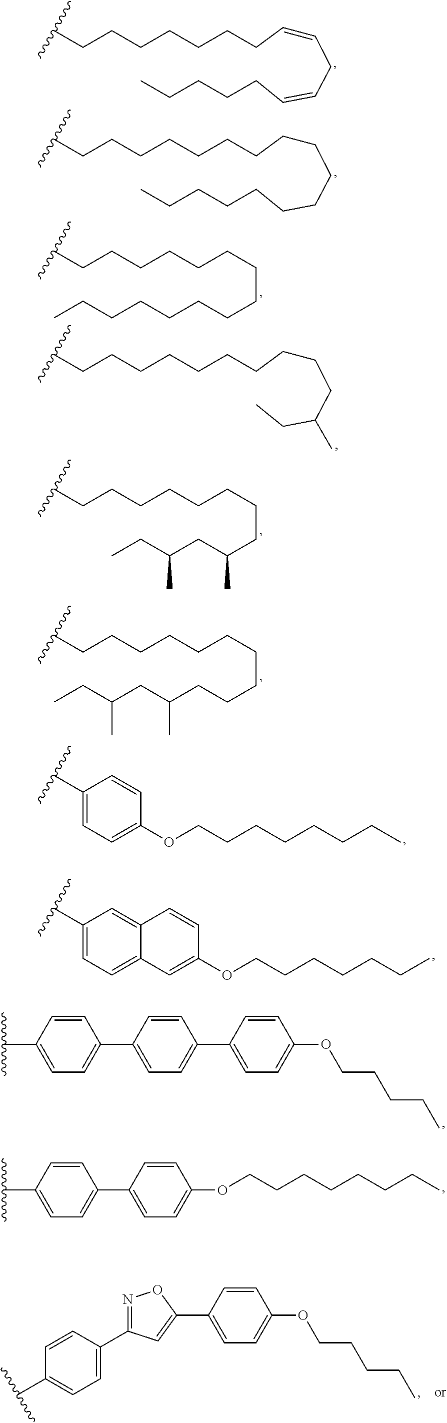

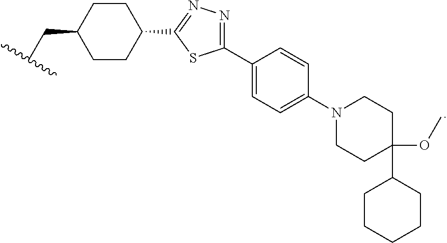 [SMILES: *C[C@H]1CC[C@H](c2nnc(-c3ccc(N4CCC(OC)(C5CCCCC5)CC4)cc3)s2)CC1]

Other β-1,3-glucan synthase inhibitors are known in the art. Three antifungal drugs approved by the United States Food and Drug Administration, caspofungin, anidulafungin, and micafungin, are known to inhibit β-1,3-glucan synthase which have the structures shown below.

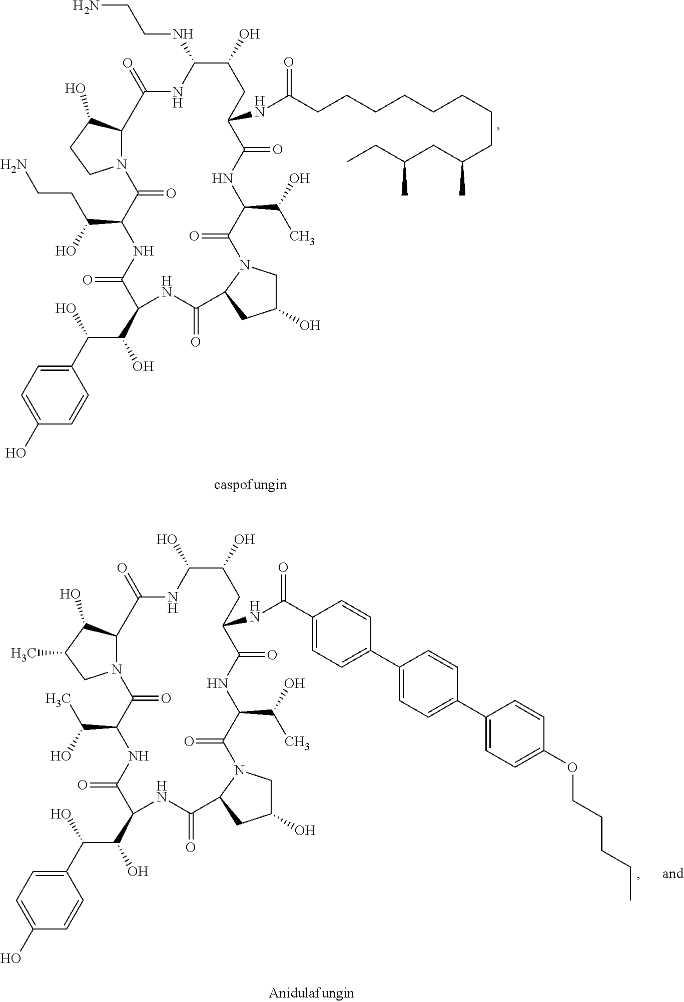 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CCCCCOc1ccc(-c2ccc(-c3ccc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1]

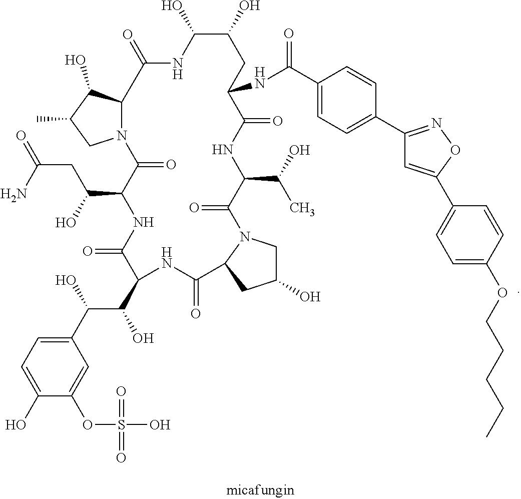 [SMILES: CCCCCOc1ccc(-c2cc(-c3ccc(C(=O)N[C@H]4C[C@@H](O)[C@@H](O)NC(=O)[C@@H]5[C@@H](O)[C@@H](C)CN5C(=O)[C@H]([C@H](O)CC(N)=O)NC(=O)[C@H]([C@H](O)[C@@H](O)c5ccc(O)c(OS(=O)(=O)O)c5)NC(=O)[C@@H]5C[C@@H](O)CN5C(=O)[C@H]([C@@H](C)O)NC4=O)cc3)no2)cc1]

Other exemplary β-1,3-glucan synthase inhibitors include, without limitation:

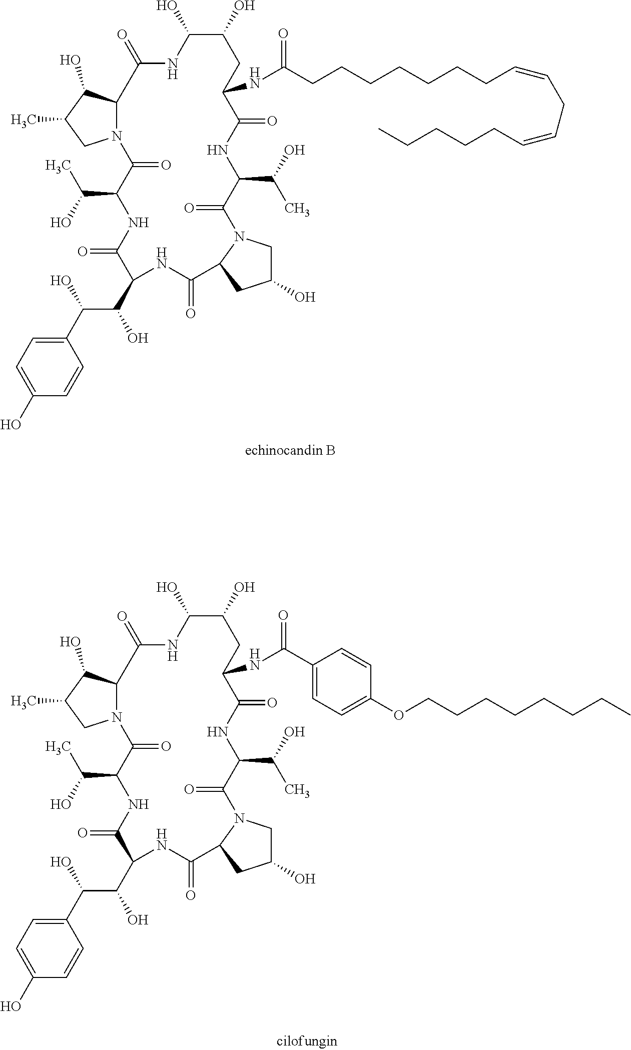 [SMILES: CCCCC/C=C\C/C=C\CCCCCCCC(=O)N[C@H]1C[C@@H](O)[C@@H](O)NC(=O)[C@@H]2[C@@H](O)[C@@H](C)CN2C(=O)[C@H]([C@@H](C)O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CCCCCCCCOc1ccc(C(=O)N[C@H]2C[C@@H](O)[C@@H](O)NC(=O)[C@@H]3[C@@H](O)[C@@H](C)CN3C(=O)[C@H]([C@@H](C)O)NC(=O)[C@H]([C@H](O)[C@@H](O)c3ccc(O)cc3)NC(=O)[C@@H]3C[C@@H](O)CN3C(=O)[C@H]([C@@H](C)O)NC2=O)cc1]

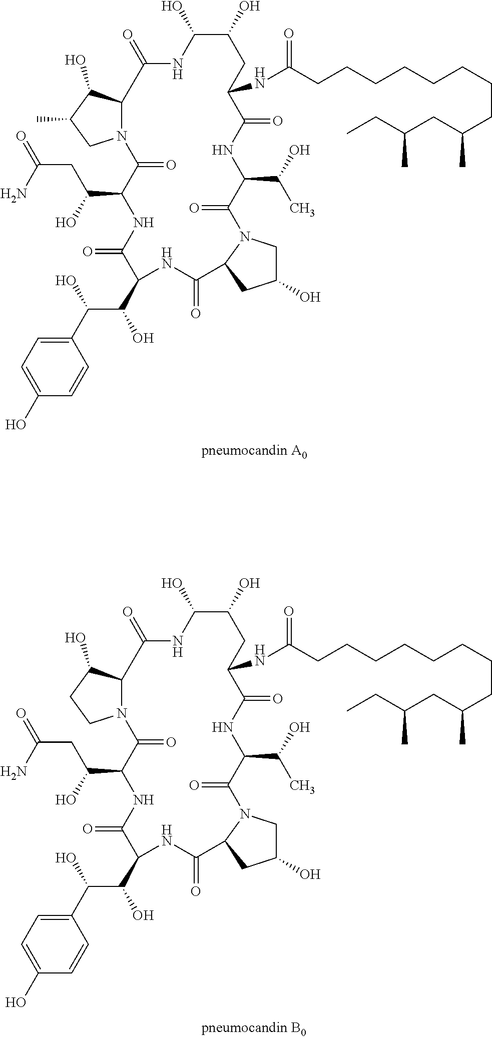 [SMILES: CC[C@H](C)C[C@H](C)CCCCCCCCC(=O)N[C@H]1C[C@@H](O)[C@@H](O)NC(=O)[C@@H]2[C@@H](O)[C@@H](C)CN2C(=O)[C@H]([C@H](O)CC(N)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CC[C@H](C)C[C@H](C)CCCCCCCCC(=O)N[C@H]1C[C@@H](O)[C@@H](O)NC(=O)[C@@H]2[C@@H](O)CCN2C(=O)[C@H]([C@H](O)CC(N)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

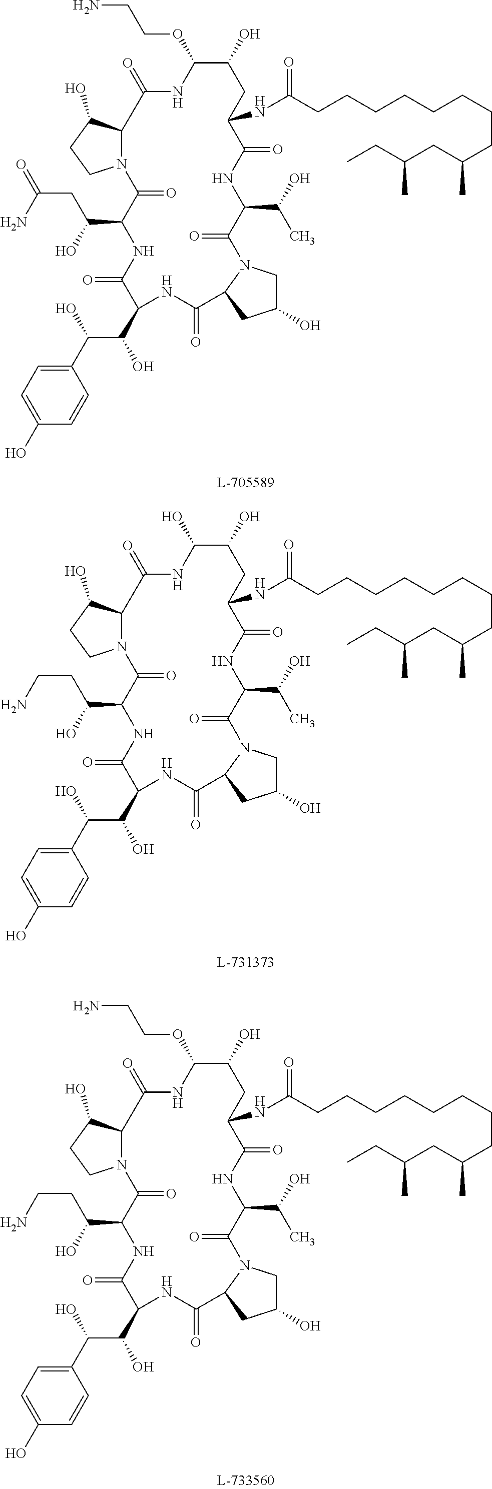

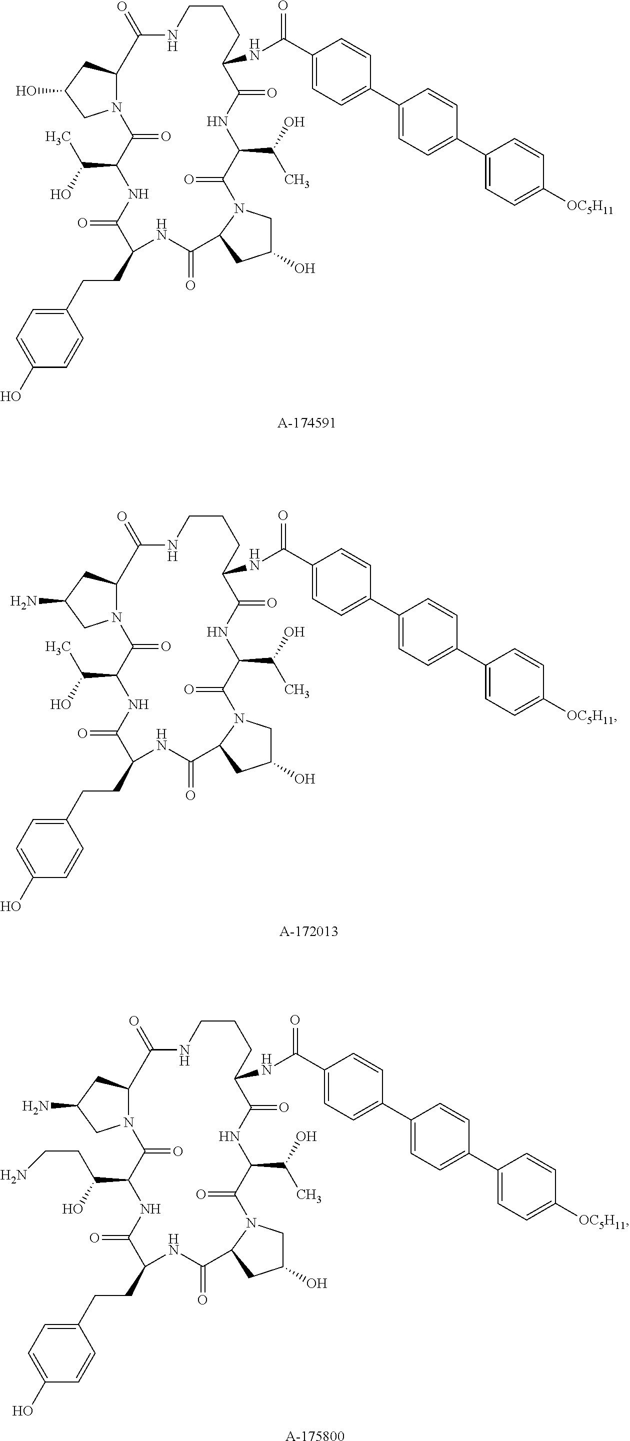

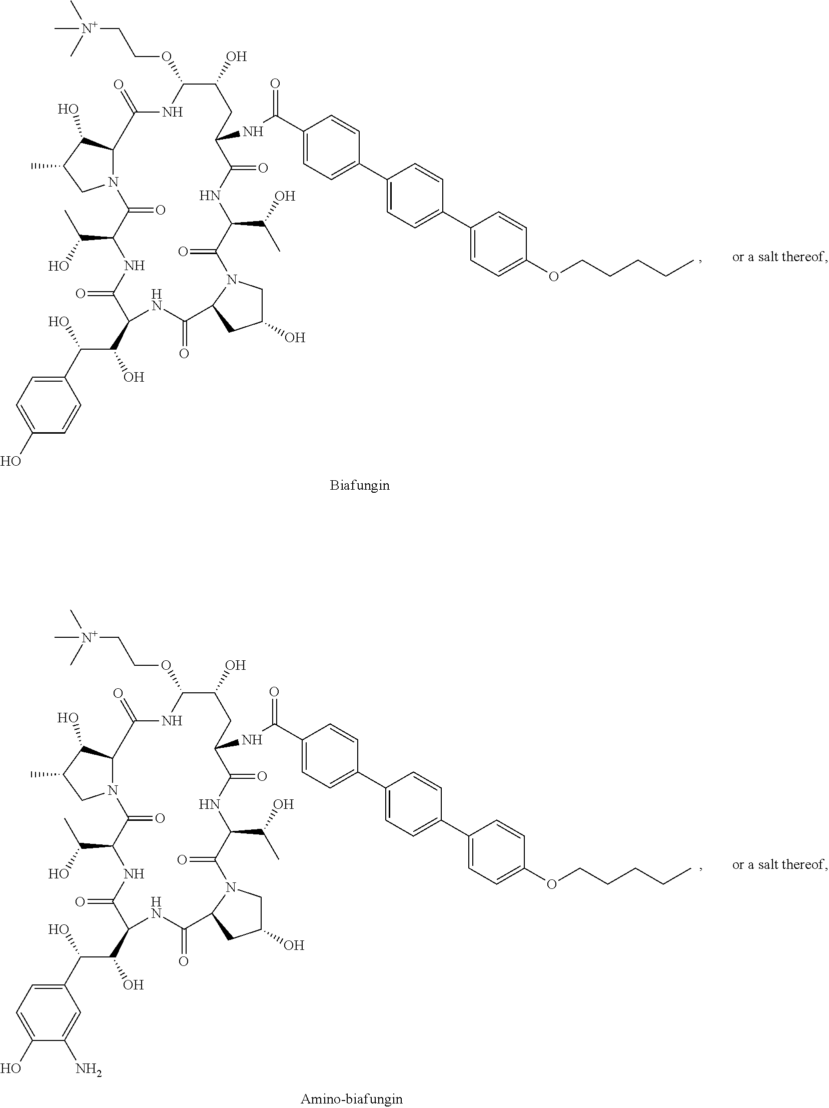 [SMILES: CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1 ; CCCCCOc1ccc(-c2ccc(-c3ccc(C(=O)N[C@H]4C[C@@H](O)[C@@H](OCC[N+](C)(C)C)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

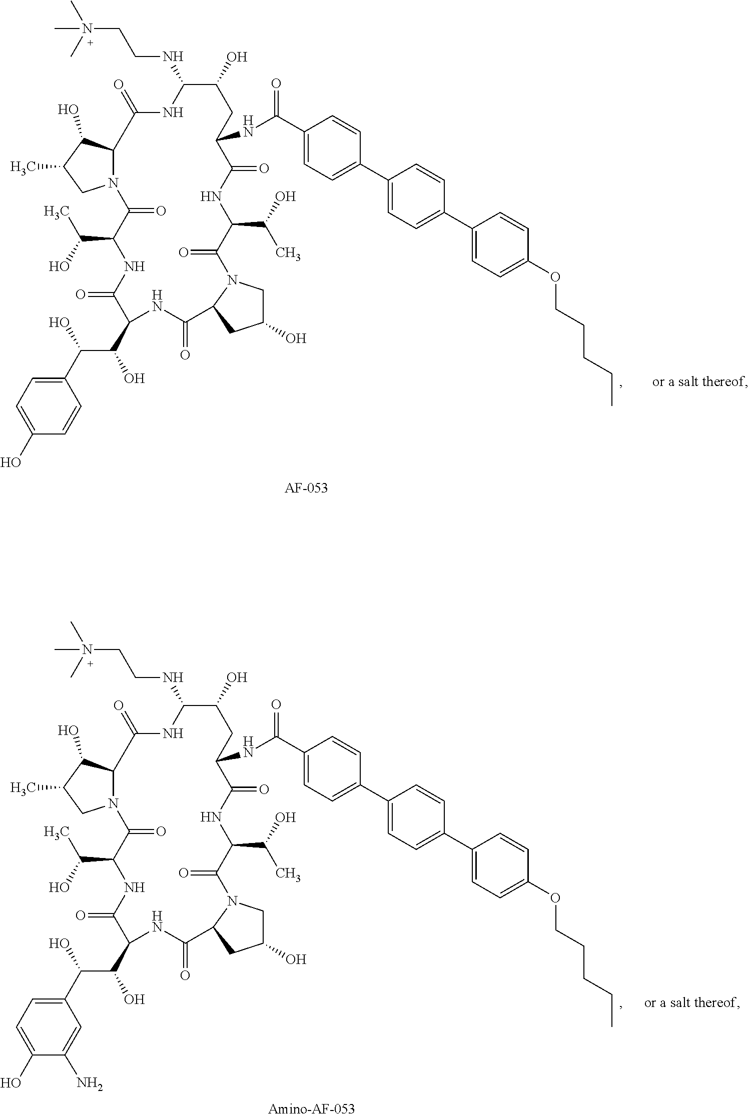

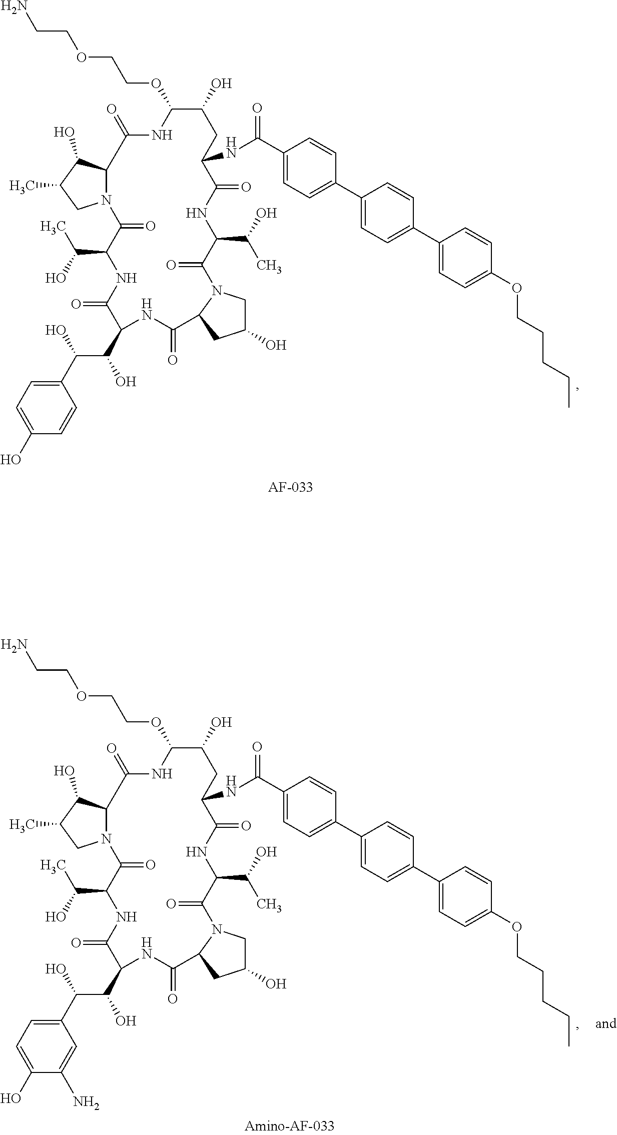 [SMILES: CCCCCOc1ccc(-c2ccc(-c3ccc(C(=O)N[C@H]4C[C@@H](O)[C@@H](OCCOCCN)NC(=O)[C@@H]5[C@@H](O)[C@@H](C)CN5C(=O)[C@H]([C@@H](C)O)NC(=O)[C@H]([C@H](O)[C@@H](O)c5ccc(O)cc5)NC(=O)[C@@H]5C[C@@H](O)CN5C(=O)[C@H]([C@@H](C)O)NC4=O)cc3)cc2)cc1 ; CCCCCOc1ccc(-c2ccc(-c3ccc(C(=O)N[C@H]4C[C@@H](O)[C@@H](OCCOCCN)NC(=O)[C@@H]5[C@@H](O)[C@@H](C)CN5C(=O)[C@H]([C@@H](C)O)NC(=O)[C@H]([C@H](O)[C@@H](O)c5ccc(O)c(N)c5)NC(=O)[C@@H]5C[C@@H](O)CN5C(=O)[C@H]([C@@H](C)O)NC4=O)cc3)cc2)cc1]

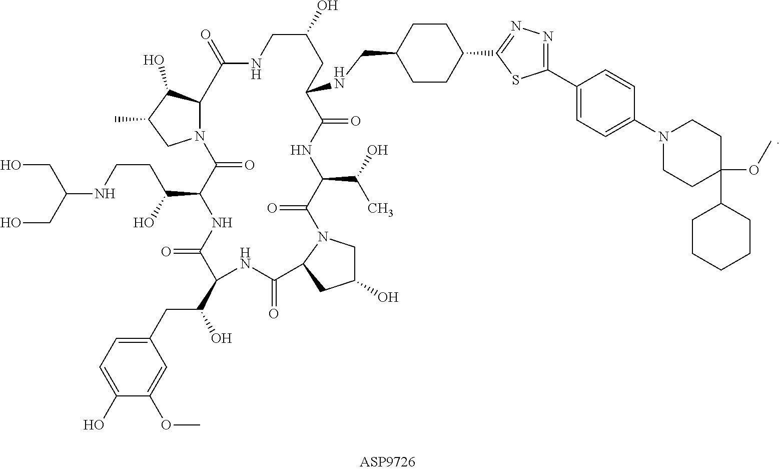 [SMILES: COc1cc(C[C@@H](O)[C@@H]2NC(=O)[C@@H]3C[C@@H](O)CN3C(=O)[C@H]([C@@H](C)O)NC(=O)[C@@H](NC[C@H]3CC[C@H](c4nnc(-c5ccc(N6CCC(OC)(C7CCCCC7)CC6)cc5)s4)CC3)C[C@@H](O)CNC(=O)[C@@H]3[C@@H](O)[C@@H](C)CN3C(=O)[C@H]([C@H](O)CCNC(CO)CO)NC2=O)ccc1O]

Yet other exemplary β-1,3-glucan synthase inhibitors include, without limitation:

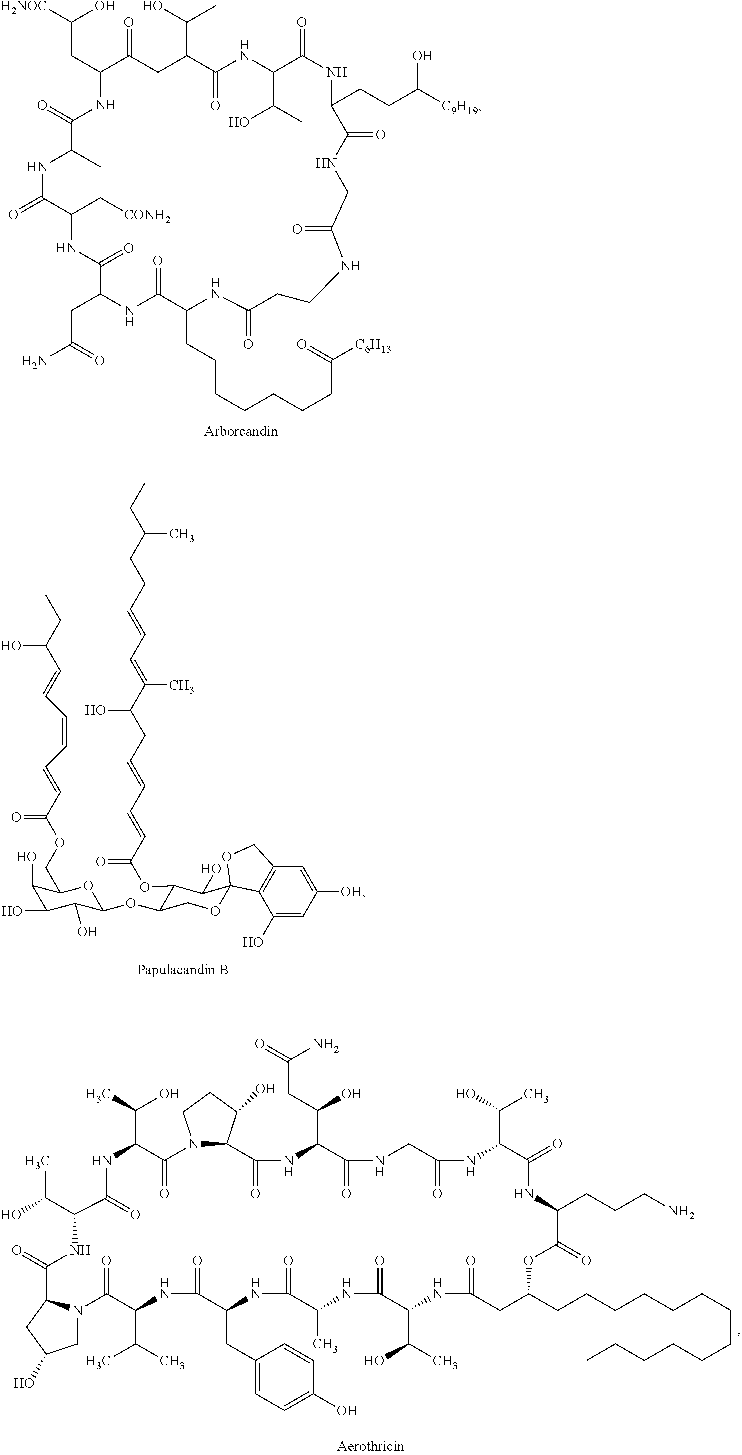

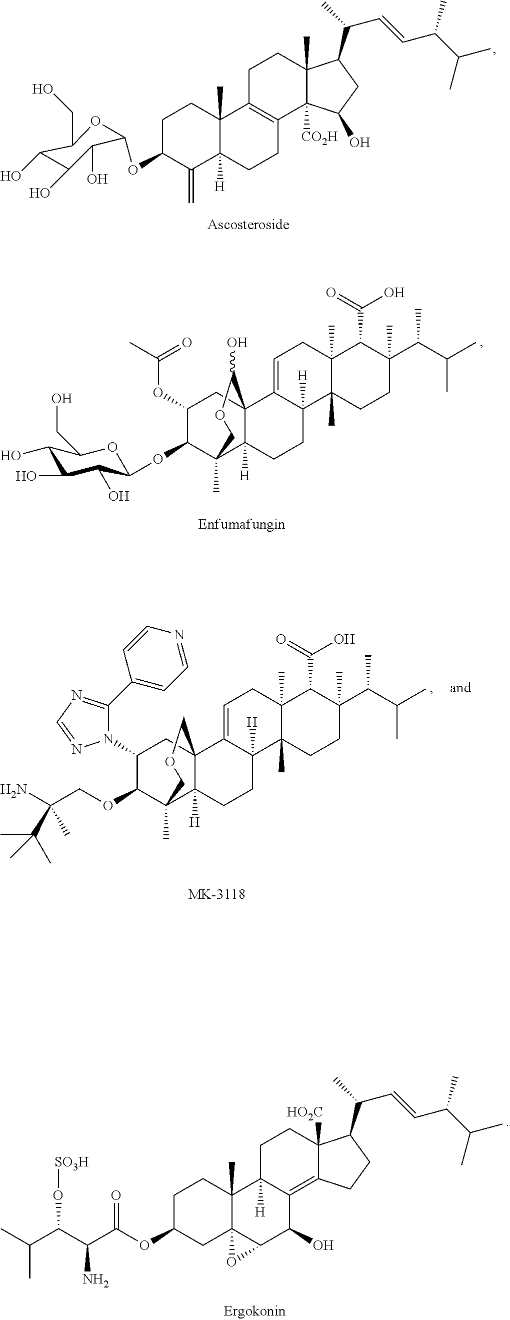

or amine derivatives thereof.

In some embodiments of the invention, any of the above compounds could be utilized as a β-1,3-glucan synthase inhibitor. The conjugate may be synthesized using an available amine in the β-1,3-glucan synthase inhibitor to form an amide bond with the C-terminus of a pathogen pattern recognition receptor ligand (e.g., a peptide of any of Formulas III and IV) or with the C-terminus of a linker (e.g., a linker of any of Formulas V-XVI). In cases where the β-1,3-glucan synthase inhibitor does not possess an amino group to allow conjugation to a pathogen pattern recognition receptor ligand or to a linker, the inhibitor may be derivatized using conventional chemical synthesis techniques that are well known in the art. In some embodiments, the inhibitor may be derivatized as illustrated by Example 30b in the synthesis of Compound 31.

In some embodiments, a conjugate may be formed by joining a pathogen pattern recognition receptor ligand bearing a carboxylate or activated carboxyl group to a primary or secondary amine present in the glucan synthase inhibitor via an amide bond as illustrated in Example 2 (Compound 1) or Example 3 (Compound 2). The glucan synthase inhibitor and pathogen pattern recognition receptor ligand may be joined via a linker by first joining the linker to the pathogen pattern recognition receptor ligand and activating the carboxyl group as an N-hydroxysuccinimide ester or other activated ester as illustrated by the preparation of Int-4 or Int-6 in Example 1 and then reacting the activated ester with a glucan synthase inhibitor as illustrated in Example 8 (Compound 7) and Example 15 (Compound 14). Alternatively, the glucan synthase inhibitor and pathogen pattern recognition receptor ligand may be joined via a bond by activating the carboxyl group of the pathogen pattern recognition receptor ligand as an N-hydroxysuccinimide ester or other activated ester as illustrated in Example 12 (Compound 11) or Example 13 (Compound 12).

In other embodiments, if an amine is not present in the glucan synthase inhibitor, it may be introduced by known methods before conjugation. For example, nitration of a phenyl group, followed by reduction of the nitro provides an amine suitable for conjugation via amide bond formation as illustrated in Example 30c (Compound 31). Amine functional groups may be introduced into a glucan synthase inhibitor molecule by dehydration of a primary amide to a nitrile followed by reduction to an amine or by appendage of an amine-containing tether by acid catalyzed exchange of a hemiaminal group with a protected ethanolamine moiety where both methods are illustrated in the preparation of Int-15 in Example 1.

### Pathogen Pattern Recognition Receptors

Pathogen pattern recognition receptors (PPRs) are receptors of the innate immune system. The innate immune system represents a defense mechanism that a host uses immediately or within several hours after exposure to an antigen which is non-specific to such antigen. Unlike adaptive immunity, innate immunity does not recognize every possible antigen. Instead it is designed to recognize a few highly conserved structures present in many different pathogens. The structures recognized are pathogen-associated molecular patters and include LPS from the gram-negative cell wall, peptidoglycan, lipotechoic acids from the gram-positive cell wall, the sugar mannose, fucose, N-acetyl glucosamine, bacterial DNA, N-formylmethionine found in bacterial proteins, double stranded RNA from viruses, and glucans from fungal cell walls.

Most body defense cells have pattern recognition receptors for these common pathogen associated molecular patterns. Consequently, there is an immediate response against the invading pathogen. Pathogen-associated molecular patterns can also be recognized by a series of soluble pattern recognition receptors in the blood that function as opsonins and initiate the complement pathways. Taken together, the innate immune system is thought to recognize approximately 10³ molecular patterns.

As used herein “PPRs,” refers to either surface PPRs or soluble PPRs. The cell surface PPRs can be subdivided into two functionally different classes: endocytic pattern recognition receptors and signaling pattern recognition receptors. Endocytic pattern recognition receptors are found on the surface of immune cells and promote the attachment of pathogens to phagocytes and their subsequent engulfment and destruction.

The exemplary PPRs include mannose receptors (MR), formyl peptide receptors (FPRs), toll-like receptors (TLRs), CD14, and nucleotide binding oligomerization domain proteins (NOD). Binding of ligands to these receptors also promotes the synthesis and secretion of intracellular regulatory molecules (immune modulating signals) such as cytokines that are crucial to initiating innate immunity and adaptive immunity.

Any PPR known in the art or described herein can be used in the compounds herein.

### Formyl Peptide Receptors

The formyl peptide receptor family belongs to the class of signaling PRR. FPRs are G-protein coupled receptors expressed primarily in neutrophils and some cells of macrophage or phagocyte lineage. The best characterized ligands for these receptors are peptides or protein fragments containing N-formyl methionine residues, a hallmark of proteins of prokaryotic origin. As such, these peptides serve as potent immunological homing signals for sites of bacterial infection, signaling several phases of neutrophil response and activation, including chemoattraction, stimulation of production and release of immunosignaling molecules (e.g., interleukins, cytokines), as well as degranulation, a cellular process that includes the production and release of both chemical (e.g., hydrogen peroxide and other reactive oxygen species) and enzymatic agents (e.g, elastase and other digestive enzymes) capable of mediating destruction of the foreign agent or pathogen.

In humans, five related FPR family members have been identified: formyl peptide receptor 1 (FPR1), FPR2, FPR3, formyl peptide receptor-like 1 (FPRL1), and FPRL2. A naturally occurring ligand for FPR is formyl-methionine-leucine-phenylalanine (fMLF).

The cellular response mediated by the formyl peptide receptor includes cellular polarization and transmigration, generation of superoxide 02 radicals through respiratory burst oxidase, degranulation and release of a variety of various degenerative enzymes, as well as phagocytosis. In some embodiments, compounds of the invention interact with FPR and induce at least one of the above cellular responses.

### Formyl Peptide Receptor Ligands

Formyl peptide receptor ligands are known in the art. As noted above, a naturally occurring ligand for FPR is formyl-methionine-leucine-phenylalanine (fMLF). Several synthetic mimetics of fMLF have been shown to induce an immune response. Exemplary FPR ligands include, but are not limited to peptides comprising an amino acid residue having the formula:

R¹⁴—X1-X2-X3-X4-X5-X6-X7-X8-X9 Formula III

where X9 is any amino acid; X1-X8 are any amino acid or absent; R¹⁴ is hydrogen or

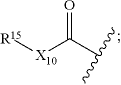

where X₁₀ is a bond, NH, or O; and R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic; where if R¹⁴ is hydrogen X1 is not absent (e.g., X1 and X2 are not absent). Exemplary peptides of Formula III include peptides having the structure:

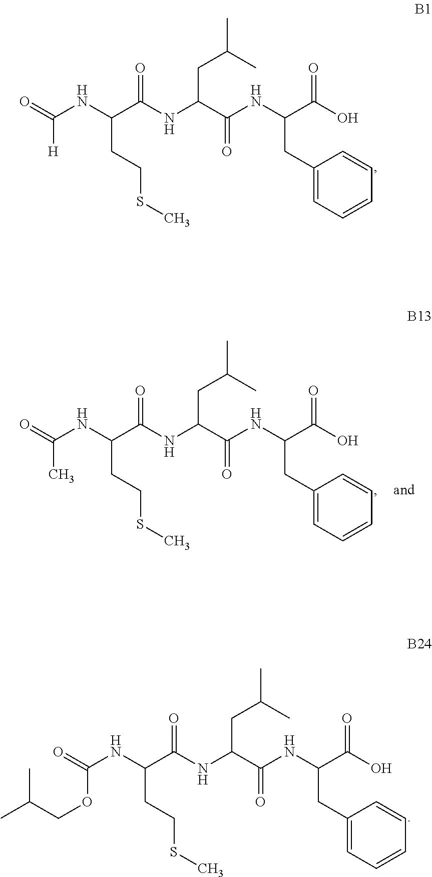 [SMILES: CSCCC(NC=O)C(=O)NC(CC(C)C)C(=O)NC(Cc1ccccc1)C(=O)O ; CSCCC(NC(C)=O)C(=O)NC(CC(C)C)C(=O)NC(Cc1ccccc1)C(=O)O ; CSCCC(NC(=O)OCC(C)C)C(=O)NC(CC(C)C)C(=O)NC(Cc1ccccc1)C(=O)O]

Methionylnorleucyl-leucyl-phenylalanyl-phenylalanine (MNIeLFF) as an FPR lignad is shown in J Exp Med 180, 2191-7 (1994) herein incorporated by reference. fMLX-OMe as FPR ligands are shown in Bioorg Med Chem, 21, 668-675 (2013), herein incorporated by reference, wherein X—OMe has the structure:

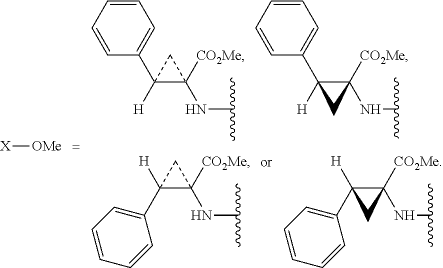

N-terminal oximic or formyl hydrazonic containing peptides as FPR ligands are shown in J. Peptide Res., 2000, 55, 102-109, herein incorporated by reference, have the structure:

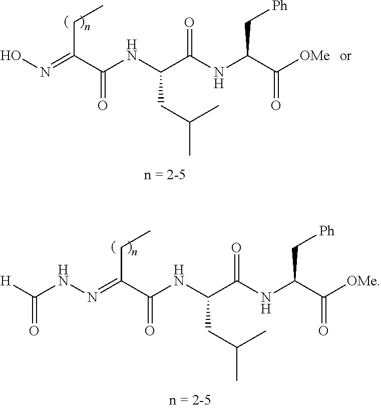

β-Peptido sulfonamides as FPR ligands are shown in IL FARMACO 59 (2004) 953-963, herein incorporated by reference, including Boc-Met-Tau-Phe-OMe; HCO-Met-Tau-Phe-OMe; Met-Tau-Phe-OMe, Boc-Met-β³-HLeu-Phe-OMe; HCO-Met-β³-HLeu-Phe-OMe; Boc-Met-Leu-ψ[CH₂SO₂]-Phe-OMe; HCO-Met-Leu-ψ[CH₂SO₂]-Phe-OMe; Boc-Met-Tau-Phe-Phe-OMe; HCO-Met-Tau-Phe-Phe-OMe. 3V,5V-dimethylphenyl-ureido containing peptides as FPR ligands are shown in European Journal of Pharmacology 436 (2002) 187-196, herein incorporated by reference, including 3V,5V-dimethylphenyl-ureido-Phe-D-Leu-Phe-D-Leu-Phe-olo; 3V,5V-dimethylphenyl-ureido-Phe-D-Leu-Phe-D-Leu-Glu; 3V,5V-dimethylphenyl-ureido-Phe-D-Leu-Phe-D-Leu-Tyr. Proline containing peptides as FPR ligands are shown in Bioorganic & Medicinal Chemistry 14 (2006) 2253-2265, herein incorporated by reference, having the structure:

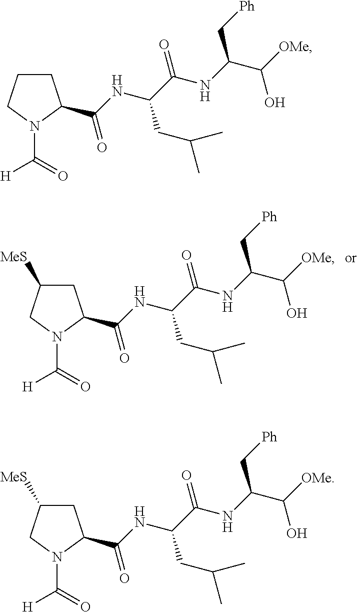 [SMILES: COC(O)[C@H](Cc1ccccc1)NC(=O)[C@H](CC(C)C)NC(=O)[C@@H]1CCCN1C=O ; COC(O)[C@H](Cc1ccccc1)NC(=O)[C@H](CC(C)C)NC(=O)[C@@H]1C[C@H](SC)CN1C=O ; COC(O)[C@H](Cc1ccccc1)NC(=O)[C@H](CC(C)C)NC(=O)[C@@H]1C[C@@H](SC)CN1C=O]

Peptides as FPR ligands are also shown in Amino Acids (2008) 35 329-338, herein incorporated by reference, having the structure:

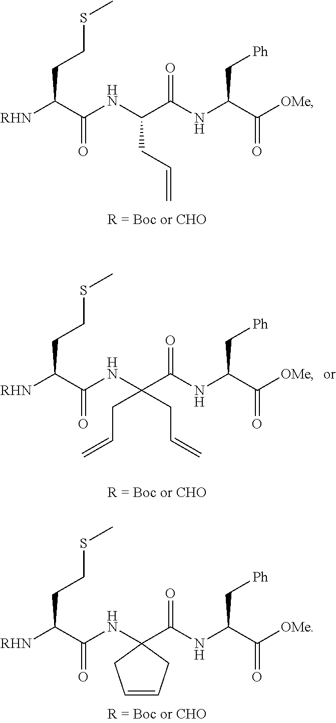

Other peptides as FPR ligands are shown in Pept Res. 6, 298-307 (1993), herein incorporated by reference, having the structure:

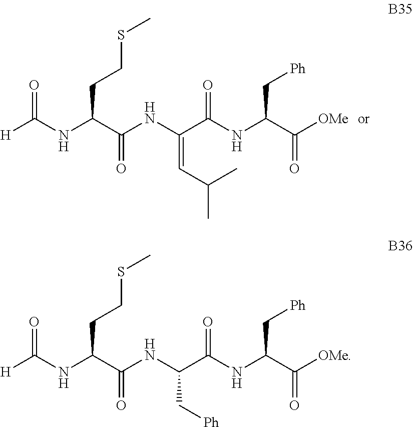 [SMILES: COC(=O)[C@H](Cc1ccccc1)NC(=O)/C(=C\C(C)C)NC(=O)[C@H](CCSC)NC=O ; COC(=O)[C@H](Cc1ccccc1)NC(=O)[C@H](Cc1ccccc1)NC(=O)[C@H](CCSC)NC=O]

Fluoromodified peptides as FPR ligands are shown in J. Peptide Sci. 10, 67-81 (2004), herein incorporated by reference, HCO-Met-(S)-DfeGly-Phe-NH₂; HCO-Met-(R)-DfeGly-Phe-NH₂; HCO-Met-(S)-(αTfm)Ala-Phe-NH₂; HCO-Met-(R)-(αTfm)Ala-Phe-NH₂; HCO-Met-Aib-Phe-NH₂; HCO-Met-(R)-(aDfm)Ala-Phe-NH₂. Further peptides as FPR ligands are shown in Biochemistry 19, 2404-2410 (1980), herein incorporated by reference, including HCONIe-Leu-Phe-OH; HCO-Nva-Leu-Phe-OH; HCO-Hep-Leu-Phe-OH; HCO-Ile-Leu-Phe-OH; HCO-Met-Ala-Leu-Phe-OH; HCO-Met-Leu-Phe-Lys-OH. More peptides as FPR ligands are shown in Eur. J. Immunol. 35, 2486-2495 (2005), herein incorporated by reference. Hybrid α/β peptides as FPR ligands are shown in Amino Acids 30, 453-459 (2006), herein incorporated by reference, including those having the structure:

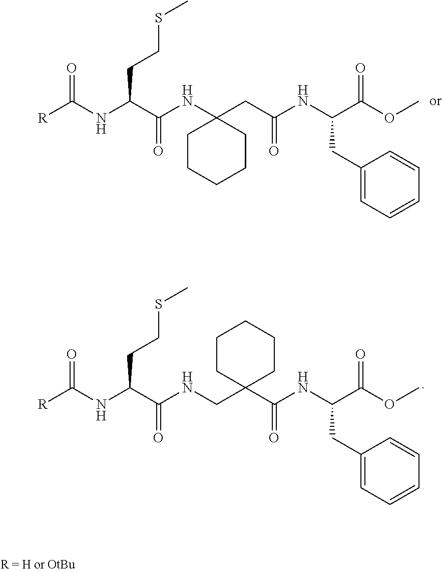

Hybrid αβ3-Peptides as FPR ligands are shown in J. Peptide Sci. 10, 510-523 (2004), herein incorporated by reference, including those having the structure:

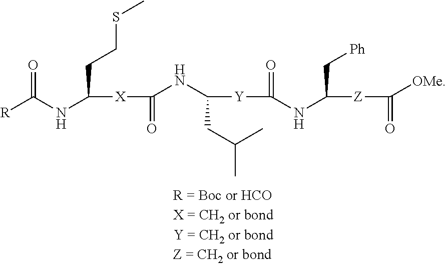

Isopeptide bond containing peptides as FPR ligands are shown in J. Peptide Res., 59, 283-291 (2002), herein incorporated by reference. Peptides containing constrained mimics of phenylalanine as FPR ligands are shown in Arch. Pharm. Pharm. Med. Chem. 331, 170-176 (1998), herein incorporated by reference, including those having the structure:

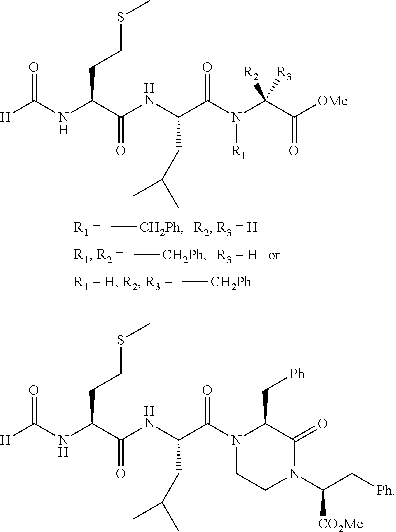

Other proline containing peptides as FPR ligands are shown in Bioorganic & Medicinal Chemistry 17, 251-259 (2009), herein incorporated by reference. More peptides as FPR ligands are shown in J. Peptide Sci. 7, 56-65 (2002), herein incorporated by reference, including those having the structure:

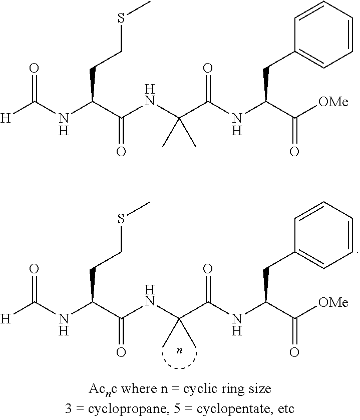 [SMILES: COC(=O)[C@H](Cc1ccccc1)NC(=O)C(C)(C)NC(=O)[C@H](CCSC)NC=O ; COC(=O)[C@H](Cc1ccccc1)NC(=O)C(C)(C)NC(=O)[C@H](CCSC)NC=O]

Pseudopeptide as FPR ligands are shown in J. Peptide Res. 58, 56-66 (2001), herein incorporated by reference, including those with the structure:

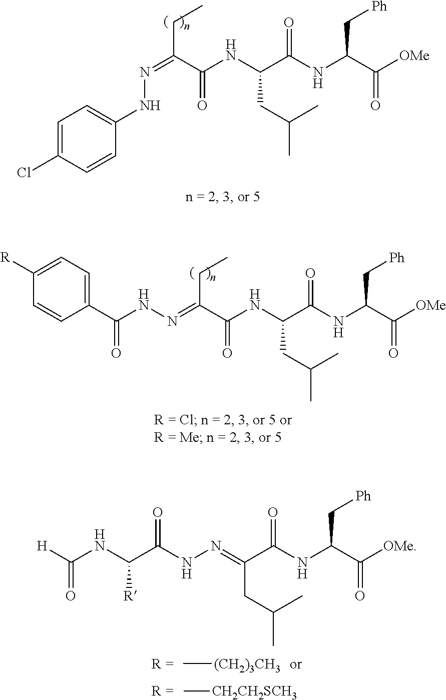

Further peptides as FPR ligands are shown in Bioorganic Chemistry 34, 298-318 (2006), herein incorporated by reference. Other peptides as FPR ligands are shown in II Farmaco 58, 1121 (2003), herein incorporated by reference, including HCO-Met-β-Ala-Phe-OMe, Boc-Met-Tau-Phe-OMe, and HCO-Met-Tau-Phe-OMe. Peptides with alkyl spacers as FPR ligands are shown in II Farmaco 56, 851-858 (2001), herein incorporated by reference, including those having the structure:

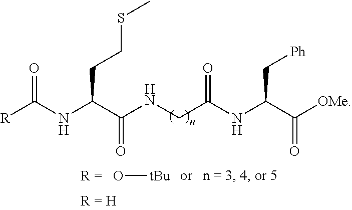

Tetrapeptides as FPR ligands are shown in Amino Acids 37, 285-295 (2009), herein incorporated by reference, including those having the structure:

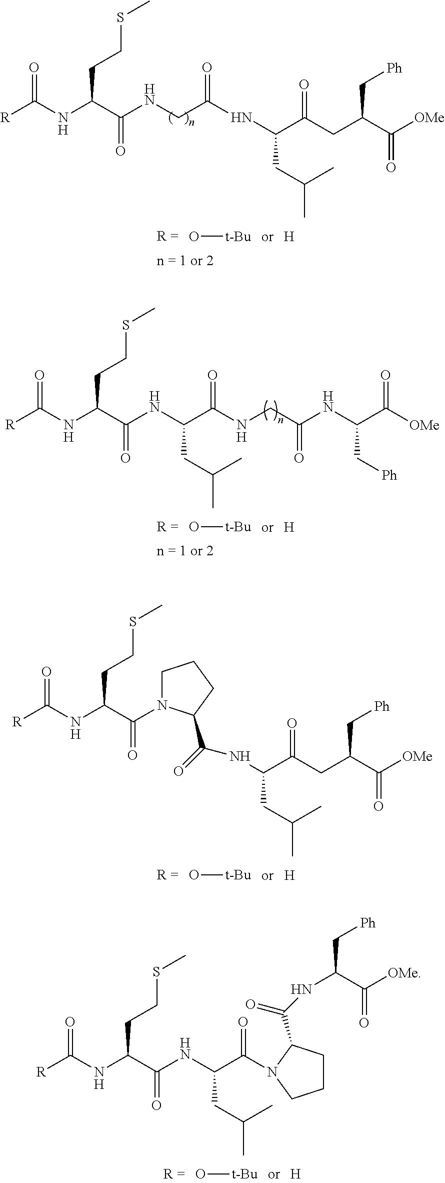

Peptides containing a substituted glycine as FPR ligands are shown in Eur. J. Med. Chem. 27, 19-26 (1992), herein incorporated by reference, including those having the structure:

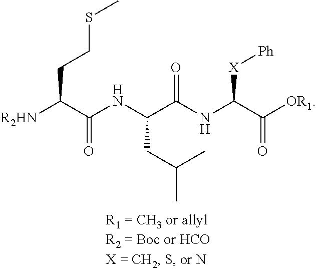

Peptides with a para-substituted phenylalanine as FPR ligands are shown in J. Pept. Sci. 18, 418-426 (2012), herein incorporated by reference. Other peptides as FPR ligands are shown in Pept Sci, 269-272 (2002), herein incorporated by reference. Centrally modified pseudopeptides as FPR ligands are shown in Amino Acids 33, 477-487 (2007), herein incorporated by reference. Further peptides as FPR ligands are shown in TRENDS in Immunology Vol. 23 No. 11 Nov. 2002, herein incorporated by reference. More peptides as FPR ligands are shown in Cytokine & Growth Factor Reviews 17 (2006) 501-519, herein incorporated by reference.

### Linkers

In some embodiments, the compounds of the invention include a linker. The linker component of the compound is, at its simplest, a bond, but typically provides a linear, cyclic, or branched molecular skeleton having pendant groups covalently linking two moieties.

Thus, linking of the two moieties is achieved by covalent means, involving bond formation with one or more functional groups located on either moiety. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of the two moieties may be effected using a linker that contains reactive moieties capable of reaction with such functional groups present in either moiety. For example, an amine group of a moiety may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X═Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in either moiety may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of one moiety with a reactive chemical group of the other without introducing additional linking material may, if desired, be used with the compounds herein.

More commonly, however, the linker will include two or more reactive moieties, as described above, connected by a spacer element. The presence of such a spacer permits bifunctional linkers to react with specific functional groups within either moiety, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the two moieties.

Spacer elements in the linker typically consist of linear or branched chains and may include a C₁₋₁₀ alkylene, C₂₋₁₀ alkenylene, C₂₋₁₀ alkynylene, C₂₋₆ heterocyclylene, C₆₋₁₂ arylene, C₇₋₁₄ alkarylene, C₃₋₁₀ alkheterocyclylene, C₂-C₁₀₀ polyethylene glycolene, or C₁₋₁₀ heteroalkylene.

In some instances, the linker is described by Formula V.

Examples of homobifunctional linkers useful in the preparation of conjugates herein include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

In certain embodiments, the pathogen pattern recognition receptor ligand or (b) is attached to said linker via an amide bond (e.g., at the C-terminus of a chemotactic peptide) and said inhibitor of β-1,3-glucan synthase or (a) is attached to said linker via an amide bond (e.g., at an R¹¹ of the inhibitor of β-1,3-glucan synthase, e.g., at R⁴, R⁶, or R⁹ of either of Formulas I and II).

### Treatment of Fungal Infections

Compositions and methods for treating or preventing a disease or condition associated with a fungal infection include administering a compound herein. Compounds herein may be administered by any appropriate route for treatment or prophylactic treatment of a disease or condition associated with a fungal infection. These may be administered to humans, domestic pets, livestock, or other animals with a pharmaceutically acceptable diluent, carrier, or excipient. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

The compounds herein can be used to treat, for example, invasive aspergillosis, pulmonary aspergillosis, intra-abdominal abscess, peritonitis, a pleural cavity infection, esophagitis, candidemia, and invasive candidiasis.

The invention features methods of treating a fungal infection in a subject by administering to the subject a compound or pharmaceutical composition herein in an amount sufficient to treat the infection. In particular embodiments, the pharmaceutical composition is administered intravenously or topically. The pharmaceutical composition can be administered to treat a blood stream infection, tissue infection (e.g., lung, kidney, or liver infection) in the subject, or any other type of infection described herein. The fungal infection being treated can be an infection selected from, invasive aspergillosis, pulmonary aspergillosis, intra-abdominal abscess, peritonitis, a pleural cavity infection, esophagitis, candidemia, and invasive candidiasis. In certain embodiments, the infection being treated is an infection by Candida albicans, C. parapsilosis, C. glabrata, C. guilliermondii, C. krusei, C. lusitaniae, C. tropicalis, Aspergillus fumigatus, A. flavus, A. terreus. A. niger, A. candidus, A. clavatus, or A. ochraceus.

Methods for the prophylactic treatment of a fungal infection in a subject by administering to the subject a compound or pharmaceutical composition herein. In particular embodiments, the pharmaceutical composition is administered at least once over a period of 1-30 days (e.g., 1, 2, 3, 4, or 5 times over a period of 1-30 days). For example, the methods herein can be used for prophylatic treatment in subjects being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised subjects (e.g., subjects with cancer, with HIV/AIDS, or taking immunosuppressive agents), or in subjects undergoing long term antibiotic therapy.

A method of stabilizing, or inhibiting the growth of fungi, or killing fungi includes contacting the fungi or a site susceptible to fungal growth with a compound or pharmaceutical composition herein, or a pharmaceutically acceptable salt thereof.

### Pharmaceutical Compositions

For use as treatment of human and animal subjects, the compounds herein can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The compounds described herein may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, gastrointesitnal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, the compounds described herein may be used alone, as mixtures of two or more compounds or in combination with other pharmaceuticals. An example of other pharmaceuticals to combine with the compounds described herein would include pharmaceuticals for the treatment of the same indication. Another example of a potential pharmaceutical to combine with the compounds described herein would include pharmaceuticals for the treatment of different yet associated or related symptoms or indications. Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The compounds herein may be prepared and used as pharmaceutical compositions comprising an effective amount of a compound described herein and a pharmaceutically acceptable carrier or excipient, as is well known in the art. In some embodiments, the composition includes at least two different pharmaceutically acceptable excipients or carriers.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds herein. Suitable forms include syrups, capsules, and tablets, as is understood in the art.

Each compound of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations for oral use include tablets containing the active ingredient(s) in a mixture with nontoxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the combination herein will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

Administration of each drug in a combination therapy, as described herein, can, independently, be one to four times daily for one day to one year, and may even be for the life of the patient. Chronic, long-term administration may be indicated.

The following Examples are intended to illustrate the synthesis of a representative number of compounds and the use of these compounds for the induction of chemotaxis and antifungal activity. Accordingly, the Examples are intended to illustrate but not to limit the invention. Additional compounds not specifically exemplified may be synthesized using conventional methods in combination with the methods described herein.

### EXAMPLES

### General Methods

Analytical HPLC was performed using the following column and conditions: Atlantis T3, 3 micron, 3.0×75 mm; 50° C., water/CH₃CN+0.1% formic acid, 5 to 95% CH₃CN over 11 min+2 min hold. Preparative HPLC was performed using the following column: Agilent ZORBAX SB-CN, 7 μm, 21.2×250 mm, CH₃CN/H₂O/0.1% Acetic Acid various linear gradients as necessary at 20 mL/min.

Rapid LC: A Waters BEH C18, 3.0×30 mm, 1.7 μm, was used at a temperature of 50° C. and at a flow rate of 1.5 mL/min, 2 μL injection, mobile phase: (A) water with 0.1% formic acid and 1% acetonitrile, mobile phase (B) methanol with 0.1% formic acid; retention time given in minutes. Method details: (I) runs on a Binary Pump G1312B with UV/Vis diode array detector G1315C and Agilent 6130 mass spectrometer in positive and negative ion electrospray mode with UV PDA detection with a gradient of 15-95% (B) in a 2.2 min linear gradient (11) hold for 0.8 min at 95% (B) (111) decrease from 95-15% (B) in a 0.1 min linear gradient (IV) hold for 0.29 min at 15% (B).

Polar Stop-Gap: An Agilent Zorbax Bonus RP, 2.1×50 mm, 3.5 μm, was used at a temperature of 50° C. and at a flow rate of 0.8 mL/min, 2 μL injection, mobile phase: (A) water with 0.1% formic acid and 1% acetonitrile, mobile phase (B) methanol with 0.1% formic acid; retention time given in minutes. Method details: (I) runs on a Binary Pump G1312B with UV/Vis diode array detector G1315C and Agilent 6130 mass spectrometer in positive and negative ion electrospray mode with UV-detection at 220 and 254 nm with a gradient of 5-95% (B) in a 2.5 min linear gradient (11) hold for 0.5 min at 95% (B) (111) decrease from 95-5% (B) in a 0.1 min linear gradient (IV) hold for 0.29 min at 5% (B).

NMR Spectra were acquired on either of two instruments: (1) Agilent (formerly Varian) Unitylnova 400 MHz NMR spectrometer equipped with a 5 mm Automation Triple Broadband (ATB) probe. The ATB probe was simultaneously tuned to 1H, 19F and 13C. (2) Agilent (formerly Varian) Unitylnova 500 MHz NMR spectrometer. Several NMR probes are available for use with the 500 MHz NMR spectrometer, including both 3 mm and 5 mm 1H13C15N probes and a 3 mm X1H19F NMR probe (usually X is tuned to 13C). For typical 1H NMR spectra, the pulse angle was 45 degrees, 8 scans were summed and the spectral width was 16 ppm (−2 ppm to 14 ppm). A total of 32768 complex points were collected during the 5.1 second acquisition time, and the recycle delay was set to 1 second. Spectra were collected at 25° C. 1H NMR Spectra are typically processed with 0.3 Hz line broadening and zero-filling to 131072 points prior to Fourier transformation.

For some of the preparations the following general conditions were utilized.

Preparative HPLC was performed using the following: Teledyne Isco HP C18, 50 g column. Eluent: CH₃CN/H₂O/0.1% formic Acid or 0.1% trifluoro acetic acid; various linear gradients as necessary at 40 mL/min on the Isco Combiflash Rf LC unit. UV Detection at 220 and 254 nm.

OR Luna 5 micron C18, 100 A°, AXIA 100×30 mm. Eluent: CH₃CN/H₂O/0.1% formic Acid or 0.1% trifluoro acetic acid; various linear gradients as necessary at 25 mL/min on the Gilson System; 215 Liquid Handler, Gilson UV-VIS 155, Gilson 305 pump and Detector. UV detection at 220 and 254 nm.

Analytical LC/MS: High resolution liquid chromatography mass spectrometry (HRES-LC/MS) was performed using a Waters Q-TOF Premier mass spectrometer with an electrospray probe coupled with an Agilent 1100 HPLC system with a diode array detector set to collect from 190 nm to 400 nm. A gradient of 0.1% formic acid in water (A) and 0.1% formic acid in acetonitrile (B) was run from 15% B to 95% over 15 min using a 100×3.0 2.6 u Phenomenex Kinetex C18 column at 30° C.

Liquid chromatography mass spectrometry was performed using an Agilent 6120 mass spectrometer an electrospray probe coupled coupled with an Agilent 1100 HPLC system with a variable wavelength detector set to either 220 nm or 254 nm. A gradient of 0.1% formic acid in water (A) and 0.1% formic acid in acetonitrile (B) was run from 15% B to 99% over 3.5 min using a 50×3.0 2.6 u Phenomenex Kinetex C18 column at 30° C.

1H-NMR spectra were acquired on a Bruker 300 MHz system using a 5 mm QNP probe.

Synthesis of peptide fragment coupling partners: In some cases, discreet coupling partners were isolated and purified. In others, the couplings partners were prepared and used directly without isolation. The description of the isolated compounds is described in this section, while the coupling partners that were used directly are embedded within the examples of final compounds.

Some peptides or derivatized peptides were obtained from a Contract Research Organization or other commercial source. In general, peptides are considered readily available from such sources by standard peptide synthesis procedures.

Compounds herein may be made using synthetic methods known in the art, including procedures analogous to those disclosed below.

### Example 1

### Synthesis of Intermediates

### Synthesis of fMLF-OSu (Int-1)

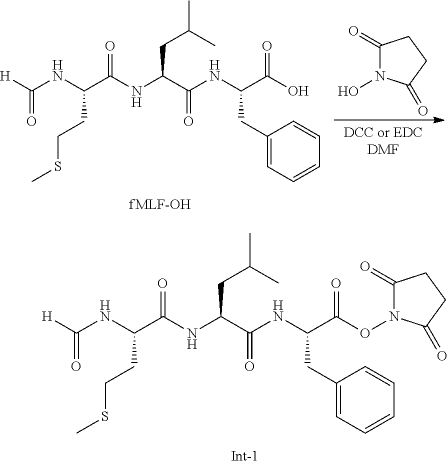 [SMILES: CSCC[C@H](NC=O)C(=O)N[C@@H](CC(C)C)C(=O)N[C@@H](Cc1ccccc1)C(=O)O ; O=C1CCC(=O)N1O ; CSCC[C@H](NC=O)C(=O)N[C@@H](CC(C)C)C(=O)N[C@@H](Cc1ccccc1)C(=O)ON1C(=O)CCC1=O]

### Procedure 1

To a mixture of fMLF-OH (0.5 g, 1.0383 mmol), N-hydroxysuccinimide (0.12 g, 1.0 eq) in dry DMF (16 mL) was added DCC (0.215 g, 1.0 eq) at 0° C. The reaction mixture was stirred at 0° C. for 8 h and then at RT for 12 h. The precipitate was filtered, washed with THF (2×5 mL) and the combined organic solution was concentrated under reduced pressure. Purification by flash chromatography (DCM to EtOAc) afforded 0.417 g of the desired N-hydroxysuccinimdyl ester fMLF-OSu (Int-1) as a white solid. ¹H-NMR (400 MHz, DMSO-d₆) δ 8.68 (d, 1H), 8.24 (d, 1H), 8.04 (d, 1H), 8.00 (s, 1H), 7.24-7.33 (m, 5H), 4.87-4.92 (m, 1H), 4.31-4.42 (m, 2H), 3.20-3.25 (m, 1H), 3.06-3.12 (m, 1H), 2.35-2.39 (m, 2H), 2.00 (s, 3H), 1.67-1.85 (m, 2H), 1.52-1.58 (m, 1H), 1.39-1.42 (m, 2H), 0.85 (d, 3H, J=8.0 Hz), 0.80 (d, 3H, J=8.0 Hz); LC/MS, 535.1 [M+H]⁺.

### Procedure 2

To a solution of (N-formyl)-MLF-OH (1.748 g, 4.00 mmol), and N-hydroxy-succinimide (0.506 g, 4.40 mmol), in DMF (12 mL) was added EDC (0.683 g, 4.40 mmol). Reaction progress was monitored by LCMS. The mixture was stirred at room temperature overnight. The reaction was diluted with ethyl acetate (50 mL), washed with water (4×15 mL), sodium bicarbonate aq. (1×15 mL), and dried with sodium sulfate, yielding 2.26 g of Int-1 as a glassy foam. LC/MS, 535.2 [M+H]⁺ calculated 535.2.

### Synthesis of fMLF-β-Ala-OSu (Int-2)

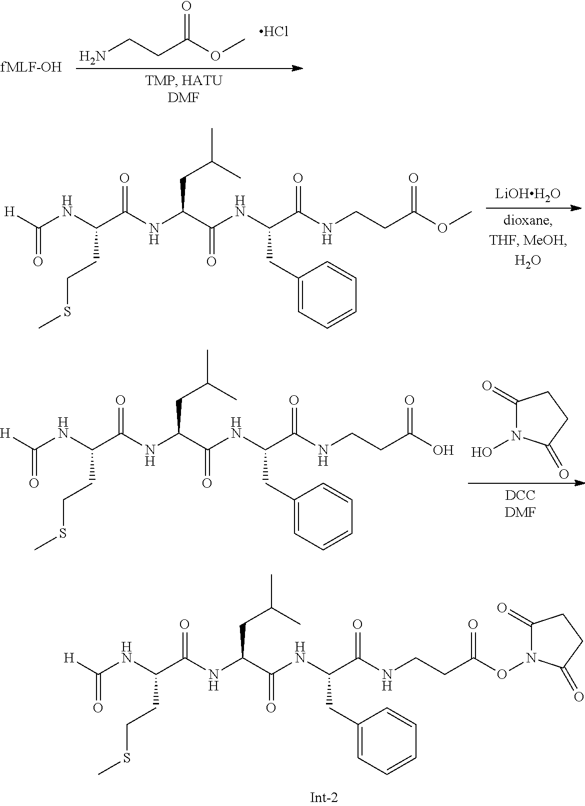

### Step a. Synthesis of fMLF-β-Ala-OMe

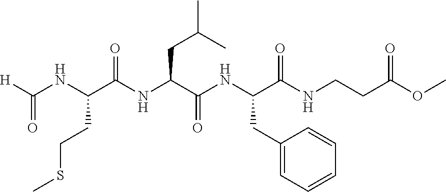 [SMILES: COC(=O)CCNC(=O)[C@H](Cc1ccccc1)NC(=O)[C@H](CC(C)C)NC(=O)[C@H](CCSC)NC=O]

A solution of fMLF-OH (0.6 g, 1.37 mmol) and beta-alanine methyl ester hydrochloride (0.19 g, 1.37 mmol) in DMF (11 mL) was added TMP (0.362 mL, 2.74 mmol) and HATU (0.52 g, 1.37 mmol) at 0° C. and the resulting mixture was stirred at 0° C. for 1 h and at room temperature for 1.5 h under nitrogen. The reaction mixture was diluted with EtOAc (75 mL), washed with 1.0 N HCl (2×15 mL), saturated NaHCO₃ (2×15 mL), saturated NaCl (2×15 mL), and dried over anhydrous sodium sulfate and then concentrated under reduced pressure. The crude product was triturated with diethyl ether and then dried under vacuum to yield 611 mg of the desired product. ¹H-NMR (400 MHz, DMSO-d₆) δ 8.28 (d, 1H), 8.05 (d, 1H), 8.01 (s, 1H), 7.978 (t, 1H), 7.91 (d, 1H), 7.16-7.26 (m, 5H), 4.38-4.44 (m, 2H), 4.22-4.28 (m, 1H), 3.59 (s, 3H), 3.17-3.30 (m, 2H), 2.78-2.95 (m, 2H), 2.36-2.42 (m, 4H), 2.01 (s, 3H), 1.69-1.90 (m, 2H), 1.47-1.56 (m, 1H), 1.33-1.42 (m, 2H), 0.85 (d, 3H, J=8.0 Hz), 0.80 (d, 3H, J=8.0 Hz); LC/MS, 523.2 [M+H]⁺.

### Step b. Synthesis of fMLF-β-Ala-OH

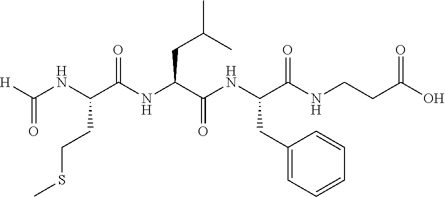 [SMILES: CSCC[C@H](NC=O)C(=O)N[C@@H](CC(C)C)C(=O)N[C@@H](Cc1ccccc1)C(=O)NCCC(=O)O]

The starting material, fMLF-β-Ala-OMe (0.6 g, 1.148 mmol) was dissolved in a solvent mixture of dioxane/THF/MeOH/water (1:1:1:1, v/v/v/v, 40 mL) and stirred at 0° C. Lithium hydroxide monohydrate (90 mg, 2.18 mmol) was added and the reaction mixture was stirred at 0° C. for 6 h. The reaction was quenched with acetic acid (0.2 mL) and silica gel (25 g) was added. The solvents were evaporated under reduced pressure and the powder was loaded onto 40 g silica gel cartridge, eluting with 2 to 10% methanol in DCM, to yield 0.37 g of the acid as a white solid. ¹H-NMR (400 MHz, DMSO-d₆) δ 12.22 (bs, 1H), 8.28 (d, 1H), 8.05 (d, 1H), 8.01 (s, 1H), 7.96 (t, 1H), 7.90 (d, 1H), 7.15-7.25 (m, 5H), 4.38-4.45 (m, 2H), 4.22-4.27 (m, 1H), 3.14-3.28 (m, 2H), 2.91-2.96 (m, 1H), 2.78-2.83 (m, 1H), 2.37-2.42 (m, 2H), 2.28-2.31 (m, 2H), 2.01 (s, 3H), 1.81-1.88 (m, 1H), 1.69-1.78 (m, 1H), 1.49-1.56 (m, 1H), 1.35-1.40 (m, 2H), 0.85 (d, 3H, J=8.0 Hz), 0.80 (d, 3H, J=8.0 Hz); LC/MS, 509.1 [M+H]⁺.

### Step c. Synthesis of fMLF-β-Ala-OSu

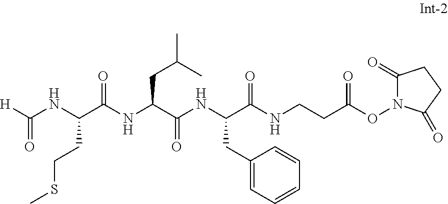 [SMILES: CSCC[C@H](NC=O)C(=O)N[C@@H](CC(C)C)C(=O)N[C@@H](Cc1ccccc1)C(=O)NCCC(=O)ON1C(=O)CCC1=O]

A mixture of f-MLF-β-Ala-OH (0.35 g, 0.688 mmol) and N-hydroxysuccinimide (0.084 g, 0.688 mmol) in DMF (10 mL) was added DCC (0.15 g, 0.688 mmol) at 0° C. The reaction mixture was stirred at 0° C. for 1 h and then at RT overnight. The precipitate was filtered, washed with EtOAc (2×5 mL) and the filtrate was diluted with EtOAc (100 mL) and washed with water (3×30 mL). Silica gel (10 g) was added and the combined organic solution was concentrated under reduced pressure. The crude product was purified by flash column chromatography (0 to 10% MeOH/DCM). Fractions containing the desired product were pooled, concentrated, and the residue was triturated with MTBE (2×10 mL) to afford 250 mg of the desired N-hydroxysuccinimdyl ester as a white solid. ¹H-NMR (400 MHz, DMSO-d₆) δ 8.28 (d, 1H), 8.16 (t, 1H), 8.05 (d, 1H), 8.01 (s, 1H), 7.93 (d, 1H), 7.16-7.27 (m, 5H), 4.38-4.46 (m, 2H), 4.23-4.28 (m, 1H), 3.35-3.42 (m, 1H), 3.22-3.29 (m, 1H), 2.94-2.99 (m, 1H), 2.79-2.85 (m, 1H), 2.74-2.77 (t, 2H), 2.35-2.42 (m, 2H), 2.28-2.31 (m, 2H), 2.01 (s, 3H), 1.81-1.90 (m, 1H), 1.69-1.78 (m, 1H), 1.47-1.56 (m, 1H), 1.36-1.40 (m, 2H), 0.85 (d, 3H, J=8.0 Hz), 0.80 (d, 3H, J=8.0 Hz); LC/MS, 606.2 [M+H]⁺.

### Synthesis of ib-MLF-β-Ala-OSu (Int-3)

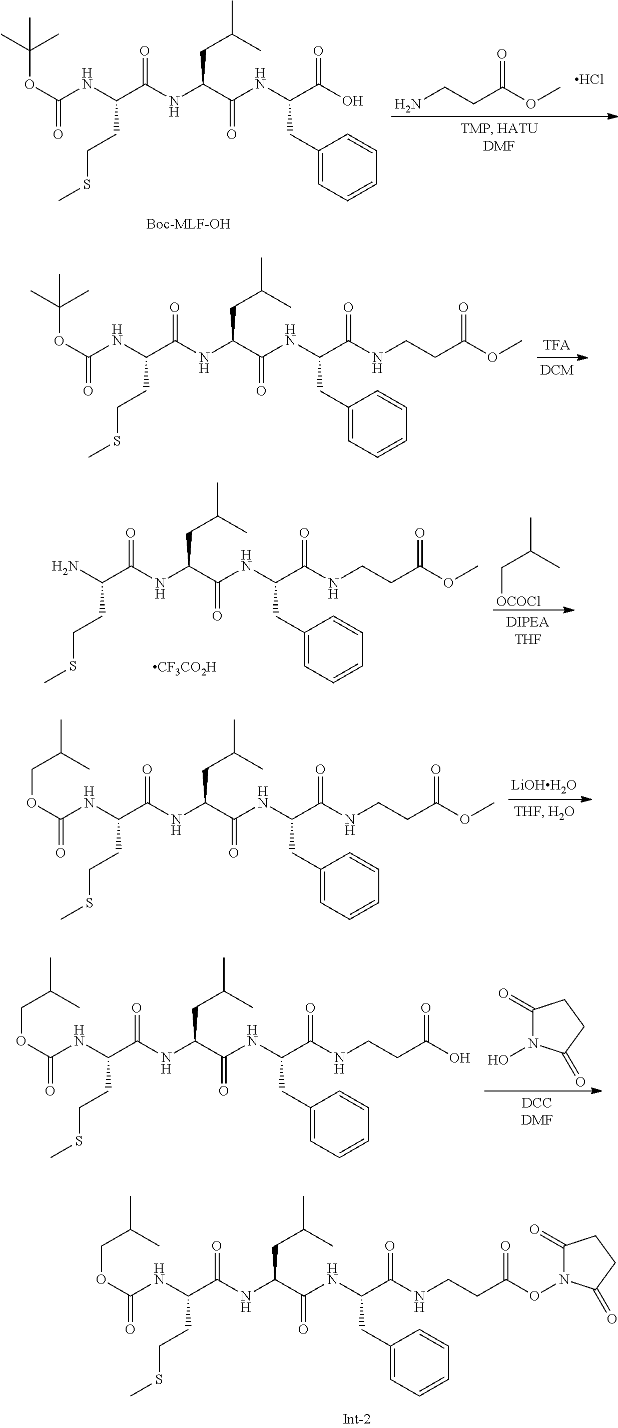

### Step a. Synthesis of Boc-MLF-β-Ala-OMe

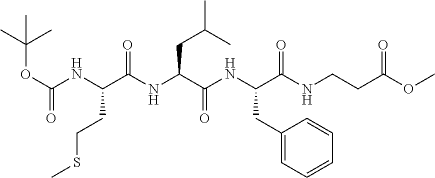 [SMILES: COC(=O)CCNC(=O)[C@H](Cc1ccccc1)NC(=O)[C@H](CC(C)C)NC(=O)[C@H](CCSC)NC(=O)OC(C)(C)C]

A solution of Boc-Met-Leu-Phe-OH (2.0 g, 3.92 mmol) and beta-alanine methyl ester hydrochloride (0.548 g, 3.92 mmol) in DMF (31 mL) was added TMP (1.04 mL, 7.85 mmol) and HATU (1.492 g, 3.92 mmol) at 0° C. and the resulting mixture was stirred at 0° C. for 1 h and at room temperature for 1.5 h under nitrogen. The reaction mixture was diluted with EtOAc (250 mL), washed with 1.0 N HCl (2×50 mL), saturated NaHCO₃ (2×50 mL), saturated NaCl (1×50 mL), dried over anhydrous sodium sulfate, and then concentrated under reduced pressure. The crude product was purified using silica gel column chromatography, eluting with 0 to 7% methanol in DCM, to yield 2.2 g of the desired product. ¹H-NMR (400 MHz, CDCl₃) δ 7.17-7.30 (m, 5H), 6.79-6.83 (m, 2H), 6.57 (bs, 1H), 5.32 (d, 1H), 4.63-4.69 (m, 1H), 4.34-4.39 (m, 1H), 4.20-4.26 (m, 1H), 3.66 (s, 3H), 3.46-3.54 (m, 1H), 3.34-3.42 (m, 1H), 3.10-3.16 (m, 1H), 2.99-3.05 (m, 1H), 2.53-2.57 (m, 2H), 2.34-2.51 (m, 2H), 2.11 (s, 3H), 2.01-2.08 (m, 1H), 1.87-1.96 (m, 1H), 1.54-1.61 (m, 2H), 1.45 (s, 9H), 0.85 (d, 3H, J=8.0 Hz), 0.80 (d, 3H, J=8.0 Hz); LC/MS, 595.2 [M+H]⁺.

### Step b. Synthesis of H₂N-Met-Leu-Phe-β-Ala-OMe TFA Salt

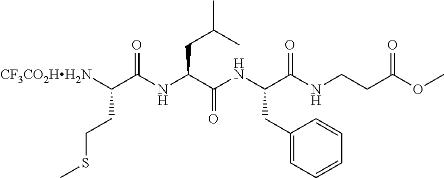

A solution of Boc-MLF-β-Ala-OMe (2.2 g, 3.7 mmol) in DCM (16 mL) was cooled to 0° C. and to this solution was added TFA (16 mL) and the resulting mixture was stirred at 0° C. for 1 h under nitrogen. The reaction mixture was concentrated under reduced pressure and the crude product was triturated with ether and then dried under vacuum to yield 2.5 g of the product as the TFA salt. This compound was used crude in subsequent reactions.

### Step c. Synthesis of ib-MLF-β-Ala-OMe

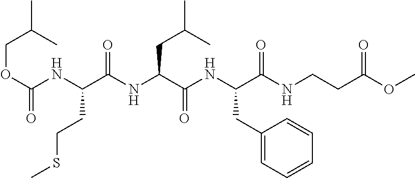 [SMILES: COC(=O)CCNC(=O)[C@H](Cc1ccccc1)NC(=O)[C@H](CC(C)C)NC(=O)[C@H](CCSC)NC(=O)OCC(C)C]

A suspension of H₂N-Met-Leu-Phe-β-Ala-OMe TFA salt (1.1 g, 1.81 mmol) in THF was cooled to 0° C. and DIPEA (0.66 mL, 3.8 mmol) and isobutyl chloroformate (0.26 mL, 1.99 mmol) were added. The resulting mixture was stirred at 0° C. for 1.5 h under nitrogen. The reaction mixture was diluted with EtOAc (150 mL), washed with 1.0 N HCl (2×50 mL), saturated NaHCO₃ (1×50 mL), saturated NaCl (1×50 mL), and dried over anhydrous sodium sulfate and then concentrated under reduced pressure to yield 0.81 g of the desired product, which was used for the next step without further purification. ¹H-NMR (400 MHz, CDCl₃) δ 7.18-7.30 (m, 5H), 6.87 (d, 1H), 6.79 (m, 1H), 6.56-6.63 (m, 1H), 5.61 (d, 1H), 4.64-4.70 (m, 1H), 4.30-4.42 (m, 2H), 3.83-3.90 (m, 2H), 3.66 (s, 3H), 3.46-3.54 (m, 1H), 3.34-3.43 (m, 1H), 3.10-3.15 (m, 1H), 3.00-3.05 (m, 1H), 2.53-2.60 (m, 2H), 2.34-2.51 (m, 2H), 2.11 (s, 3H), 2.02-2.08 (m, 1H), 1.87-2.00 (m, 2H), 1.44-1.61 (m, 3H), 1.45 (s, 9H), 0.93 (d, 6H), 0.85 (d, 3H, J=8.0 Hz), 0.80 (d, 3H, J=8.0 Hz).

### Step d. Synthesis of ib-MLF-β-Ala-OH

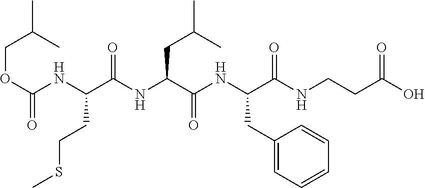 [SMILES: CSCC[C@H](NC(=O)OCC(C)C)C(=O)N[C@@H](CC(C)C)C(=O)N[C@@H](Cc1ccccc1)C(=O)NCCC(=O)O]

The starting material, ib-MLF-β-Ala-OMe (0.81 g, 1.36 mol) was dissolved in a solvent mixture of THF/water (3:1, v/v, 48 mL) and stirred at 0° C. Lithium hydroxide mono hydrate (69 mg, 1.64 mmol) was added and the reaction mixture was stirred at 0° C. for 3.5 h. The reaction was quenched with cold 1.0 N HCl (50 mL), diluted with EtOAc (150 mL) and the layers were thoroughly mixed and separated in a separatory funnel. The organic layer was washed with brine (1×50 mL) and dried over anhydrous sodium sulfate. The solvents were evaporated under reduced pressure and dried under vacuum to yield 0.692 g of the acid as a white solid, which was used for the next step without further purification. ¹H-NMR (400 MHz, DMSO-d₆) δ 12.20 (bs, 1H), 7.94-7.97 (m, 1H), 7.90 (d, 1H), 7.87 (d, 1H), 7.30 (d, 1H), 7.15-7.25 (m, 5H), 4.39-4.44 (m, 1H), 4.21-4.27 (m, 1H), 4.01-4.07 (m, 1H), 3.70-3.73 (m, 2H), 3.21-3.28 (m, 1H), 3.12-3.20 (m, 1H), 2.91-2.96 (m, 1H), 2.77-2.82 (m, 1H), 2.38-2.54 (m, 2H), 2.27-2.31 (m, 2H), 2.02 (s, 3H), 1.70-1.87 (m, 3H), 1.47-1.56 (m, 1H), 1.35-1.40 (m, 2H), 0.79-0.88 (m, 12H).

### Step e. Synthesis of ib-MLF-β-Ala-OSu

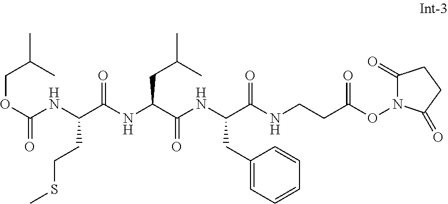 [SMILES: CSCC[C@H](NC(=O)OCC(C)C)C(=O)N[C@@H](CC(C)C)C(=O)N[C@@H](Cc1ccccc1)C(=O)NCCC(=O)ON1C(=O)CCC1=O]

A mixture of ib-MLF-β-Ala-OH (0.685 g, 1.18 mmol), N-hydroxysuccinimide (0.145 g, 1.239 mmol) in DMF (15 mL) was added DCC (0.25 g, 1.18 mmol) in DMF (1.0 ml+1.0 mL rinse) at 0° C. The reaction mixture was stirred at 0° C. for 1 h and then at RT for 48 h. The precipitate was filtered, washed with EtOAc (2×5 mL) and the filtrate was diluted with EtOAc (100 mL) and washed with water (3×30 mL). Silica gel (˜10 g) was added and the combined organic solution was concentrated under reduced pressure. The crude product was purified by flash column chromatography (DCM to EtOAc). Fractions containing the desired product were pooled, concentrated, and the residue was triturated with ether (2×10 mL) to afford 434 mg of the desired NHS ester as a white solid. ¹H-NMR (400 MHz, DMSO-d₆) δ 8.14-8.17 (m, 1H), 7.93 (d, 1H), 7.86 (d, 1H), 7.30 (d, 1H), 7.15-7.26 (m, 5H), 4.40-4.46 (m, 1H), 4.22-4.28 (m, 1H), 3.99-4.07 (m, 1H), 3.67-3.73 (m, 2H), 3.36-3.42 (m, 1H), 3.22-3.28 (m, 1H), 2.94-2.99 (m, 1H), 2.79-2.84 (m, 5H), 2.73-2.76 (m, 2H), 2.36-2.46 (m, 2H), 2.02 (s, 3H), 1.71-1.85 (m, 3H), 1.48-1.57 (m, 1H), 1.36-1.40 (m, 2H), 0.79-0.88 (m, 12H); LC/MS, 678.2 [M+H]⁺.

### Synthesis of a-MLF-β-Ala-OSu (Int-4)

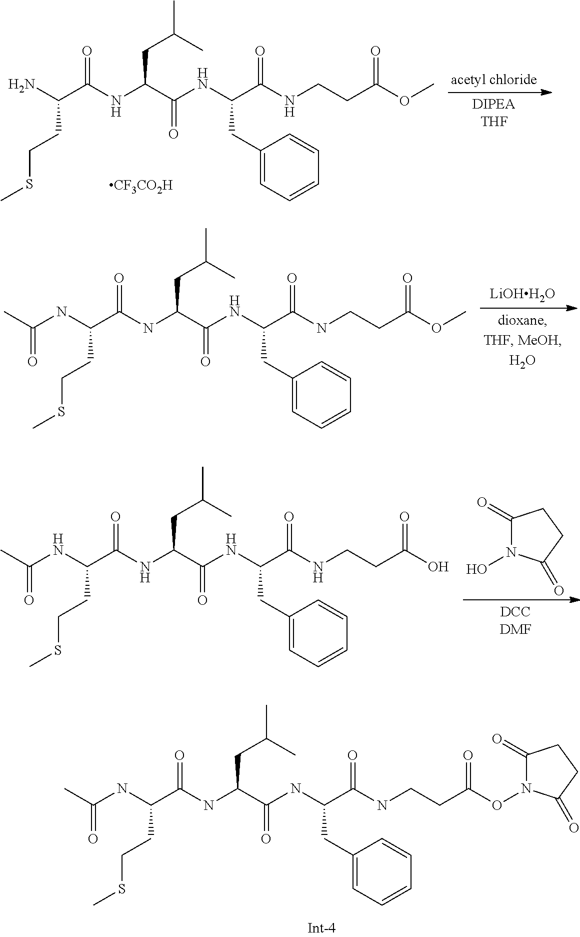

### Step a. Synthesis of a-MLF-β-Ala-OMe

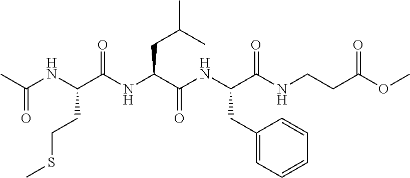 [SMILES: COC(=O)CCNC(=O)[C@H](Cc1ccccc1)NC(=O)[C@H](CC(C)C)NC(=O)[C@H](CCSC)NC(C)=O]

A suspension of H₂N-Met-Leu-Phe-β-Ala-OMe TFA salt (1.1 g, 1.81 mmol) in DCM (31 mL) was cooled to 0° C. and to this suspension was added DIPEA (0.66 mL, 3.8 mmol) and acetyl chloride (0.14 mL, 1.99 mmol). The resulting mixture was stirred at 0° C. for 1.5 h under nitrogen. The reaction mixture was diluted with EtOAc (150 mL), washed with 1.0 N HCl (2×50 mL), saturated NaHCO₃ (1×50 mL), saturated NaCl (1×50 mL), and dried over anhydrous sodium sulfate and then concentrated under reduced pressure. The crude product was purified using silica gel column chromatography, eluting first with ethyl acetate then 10% methanol in DCM to yield 0.67 g of the desired product. ¹H-NMR (400 MHz, DMSO-d₆) δ 8.04 (d, 1H), 7.96 (t, 1H), 7.93 (d, 1H), 7.86 (d, 1H), 7.16-7.26 (m, 5H), 4.38-4.44 (m, 1H), 4.26-4.32 (m, 1H), 4.18-4.24 (m, 1H), 3.59 (s, 3H), 3.15-3.29 (m, 2H), 2.91-2.95 (m, 1H), 2.77-2.83 (m, 1H), 2.36-2.43 (m, 4H), 2.02 (s, 3H), 1.84 (s, 3H), 1.80-1.89 (m, 1H), 1.68-1.77 (m, 1H), 1.46-1.54 (m, 1H), 1.34-1.39 (m, 2H), 0.85 (d, 3H, J=8.0 Hz), 0.80 (d, 3H, J=8.0 Hz); LC/MS, 537.2 [M+H]⁺.

### Step b. Synthesis of a-MLF-β-Ala-OH

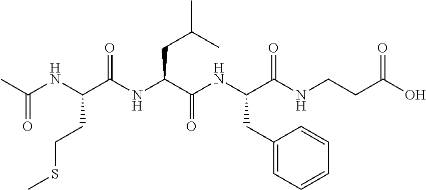 [SMILES: CSCC[C@H](NC(C)=O)C(=O)N[C@@H](CC(C)C)C(=O)N[C@@H](Cc1ccccc1)C(=O)NCCC(=O)O]

The starting material, a-MLF-β-Ala-OMe (0.67 g, 1.25 mmol) was dissolved in a solvent mixture of dioxane/THF/MeOH/water (1:1:1:1, v/v/v/v, 40 mL) and stirred at 0° C. Lithium hydroxide mono hydrate (97 mg, 2.31 mmol) was added and the reaction mixture was stirred at 0° C. for 3.5 h. The reaction was quenched with acetic acid (0.25 mL) and silica gel (10 g) was added. The solvents were evaporated under reduced pressure and the powder was loaded onto 40 g silica gel cartridge, eluting with 2 to 10% methanol in DCM, to yield 0.53 g of the acid as a white solid. ¹H-NMR (400 MHz, DMSO-d₆) δ 8.10 (d, 1H), 7.99 (d, 1H), 7.95 (t, 1H), 7.89 (d, 1H), 7.15-7.25 (m, 5H), 4.38-4.44 (m, 1H), 4.26-4.32 (m, 1H), 4.18-4.24 (m, 1H), 3.12-3.29 (m, 2H), 2.92-2.97 (m, 1H), 2.77-2.83 (m, 1H), 2.38-2.43 (m, 2H), 2.24-2.27 (m, 2H), 2.02 (s, 3H), 1.84 (s, 3H), 1.80-1.87 (m, 1H), 1.70-1.78 (m, 1H), 1.46-1.53 (m, 1H), 1.35-1.39 (m, 2H), 0.84 (d, 3H, J=8.0 Hz), 0.79 (d, 3H, J=8.0 Hz); LC/MS, 523.2 [M+H]⁺.

### Step c. Synthesis of a-MLF-β-Ala-OSu

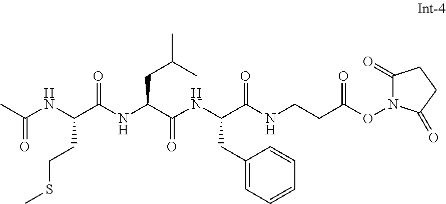 [SMILES: CSCC[C@H](NC(C)=O)C(=O)N[C@@H](CC(C)C)C(=O)N[C@@H](Cc1ccccc1)C(=O)NCCC(=O)ON1C(=O)CCC1=O]

A mixture of a-MLF-β-Ala-OH (0.525 g, 1.00 mmol), N-hydroxysuccinimide (0.121 g, 1.055 mmol) in DMF (13 mL) was added DCC (0.21 g, 1.00 mmol) in DMF (1.0 ml+1.0 mL rinse) at 0° C. The reaction mixture was stirred at 0° C. for 1 h and then at RT for 24 h. The precipitate was filtered, washed with EtOAc (2×5 mL) and the filtrate was diluted with EtOAc (100 mL) and washed with water (3×30 mL). Silica gel (˜10 g) was added and the combined organic solution was concentrated under reduced pressure. The crude product was purified by flash column chromatography (DCM to EtOAc). Fractions containing the desired product were pooled, concentrated, and the residue was triturated with ether (2×10 mL) to afford 350 mg of the desired NHS ester as a white solid. ¹H-NMR (400 MHz, DMSO-d₆) δ 8.14-8.17 (m, 1H), 8.03 (d, 1H, J=8 Hz), 7.94 (d, 1H, J=8 Hz), 7.89 (d, 1H, J=8 Hz), 7.16-7.27 (m, 5H), 4.40-4.46 (m, 1H), 4.19-4.32 (m, 2H), 3.22-3.43 (m, 2H), 2.94-2.99 (m, 1H), 2.79-2.85 (m, 5H), 2.74-2.77 (m, 2H), 2.37-2.43 (m, 2H), 2.02 (s, 3H), 1.84 (s, 3H), 1.80-1.89 (m, 1H), 1.68-1.77 (m, 2H), 1.59-1.64 (m, 1H), 1.35-1.39 (m, 2H), 0.85 (d, 3H, J=8.0 Hz), 0.79 (d, 3H, J=8.0 Hz); LC/MS, 620.2 [M+H]⁺.

Int-4 was also prepared by the following method.

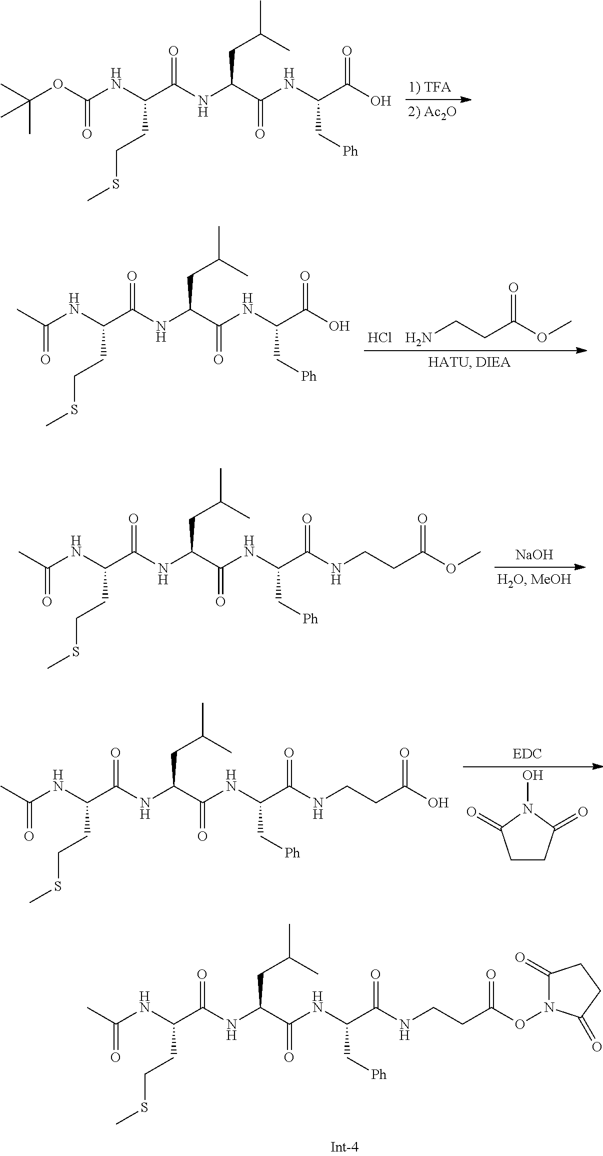

### Step a. Synthesis of Ac-MLF-OH

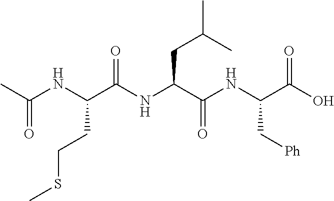 [SMILES: CSCC[C@H](NC(C)=O)C(=O)N[C@@H](CC(C)C)C(=O)N[C@@H](Cc1ccccc1)C(=O)O]

Boc-MLF-OH (500 mg, 0.982 mmol) was dissolved in 30% TFA/DCM (10 ml) at room temperature. After 30 min LCMS showed no starting material. The crude reaction was concentrated to an oil, dissolved in DCM (5 mL), and treated with N-methylmorpholine (431 μL, 3.93 mmol) and acetic anhydride (185 μL, 1.96 mmol) at room temperature. LCMS at 30 min showed complete conversion. The reaction was concentrated then purified by RPLC using an Isco CombiFlash liquid chromatograph eluted with 10% to 100% acetonitrile and water, using 0.1% formic acid as the modifier. Removal of the volatiles in vacuo gave 230 mg of a white powder. LC/MS, 452.2 [M+H]+ calculated 452.2.

### Step b. Synthesis of Ac-MLF-β-ala-OH

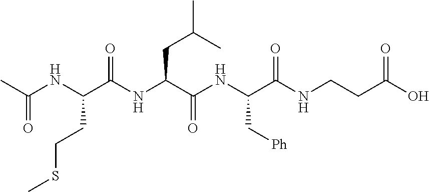 [SMILES: CSCC[C@H](NC(C)=O)C(=O)N[C@@H](CC(C)C)C(=O)N[C@@H](Cc1ccccc1)C(=O)NCCC(=O)O]

HATU (577 mg, 1.518 mmol) was added to a stirring solution of Ac-MLF-OH (685 mg, 1.518 mmol), β-ala-OMe HCl (212 mg, 1.518 mmol), DIPEA (793 μL, 4.55 mmol), and DMF (4.5 mL), at room temperature. After 15 min the reaction solidified to a gel and stopped stirring. The addition of methanol did not help stirring. LCMS at this time showed no remaining starting material. The crude reaction was taken on to the next step without purification. A solution of sodium hydroxide (304 mg, 7.59 mmol), and water (6 mL), was added to the mixture, which dissolved the gel and permitted stirring. LCMS at 30 min showed no starting material remaining. The reaction was neutralized with acetic acid, concentrated, and purified by RPLC using an Isco CombiFlash liquid chromatograph eluted with 10% to 100% acetonitrile and water, using 0.1% formic acid as the modifier. Removal of the volatiles in vacuo gave 810 mg of white powder. LC/MS, 523.2 [M+H]⁺ calculated 523.3.

### Step c. Synthesis of Ac-MLF-β-ala-OSu (Int-4)

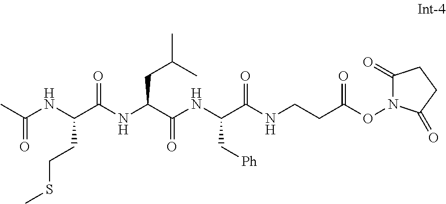 [SMILES: CSCC[C@H](NC(C)=O)C(=O)N[C@@H](CC(C)C)C(=O)N[C@@H](Cc1ccccc1)C(=O)NCCC(=O)ON1C(=O)CCC1=O]

A flask was charged with Ac-MLF-β-ala-OH (800 mg, 1.532 mmol), N-(3-dimethylaminopropyl)—N′-ethylcarbodiimide hydrochloride (EDC) (261 mg, 1.685 mol), N-hydroxysuccinimide (194 mg, 1.685 mmol), and DMF (5 ml) at room temperature. After 40 min, additional EDC (261 mg, 1.685 mmol), and N-hydroxysuccinimide (194 mg, 1.685 mmol), were added. LCMS after an additional 15 min shows no starting material. The reaction was diluted with ethyl acetate and washed with water and sat. NaHCO₃. Upon standing a white precipitate formed which was collected by filtration. 664 mg of the product was obtained as a white powder after drying. LC/MS, 620.2 [M+H]⁺ calculated 620.7.

### Synthesis of p-Cl-Ph-NHCONHMLF-OSu (Int-5)

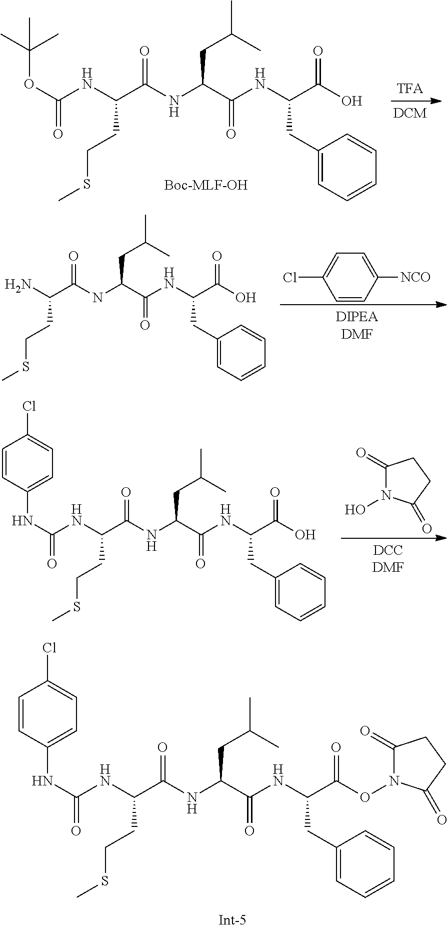

### Step a. Synthesis of p-Cl-Ph-NHCONHMLF-OH

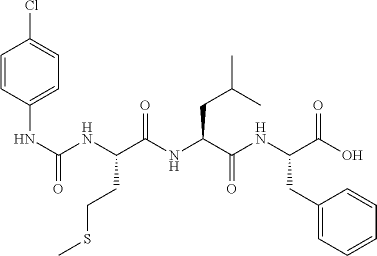 [SMILES: CSCC[C@H](NC(=O)Nc1ccc(Cl)cc1)C(=O)N[C@@H](CC(C)C)C(=O)N[C@@H](Cc1ccccc1)C(=O)O]

A solution of Boc-Met-Leu-Phe-OH (0.5 g, 0.98 mmol) in DCM (5 mL) was cooled to 0° C. and TFA (5 mL) was added. The mix was stirred at 0° C. for 2 h and then concentrated. The residue was azeotropically dried with toluene (2×5 mL) and then dissolved in dry DMF (30 mL). DIPEA (0.342 mL, 1.96 mmol) and 4-chlorophenyl isocyanate (0.188 mL, 1.47 mmol) were added and the resulting mixture was stirred at room temperature overnight under nitrogen. The reaction mixture was diluted with EtOAc (150 mL), washed with 1.0 N HCl (2×50 mL), water (3×50 mL), sat-NaCl (1×50 mL), and dried over anhydrous sodium sulfate and then concentrated under reduced pressure. The crude product was triturated with diethyl ether (2×15 mL) to yield 0.325 g of the desired product, which was used for the next step without further purification. ¹H-NMR (400 MHz, DMSO-d₆) δ 8.91 (s, 1H), 8.15 (d, 1H, J=8.4 Hz), 8.00 (bs, 1H), 7.16-7.41 (m, 9H), 6.55 (d, 1H, J=7.9 Hz), 4.28-4.39 (m, 3H), 3.31 (m, 1H), 3.04-3.08 (m, 1H), 2.89-2.94 (m, 1H), 2.41 (t, 2H, J=7.9 Hz), 2.00 (s, 3H), 1.83-1.91 (m, 1H), 1.70-1.79 (m, 1H), 1.54-1.61 (m, 1H), 1.40-1.43 (m, 2H), 0.86 (d, 3H, J=8.0 Hz), 0.82 (d, 3H, J=8.0 Hz); LC/MS, 563.1 [M+H]⁺.

### Step b. Synthesis of p-Cl-Ph-NHCONHMLF-OSu

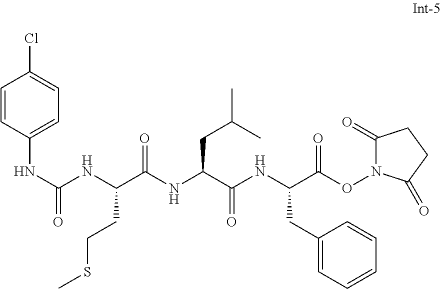 [SMILES: CSCC[C@H](NC(=O)Nc1ccc(Cl)cc1)C(=O)N[C@@H](CC(C)C)C(=O)N[C@@H](Cc1ccccc1)C(=O)ON1C(=O)CCC1=O]

A mixture of p-Cl-Ph-NHCONHMLF-OH (0.139 g, 0.2468 mmol), N-hydroxysuccinimide (0.030 g, 1.0 eq) in DMF (3 mL) was added DCC (0.051 g, 1.0 eq). The reaction mixture was stirred at RT overnight. The precipitate was filtered and the product in DMF was used directly in subsequent reactions.

### Synthesis of fMLF-(Z)^{ε}—K-OSu (Int-6)

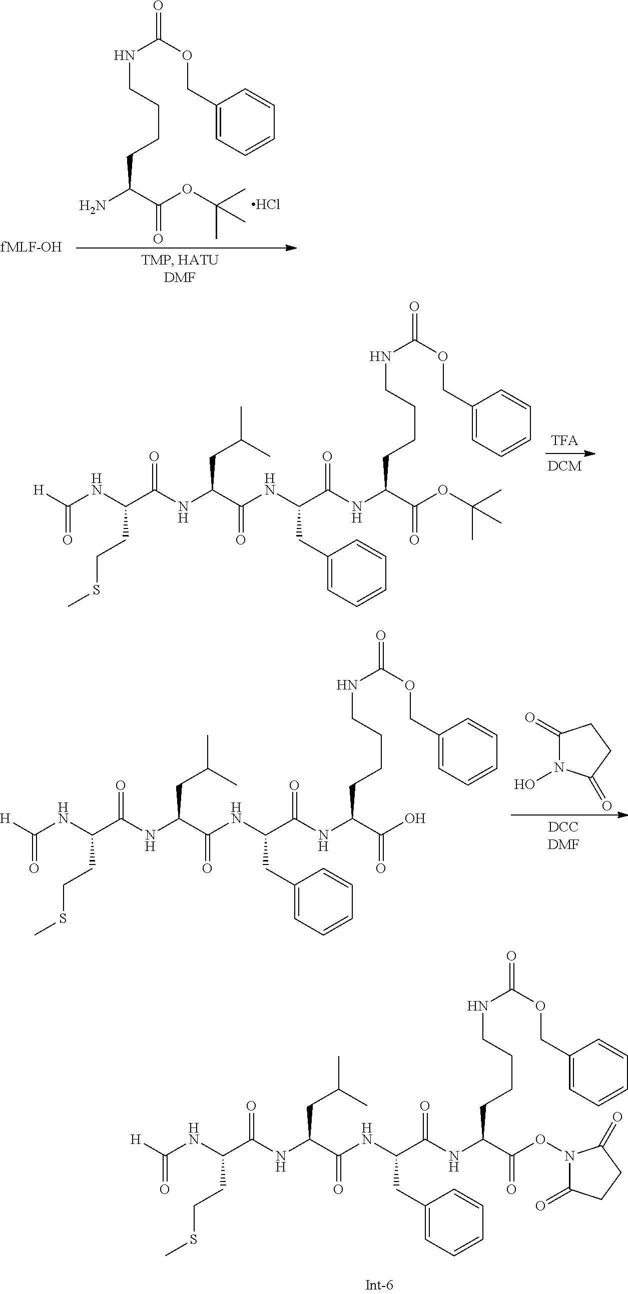

### Step a. Synthesis of fMLF-(Z)^{ε}—K-OBu-t

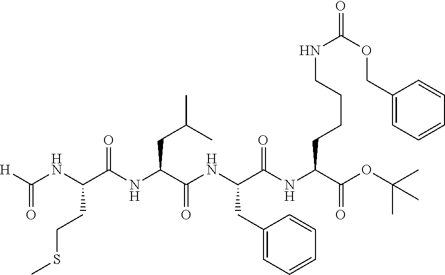 [SMILES: CSCC[C@H](NC=O)C(=O)N[C@@H](CC(C)C)C(=O)N[C@@H](Cc1ccccc1)C(=O)N[C@@H](CCCCNC(=O)OCc1ccccc1)C(=O)OC(C)(C)C]

A solution of formyl-Met-Leu-Phe-OH (0.5 g, 1.14 mmol) and H-Lys(Z)-OtBu.HCl (0.426 g, 1.14 mmol) in DMF (9.0 mL) was added TMP (0.3 mL, 2.28 mmol) and HATU (0.435 g, 1.14 mmol) at 0° C. and the resulting mixture was stirred at 0° C. for 1 h and at room temperature for 1.5 h under nitrogen. The reaction mixture was diluted with EtOAc (150 mL), washed with 1.0 N HCl (2×50 mL), sat-NaHCO₃ (2×50 mL), sat-NaCl (1×50 mL), dried over anhydrous sodium sulfate, and then concentrated under reduced pressure. The crude product was purified using silica gel column chromatography, eluting with 0 to 100% ethyl acetate in DCM, to yield 0.7 g of the desired product. ¹H-NMR (400 MHz, DMSO-d₆) δ 8.26 (d, 1H, J=8.3 Hz), 8.19 (d, 1H, J=7.5 Hz), 8.02 (d, 1H, J=9.7 Hz), 8.00 (s, 1H), 7.91 (d, 1H, J=8.0 Hz), 7.15-7.38 (m, 10H), 5.00 (s, 2H), 4.54-4.59 (m, 1H), 4.37-4.42 (m, 1H), 4.22-4.28 (m, 1H), 4.02-4.08 (m, 1H), 2.96-3.05 (m, 3H), 2.76-2.82 (m, 1H), 2.35-2.40 (m, 2H), 2.00 (s, 3H), 1.83-1.91 (m, 1H), 1.23-1.88 (m, 11H), 1.39 (s, 9H), 0.84 (d, 3H, J=6.5 Hz), 0.80 (d, 3H, J=6.5 Hz).

### Step b. Synthesis of fMLF-(Z)^{ε}—K—OH

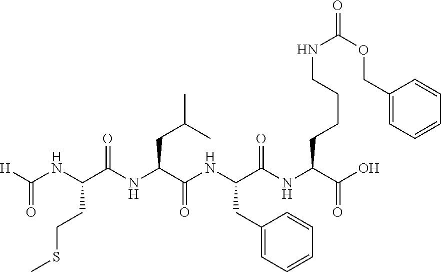 [SMILES: CSCC[C@H](NC=O)C(=O)N[C@@H](CC(C)C)C(=O)N[C@@H](Cc1ccccc1)C(=O)N[C@@H](CCCCNC(=O)OCc1ccccc1)C(=O)O]

A solution of fMLF-(Z)^{ε}—K-OBu-t in DCM (5 mL) was added TFA (5 mL) at 0° C. and the resulting mixture was stirred at 0° C. for 1 h and at room temperature for 3 h under nitrogen. The solvents were removed under reduced pressure and the crude product was purified using silica gel column chromatography, eluting with 0 to 10% methanol in DCM, to yield 0.45 g of the desired product. ¹H-NMR (400 MHz, DMSO-d₆) δ 8.27 (d, 1H, J=8.7 Hz), 8.02-8.05 (m, 1H), 8.00 (s, 1H), 7.95 (d, 1H, J=8.1 Hz), 7.14-7.38 (m, 11H), 5.00 (s, 2H), 4.51-4.57 (m, 1H), 4.37-4.42 (m, 1H), 4.23-4.29 (m, 1H), 4.08-4.13 (m, 1H), 2.94-3.06 (m, 3H), 2.77-2.83 (m, 1H), 2.35-2.40 (m, 2H), 2.00 (s, 3H), 1.24-1.88 (m, 11H), 0.84 (d, 3H, J=6.5 Hz), 0.80 (d, 3H, J=6.5 Hz).

### Step c. Synthesis of Synthesis of fMLF-(Z)^{ε}—K-OSu

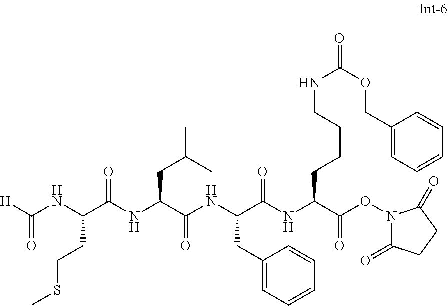 [SMILES: CSCC[C@H](NC=O)C(=O)N[C@@H](CC(C)C)C(=O)N[C@@H](Cc1ccccc1)C(=O)N[C@@H](CCCCNC(=O)OCc1ccccc1)C(=O)ON1C(=O)CCC1=O]

A mixture of fMLF-(Z)—K—OH (0.288 g, 0.4115 mmol), N-hydroxysuccinimide (0.050 g, 1.05 eq) in DMF (5 mL) was added DCC (0.085 g, 1.0 eq). The reaction mixture was stirred at RT overnight. The precipitate was filtered and the product was used directly in subsequent reactions as a solution in DMF.

### Synthesis of fMLF-(Z)^{α}—K-OSu (Int-7)

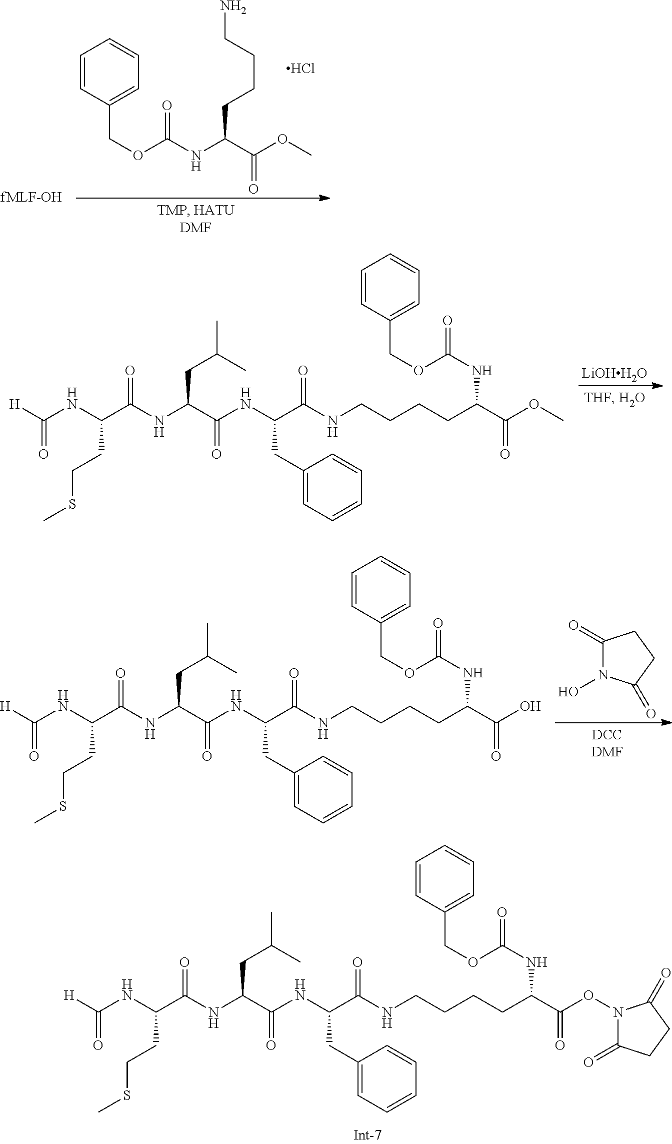

### Step a. Synthesis of fMLF-(Z)^{α}—K-OMe

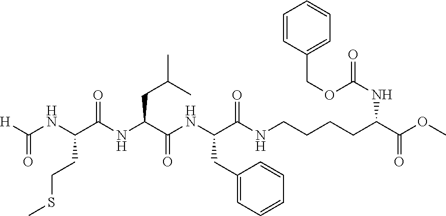 [SMILES: COC(=O)[C@H](CCCCNC(=O)[C@H](Cc1ccccc1)NC(=O)[C@H](CC(C)C)NC(=O)[C@H](CCSC)NC=O)NC(=O)OCc1ccccc1]

A solution of Formyl-Met-Leu-Phe-OH (0.5 g, 1.14 mmol) and (Z)^{α}-Lys-OMe hydrochloride (0.378 g, 1.14 mmol) in DMF (9.0 mL) was added TMP (0.3 mL, 2.28 mol) and HATU (0.435 g, 1.14 mmol) at 0° C. and the resulting mixture was stirred at 0° C. for 1 h and at room temperature for 1.5 h under nitrogen. The reaction mixture was diluted with EtOAc (75 mL), washed with 1.0 N HCl (2×15 mL), sat-NaHCO₃ (2×15 mL), sat-NaCl (2×15 mL), and dried over anhydrous sodium sulfate and then concentrated under reduced pressure. The crude product was triturated with diethyl ether and then dried under vacuum to yield 700 mg of the desired product. ¹H-NMR (400 MHz, DMSO-d₆) δ 8.29 (d, 1H, J=8.6 Hz), 8.06 (d, 1H, J=8.2 Hz), 8.01 (s, 1H), 7.91 (d, 1H, J=8.5 Hz), 7.80-7.83 (m, 1H), 7.69 (d, 1H, J=7.7 Hz), 7.15-7.38 (m, 10H), 4.99-5.06 (m, 2H), 4.37-4.44 (m, 2H), 4.22-4.27 (m, 1H), 3.95-3.99 (m, 1H), 3.63 (s, 3H), 2.88-3.07 (m, 3H), 2.78-2.84 (m, 1H), 2.38-2.42 (m, 2H), 2.01 (s, 3H), 1.15-1.90 (m, 11H), 1.39 (s, 9H), 0.85 (d, 3H, J=6.6 Hz), 0.80 (d, 3H, J=6.6 Hz).

### Step b. Synthesis of fMLF-(Z)^{α}—K—OH

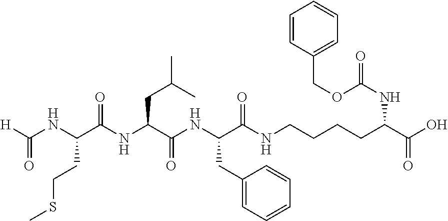 [SMILES: CSCC[C@H](NC=O)C(=O)N[C@@H](CC(C)C)C(=O)N[C@@H](Cc1ccccc1)C(=O)NCCCC[C@H](NC(=O)OCc1ccccc1)C(=O)O]

The starting material, fMLF-(Z)^{α}—K-OMe 0.7 g, 0.98 mmol), was dissolved in a solvent mixture of THF (32 mL)/water (16 mL) and stirred at 0° C. Lithium hydroxide mono hydrate (90 mg, 2.15 mmol) was added and the reaction mixture was stirred at 0° C. for 3.5 h. The reaction was quenched with 1.0 N HCl (5.0 mL), diluted with water (50 mL), and extracted with ethyl acetate (1×100 mL). The organic extract was washed with brine (1×50 mL) and dried over anhydrous sodium sulfate. The solvents were evaporated under reduced pressure and the product was triturated with diethyl ether to yield 0.68 g of the acid as a white solid. ¹H-NMR (400 MHz, DMSO-d₆) δ 8.28 (d, 1H, J=8.5 Hz), 8.06 (d, 1H, J=8.0 Hz), 8.01 (s, 1H), 7.91 (d, 1H, J=8.2 Hz), 7.81-7.83 (m, 1H), 7.52 (d, 1H, J=8.1 Hz), 7.14-7.38 (m, 10H), 4.99-5.06 (m, 2H), 4.38-4.44 (m, 2H), 4.22-4.28 (m, 1H), 3.86-3.91 (m, 1H), 3.02-3.07 (m, 1H), 2.88-2.96 (m, 2H), 2.79-2.84 (m, 1H), 2.37-2.42 (m, 2H), 2.01 (s, 3H), 1.15-1.91 (m, 11H), 0.84 (d, 3H, J=6.6 Hz), 0.80 (d, 3H, J=6.6 Hz).

### Step c. Synthesis of Synthesis of fMLF-(Z)^{α}—K-OSu

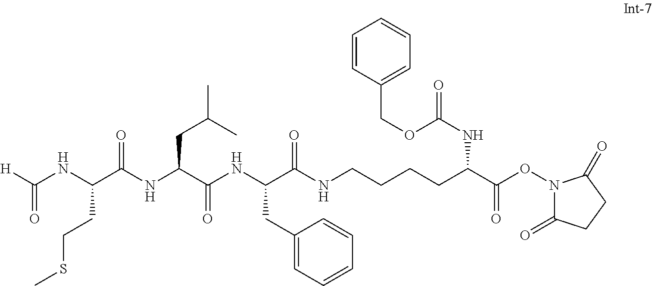 [SMILES: CSCC[C@H](NC=O)C(=O)N[C@@H](CC(C)C)C(=O)N[C@@H](Cc1ccccc1)C(=O)NCCCC[C@H](NC(=O)OCc1ccccc1)C(=O)ON1C(=O)CCC1=O]

A mixture of fMLF-(Z)^{α}—K—OH (0.173 g, 0.2472 mmol), HOSu (0.030 g, 1.05 eq) in DMF (3 mL) was added DCC (0.051 g, 1.0 eq). The reaction mixture was stirred at RT overnight (or until most of the acid is consumed as monitored by TLC and LC-MS analyses). The precipitate was filtered and the product as a solution in DMF was used directly in subsequent reactions.

### Synthesis of fMLF-(PEG)₂-Suc-OSu (Int-8)

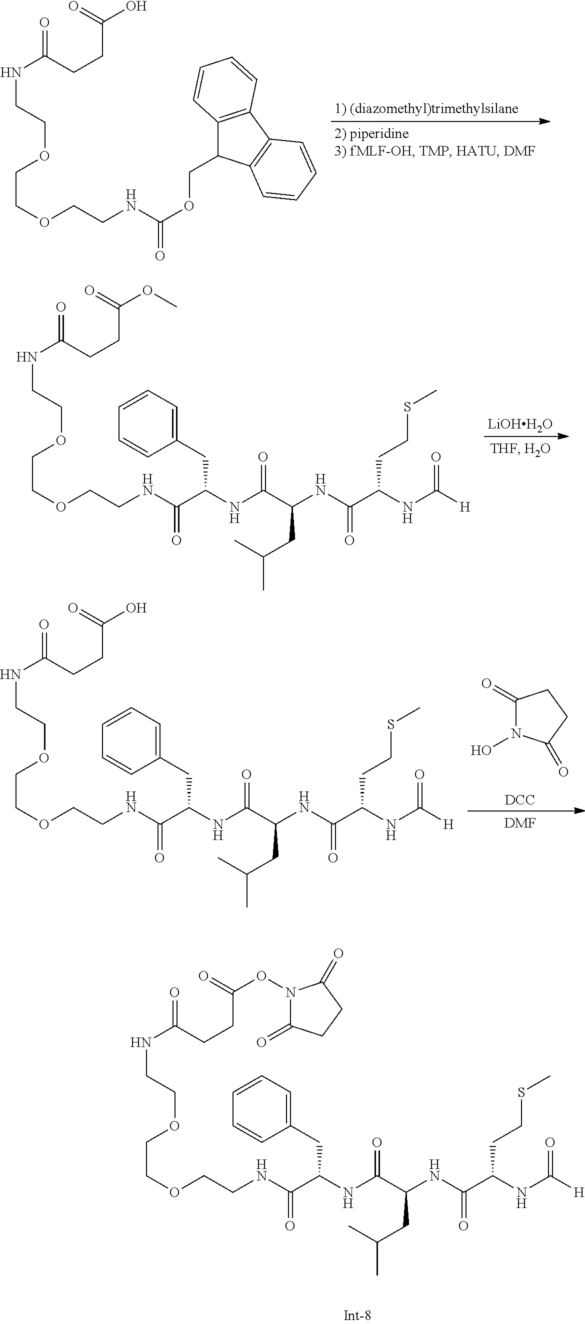

### Step a. Synthesis of fMLF-(PEG)₂-Suc-OMe

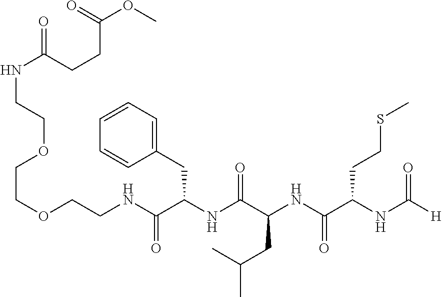 [SMILES: COC(=O)CCC(=O)NCCOCCOCCNC(=O)[C@H](Cc1ccccc1)NC(=O)[C@H](CC(C)C)NC(=O)[C@H](CCSC)NC=O]

Fmoc-(PEG)₂-Suc-OH (1.0 g, 2.1 mmol) was dissolved in a solvent mixture of toluene (5 mL) and MeOH (2 mL) and stirred at RT under nitrogen. To this solution was added dropwise (diazomethyl)trimethylsilane (2M/ether) (1.8 mL, 3.6 mmol). Gas evolution was observed. The reaction mixture was stirred under nitrogen at room temperature for 40 min. The solvent was removed under reduced pressure to yield 1.05 g of the methyl ester, which was dissolved in DMF (18 mL). To this solution was added piperidine (2 mL) and the reaction mixture was stirred at room temperature for 30 min. The solvent was removed under reduced pressure and the crude product was treated with 4.0 N HCl in dioxane (2.0 mL). The solvent was removed again under reduced pressure to yield the amine product as its HCl salt. A solution of Formyl-Met-Leu-Phe-OH (0.3 g, 0.686 mmol) and the above amine salt (0.21 g, 0.686 mmol) in DMF (5.5 mL) was added TMP (0.18 mL, 1.37 mmol) and HATU (0.261 g, 0.686 mmol) at 0° C. and the resulting mixture was stirred at 0° C. for 1 h and at room temperature for 1.5 h under nitrogen. The reaction mixture was diluted with EtOAc (75 mL), washed with 1.0 N HCl (2×15 mL), sat-NaHCO₃ (2×15 mL), sat-NaCl (2×15 mL), and dried over anhydrous sodium sulfate and then concentrated under reduced pressure. The crude product was triturated with diethyl ether and then dried under vacuum to yield 470 mg of the desired product. ¹H-NMR (400 MHz, DMSO-d₆) δ 8.28 (d, 1H, J=8.4 Hz), 8.06 (d, 1H, J=8.1 Hz), 8.01 (s, 1H), 7.89-7.96 (m, 3H), 7.15-7.26 (m, 5H), 4.38-4.49 (m, 2H), 4.22-4.28 (m, 1H), 3.56 (s, 3H), 3.48-3.52 (m, 5H), 3.33-3.40 (m, 3H), 3.11-3.24 (m, 4H), 2.92-2.97 (m, 1H), 2.79-2.84 (m, 1H), 2.33-2.42 (m, 4H), 2.01 (s, 3H), 1.69-1.90 (m, 2H), 1.31-1.43 (m, 2H), 0.85 (d, 3H, J=6.6 Hz), 0.80 (d, 3H, J=6.6 Hz); LC/MS, 682.2 [M+H]⁺.

### Step b. Synthesis of fMLF-(PEG)₂-Suc-OH

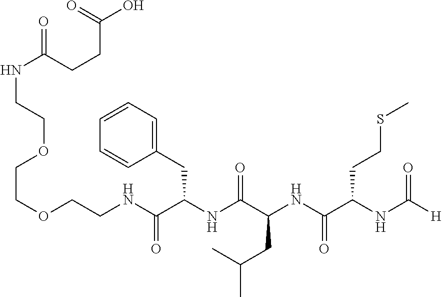 [SMILES: CSCC[C@H](NC=O)C(=O)N[C@@H](CC(C)C)C(=O)N[C@@H](Cc1ccccc1)C(=O)NCCOCCOCCNC(=O)CCC(=O)O]

The starting material (0.47 g, 0.689 mmol) was dissolved in a solvent mixture of THF (18 mL) and water (6 mL) and stirred at 0° G. Lithium hydroxide mono hydrate (55 mg) was added and the reaction mixture was stirred at 0° C. for 3 h. The reaction was quenched with 1.0 N HCl (5.0 mL) and extracted with ethyl acetate (3×50 mL). The combined organic extracts were washed with brine (1×25 mL), drived over anhydrous sodium sulfate, evaporated under reduced pressure and dried under vacuum to yield 0.37 g of the acid as a white solid. ¹H-NMR (400 MHz, DMSO-d₆) δ 8.28 (d, 1H, J=8.3 Hz), 8.05 (d, 1H, J=7.8 Hz), 8.01 (s, 1H), 7.87-7.96 (m, 3H), 7.15-7.26 (m, 5H), 4.38-4.49 (m, 2H), 4.22-4.28 (m, 1H), 3.47-3.51 (m, 4H), 3.38 (t, 2H, J=6.0 Hz), 3.11-3.24 (m, 5H), 2.92-2.97 (m, 1H), 2.79-2.84 (m, 1H), 2.37-2.42 (m, 4H), 2.28-2.32 (m, 2H), 2.01 (s, 3H), 1.69-1.90 (m, 2H), 1.31-1.55 (m, 3H), 0.85 (d, 3H, J=6.5 Hz), 0.80 (d, 3H, J=6.5 Hz).

### Step c. Synthesis of fMLF-(PEG)₂-Suc-OSu

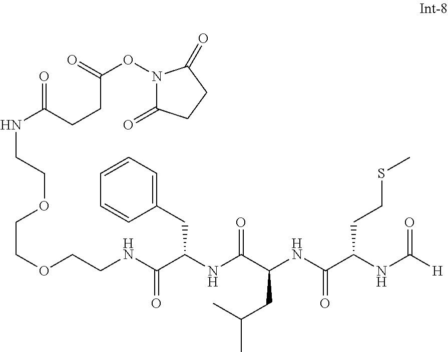 [SMILES: CSCC[C@H](NC=O)C(=O)N[C@@H](CC(C)C)C(=O)N[C@@H](Cc1ccccc1)C(=O)NCCOCCOCCNC(=O)CCC(=O)ON1C(=O)CCC1=O]

A mixture of fMLF-(PEG)₂-Suc-OH (0.165 g, 0.2471 mmol), N-hydroxysuccinimide (0.030 g, 0.259 mmol) in DMF (3 mL) was added DCC (0.051 g, 0.2471 mmol). The reaction mixture was stirred at RT overnight. The precipitate was filtered and the product as a solution in DMF was used directly in subsequent reactions.

### Synthesis of Pf^{α}-(Z)^{ε}—K-OSu (Int-9)

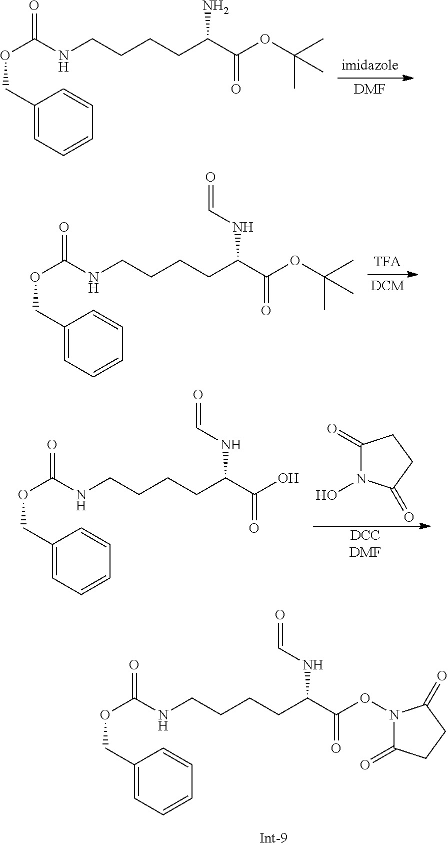

### Step a. Synthesis of f^{α}-(Z)—K-OBu-t

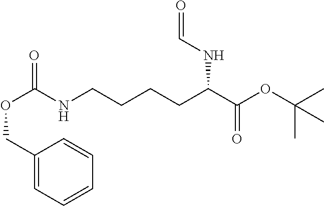

To a solution of imidazole (548 mg, 8.05 mmol) in DMF (4 mL) was added H-Lys(Z)-OBu-t.HCl (1.5 g, 4.02 mmol). The reaction mixture was stirred at 60° C. for 48 h. DMF was evaporated and the residue was dissolved in saturated NaHCO₃ (80 mL) and extracted by EtOAc (3×80 mL). The combined organic extracts were dried and concentrated; the residue was purified by FCC on SiO₂ (5% methanol/DCM). Evaporation of solvents afforded the N-formyl amino acid ester (1.0 g product). ¹H-NMR (400 MHz, CDCl₃) δ 8.18 (s, 1H), 7.30-7.39 (m, 5H), 6.17-6.19 (m, 1H), 5.10 (s, 2H), 4.53-4.58 (m, 1H), 3.17-3.22 (m, 2H), 1.82-1.91 (m, 1H), 1.65-1.74 (m, 1H), 1.50-1.59 (m, 2H), 1.47 (s, 9H), 1.31-1.44 (m, 2H); LC/MS, 365.1 [M+H]⁺.

### Step b. Synthesis of f^{α}-(Z)^{ε}—K—OH

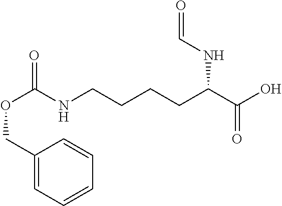

The starting material, f^{α}-(Z)^{ε}—K-OBu-t (1.0 g, 2.74 mmol), was dissolved in DCM (21 mL) and stirred at 0° C. Trifluoroacetic acid (21 mL) was added and the reaction mixture was stirred at 0° C. for 1 h and at room temperature for 3 h. The solvents were removed under reduced pressure and the crude product was purified using silica gel column chromatography, eluting with 0 to 5% methanol in DCM, to yield 0.96 g of the desired product. ¹H-NMR (400 MHz, DMSO-d₆) δ 12.66 (bs, 1H), 8.33 (d, 1H, J=7.5 Hz), 8.03 (s, 1H), 7.29-7.38 (m, 5H), 7.22-7.24 (m, 1H), 5.00 (s, 2H), 4.20-4.25 (m, 1H), 2.95-2.99 (m, 2H), 1.66-1.75 (m, 1H), 1.52-1.62 (m, 1H), 1.36-1.45 (m, 2H), 1.24-1.34 (m, 2H); LC/MS, 309.2 [M+H]⁺.

### Step c. Synthesis of Synthesis of f^{α}-(Z)^{ε}—K-OSu

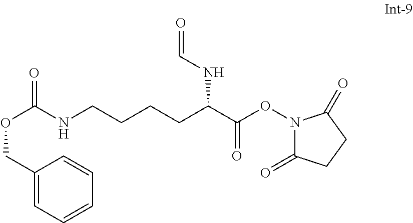

A mixture of f^{α}-(Z)^{ε}—K—OH (0.102 g, 0.3292 mmol), HOSu (0.040 g, 1.05 eq) in DMF (4 mL) was added DCC (0.068 g, 1.0 eq). The reaction mixture was stirred at RT overnight. The precipitate was filtered and the product as a solution in DMF was used directly for the next step.

### Synthesis of fML-OSu (Int-10)

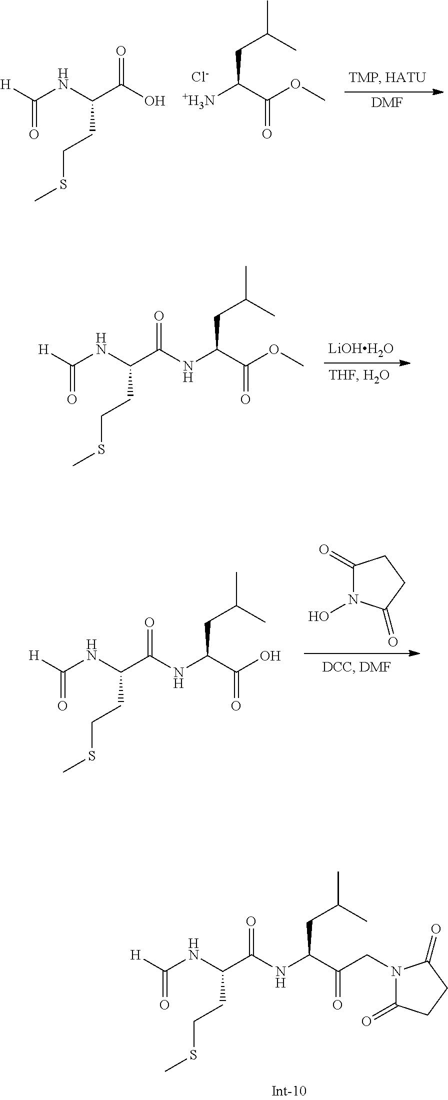

### Step a. Synthesis of fML-OMe

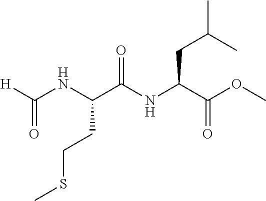 [SMILES: COC(=O)[C@H](CC(C)C)NC(=O)[C@H](CCSC)NC=O]

A solution of formyl-Met-OH (0.8 g, 4.51 mmol) and H-Leu-OMe.HCl (0.82 g, 4.51 mmol) in DMF (36 mL) was added TMP (1.2 mL, 9.03 mmol) and HATU (1.72 g, 4.51 mmol) at 0° C. and the resulting mixture was stirred at 0° C. for 1 h and at room temperature for 1.5 h under nitrogen. The reaction mixture was diluted with EtOAc (150 mL), washed with 1.0 N HCl (1×100 mL, 1×50 mL), sat-NaHCO₃ (1×100 mL, 1×50 mL), sat-NaCl (1×100 mL), dried over anhydrous sodium sulfate, and then concentrated under reduced pressure to yield 1.28 g of the desired product, which was used for the next step without further purification. ¹H-NMR (400 MHz, DMSO-d₆) δ 8.39 (d, 1H, J=7.63 Hz), 8.28 (d, 1H, J=8.45 Hz), 8.00 (s, 1H), 4.43-4.48 (m, 1H), 4.24-4.30 (m, 1H), 3.62 (s, 3H), 2.44 (t, 2H, J=8.3 Hz), 2.04 (s, 3H), 1.73-1.94 (m, 2H), 1.46-1.66 (m, 3H), 0.89 (d, 3H, J=6.43 Hz), 0.83 (d, 3H, J=6.43 Hz).

### Step b. Synthesis of fML-OH

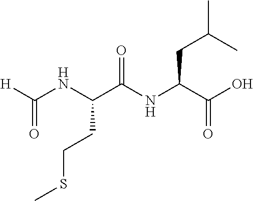 [SMILES: CSCC[C@H](NC=O)C(=O)N[C@@H](CC(C)C)C(=O)O]

The starting material, fML-OMe (1.28 g, 4.2 mmol) was dissolved in a solvent mixture of THF (45 mL) and water (15 mL) and stirred at 0° C. Lithium hydroxide mono hydrate (326 mg, 7.78 mmol) was added and the reaction mixture was stirred at 0° C. for 3.5 h. The reaction was quenched with 1.0 N HCl (25.0 mL) and extracted with ethyl acetate (3×100 mL). The combined organic extracts were washed with brine (1×50 mL), dried over anhydrous sodium sulfate, evaporated under reduced pressure and dried in vacuo to yield 0.85 g of the acid as a white solid. ¹H-NMR (400 MHz, DMSO-d₆) δ 8.28 (d, 1H, J=7.63 Hz), 8.25 (d, 1H, J=8.22 Hz), 8.00 (s, 1H), 4.43-4.48 (m, 1H), 4.17-4.23 (m, 1H), 3.62 (s, 3H), 2.44 (t, 2H, J=7.76 Hz), 2.03 (s, 3H), 1.73-1.94 (m, 2H), 1.46-1.66 (m, 3H), 0.89 (d, 3H, J=6.55 Hz), 0.83 (d, 3H, J=6.55 Hz).

### Step c. Synthesis of Synthesis of fML-OSu

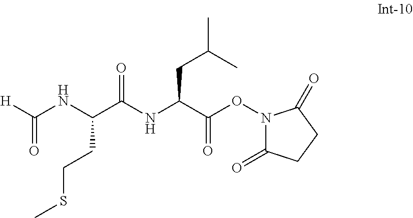 [SMILES: CSCC[C@H](NC=O)C(=O)N[C@@H](CC(C)C)C(=O)ON1C(=O)CCC1=O]

A mixture of fML-OH (0.072 g, 0.2473 mmol), N-hydroxysuccinimide (0.030 g, 1.05 eq) in DMF (3 mL) was added DCC (0.051 g, 1.0 eq). The reaction mixture was stirred at RT overnight. The precipitate was filtered and the product as a solution in DMF was used directly for the next step.

### Synthesis of fMLM-OSu (Int-11)

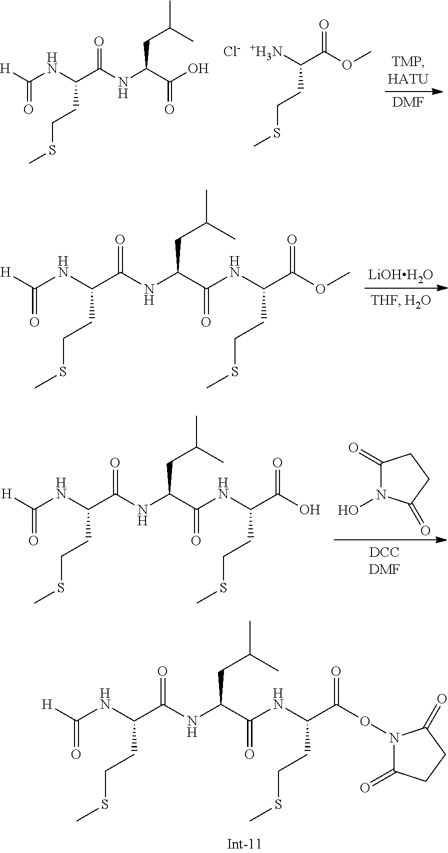

### Step a. Synthesis of fMLM-OMe

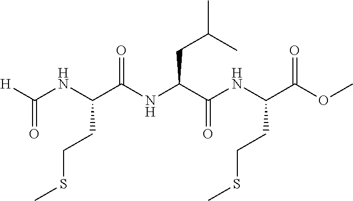 [SMILES: COC(=O)[C@H](CCSC)NC(=O)[C@H](CC(C)C)NC(=O)[C@H](CCSC)NC=O]

A solution of formyl-Met-Leu-OH (Int-10, Step b, 0.38 g, 1.31 mmol) and Met-OMe.HCl (0.26 g, 1.31 mmol) in DMF (10 mL) was added TMP (0.35 mL, 2.62 mmol) and HATU (0.50 g, 1.31 mmol) at 0° C. and the resulting mixture was stirred at 0° C. for 1 h and at room temperature for 1.5 h under nitrogen. The reaction mixture was diluted with EtOAc (100 mL), washed with 1.0 N HCl (2×25 mL), sat-NaHCO₃ (2×50 mL), sat-NaCl (1×25 mL), dried over anhydrous sodium sulfate, and then concentrated under reduced pressure to yield 0.433 g of the desired product, which was used for the next step without further purification. ¹H-NMR (400 MHz, DMSO-d₆) δ 8.26-8.28 (m, 2H), 8.09 (d, 1H, J=7.95 Hz), 8.00 (s, 1H), 4.36-4.44 (m, 2H), 4.30 (q, 1H, J=7.75 Hz), 3.62 (s, 3H), 2.39-2.55 (m, 4H), 2.03 (s, 3H), 2.02 (s, 3H), 1.71-1.99 (m, 4H), 1.57-1.66 (m, 1H), 1.44-1.47 (m, 2H), 0.90 (d, 3H, J=6.55 Hz), 0.85 (d, 3H, J=6.55 Hz).

### Step b. Synthesis of fMLM-OH

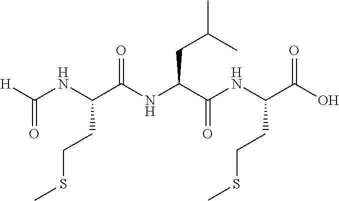 [SMILES: CSCC[C@H](NC(=O)[C@H](CC(C)C)NC(=O)[C@H](CCSC)NC=O)C(=O)O]

The starting material, fMLM-OMe (0.425 g, 0.976 mmol) was dissolved in a solvent mixture of THF (18 mL) and water (6 mL) and stirred at 0° C. Lithium hydroxide mono hydrate (76 mg, 1.81 mmol) was added and the reaction mixture was stirred at 0° C. for 3.5 h. The reaction was quenched with 1.0 N HCl (25.0 mL) and extracted with ethyl acetate (3×50 mL). The combined organic extracts were washed with brine (1×50 mL), dried over anhydrous sodium sulfate, evaporated under reduced pressure and dried in vacuo to yield 0.40 g of the acid as a white solid. ¹H-NMR (400 MHz, DMSO-d₆) δ 12.06 (bs, 1H), 8.28 (d, 1H, J=8.47 Hz), 8.11 (d, 1H, J=7.96 Hz), 8.08 (d, 1H, J=7.95 Hz), 8.00 (s, 1H), 4.39-4.44 (m, 1H), 4.27-4.34 (m, 2H), 3.62 (s, 3H), 2.38-2.54 (m, 4H), 2.03 (s, 3H), 2.03 (s, 3H), 1.71-2.01 (m, 4H), 1.57-1.66 (m, 1H), 1.44-1.48 (m, 2H), 0.89 (d, 3H, J=6.68 Hz), 0.85 (d, 3H, J=6.68 Hz).

### Step c. Synthesis of Synthesis of fMLM-OSu

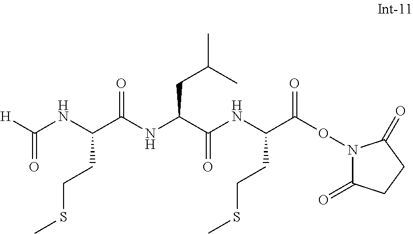 [SMILES: CSCC[C@H](NC=O)C(=O)N[C@@H](CC(C)C)C(=O)N[C@@H](CCSC)C(=O)ON1C(=O)CCC1=O]

A mixture of fMLM-OH (0.104 g, 0.247 mmol), N-hydroxysuccinimide (0.030 g, 1.05 eq) in DMF (3 mL) was added DCC (0.051 g, 1.0 eq). The reaction mixture was stirred at RT overnight. The precipitate was filtered and the product as a solution in DMF was used directly for the next step.

### Synthesis of fMLS(OBn)-OSu (Int 12)

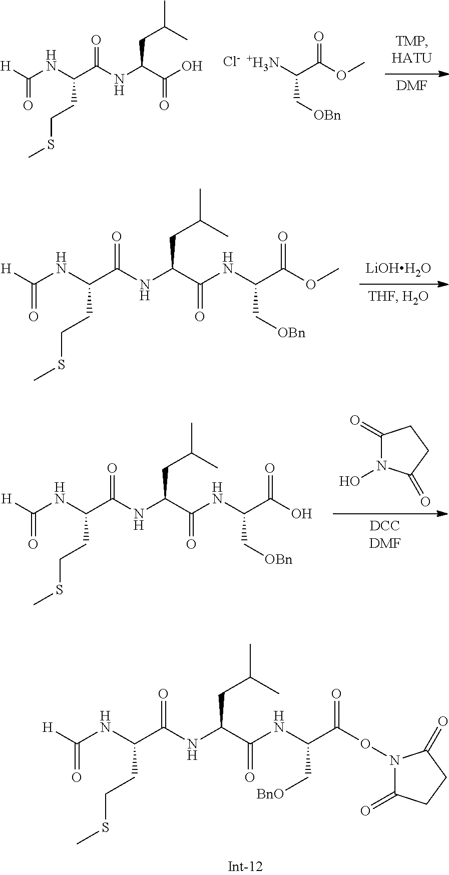

### Step a. Synthesis of fMLS(OBn)-OMe

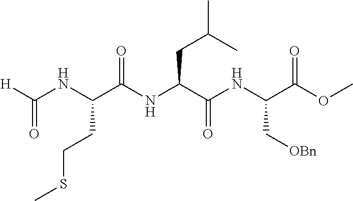 [SMILES: COC(=O)[C@H](COCc1ccccc1)NC(=O)[C@H](CC(C)C)NC(=O)[C@H](CCSC)NC=O]

A solution of formyl-Met-Leu-OH (Int-10, Step b, 0.60 g, 2.07 mmol) and Ser(OBn)-OMe.HCl (0.51 g, 2.07 mmol) in DMF (16 mL) was added TMP (0.55 mL, 4.13 mmol) and HATU (0.79 g, 2.07 mmol) at 0° C. and the resulting mixture was stirred at 0° C. for 1 h and at room temperature for 1.5 h under nitrogen. The reaction mixture was diluted with EtOAc (100 mL), washed with 1.0 N HCl (2×25 mL), sat-NaHCO₃ (2×50 mL), sat-NaCl (1×25 mL), dried over anhydrous sodium sulfate, and then concentrated under reduced pressure to yield 0.93 g of the desired product, which was used for the next step without further purification. ¹H-NMR (400 MHz, DMSO-d₆) δ 8.36 (d, 1H, J=7.56 Hz), 8.28 (d, 1H, J=8.12 Hz), 8.09 (d, 1H, J=8.49 Hz), 8.01 (s, 1H), 7.27-7.37 (m, 5H), 4.39-4.55 (m, 5H), 3.62-3.75 (m, 2H), 2.39-2.44 (m, 2H), 2.01 (s, 3H), 1.71-1.93 (m, 2H), 1.59-1.66 (m, 1H), 1.45-1.48 (m, 2H), 0.89 (d, 3H, J=6.42 Hz), 0.84 (d, 3H, J=6.42 Hz).

### Step b. Synthesis of fMLS(OBn)-OH

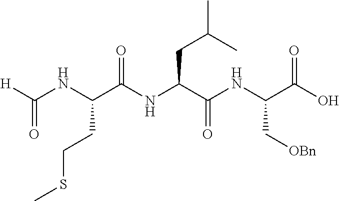 [SMILES: CSCC[C@H](NC=O)C(=O)N[C@@H](CC(C)C)C(=O)N[C@@H](COCc1ccccc1)C(=O)O]

The starting material, fMLS(OBn)-OMe (0.92 g, 1.91 mmol) was dissolved in a solvent mixture of THF (45 mL) and water (15 mL) and stirred at 0° G. Lithium hydroxide mono hydrate (148 mg, 3.53 mmol) was added and the reaction mixture was stirred at 0° C. for 1.5 h. The reaction was quenched with 1.0 N HCl (25.0 mL) and extracted with ethyl acetate (3×50 mL). The combined organic extracts were washed with brine (1×50 mL), dried over anhydrous sodium sulfate, evaporated under reduced pressure and dried in vacuo to yield 0.85 g of the acid as a white solid. ¹H-NMR (400 MHz, DMSO-d₆) δ 12.75 (bs, 1H), 8.28 (d, 1H, J=8.26 Hz), 8.16 (d, 1H, J=7.83 Hz), 8.11 (d, 1H, J=8.26 Hz), 8.01 (s, 1H), 7.26-7.37 (m, 5H), 4.38-4.54 (m, 5H), 3.60-3.79 (m, 2H), 2.40-2.44 (m, 2H), 2.01 (s, 3H), 1.71-1.93 (m, 2H), 1.59-1.66 (m, 1H), 1.46-1.49 (m, 2H), 0.89 (d, 3H, J=6.68 Hz), 0.84 (d, 3H, J=6.52 Hz).

### Step c. Synthesis of Synthesis of fMLS(OBn)-OSu

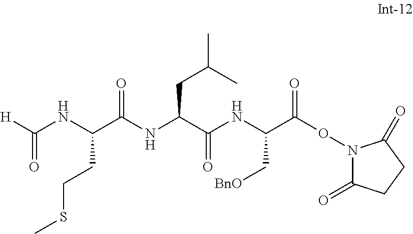 [SMILES: CSCC[C@H](NC=O)C(=O)N[C@@H](CC(C)C)C(=O)N[C@@H](COCc1ccccc1)C(=O)ON1C(=O)CCC1=O]

A mixture of fMLS(OBn)-OH (0.086 g, 0.184 mmol), N-hydroxysuccinimide (0.023 g, 1.05 eq) in DMF (3 mL) was added DCC (0.038 g, 1.0 eq). The reaction mixture was stirred at RT overnight. The precipitate was filtered and the product as a solution in DMF was used directly for the next step.

### Synthesis of fMLF-(Z)^{α}-Orn-OSu (Int-13)

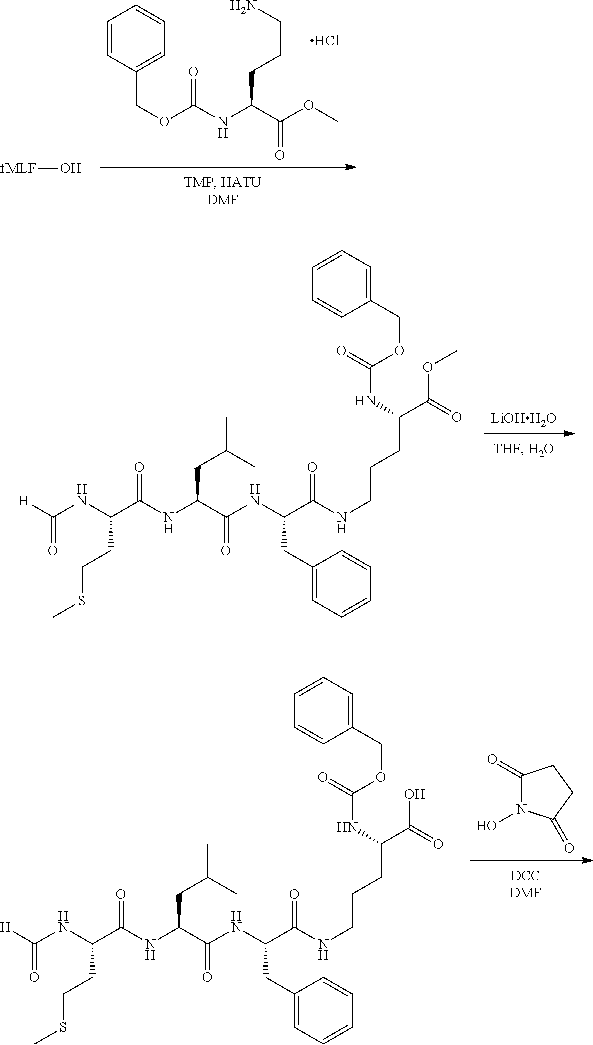

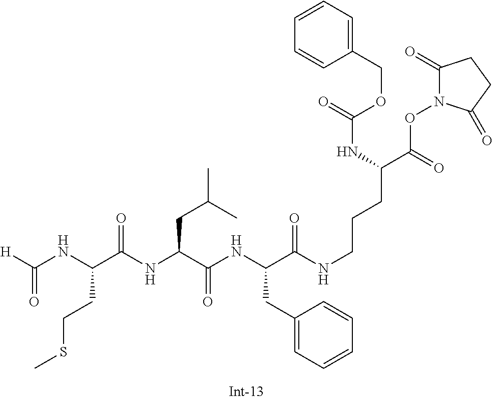 [SMILES: CSCC[C@H](NC=O)C(=O)N[C@@H](CC(C)C)C(=O)N[C@@H](Cc1ccccc1)C(=O)NCCC[C@H](NC(=O)OCc1ccccc1)C(=O)ON1C(=O)CCC1=O]

### Step a. Synthesis of fMLF-(Z)^{α}-Orn-OMe

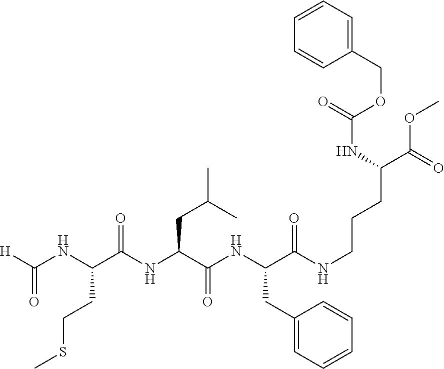 [SMILES: COC(=O)[C@H](CCCNC(=O)[C@H](Cc1ccccc1)NC(=O)[C@H](CC(C)C)NC(=O)[C@H](CCSC)NC=O)NC(=O)OCc1ccccc1]

To a solution of formyl-Met-Leu-Phe-OH (0.5 g, 1.14 mmol) and (Z)-Orn-OMe hydrochloride (0.361 g, 1.14 mmol) in DMF (9.0 mL) is added TMP (0.3 mL, 2.28 mol) and HATU (0.435 g, 1.14 mmol) at 0° C. and the resulting mixture is stirred at 0° C. for 1 h and then at room temperature for 1.5 h under nitrogen or until analysis shows substantial formation of product. The reaction mixture is diluted with EtOAc (75 mL), washed with 1.0 N HCl (2×15 mL), sat-NaHCO₃ (2×15 mL), sat-NaCl (2×15 mL), and dried over anhydrous sodium sulfate and concentrated under reduced pressure. The crude product is triturated with diethyl ether and then dried under vacuum to yield the desired product with a mass of 699.33.

### Step b. Synthesis of fMLF-(Z)^{α}-Orn-OH

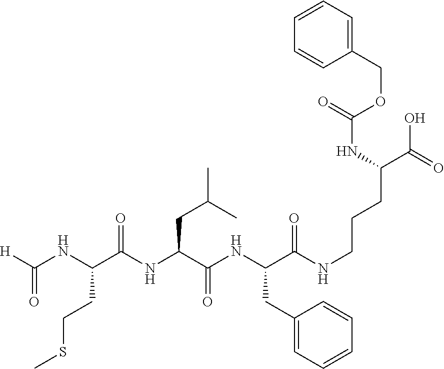 [SMILES: CSCC[C@H](NC=O)C(=O)N[C@@H](CC(C)C)C(=O)N[C@@H](Cc1ccccc1)C(=O)NCCC[C@H](NC(=O)OCc1ccccc1)C(=O)O]

The starting material, fMLF-(Z)^{α}-Orn-OMe 0.7 g, 1 mmol), is dissolved in a solvent mixture of THF (32 mL)/water (16 mL) and stirred at 0° C. Lithium hydroxide mono hydrate (90 mg, 2.15 mmol) is added and the reaction mixture is stirred at 0° C. for 3.5 h or until analysis shows substantial formation of product. The reaction is quenched with 1.0 N HCl (5.0 mL), diluted with water (50 mL), and extracted with ethyl acetate (1×100 mL). The organic extract is washed with brine (1×50 mL) and dried over anhydrous sodium sulfate. The solvents are evaporated under reduced pressure and the product is triturated with diethyl ether to yield the desired product with a mass of 685.31.

### Step c. Synthesis of fMLF-(Z)^{α}-Orn-OSu

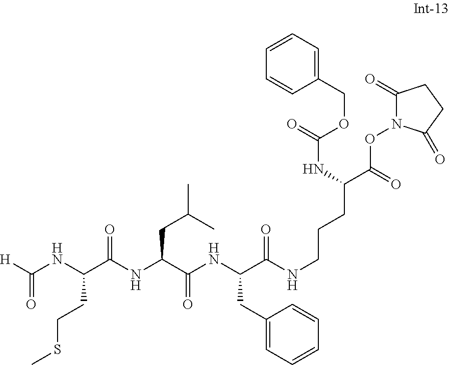 [SMILES: CSCC[C@H](NC=O)C(=O)N[C@@H](CC(C)C)C(=O)N[C@@H](Cc1ccccc1)C(=O)NCCC[C@H](NC(=O)OCc1ccccc1)C(=O)ON1C(=O)CCC1=O]

A mixture of fMLF-(Z)^{α}-Orn-OH (0.169 g, 0.247 mmol), HOSu (0.030 g, 1.05 eq) in DMF (3 mL) is added DCC (0.051 g, 1.0 eq). The reaction mixture is stirred at RT overnight (or until most of the acid is consumed as monitored by TLC and LC-MS analyses). The precipitate is filtered and the product with a mass of 782.33 is used directly in subsequent reactions as a solution in DMF.

### Synthesis of fMLF-L-(Z)^{α}-Dap-OSu (Int-14)

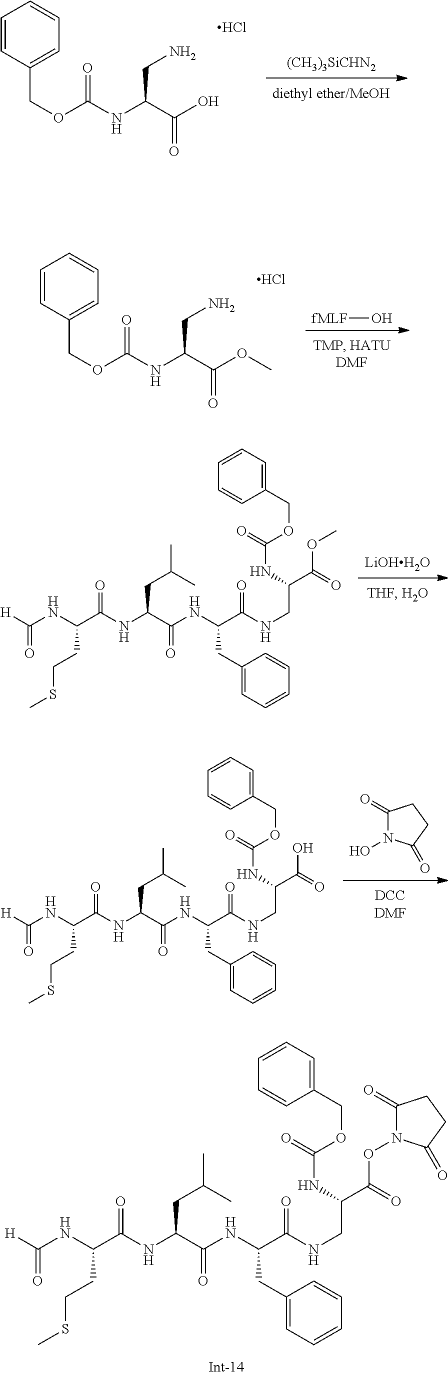

### Step a. Synthesis of L-(Z)^{α}-Dap-OMe

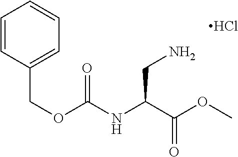 [SMILES: COC(=O)[C@H](CN)NC(=O)OCc1ccccc1 ; Cl]

To a solution of L-(Z)^{α}-Dap-OH hydrochloride (1.44 g, 5.00 mmol) in methanol (20 mL), a 2.0 M solution of trimethylsilyldiazomethane in diethyl ether (2.8 mL, 5.6 mmol) is slowly added at 0° C. After 1.5 h or until analysis shows no further disappearance of starting carboylic acid, glacial acetic acid (0.3 mL) is added and the mixture stirred until any yellow color has dissipated. The solvent is removed under reduced pressure and the residue is dissolved in water (20 mL) and washed with ethyl acetate (2×20 mL), Lyophilization of the aqueous layer followed by trituration of the residue with diethyl ether gives the crude product with a parent mass of 252.11 used in the subsequent reactions.

### Step b. Synthesis of fMLF-L-(Z)^{α}-Dap-OMe

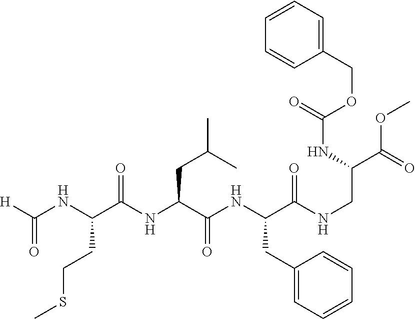 [SMILES: COC(=O)[C@H](CNC(=O)[C@H](Cc1ccccc1)NC(=O)[C@H](CC(C)C)NC(=O)[C@H](CCSC)NC=O)NC(=O)OCc1ccccc1]

To a solution of formyl-Met-Leu-Phe-OH (0.5 g, 1.14 mmol) and L-(Z)-Dap-OMe hydrochloride (0.433 g, 1.37 mmol) in DMF (9.0 mL) is added TMP (0.3 mL, 2.28 mol) and HATU (0.435 g, 1.14 mmol) at 0° C. and the resulting mixture is stirred at 0° C. for 1 h and then at room temperature for 1.5 h under nitrogen or until analysis shows substantial reaction. The reaction mixture is diluted with EtOAc (75 mL), washed with 1.0 N HCl (2×15 mL), sat-NaHCO₃ (2×15 mL), sat-NaCl (2×15 mL), and dried over anhydrous sodium sulfate and concentrated under reduced pressure. The crude product is triturated with diethyl ether and then dried under vacuum to yield the desired product with a mass of 699.33.

### Step c. Synthesis of fMLF-L-(Z)^{α}-Dap-OH

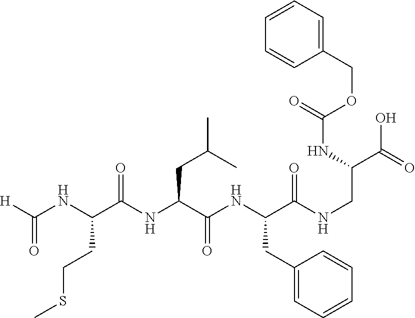 [SMILES: CSCC[C@H](NC=O)C(=O)N[C@@H](CC(C)C)C(=O)N[C@@H](Cc1ccccc1)C(=O)NC[C@H](NC(=O)OCc1ccccc1)C(=O)O]

The starting material, fMLF-L-(Z)^{α}-Orn-OMe 0.7 g, 1 mmol), is dissolved in a solvent mixture of THF (32 mL)/water (16 mL) and stirred at 0° C. Lithium hydroxide mono hydrate (90 mg, 2.15 mmol) is added and the reaction mixture is stirred at 0° C. for 3.5 h or until analysis shows substantial reaction. The reaction is quenched with 1.0 N HCl (5.0 mL), diluted with water (50 mL), and extracted with ethyl acetate (1×100 mL). The organic extract is washed with brine (1×50 mL) and dried over anhydrous sodium sulfate. The solvents are evaporated under reduced pressure and the product is triturated with diethyl ether to yield the desired product with a mass of 685.31.

### Step d. Synthesis of fMLF-L-(Z)^{α}-Dap-OSu

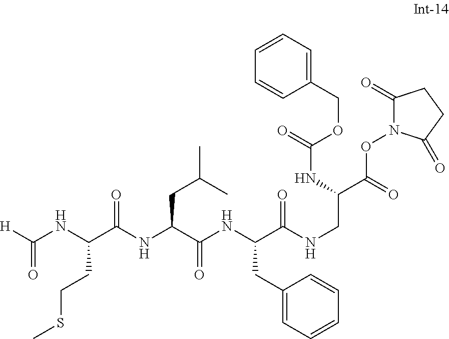 [SMILES: CSCC[C@H](NC=O)C(=O)N[C@@H](CC(C)C)C(=O)N[C@@H](Cc1ccccc1)C(=O)NC[C@H](NC(=O)OCc1ccccc1)C(=O)ON1C(=O)CCC1=O]

A mixture of fMLF-L-(Z)^{α}-Orn-OH (0.169 g, 0.247 mmol), HOSu (0.030 g, 1.05 eq) in DMF (3 mL) is added DCC (0.051 g, 1.0 eq). The reaction mixture is stirred at RT overnight (or until a significant amount of the acid is consumed as monitored by TLC and LC-MS analyses). The precipitate is filtered and the product with a mass of 782.33 is used directly in subsequent reactions as a solution in DMF.

### Synthesis of Int-15

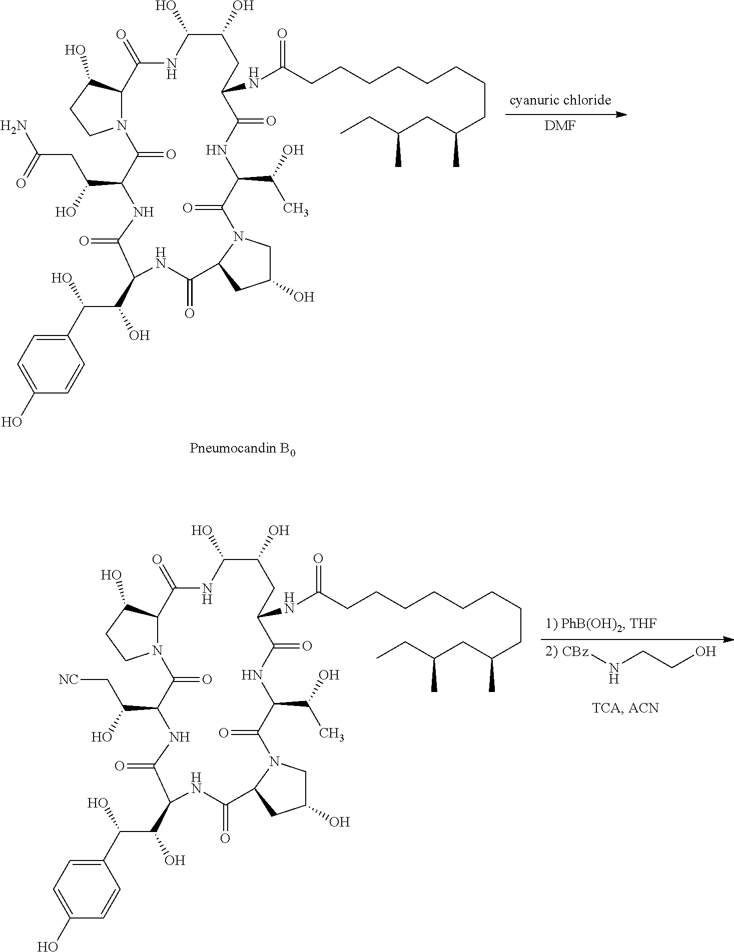

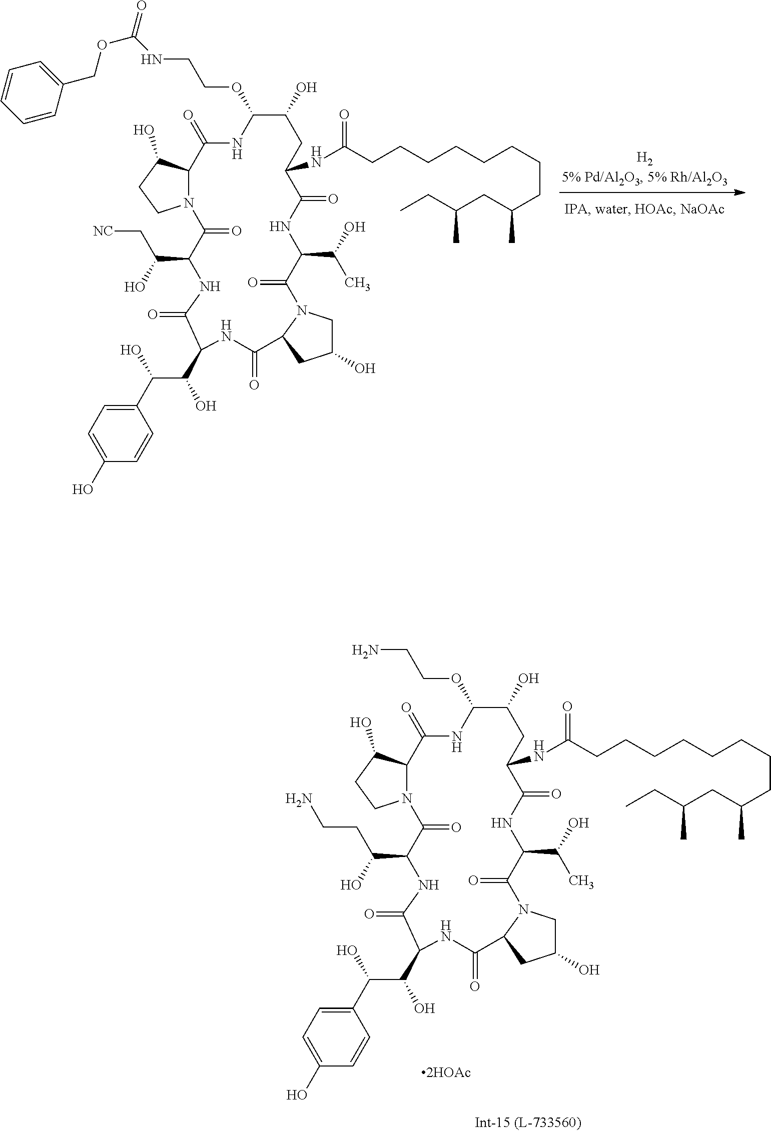 [SMILES: CC[C@H](C)C[C@H](C)CCCCCCCCC(=O)N[C@H]1C[C@@H](O)[C@@H](OCCNC(=O)OCc2ccccc2)NC(=O)[C@@H]2[C@@H](O)CCN2C(=O)[C@H]([C@H](O)CC#N)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

### Step a. Synthesis of Cyano Intermediate

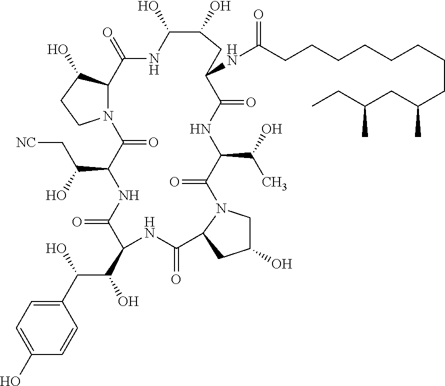 [SMILES: CC[C@H](C)C[C@H](C)CCCCCCCCC(=O)N[C@H]1C[C@@H](O)[C@@H](O)NC(=O)[C@@H]2[C@@H](O)CCN2C(=O)[C@H]([C@H](O)CC#N)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Pneumocandin B₀ (2.5 g, 2.345 mmol) was dissolved in dry N,N-dimethylformamide (30 ml). To this solution was added cyanuric chloride (1.0 g, 5.4 mmol) in one portion. The mixture was stirred at room temperature for 5.5 min. Sodium acetate in water (2.0 M, 20.0 mL) was added and the product was purified via preparative HPLC to yield 1.25 g of product. ¹H-NMR (400 MHz, Methanol-d₄) δ 7.54 (d, 1H, J=8.0 Hz), 7.13 (d, 2H, J=8.0 Hz), 5.31 (d, 1H, J=4.0 Hz), 4.97-5.04 (m, 2H), 4.53-4.59 (m, 3H), 4.19-4.44 (m, 7H), 3.95-4.00 (m, 2H), 3.88-3.93 (m, 1H), 3.76-3.82 (m, 2H), 2.79-2.85 (m, 1H), 2.67-2.73 (m, 1H), 2.40-2.46 (m, 1H), 2.22-2.32 (m, 3H), 1.94-2.10 (m, 4H), 1.57-1.65 (m, 2H), 1.20-1.50 (m, 18H), 1.04-1.14 (m, 2H), 0.82-0.95 (m, 10H); LC/MS, 1047.4 [M+H]⁺.

### Step b. Synthesis of N-CBz-Aminoethyl ether Intermediate

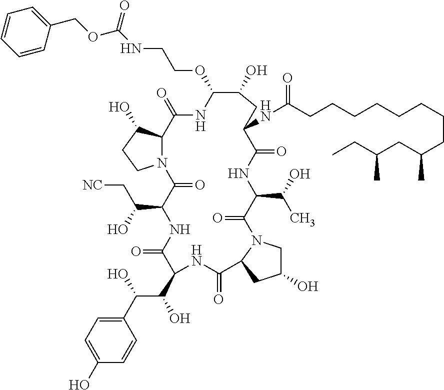 [SMILES: CC[C@H](C)C[C@H](C)CCCCCCCCC(=O)N[C@H]1C[C@@H](O)[C@@H](OCCNC(=O)OCc2ccccc2)NC(=O)[C@@H]2[C@@H](O)CCN2C(=O)[C@H]([C@H](O)CC#N)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a solution of the product from step a (1.25 g, 1.19 mmol) in dry THF (24 mL) was added phenylboronic acid (0.21 g, 1.67 mmol) at room temperature and the solution was concentrated to dryness to remove the water. This azeotropic distillation was repeated twice. The resulting borate was slurried in dry acetonitrile (42 mL) with N—Z-ethanolamine (1.87 g, 9.55 mmol) and the reaction mixture was cooled to 0° C. A solution of trichloroacetic acid (14.3 g, 87.6 mmol) in dry ACN (18 mL) was added over 5 min and the resulting mixture was stirred at 0° C. for 48 h under nitrogen. The reaction was quenched into water (90 mL) at 0° C. The mixture was loaded on YMC ODS-A C-18 (25 g) and washed with a 60:40 mixture of water/acetonitrile (500 mL) to remove the excess N—Z-ethanolamine and phenylboric acid. The product was eluted with methanol (150 mL) and then concentrated under reduced pressure. The crude product was purified using preparative HPLC to yield 710 mg of the desired product. ¹H-NMR (400 MHz, Methanol-d₄)δ 7.28-7.35 (m, 5H), 7.11-7.15 (m, 2H), 6.73-6.77 (m, 2H), 5.21 (d, 1H, J=2.3 Hz), 5.07 (s, 2H), 5.03 (d, 1H, J=3.7 Hz), 5.00 (d, 1H, J=3.7 Hz), 4.52-4.59 (m, 3H), 4.40-4.44 (m, 1H), 4.21-4.35 (m, 6H), 4.03-4.07 (m, 1H), 3.96-3.99 (m, 1H), 3.86-3.91 (m, 1H), 3.77-3.83 (m, 2H), 3.61-3.66 (m, 1H), 3.50-3.56 (m, 1H), 2.68-2.83 (m, 2H), 2.40-2.46 (m, 1H), 2.19-2.28 (m, 3H), 1.87-2.10 (m, 4H), 1.57-1.63 (m, 2H), 1.19-1.51 (m, 18H), 1.04-1.13 (m, 2H), 0.85-0.95 (m, 10H); LC/MS, 1248.5 [M+Na]⁺.

### Step c. Synthesis of Intermediate 15

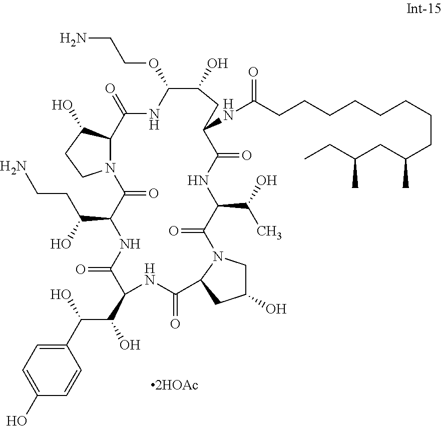 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

The product from step b (0.7 g, 0.57 mmol) was dissolved in a 9:1 mixture of iso-propanol/water (15 mL) and to this solution was added acetic acid (0.75 mL), ammonium acetate (1.55 g), Pd/Al₂O₃ (5% Pd, 60 mg), and Rh/Al₂O₃ (5% Rh, 120 mg). The resulting black slurry was treated with hydrogen (40 psi) at room temperature for 12 h, diluted with water (46 mL), and filtered through 0.45 mm PTFE ACRODISC syringe filter. The filtrate was loaded on YMC ODS-A C-18 (10 g) and washed with a 90:10 mixture of water/methanol (200 mL). The product was eluted with methanol (100 mL) and then concentrated under reduced pressure and dried via lyophilization to yield 630 mg of the desired product (Int-15) as the bis-acetate salt. ¹H-NMR (400 MHz, Methanol-d₄) δ 7.11-7.14 (m, 2H), 6.74-6.77 (m, 2H), 5.21 (d, 1H, J=2.2 Hz), 4.98 (d, 1H, J=3.3 Hz), 4.91 (d, 1H, J=6.3 Hz), 4.53-4.64 (m, 3H), 4.44-4.49 (m, 1H), 4.21-4.36 (m, 5H), 4.12-4.17 (m, 1H), 4.03-4.08 (m, 1H), 3.95-3.99 (m, 1H), 3.78-3.86 (m, 3H), 3.69-3.75 (m, 1H), 3.60-3.65 (m, 1H), 3.06 (t, 2H, J=7.1 Hz), 3.00-3.02 (m, 2H), 2.41-2.46 (m, 1H), 2.23-2.31 (m, 3H), 1.98-2.10 (m, 5H), 1.90 (s, 6H), 1.76-1.86 (m, 1H), 1.56-1.64 (m, 2H), 1.05-1.55 (m, 22H), 0.85-0.95 (m, 10H); LC/MS, 1094.5 [M+H]⁺.

### Synthesis of (N-formyl)Met-Ile-Phe-Leu N-Hydroxysuccinimide Ester (Int-16)

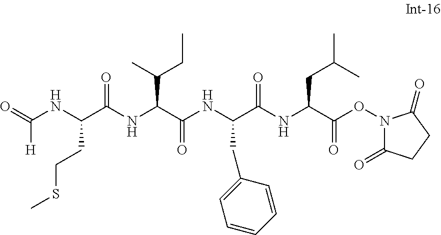 [SMILES: CCC(C)[C@H](NC(=O)[C@H](CCSC)NC=O)C(=O)N[C@@H](Cc1ccccc1)C(=O)N[C@@H](CC(C)C)C(=O)ON1C(=O)CCC1=O]

In a procedure similar to that used for the synthesis of Int-1, fMIFL-OSu may be prepared starting with fMIFL and N-hydroxysuccinimide and treating with DCC in DMF. The exact mass of the product is 647.30.

### Synthesis of (N-formyl)Met-Ile-Val-Ile-Leu N-Hydroxysuccinimide Ester (Int-17)

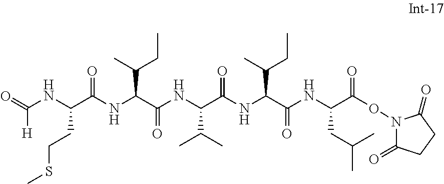 [SMILES: CCC(C)[C@H](NC(=O)[C@H](CCSC)NC=O)C(=O)N[C@H](C(=O)N[C@H](C(=O)N[C@@H](CC(C)C)C(=O)ON1C(=O)CCC1=O)C(C)CC)C(C)C]

In a procedure similar to that used for the synthesis of Int-1, fMIVIL-OSu may be prepared starting with fMIVIL and N-hydroxysuccinimide and treating with DCC in DMF. The exact mass of the product is 712.38

### Synthesis of (N-iso-BOC)-MLF-OSu (Int-18)

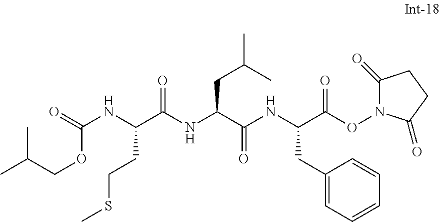 [SMILES: CSCC[C@H](NC(=O)OCC(C)C)C(=O)N[C@@H](CC(C)C)C(=O)N[C@@H](Cc1ccccc1)C(=O)ON1C(=O)CCC1=O]

(N-iso-BOC)-MLF-OH (50 mg, 0.1 mmol, 1 eq) was dissolved in 1 mL dry DMF. EDCI (28 mg, 0.15 mmol, 1.5 eq) and N-hydroxysuccinimide (12 mg, 0.1 mmol, 1.05 eq) were added. When HPLC analysis indicated no (N-iso-BOC)-MLF-OH remained, the reaction was used directly in subsequent reactions without purification.

### Synthesis of N^{ε}-fMLF-N^{α}-FMOC-K (Int-19)

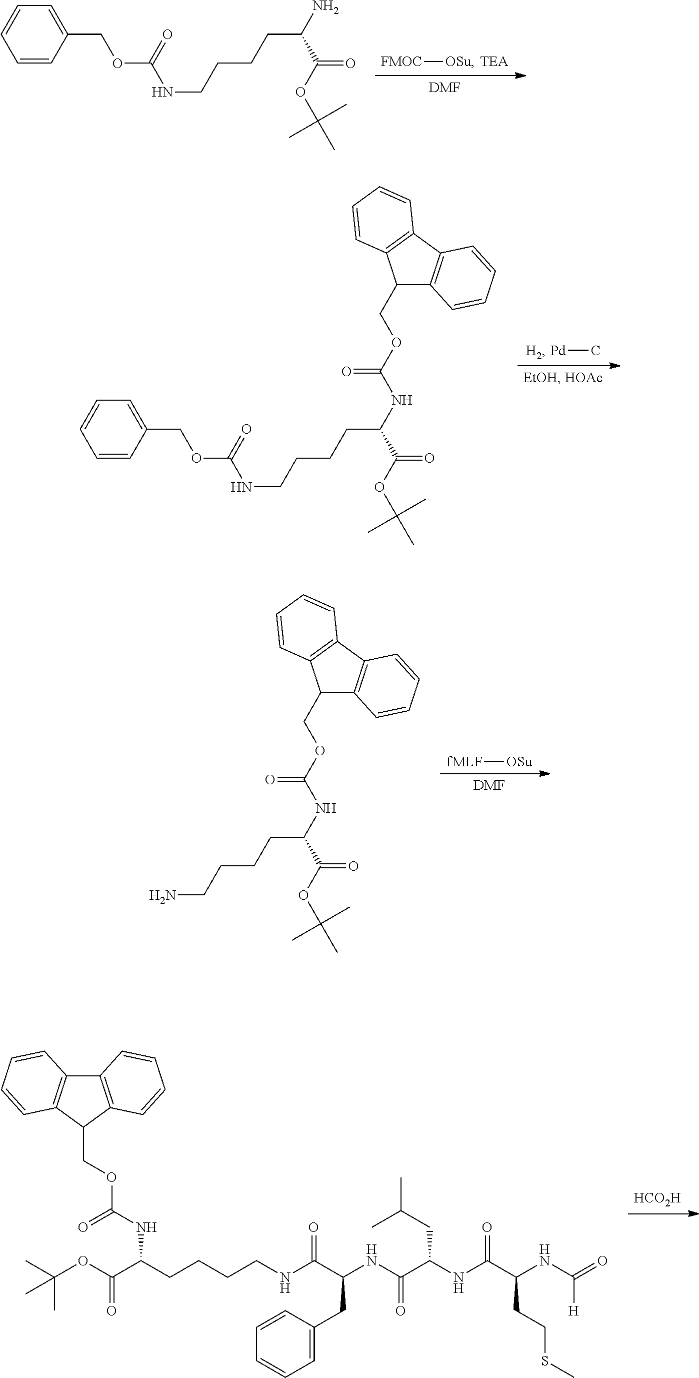

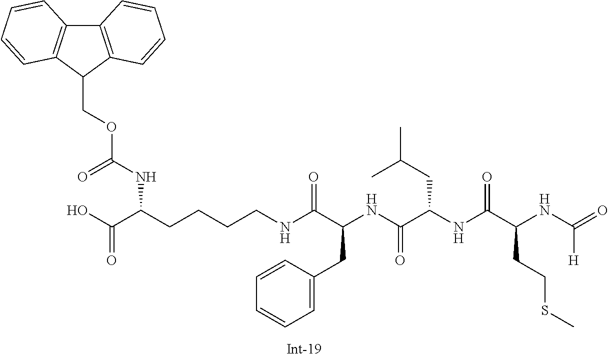 [SMILES: CSCC[C@H](NC=O)C(=O)N[C@@H](CC(C)C)C(=O)N[C@@H](Cc1ccccc1)C(=O)NCCCC[C@@H](NC(=O)OCC1c2ccccc2-c2ccccc21)C(=O)O]

### Step a) Preparation of N^{α}-FMOC-N^{ε}—Z-Lysine tert-Butyl Ester

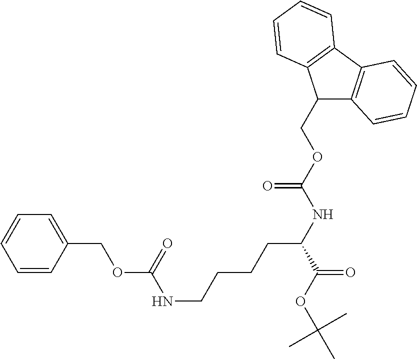 [SMILES: CC(C)(C)OC(=O)[C@H](CCCCNC(=O)OCc1ccccc1)NC(=O)OCC1c2ccccc2-c2ccccc21]

N^{ε}—Z-Lysine tert-butyl ester (300 mg, 0.81 mmoles) and FMOC-OSu (299 mg, 0.89 mmoles, 1.1 eq) were dissolved in 3 mL dry DMF and TEA (112 μL, 0.81 mmoles, 1 eq) was added. The reaction was stirred at room temperature for 1 hour. The reaction was diluted with 10% aqueous LiCl and extracted 3 times with 10% heptanes in ethyl acetate. The organic extractions were combined and back extracted with 10% aqueous LiCl. The organic solution was dried over sodium sulfate, filtered, and evaporated. Yield 519 mg. Target Yield 452 mg. LC/MS, 559.0 [M+H]⁺. ¹H NMR d₆-DMSO is consistent with the desired product and indicated trace amounts of DMF and ethyl acetate. ¹H NMR (400 MHz, DMSO-d₆) δ ppm 1.38 (s, 10H) 1.50-1.72 (m, 1H) 2.92-3.05 (m, 1H) 3.79-3.90 (m, 1H) 4.16-4.25 (m, 1H) 4.26-4.33 (m, 1H) 5.00 (s, 1H) 7.20-7.26 (m, 1H) 7.29-7.37 (m, 7H) 7.41 (s, 2H) 7.59-7.66 (m, 1H) 7.72 (d, J=7.42 Hz, 2H) 7.89 (d, J=7.42 Hz, 2H)

### Step b) Removal of Z-group

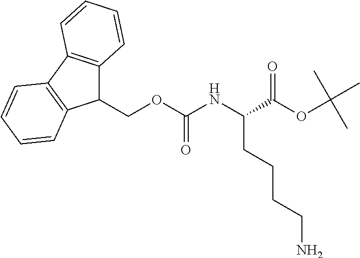 [SMILES: CC(C)(C)OC(=O)[C@H](CCCCN)NC(=O)OCC1c2ccccc2-c2ccccc21]

The product from Step a) was dissolved in 20 mL ethanol and 2 mL acetic acid. 150 mg of 5% Pd/C was added and the reaction was placed under a hydrogen atmosphere (balloon pressure). After stirring overnight, the reaction was filtered and the solvent was removed under reduced pressure. LC/MS, 425.2 [M+H]⁺. The crude product was used without purification.

### Step c) Preparation of N^{ε}-fMLF-N^{α}-FMOC-K tert-Butyl Ester

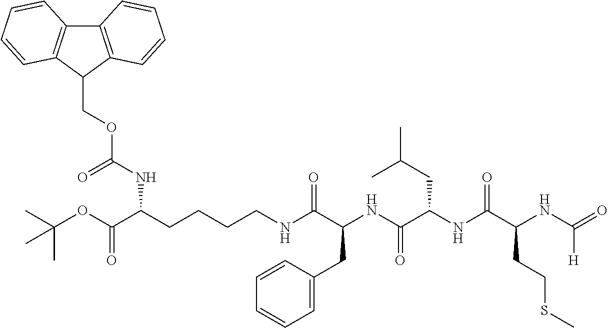 [SMILES: CSCC[C@H](NC=O)C(=O)N[C@@H](CC(C)C)C(=O)N[C@@H](Cc1ccccc1)C(=O)NCCCC[C@@H](NC(=O)OCC1c2ccccc2-c2ccccc21)C(=O)OC(C)(C)C]

The product from Step b) above (344 mg, 0.81 mmol, 1.7 eq) was dissolved in 7 mL dry DMF and fMLF-OSu (Int-1) (246 mg, 0.46 mmol) dissolved in 2 mL dry DMF was added dropwise. After 10 minutes the product was purified by prep-HPLC using a 60% to 100% methanol in water gradient over 5 minutes. Yield 120 mg. LC/MS, 844.2 [M+H]⁺. ¹H NMR (400 MHz, DMSO-d₆) δ ppm 0.80 (d, J=6.64 Hz, 3H) 0.85 (d, J=6.64 Hz, 3H) 1.09-1.34 (m, 5H) 1.34-1.44 (m, 12H) 1.44-1.64 (m, 3H) 1.67-1.81 (m, 1H) 1.81-1.92 (m, 1H) 2.01 (s, 3H) 2.34-2.44 (m, 2H) 2.76-2.86 (m, 1H) 2.87-2.98 (m, 2H) 2.99-3.14 (m, 1H) 3.77-3.88 (m, 1H) 4.16-4.36 (m, 4H) 4.36-4.48 (m, 2H) 6.28 (s, 1H) 7.17 (d, J=7.22 Hz, 3H) 7.22 (d, J=6.25 Hz, 3H) 7.33 (dd, J=15.23, 7.61 Hz, 3H) 7.42 (td, J=7.42, 0.98 Hz, 3H) 7.61 (d, J=7.81 Hz, 1H) 7.72 (d, J=7.22 Hz, 2H) 7.84 (d, J=7.42 Hz, 2H) 7.89 (dd, J=7.52, 2.44 Hz, 3H) 7.93 (d, J=8.20 Hz, 1H) 7.99-8.03 (m, 1H) 8.08 (d, J=8.00 Hz, 1H) 8.30 (d, J=8.00 Hz, 1H)

### Step d) Removal of the tert-Butyl Ester

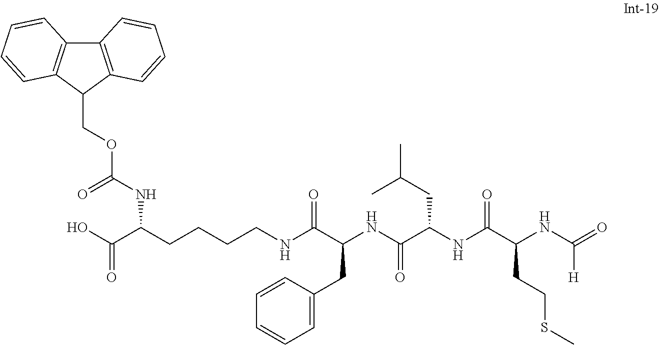 [SMILES: CSCC[C@H](NC=O)C(=O)N[C@@H](CC(C)C)C(=O)N[C@@H](Cc1ccccc1)C(=O)NCCCC[C@@H](NC(=O)OCC1c2ccccc2-c2ccccc21)C(=O)O]

The product from Step c) above (120 mg, 0.14 mmol) was dissolved in 20 mL formic acid and stirred at room temperature for two hours. The formic acid was removed under reduced pressure. The residue was diluted twice with toluene and evaporated to remove residual formic acid. The product was used without further purification. Yield 134 mg. LC/MS, 788.2 [M+H]⁺.

### Synthesis of fMLF-N^{ε}-FMOC-Lysine (Int-20)

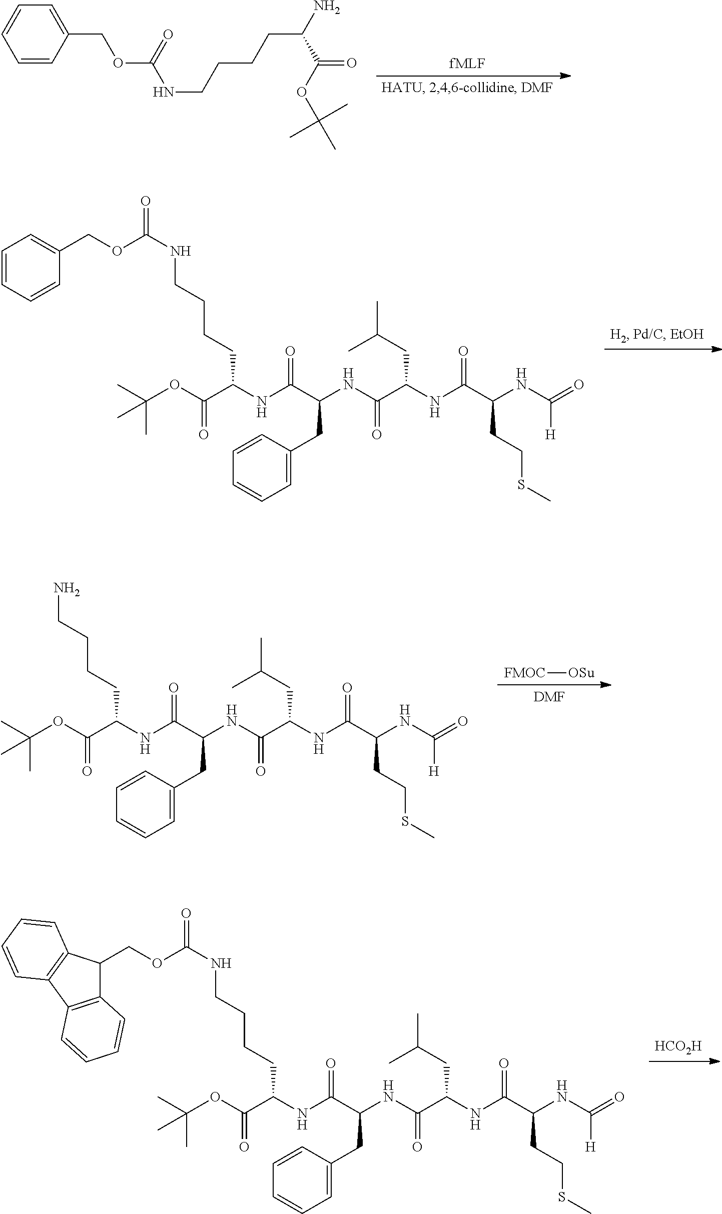

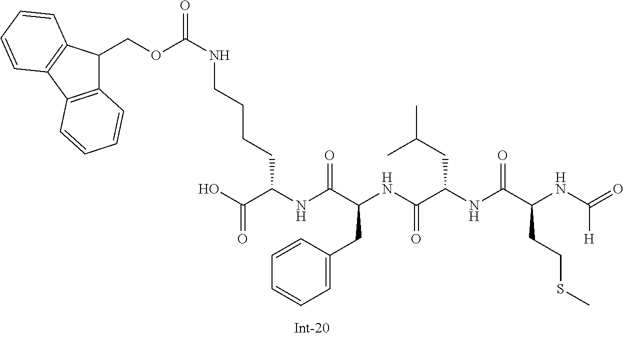 [SMILES: CSCC[C@H](NC=O)C(=O)N[C@@H](CC(C)C)C(=O)N[C@@H](Cc1ccccc1)C(=O)N[C@@H](CCCCNC(=O)OCC1c2ccccc2-c2ccccc21)C(=O)O]

### Step a) Preparation of fMLF-(N^{ε}—Z-Lysine) tert-Butyl Ester

A solution of N^{ε}—Z-Lysine tert-butyl ester 0.852 g, 2.29 mmol) and fMLF (1.00 g, 2.29 mmol) in DMF (20 mL) was charged with HATU (0.869 g, 2.29 mmol) and 2,4,6-collidine (0.554 g, 4.57 mmol). After stirring at room temperature for 40 min, the reaction was diluted with EtOAc (200 mL) and washed sequentially with saturated LiCl (3×50 mL), saturated NaHCO₃ (2×50 mL), and saturated NaCl (2×50 mL), then dried over Na₂SO₄, filtered, and concentrated under reduced pressure. The crude product was chromatographed on a 40 g Isco RediSep silica cartridge eluting with a gradient from 100% heptane to 100% EtOAc, and then to 100% MeOH Yield: 1.49 g as a white solid. ¹H NMR (400 MHz, Chloroform-d) δ ppm 0.84 (d, J=5.22 Hz, 3H) 0.87 (d, J=6.20 Hz, 3H) 1.20-2.06 (m, 9H) 1.44 (s, 9H) 2.08 (s, 3H) 2.50 (t, J=7.35 Hz, 2H) 2.81 (s, 1H) 2.92-3.37 (m, 4H) 4.30-4.75 (m, 4H) 5.04-5.19 (m, 2H) 5.22-5.29 (m, 1H) 6.32-6.52 (m, 1H) 6.74 (d, J=7.13 Hz, 1H) 6.83-7.00 (m, 2H) 7.10-7.47 (m, 10H) 7.69-7.85 (m, 1H) 8.04-8.20 (m, 1H). MS (M+H) 756.2.

### Step b) Preparation of fMLFK-O-tert-Butyl Ester

A solution of fMLF-(N^{ε}—Z-lysine) tert-butyl ester (1.96 g, 2.60 mmol) in EtOH (300 mL) was charged with 5% Pd on carbon (5.52 g), then evacuated and blanketed under H₂ via balloon 3 times. After stirring at rt for 19 h, the reaction was filtered through celite, and the filter cake was eluted with 9:1 EtOAc:MeOH, collecting 4×500 mL fractions. Fractions containing product (1 and 2) were pooled and concentrated under reduced pressure, and the isolated product was not purified further. Yield: 1.38 g as a white solid. ¹H NMR (400 MHz, DMSO-d₆) δ ppm 0.79 (d, J=6.54 Hz, 3H) 0.85 (d, J=6.54 Hz, 3H) 1.21-1.90 (m, 9H) 1.40 (s, 9H) 2.01 (s, 3H) 2.31-2.44 (m, 2H) 2.58 (t, J=6.76 Hz, 2H) 2.75-3.10 (m, 2H) 3.19-3.50 (m, 2H) 3.99-4.70 (m, 6H) 7.11-7.26 (m, 5H) 7.93 (d, J=8.25 Hz, 1H) 8.00 (s, 1H) 8.04 (d, J=8.25 Hz, 1H) 8.21 (d, J=7.52 Hz, 1H) 8.29 (d, J=8.00 Hz, 1H). MS (M+H) 622.2.

### Step c) Preparation of fMLF-(N^{ε}-FMOC-Lysine) tert-Butyl Ester

A solution of fMLFK-O-tert-butyl ester (1.37 g, 2.20 mmol) in DMF (20 mL) was charged with Fmoc-OSu (0.818 g, 2.42 mmol). After stirring at rt for 10 min, the reaction was diluted with EtOAc (250 mL) and washed with saturated LiCl (3×50 mL, with MeOH added to break an emulsion when needed), then dried over Na₂SO₄, filtered, and concentrated under reduced pressure. The crude product was then chromatographed on a 40 g Isco RediSep silica cartridge eluting with a gradient from 100% heptane to 100% EtOAc, and then to 100% MeOH. Yield: 1.62 g as a white solid. ¹H NMR (400 MHz, DMSO-d₆) δ ppm 0.77 (d, J=6.54 Hz, 3H) 0.82 (d, J=6.54 Hz, 3H) 1.15-1.90 (m, 9H) 1.38 (s, 9H) 1.99 (s, 3H) 2.28-2.44 (m, 2H) 2.76-3.10 (m, 2H) 3.94-4.70 (m, 11H) 7.09-7.27 (m, 5H) 7.28-7.37 (m, 3H) 7.40 (t, J=7.49 Hz, 2H) 7.68 (d, J=7.52 Hz, 2H) 7.88 (d, J=7.47 Hz, 2H) 8.00 (s, 1H) 8.04 (d, J=8.30 Hz, 1H) 8.15 (d, J=8.39 Hz, 1H) 8.28 (d, J=7.17 Hz, 1H) 8.33-8.46 (m, 1H). MS (M+H) 844.2.

### Step d) Removal of the tert-Butyl Ester to Provide Int-20

A solution of fMLF-(N^{ε}-FMOC-Lysine) tert-butyl ester (1.61 g, 1.91 mmol) in formic acid (20 mL) was stirred at rt for 3 h. The reaction was then concentrated under reduced pressure, chromatographed on a 50 g Isco Gold RediSep C-18 reversed phase silica cartridge, eluting with a gradient from 99.9:0.1 H₂O:HOAc to 99.9:0.1 MeOH:HOAc. The isolated product was then lyophilized from 1:1 H₂O:CH₃CN (8 mL). Yield: 0.304 g. ¹H NMR (400 MHz, DMSO-d₆) δ ppm 0.79 (d, J=6.39 Hz, 3H) 0.84 (d, J=6.49 Hz, 3H) 1.12-1.92 (m, 9H) 2.00 (s, 3H) 2.38 (t, J=6.66 Hz, 2H) 2.73-3.10 (m, 4H) 3.31 (br. s., 2H) 4.07-4.63 (m, 7H) 7.09-7.36 (m, 8H) 7.37-7.46 (m, 2H) 7.68 (d, J=7.27 Hz, 2H) 7.83-7.97 (m, 3H) 7.98-8.06 (m, 2H) 8.07-8.14 (m, 1H) 8.27 (d, J=7.71 Hz, 1H) 12.20-12.95 (m, 1H). MS (M+H) 788.2.

### Example 2

### Synthesis of Compound 1

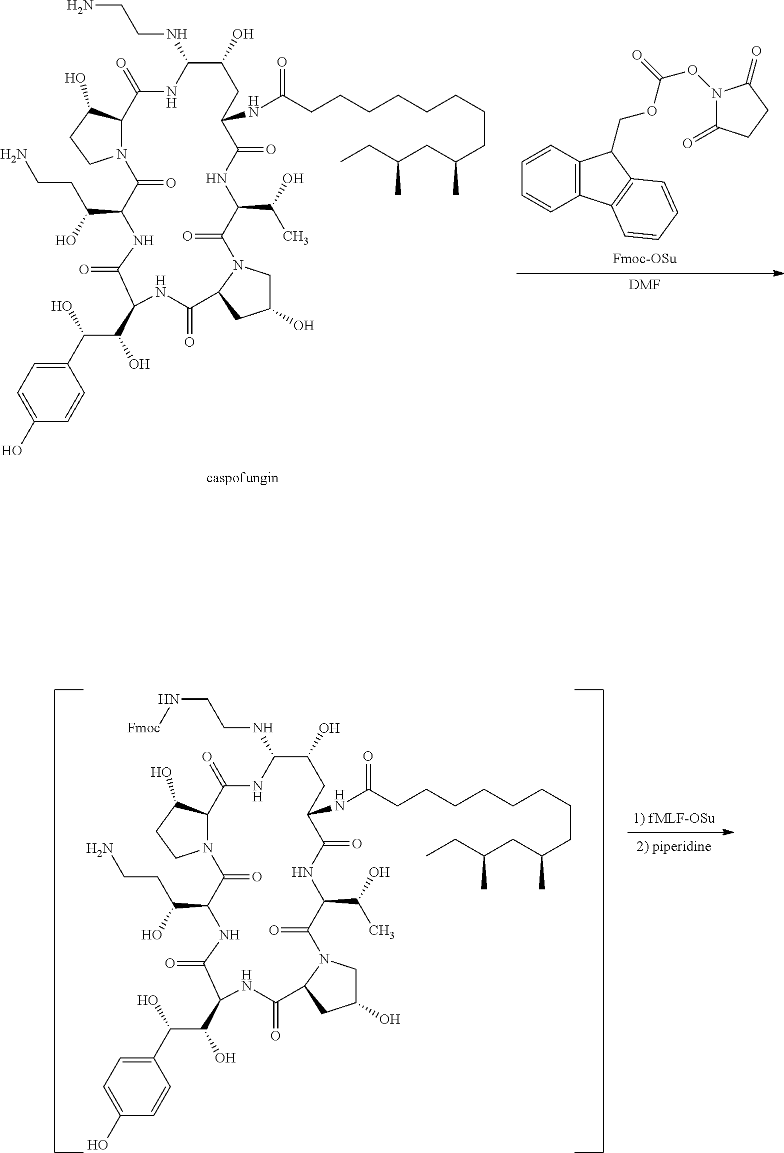

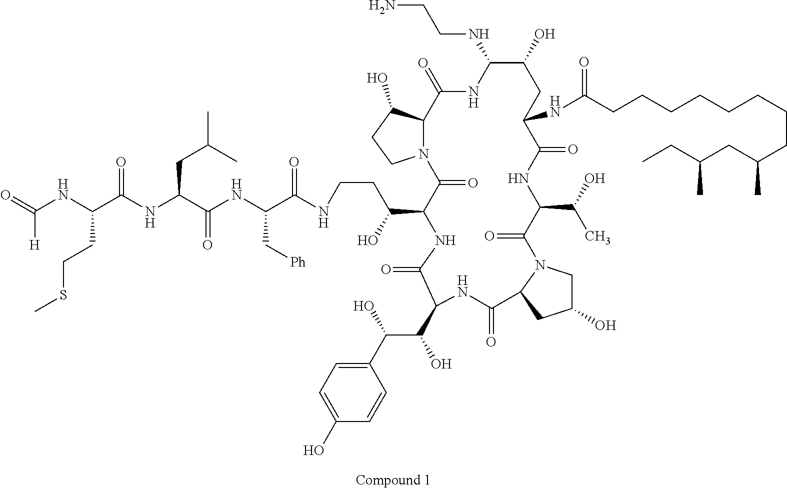 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a solution of caspofungin acetate (71 mg) in 2.0 mL dry DMF was added Fmoc-OSu (20 mg) in DMF (0.5 mL+0.5 mL rinse) and the resulting mixture was stirred at 0 to 5° C. for 5 hours. LC-MS analysis showed the formation of single mono-Fmoc product and some bis-Fmoc product with no starting material left. fMLF-OSu (35 mg) was then added and the reaction mixture was stirred at 0° C. to room temperature overnight. LC-MS analysis showed the disappearance of the mono-Fmoc product and the formation of the desired coupling product. 10% Piperidine in DMF (0.6 mL) was then added and the reaction mixture was stirred at room temperature for 1 h. LC-MS analysis showed the disappearance of the Fmoc material and the formation of the coupling product and caspofungin. The bulk volume of DMF was removed by blowing with a gentle stream of nitrogen and the product in DMF (˜1.2 mL) was purified via preparative HPLC to yield 43 mg of product after solvent removal at reduced pressure at 30° C. and then drying via lyophilization. HPLC (UV at 220 nm), T_{R} 6.962 min (92.9% purity); ¹H-NMR (400 MHz, Methanol-d₄) δ 8.10 (s, 1H), 7.18-7.29 (m, 5H), 7.13-7.16 (m, 2H), 6.74-6.78 (m, 2H), 5.00 (m, 1H), 4.90 (d, 1H, J=2.5 Hz), 4.48-4.61 (m, 6H), 4.26-4.36 (m, 4H), 4.18-4.22 (m, 2H), 3.97-4.01 (m, 2H), 3.94-3.97 (m, 1H), 3.77-3.87 (m, 3H), 3.11-3.28 (m, 3H), 2.79-3.01 (m, 3H), 2.42-2.60 (m, 2H), 2.19-2.30 (m, 3H), 2.10 (s, 3H), 1.90-2.12 (m, 4H), 1.21-1.83 (m, 25H), 1.04-1.14 (m, 2H), 0.84-0.96 (m, 16H); LC/MS, 1513.7 [M+H]⁺.

In a similar fashion, the following procedure was used to prepare Compound 1 in which intermediates were isolated and purified.

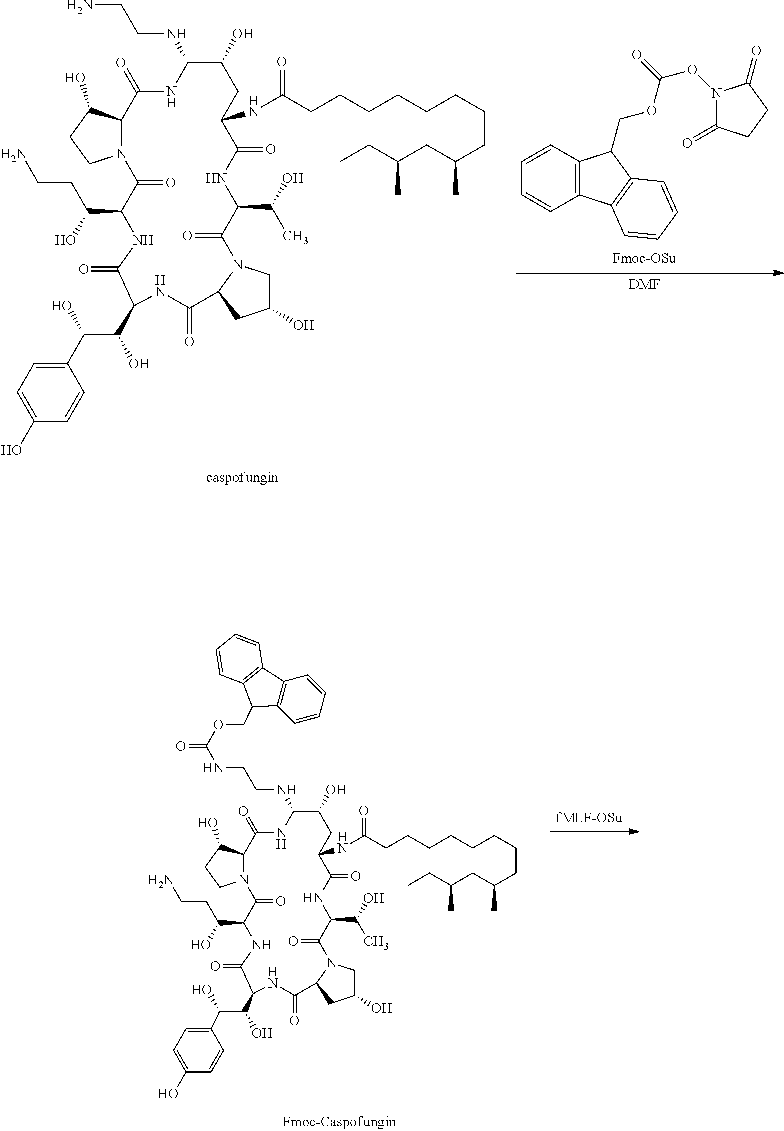

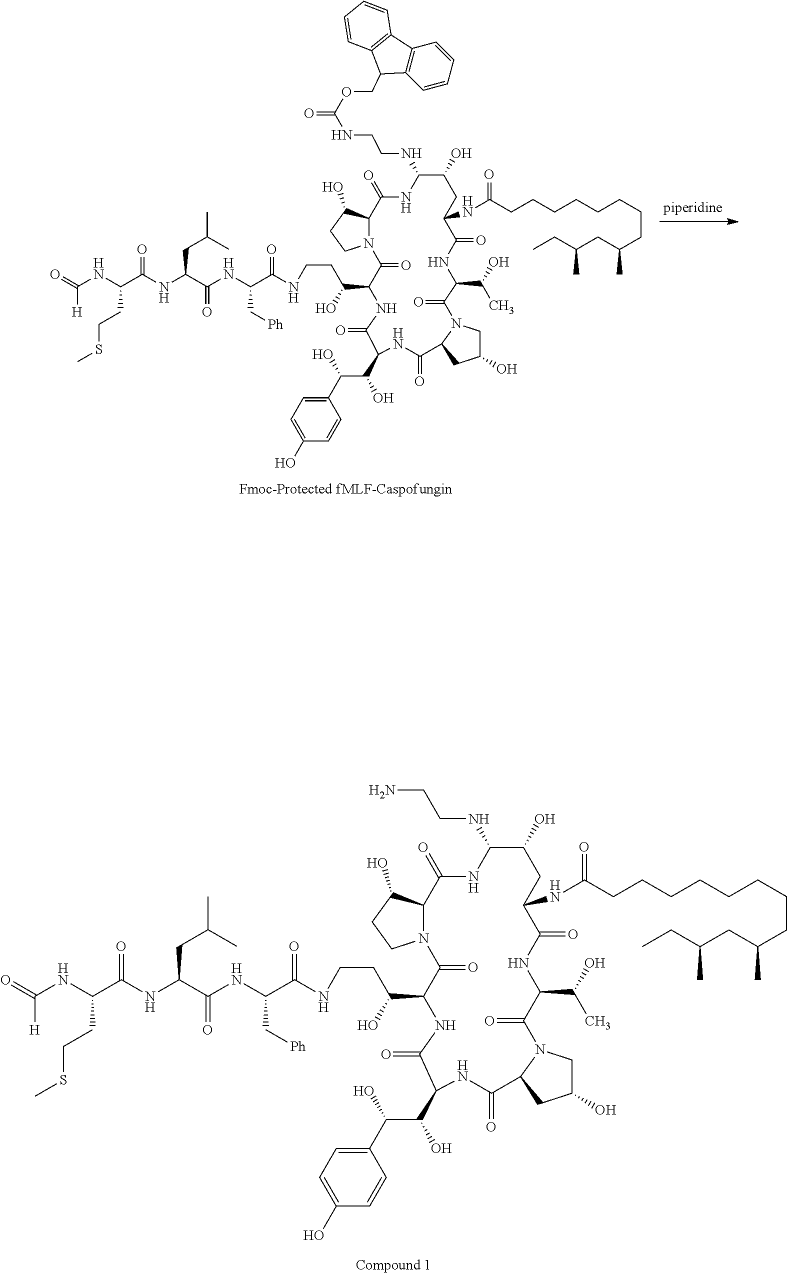

Step a. Preparation of Fmoc-Caspofungin.

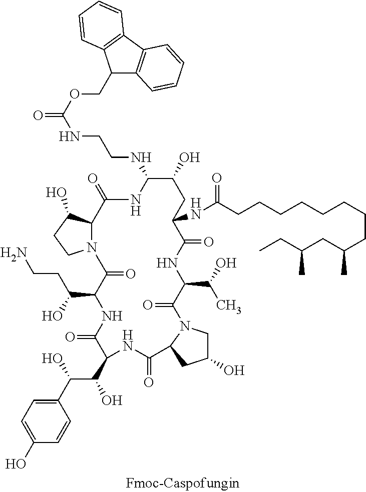 [SMILES: CC[C@H](C)C[C@H](C)CCCCCCCCC(=O)N[C@H]1C[C@@H](O)[C@@H](NCCNC(=O)OCC2c3ccccc3-c3ccccc32)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

FMOC-OSu (262 mg, 0.78 mmol, in 3 mL of DMF) was added, dropwise, to a stirring solution of caspofungin acetate (860 mg, 0.71 mmol) in DMF (12 mL), cooled to −45° C. (acetonitrile dry ice bath) under an atmosphere of nitrogen. The reaction was stirred for 90 minutes while gradually rising to room temperature at which point both mono and bis FMOC adducts were observed by LC/MS. The solvent was reduced to approximately 6 mL on the rotovap and applied directly to reversed phase HPLC purification (Isco CombiFlash Rf; 50 g Redisep C18 column; 20 to 95% acetonitrile in DI water containing 0.1% trifluoroacetic acid: 15 minute gradient). The pure fractions were pooled and concentrated on the rotovap to afford 660 mg of the product as a white solid, trifluoracetate salt. Yield: 65%. LC/MS, [M/2+H]+ 658.4, 658.3 calculated.

### Step b. Conjugation of Fmoc-Caspofungin with fMLF

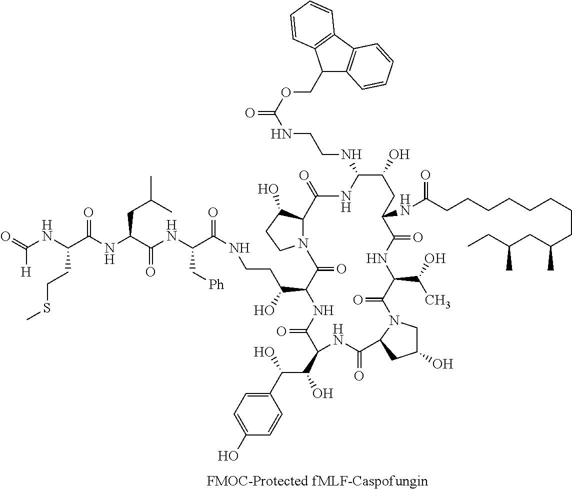 [SMILES: CC[C@H](C)C[C@H](C)CCCCCCCCC(=O)N[C@H]1C[C@@H](O)[C@@H](NCCNC(=O)OCC2c3ccccc3-c3ccccc32)NC(=O)[C@@H]2[C@@H](O)CCN2C(=O)[C@H]([C@H](O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a stirring solution of the mono-FMOC-protected-caspofungin trifluoroacetate (1.23 g, 0.87 mmol) dissolved in DMF (8 mL) was added N-methyl morpholine (296 μL, 3 mmol) and formyl-Met-Leu-Phe-OSu (534 mg, 1 mmol, in 4 mL DMF) in one aliquot. The reaction was stirred for 12 hours and then applied directly to reversed phase HPLC purification (Isco CombiFlash Rf; 50 g Redisep C18 column; 20 to 95% acetonitrile in DI water containing 0.1% trifluoroacetic acid: 15 minute gradient). The pure fractions were combined and concentrated on the rotovap to afford 975 mg of the product as a white solid. LC/MS, [M/2+H]+ 867.9, 867.5 calculated.

### Step c. Deprotection of FMOC-Protected fMLF-Caspofungin to Yield Compound 1

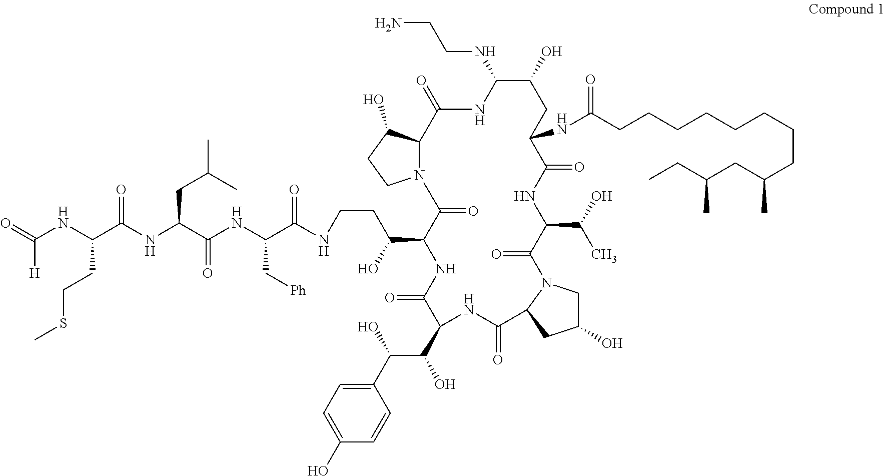 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a stirring solution of FMOC-protected-fMLF-Caspofungin (975 mg, 0.56 mmol, in 3 mL of DMF) was added 10% piperidine solution in DMF (3 mL). The reaction was stirred at ambient temperature for 45 minutes at which point complete conversion was observed by LC/MS analysis. The mixture was applied directly to reversed phase HPLC purification (Isco CombiFlash Rf; 50 g Redisep C18 column; 5 to 95% acetonitrile in DI water containing 0.1% formic acid: 15 minute gradient). The pure fractions were pooled and most of the acetonitrile was removed on the rotovap and then lyophilized to afford 535 mg of the product as a white solid, formate salt. LC/MS, [M/2+H]+ 757.0, 757.0 calculated.

### Example 3

### Synthesis of Compound 2

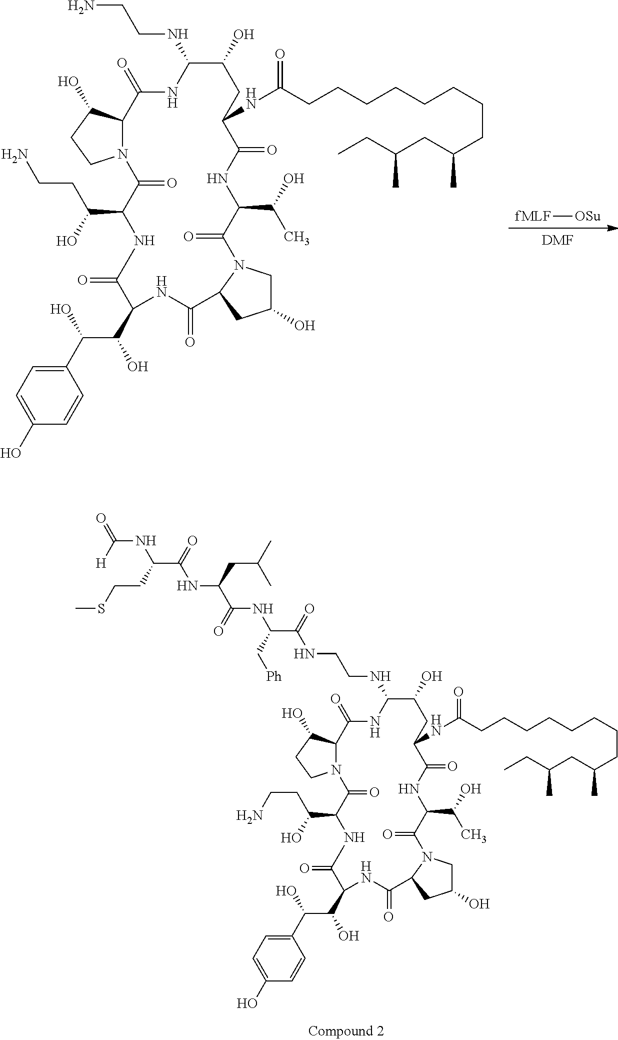

To a solution of caspofungin acetate (150 mg, 0.1235 mmol) in 8.0 mL dry DMF was added FMLF-OSu (66 mg, 0.1235 mmol) and the resulting mixture was stirred at 0 to 5° C. for 8 hours and then at room temperature overnight. LC-MS analysis showed the formation of one mono-coupling product as the predominant product and a small amount of bis-coupling product with no starting material left. The bulk volume of DMF was removed via blowing a gentle stream of nitrogen at room temperature and the crude product was purified via preparative HPLC to yield 100 mg of product after solvent removal at reduced pressure at 30° C. and drying via lyophilization. HPLC (UV at 220 nm), T_{R} 6.444 min (93.9%); ¹H-NMR (400 MHz, Methanol-d₄) δ8.13 (s, 1H), 7.18-7.30 (m, 5H), 7.11-7.14 (m, 2H), 6.74-6.77 (m, 2H), 4.99 (d, 1H, J=3.2 Hz), 4.92 (d, 1H, J=6.3 Hz), 4.42-4.63 (m, 7H), 4.19-4.36 (m, 6H), 4.03-4.08 (m, 2H), 3.94-3.98 (m, 1H), 3.78-3.87 (m, 3H), 3.22-3.29 (m, 2H), 3.14-3.18 (m, 1H), 3.06-3.14 (m, 2H), 2.94-2.99 (m, 1H), 2.75-2.81 (m, 1H), 2.41-2.66 (m, 4H), 2.22-2.29 (m, 3H), 2.09 (s, 3H), 1.91 (s, 3H), 1.80-2.12 (m, 8H), 1.21-1.63 (m, 20H), 1.18 (d, 3H, J=6.1 Hz), 1.02-1.13 (m, 2H), 0.84-0.95 (m, 16H); LC/MS, 1513.7 [M+H]⁺.

### Example 4

### Synthesis of Compound 3

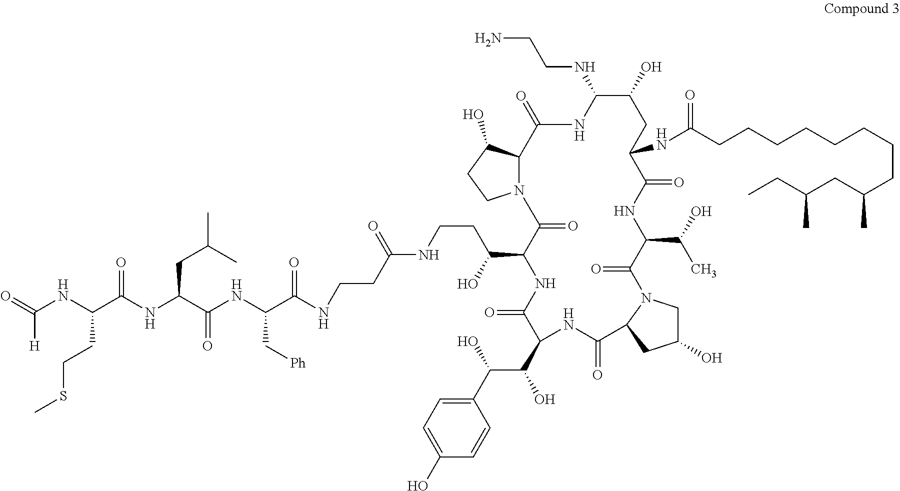 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLF-3-Ala-OSu as starting materials, Compound 3 was synthesized using an analogous procedure as used for Compound 1. HPLC (UV at 220 nm), T_{R} 6.846 min (99%); ¹H-NMR (400 MHz, Methanol-d₄) δ 8.11 (s, 1H), 7.18-7.29 (m, 5H), 7.12-7.16 (m, 2H), 6.73-6.77 (m, 2H), 5.00 (d, 1H, J=3.0 Hz), 4.96 (d, 1H, J=4.1 Hz), 4.48-4.59 (m, 6H), 4.25-4.37 (m, 5H), 4.18 (d, 1H, J=4.7 Hz), 3.90-4.00 (m, 3H), 3.72-3.85 (m, 3H), 3.21-3.48 (m, 3H), 3.06-3.17 (m, 2H), 2.78-2.99 (m, 4H), 2.39-2.57 (m, 3H), 2.32 (t, 2H, J=7.0 Hz), 2.23-2.28 (m, 3H), 2.08 (s, 3H), 1.90 (s, 3H), 1.74-2.20 (m, 5H), 1.16-1.72 (m, 23H), 1.04-1.13 (m, 2H), 0.85-0.95 (m, 16H); LC/MS, 1583.8 [M+H]⁺.

### Example 5

### Synthesis of Compound 4

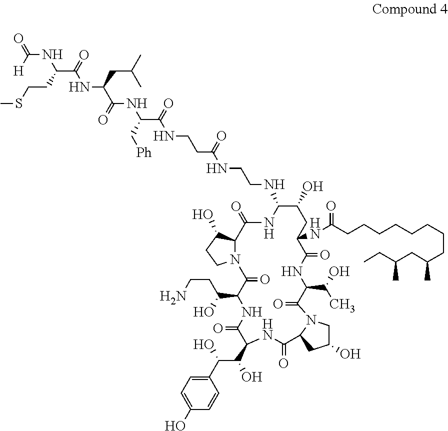 [SMILES: CC[C@H](C)C[C@H](C)CCCCCCCCC(=O)N[C@H]1C[C@@H](O)[C@@H](NCCNC(=O)CCNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLF-β-Ala-OSu as starting materials, Compound 4 was synthesized using an analogous procedure as used for Compound 2. HPLC (UV at 220 nm), T_{R} 6.079 min (98.6%); ¹H-NMR (400 MHz, Methanol-d₄) δ 8.11 (s, 1H), 7.18-7.29 (m, 5H), 7.11-7.14 (m, 2H), 6.74-6.77 (m, 2H), 4.99 (d, 1H, J=3.2 Hz), 4.92 (d, 1H, J=6.4 Hz), 4.43-4.63 (m, 7H), 4.29-4.35 (m, 4H), 4.18-4.24 (m, 2H), 4.03-4.10 (m, 2H), 3.94-3.98 (m, 1H), 3.79-3.86 (m, 3H), 3.38 (t, 3H, J=7.1 Hz), 3.24-3.33 (m, 1H), 3.03-3.14 (m, 3H), 2.93-2.98 (m, 1H), 2.76-2.82 (m, 1H), 2.64-2.70 (m, 1H), 2.41-2.55 (m, 3H), 2.22-2.36 (m, 5H), 2.08 (s, 3H), 1.90 (s, 3H), 1.81-2.11 (m, 8H), 1.21-1.65 (m, 20H), 1.18 (d, 3H, J=6.2 Hz), 1.04-1.13 (m, 2H), 0.85-0.95 (m, 16H); LC/MS, 1583.8 [M+H]⁺.

### Example 6

### Synthesis of Compound 5

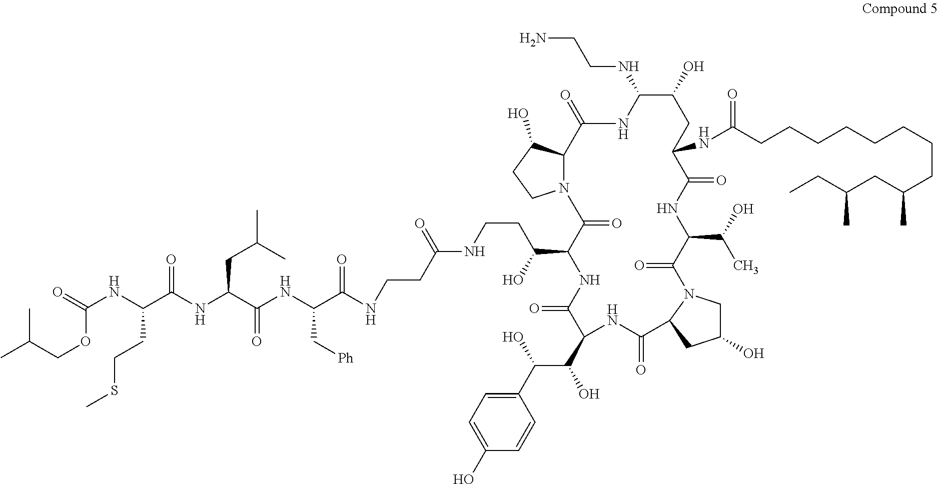 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC(=O)OCC(C)C)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and ib-MLF-β-Ala-OSu as starting materials, Compound 5 was synthesized using an analogous procedure as used for Compound 1. HPLC (UV at 220 nm), T_{R} 7.781 min (98.6%); ¹H-NMR (400 MHz, Methanol-d₄)δ 7.18-7.29 (m, 5H), 7.12-7.16 (m, 2H), 6.73-6.77 (m, 2H), 5.01 (d, 1H, J=3.2 Hz), 4.96 (d, 1H, J=3.8 Hz), 4.48-4.59 (m, 5H), 4.37 (d, 1H, J=1.6 Hz), 4.25-4.34 (m, 4H), 4.17-4.21 (m, 2H), 3.90-4.00 (m, 3H), 3.74-3.88 (m, 5H), 3.22-3.46 (m, 3H), 3.05-3.16 (m, 2H), 2.77-2.98 (m, 4H), 2.43-2.59 (m, 3H), 2.20-2.34 (m, 5H), 2.08 (s, 3H), 1.90 (s, 3H), 1.76-2.20 (m, 7H), 1.20-1.71 (m, 21H), 1.18 (d, 3H, J=6.3 Hz), 1.04-1.13 (m, 2H), 0.85-0.94 (m, 22H); LC/MS, 1557.6 [M+H]⁺.

### Example 7

### Synthesis of Compound 6

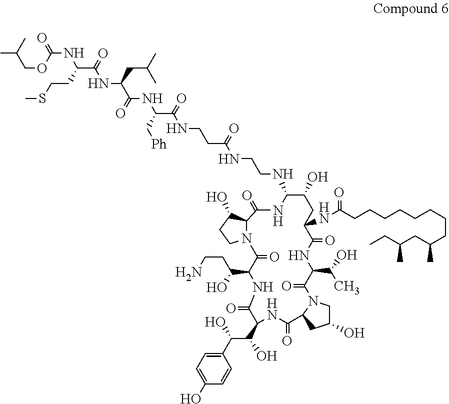 [SMILES: CC[C@H](C)C[C@H](C)CCCCCCCCC(=O)N[C@H]1C[C@@H](O)[C@@H](NCCNC(=O)CCNC(=O)[C@H](Cc2ccccc2)NC(=O)[C@H](CC(C)C)NC(=O)[C@H](CCSC)NC(=O)OCC(C)C)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and ib-MLF-β-Ala-OSu as starting materials, Compound 6 was synthesized using an analogous procedure as used for Compound 2. HPLC (UV at 220 nm), T_{R} 7.023 min (100%); ¹H-NMR (400 MHz, Methanol-d₄) δ7.18-7.29 (m, 5H), 7.11-7.14 (m, 2H), 6.73-6.77 (m, 2H), 4.99 (d, 1H, J=3.3 Hz), 4.92 (d, 1H, J=6.3 Hz), 4.43-4.63 (m, 6H), 4.29-4.35 (m, 4H), 4.18-4.24 (m, 3H), 4.03-4.10 (m, 2H), 3.94-3.98 (m, 1H), 3.79-3.86 (m, 5H), 3.38 (t, 3H, J=7.1 Hz), 3.24-3.33 (m, 1H), 3.04-3.16 (m, 3H), 2.92-2.98 (m, 1H), 2.76-2.82 (m, 1H), 2.64-2.70 (m, 1H), 2.41-2.57 (m, 3H), 2.22-2.36 (m, 5H), 2.08 (s, 3H), 1.90 (s, 3H), 1.79-2.11 (m, 8H), 1.21-1.65 (m, 20H), 1.18 (d, 3H, J=6.2 Hz), 1.04-1.13 (m, 2H), 0.85-0.95 (m, 22H); LC/MS, 1557.5 [M+H]⁺.

### Example 8

### Synthesis of Compound 7

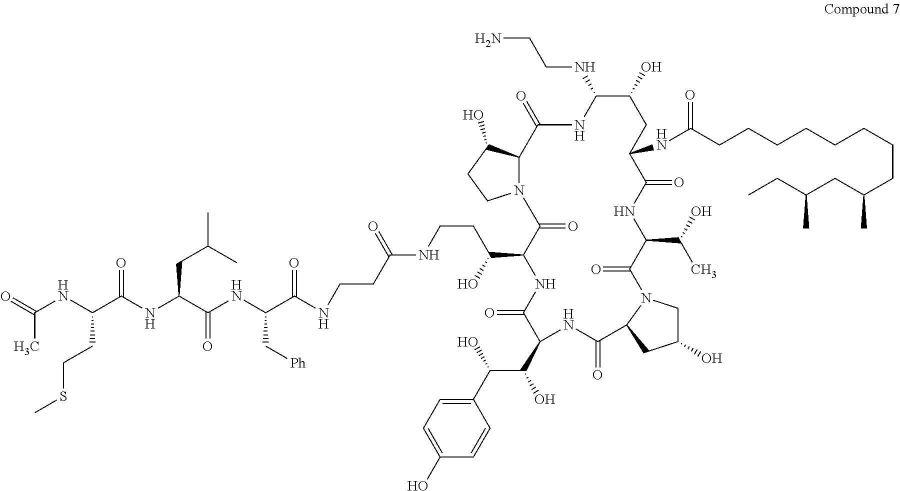 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC(C)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and a-MLF-β-Ala-OSu as starting materials, Compound 7 was synthesized using an analogous procedure as used for Compound 1. HPLC (UV at 254 nm), T_{R} 7.102 min (100%); ¹H-NMR (400 MHz, Methanol-d₄) δ7.18-7.29 (m, 5H), 7.12-7.16 (m, 2H), 6.73-6.77 (m, 2H), 5.01 (d, 1H, J=3.2 Hz), 4.96 (d, 1H, J=4.0 Hz), 4.48-4.59 (m, 5H), 4.37-4.40 (m, 2H), 4.25-4.34 (m, 4H), 4.18 (d, 1H, J=4.7 Hz), 3.91-3.99 (m, 3H), 3.74-3.83 (m, 3H), 3.22-3.46 (m, 3H), 3.04-3.16 (m, 2H), 2.77-2.99 (m, 4H), 2.41-2.56 (m, 3H), 2.33 (t, 2H, J=6.8 Hz), 2.14-2.29 (m, 4H), 2.08 (s, 3H), 1.99 (s, 3H), 1.90 (s, 3H), 1.78-2.11 (m, 5H), 1.20-1.71 (m, 21H), 1.18 (d, 3H, J=6.3 Hz), 1.00-1.13 (m, 2H), 0.85-0.95 (m, 16H); LC/MS, 1598.8 [M+H]⁺.

Alternatively, Compound 7 was prepared by the following two step method.

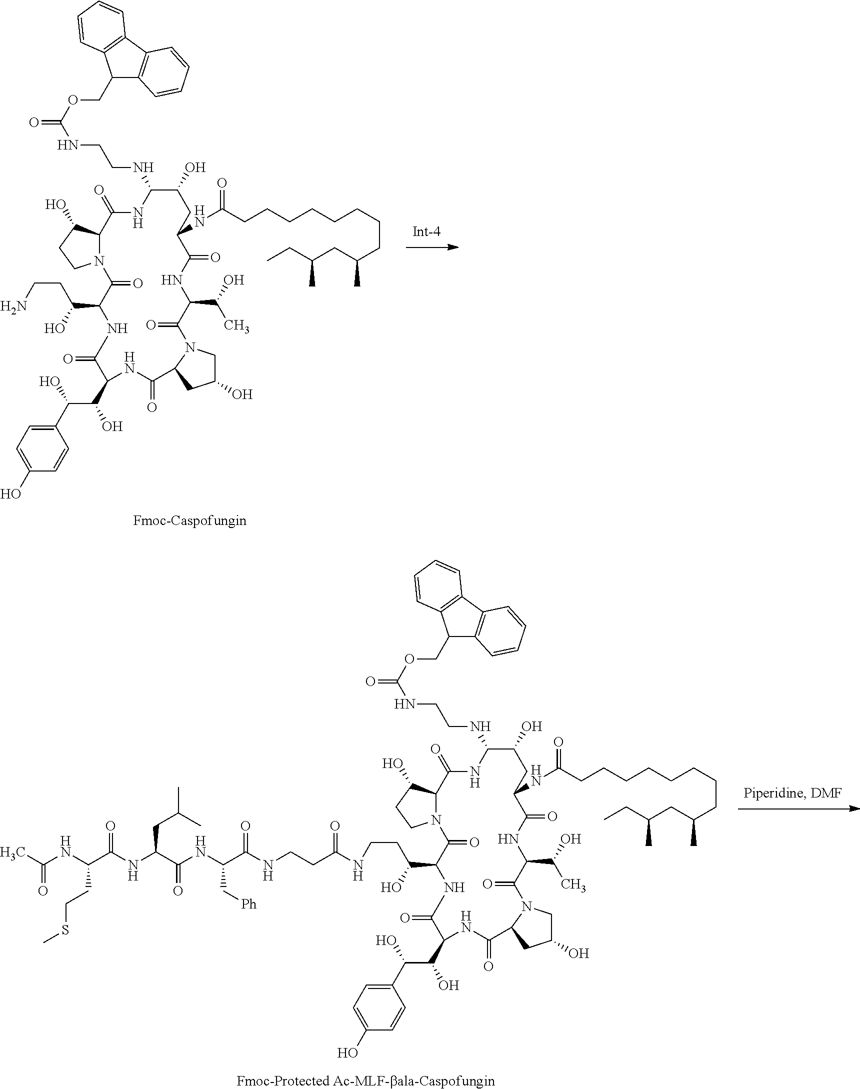

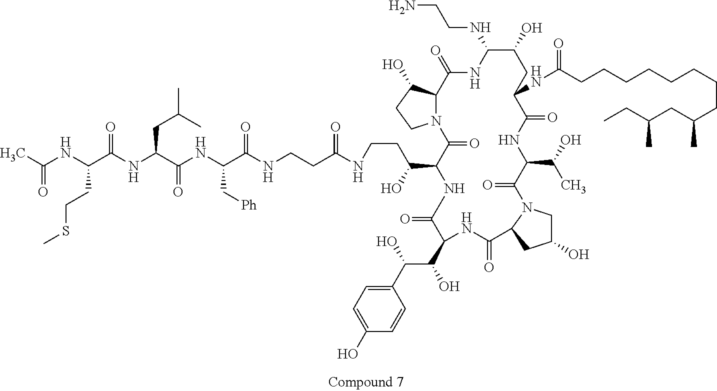 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC(C)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

### Step a. Synthesis of Ac-MLF-β-ala-Fmoc-Caspofungin

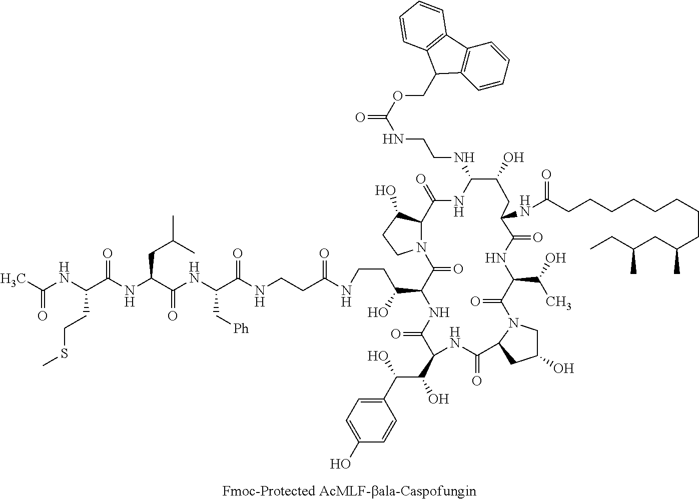 [SMILES: CC[C@H](C)C[C@H](C)CCCCCCCCC(=O)N[C@H]1C[C@@H](O)[C@@H](NCCNC(=O)OCC2c3ccccc3-c3ccccc32)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC(C)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

A flask was charged with Ac-MLF-β-ala-OSu (Int-4) (198 mg, 0.320 mmol), mono-Fmoc caspofungin (300 mg, 0.228 mmol), N-methylmorpholine (75 μL, 0.685 mmol), and DMF (1.0 mL). Reaction progress was monitored by LCMS. After 3 h starting material was consumed. The product was isolated by RPLC using an Isco CombiFlash liquid chromatograph eluted with 10% to 100% acetonitrile and water, using 0.1% formic acid as the modifier. Removal of the volatiles in vacuo yielded 298 mg of a white powder. LC/MS, 910.7 [M/2+H]+ calculated 910.5.

### Step b. Synthesis of Compound 7

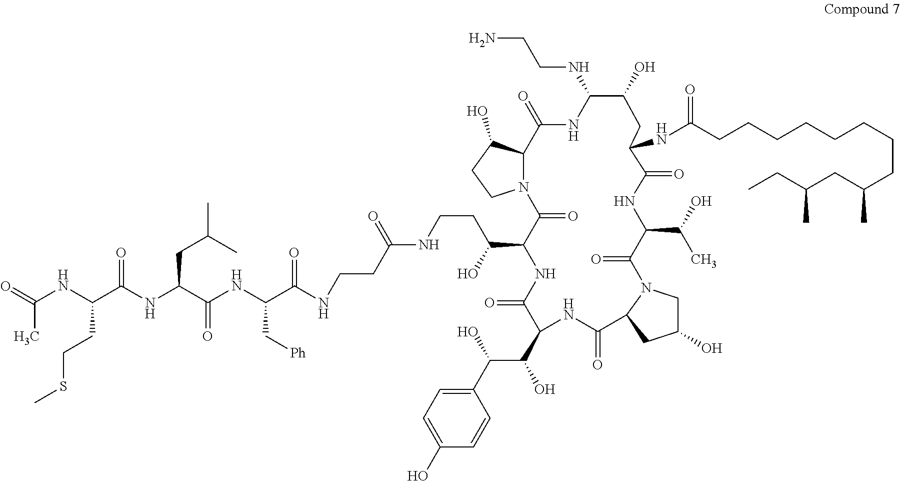 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC(C)=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Fmoc-protected AcMLF-βala-caspofungin (298 mg, 0.1638 mmol) was dissolved in DMF (2.0 mL), and then piperidine (240 μL) was added. Reaction progress was monitored by LCMS. Sufficient starting material was consumed after 1 h. The reaction mixture was purified by RPLC using an Isco CombiFlash liquid chromatograph eluted with 10% to 100% acetonitrile and water, using 0.1% formic acid as the modifier. Removal of the volatiles in vacup gave 231 mg of the product. High resolution MS, 1597.8901 [M+H]+ calculated 1597.8837.

### Example 9

### Synthesis of Compound 8

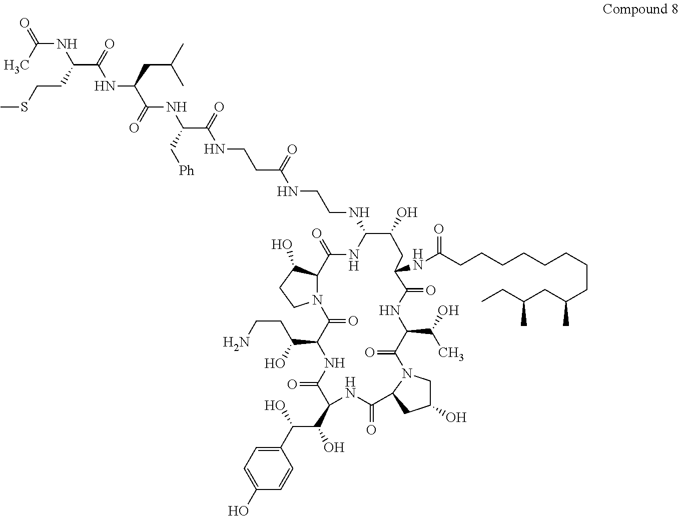 [SMILES: CC[C@H](C)C[C@H](C)CCCCCCCCC(=O)N[C@H]1C[C@@H](O)[C@@H](NCCNC(=O)CCNC(=O)[C@H](Cc2ccccc2)NC(=O)[C@H](CC(C)C)NC(=O)[C@H](CCSC)NC(C)=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and a-MLF-β-Ala-OSu as starting materials, Compound 8 was synthesized using an analogous procedure as used for Compound 2. HPLC (UV at 254 nm), T_{R} 6.420 min (100%); ¹H-NMR (400 MHz, Methanol-d₄) δ7.18-7.29 (m, 5H), 7.11-7.14 (m, 2H), 6.73-6.77 (m, 2H), 4.99 (d, 1H, J=3.4 Hz), 4.92 (d, 1H, J=6.3 Hz), 4.38-4.64 (m, 7H), 4.28-4.35 (m, 4H), 4.18-4.24 (m, 2H), 4.03-4.10 (m, 2H), 3.94-3.98 (m, 1H), 3.79-3.86 (m, 3H), 3.38 (t, 3H, J=6.9 Hz), 3.24-3.33 (m, 1H), 3.04-3.15 (m, 3H), 2.91-3.00 (m, 1H), 2.76-2.82 (m, 1H), 2.64-2.70 (m, 1H), 2.42-2.58 (m, 3H), 2.21-2.36 (m, 5H), 2.08 (s, 3H), 2.00 (s, 3H), 1.90 (s, 3H), 1.81-2.12 (m, 8H), 1.21-1.64 (m, 20H), 1.18 (d, 3H, J=6.2 Hz), 1.04-1.13 (m, 2H), 0.85-0.95 (m, 16H); LC/MS, 1598.8 [M+H]⁺.

### Example 10

### Synthesis of Compound 9

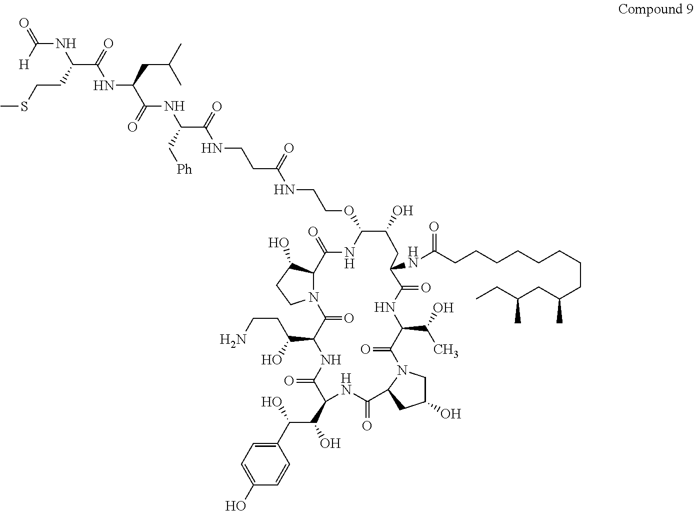 [SMILES: CC[C@H](C)C[C@H](C)CCCCCCCCC(=O)N[C@H]1C[C@@H](O)[C@@H](OCCNC(=O)CCNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Int-15 was treated with fMLF-β-Ala-OSu using an analogous procedure as used for Compound 2 to yield Compound 9. HPLC (UV at 220 nm), T_{R} 6.917 min (100%); ¹H-NMR (400 MHz, Methanol-d₄) δ 8.12 (s, 1H), 7.18-7.29 (m, 5H), 7.11-7.15 (m, 2H), 6.74-6.77 (m, 2H), 5.08 (d, 1H, J=2.4 Hz), 4.98 (d, 1H, J=3.1 Hz), 4.91 (d, 1H, J=6.3 Hz), 4.44-4.63 (m, 6H), 4.30-4.35 (m, 4H), 4.21-4.27 (m, 2H), 4.09-4.13 (m, 1H), 4.03-4.08 (m, 1H), 3.94-3.97 (m, 1H), 3.79-3.87 (m, 3H), 3.60-3.66 (m, 1H), 3.51-3.56 (m, 1H), 3.33-3.43 (m, 4H), 3.10-3.15 (m, 1H), 3.05 (t, 2H, J=7.1 Hz), 2.93-2.99 (m, 1H), 2.41-2.55 (m, 2H), 2.31-2.35 (m, 2H), 2.21-2.29 (m, 3H), 2.07 (s, 3H), 1.90 (s, 3H), 1.76-2.10 (m, 8H), 1.18-1.64 (m, 24H), 1.10-1.13 (m, 2H), 0.85-0.95 (m, 16H); LC/MS, 1584.9 [M+H]⁺.

### Example 11

### Synthesis of Compound 10

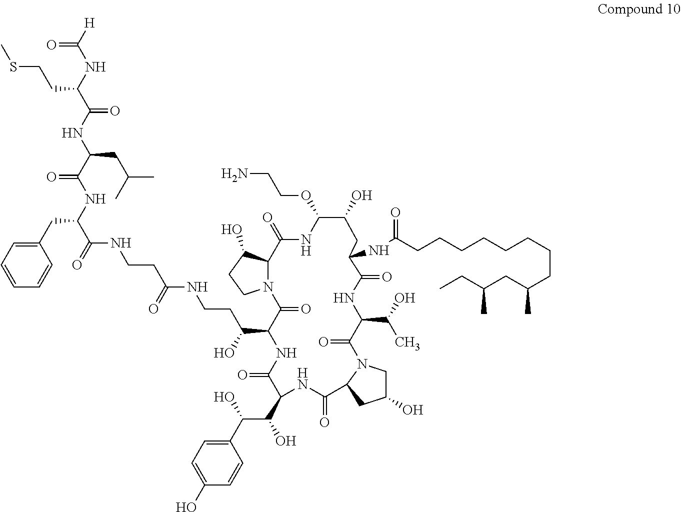 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Int-15 was treated with fMLF-β-Ala-OSu using an analogous procedure as Compound 1 to yield Compound 10. HPLC (UV at 220 nm), T_{R} 7.011 min (100%); ¹H-NMR (400 MHz, Methanol-d₄) δ 8.11 (s, 1H), 7.21-7.29 (m, 5H), 7.11-7.15 (m, 2H), 6.74-6.77 (m, 2H), 5.22 (d, 1H, J=2.4 Hz), 4.94-4.96 (m, 2H), 4.48-4.59 (m, 6H), 4.25-4.39 (m, 6H), 4.09-4.13 (m, 1H), 4.01-4.05 (m, 1H), 3.95-3.99 (m, 1H), 3.79-3.87 (m, 3H), 3.71-3.74 (m, 1H), 3.63-3.67 (m, 1H), 3.51-3.56 (m, 1H), 3.35-3.45 (m, 4H), 3.22-3.29 (m, 1H), 3.03-3.06 (m, 2H), 2.94-2.98 (m, 1H), 2.41-2.55 (m, 4H), 2.31-2.35 (m, 2H), 2.21-2.29 (m, 3H), 2.08 (s, 3H), 1.90 (s, 3H), 1.76-2.10 (m, 8H), 1.18-1.64 (m, 24H), 1.10-1.13 (m, 2H), 0.85-0.95 (m, 16H); LC/MS, 1584.7 [M+H]⁺.

### Example 12

### Synthesis of Compound 11

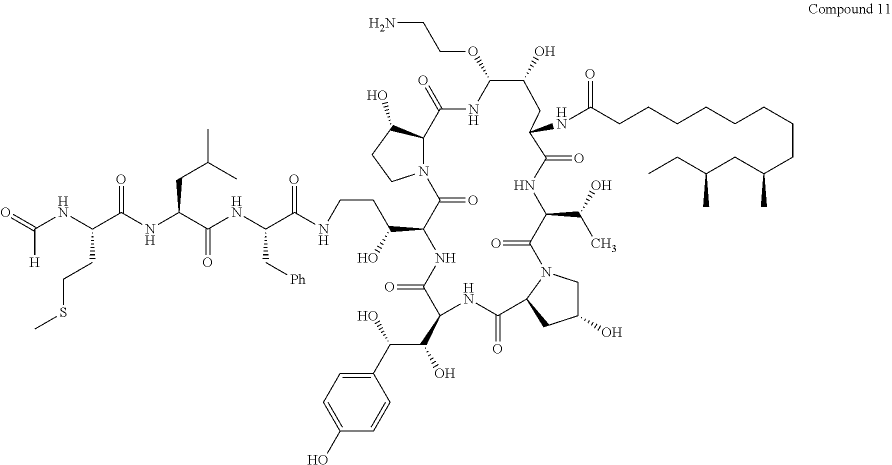 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Int-15 was treated with fMLF-OSu using an analogous procedure as Compound 1 to yield Compound 11. HPLC (UV at 220 nm), T_{R} 7.812 min (100%); H-NMR (400 MHz, Methanol-d₄) δ 8.09 (s, 1H), 7.20-7.29 (m, 5H), 7.14-7.16 (m, 2H), 6.74-6.77 (m, 2H), 5.18 (d, 1H, J=3.9 Hz), 4.98 (d, 1H, J=2.9 Hz), 4.90-4.91 (m, 1H), 4.44-4.63 (m, 7H), 4.22-4.34 (m, 5H), 3.92-4.01 (m, 2H), 3.69-3.83 (m, 3H), 3.60-3.66 (m, 1H), 3.51-3.56 (m, 1H), 3.33-3.40 (m, 1H), 3.14-3.22 (m, 2H), 2.95-3.07 (m, 3H), 2.41-2.58 (m, 2H), 2.22-2.30 (m, 2H), 2.22-2.30 (m, 3H), 2.10 (s, 3H), 1.90 (s, 3H), 1.67-2.10 (m, 6H), 1.17-1.67 (m, 22H), 1.05-1.13 (m, 2H), 0.85-0.95 (m, 16H); LC/MS, 1513.7 [M+H]⁺.

### Example 13

### Synthesis of Compound 12

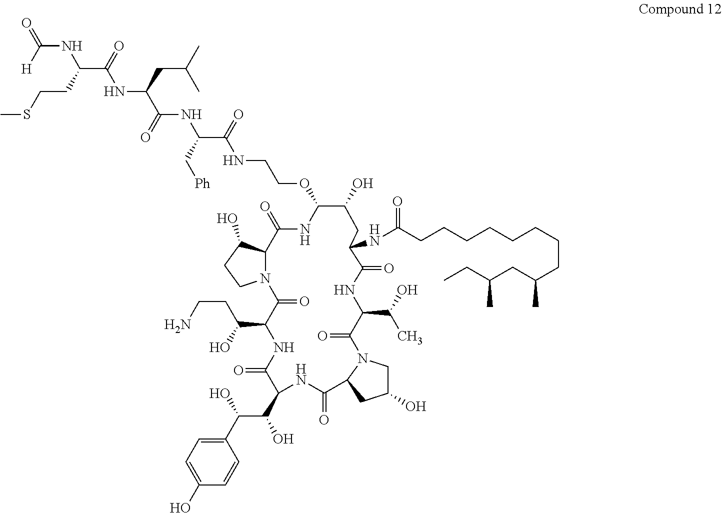 [SMILES: CC[C@H](C)C[C@H](C)CCCCCCCCC(=O)N[C@H]1C[C@@H](O)[C@@H](OCCNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Int-15 was treated with fMLF-OSu using an analogous procedure as Compound 2 to yield Compound 12. HPLC (UV at 220 nm), T_{R} 7.137 min (98.8%); H-NMR (400 MHz, Methanol-d₄) δ 8.12 (s, 1H), 7.18-7.29 (m, 5H), 7.11-7.15 (m, 2H), 6.74-6.77 (m, 2H), 5.04 (d, 1H, J=2.4 Hz), 4.97 (d, 1H, J=3.4 Hz), 4.91 (d, 1H, J=6.3 Hz), 4.42-4.62 (m, 6H), 4.22-4.36 (m, 6H), 4.03-4.13 (m, 2H), 3.94-3.97 (m, 1H), 3.79-3.88 (m, 3H), 3.59-3.65 (m, 1H), 3.45-3.50 (m, 1H), 3.33-3.36 (m, 2H), 3.15-3.20 (m, 1H), 3.06 (t, 2H, J=6.9 Hz), 2.94-3.00 (m, 1H), 2.41-2.58 (m, 3H), 2.23-2.29 (m, 3H), 2.08 (s, 3H), 1.90 (s, 3H), 1.76-2.10 (m, 8H), 1.04-1.62 (m, 24H), 0.84-0.95 (m, 16H); LC/MS, 1513.7 [M+H]⁺.

### Example 14

### Synthesis of Compound 13a and 13b

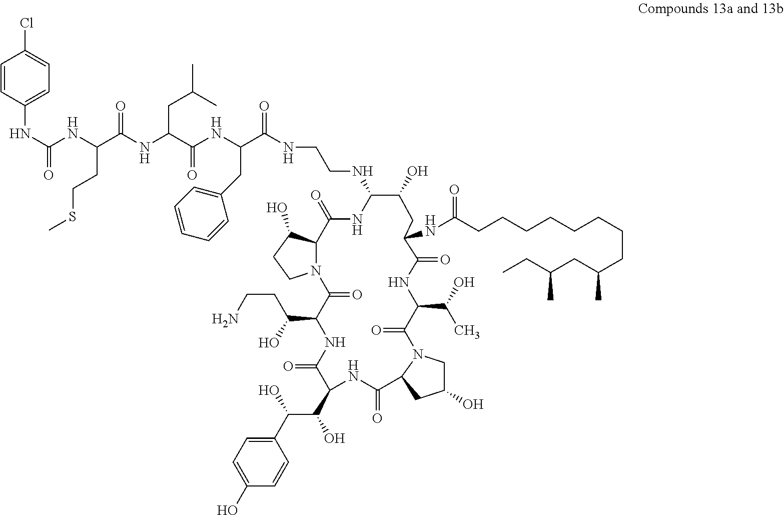 [SMILES: CC[C@H](C)C[C@H](C)CCCCCCCCC(=O)N[C@H]1C[C@@H](O)[C@@H](NCCNC(=O)C(Cc2ccccc2)NC(=O)C(CC(C)C)NC(=O)C(CCSC)NC(=O)Nc2ccc(Cl)cc2)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and p-Cl-Ph-NHCONHMLF-OSu as starting materials, Compound 13 was synthesized using an analogous procedure as Compound 2 as a mixture of diasteromers, separable by preparative HPLC. Diastereomer A (13a): HPLC (UV at 220 nm), T_{R} 7.094 min (89.0%); ¹H-NMR (400 MHz, Methanol-d₄) δ7.39-7.41 (m, 2H), 7.06-7.24 (m, 9H), 6.74-6.77 (m, 2H), 4.99 (d, 1H, J=3.2 Hz), 4.91 (d, 1H, J=6.2 Hz), 4.51-4.62 (m, 5H), 4.44-4.46 (m, 1H), 4.18-4.37 (m, 7H), 3.94-4.10 (m, 3H), 3.78-3.84 (m, 3H), 3.14-3.20 (m, 1H), 3.04-3.08 (m, 2H), 2.78-2.90 (m, 2H), 2.41-2.46 (m, 1H), 2.22-2.26 (m, 3H), 2.10 (s, 3H), 1.90 (s, 3H), 1.75-2.09 (m, 8H), 1.18-1.67 (m, 23H), 1.06-1.13 (m, 2H), 0.82-0.95 (m, 16H).; LC/MS, 1539.6 [M+H]+;

Diastereomer B (13b): HPLC (UV at 220 nm), T_{R} 7.227 min (100.0%); H-NMR (400 MHz, Methanol-d₄) δ 7.20-7.33 (m, 9H), 7.12-7.14 (m, 2H), 6.74-6.77 (m, 2H), 5.01 (d, 1H, J=3.4 Hz), 4.92 (d, 1H, J=5.9 Hz), 4.56-4.62 (m, 5H), 4.44-4.47 (m, 1H), 4.23-4.36 (m, 5H), 3.96-4.14 (m, 4H), 3.79-3.84 (m, 3H), 3.03-3.07 (m, 2H), 2.72-2.84 (m, 3H), 2.51-2.56 (m, 2H), 2.41-2.47 (m, 1H), 2.20-2.28 (m, 3H), 2.06 (s, 3H), 1.90 (s, 3H), 1.74-2.16 (m, 7H), 1.19-1.57 (m, 22H), 1.03-1.13 (m, 2H), 0.77-0.95 (m, 16H).; LC/MS, 1539.6 [M+H]⁺.

### Example 15

### Synthesis of Compound 14

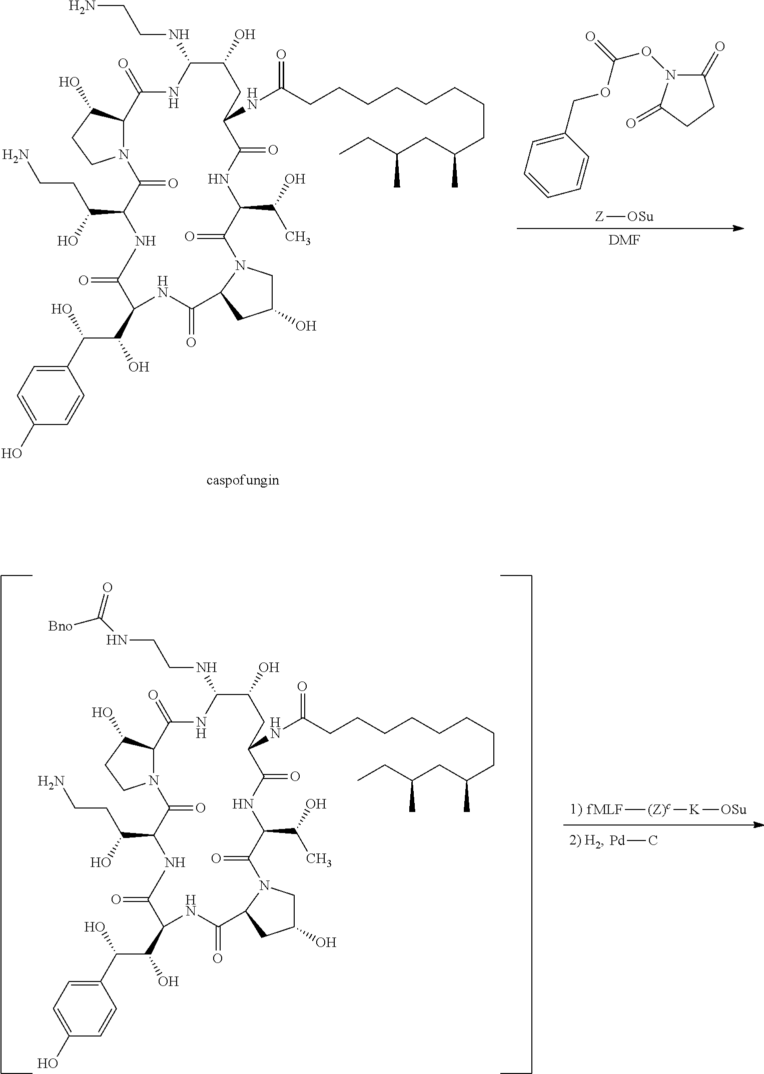

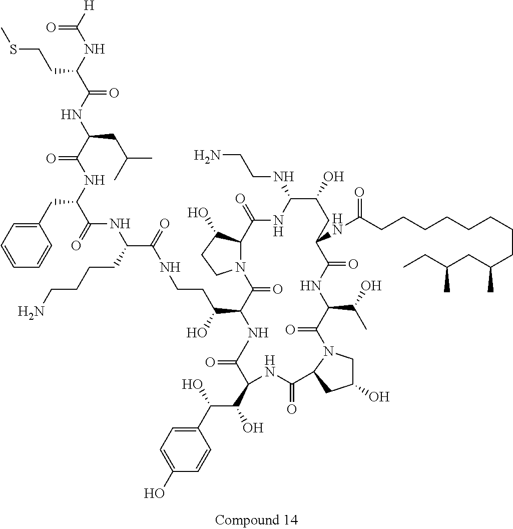 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCCCN)NC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a solution of caspofungin acetate (300 mg, 0.247 mmol) in 13.0 mL of dry DMF was added Z-OSu (62 mg, 0.247) in DMF and the resulting mixture was stirred at 0 to 5° C. for 5 hours. fMLF-(Z)^{ε}—K-OSu (0.247 mmol) in DMF (3.0 mL) was then added and the reaction mixture was stirred at 0° C. to room temperature overnight. LC-MS analysis showed the formation of the desired coupling product. The bulk volume of DMF was removed by blowing with a gentle stream of nitrogen and the product in DMF (˜5.0 mL) was purified via preparative HPLC to yield 195 mg of product after solvent removal at reduced pressure at 30° C. and then lyophilization. To a solution of the above intermediate in a solvent mixture of iso-propanol (27 mL) and water (3 mL) containing acetic acid (1.35 mL) was added palladium on carbon (5 wt %, 0.23 g, 0.107 mmol) and the resulting mixture was stirred under hydrogen (balloon) at room temperature for 2 days. The catalyst was removed via filtration through a short path of celite and the bulk volume of solvent was removed under reduced pressure. The crude product was purified three times via prep-HPLC to yield 20 mg of product as the diacetate salt after solvent removal at reduced pressure at 30° C. and then lyophilization. HPLC (UV at 220 nm), T_{R} 5.916 min (100.0%); ¹H-NMR (400 MHz, Methanol-d₄) δ 8.12 (s, 1H), 7.22-7.32 (m, 5H), 7.13-7.15 (m, 2H), 6.73-6.76 (m, 2H), 4.98-5.08 (m, 2H), 4.45-4.60 (m, 5H), 4.19-4.39 (m, 6H), 4.09-4.14 (m, 1H), 3.87-4.02 (m, 3H), 3.73-3.85 (m, 3H), 3.25-3.30 (m, 1H), 2.71-3.20 (m, 6H), 2.39-2.58 (m, 3H), 2.14-2.32 (m, 4H), 2.08 (s, 1.5H), 2.06 (s, 1.5H), 1.90 (s, 6H), 1.76-2.12 (m, 6H), 1.20-1.75 (m, 28H), 1.04-1.15 (m, 3H), 0.85-0.97 (m, 16H); LC/MS, 1640.9 [M+H]⁺.

### Example 16

### Synthesis of Compound 15

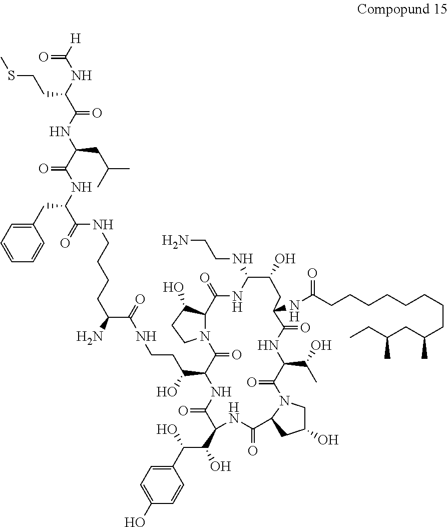 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@@H](N)CCCCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLF-(Z)^{α}—K-OSu as starting materials, Compound 15 was synthesized using an analogous procedure as Compound 14. HPLC (UV at 220 nm), T_{R} 6.482 min (100.0%); ¹H-NMR (400 MHz, Methanol-d₄)δ 8.13 (s, 1H), 7.18-7.29 (m, 5H), 7.11-7.14 (m, 2H), 6.74-6.77 (m, 2H), 5.02 (d, 1H, J=3.3 Hz), 4.97 (d, 1H, J=3.7 Hz), 4.48-4.59 (m, 6H), 4.18-4.35 (m, 6H), 4.03-4.10 (m, 2H), 3.90-4.00 (m, 3H), 3.77-3.83 (m, 3H), 3.40-3.46 (m, 1H), 3.07-3.24 (m, 5H), 2.78-3.00 (m, 3H), 2.64-2.70 (m, 1H), 2.41-2.57 (m, 3H), 2.14-2.29 (m, 3H), 2.09 (s, 3H), 1.91 (s, 6H), 1.81-2.11 (m, 4H), 1.20-1.75 (m, 29H), 1.04-1.13 (m, 2H), 0.85-0.95 (m, 16H); LC/MS, 1641.0 [M+H].

### Example 17

### Synthesis of Compound 16

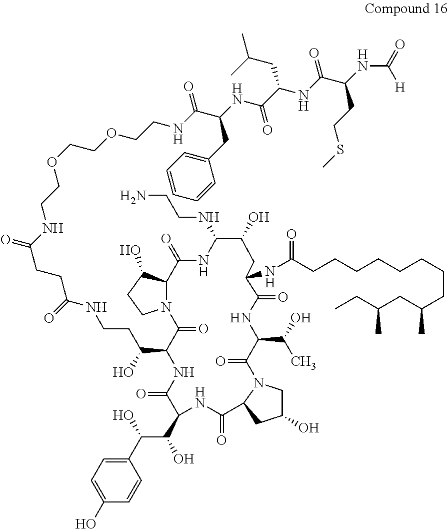 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)CCC(=O)NCCOCCOCCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLF-(PEG)₂-Suc-OSu as starting materials, Compound 16 was synthesized using an analogous procedure as Compound 1. HPLC (ELSD), T_{R} 7.245 min (100.0%); H-NMR (400 MHz, Methanol-d₄)δ 8.11 (s, 1H), 7.18-7.29 (m, 6H), 7.13-7.15 (m, 2H), 6.74-6.76 (m, 2H), 5.02 (d, 1H, J=3.3 Hz), 4.94 (d, 1H, J=4.2 Hz), 4.50-4.59 (m, 6H), 4.29-4.36 (m, 4H), 4.23-4.25 (m, 1H), 4.18-4.19 (m, 1H), 3.93-3.99 (m, 2H), 3.77-3.82 (m, 2H), 3.22-3.70 (m, 15H), 3.04-3.14 (m, 2H), 2.78-2.99 (m, 3H), 2.8 (s, 2H), 2.41-2.56 (m, 6H), 2.21-2.31 (m, 2H), 2.08 (s, 3H), 1.90 (s, 6H), 1.77-2.21 (m, 6H), 1.19-1.75 (m, 20H), 1.03-1.13 (m, 2H), 0.85-0.95 (m, 16H); LC/MS, 872.0 {[M+H]⁺/2}.

### Example 18

### Synthesis of Compound 17

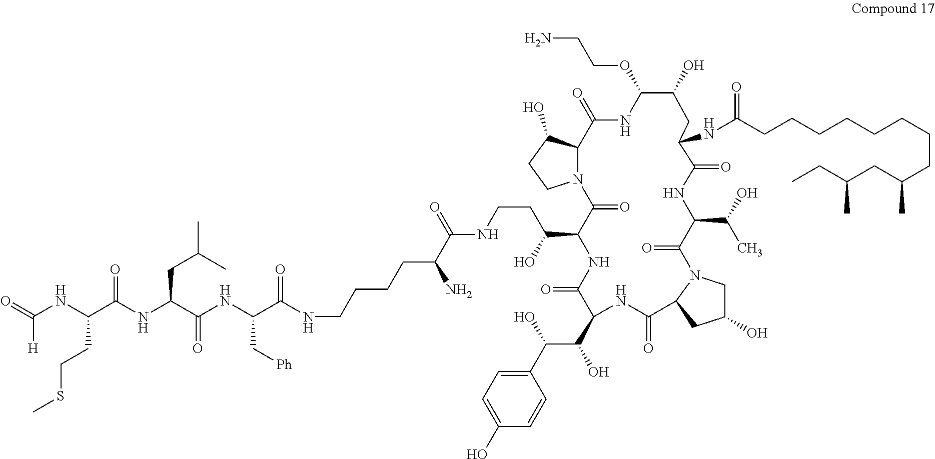 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)[C@@H](N)CCCCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using Int-15 and fMLF-(Z)^{α}—K-OSu as starting materials, Compound 17 is synthesized in an analogous manner as Compound 15. Compound 17 has a parent mass of 1640.91 amu.

### Example 19

### Synthesis of Compound 18

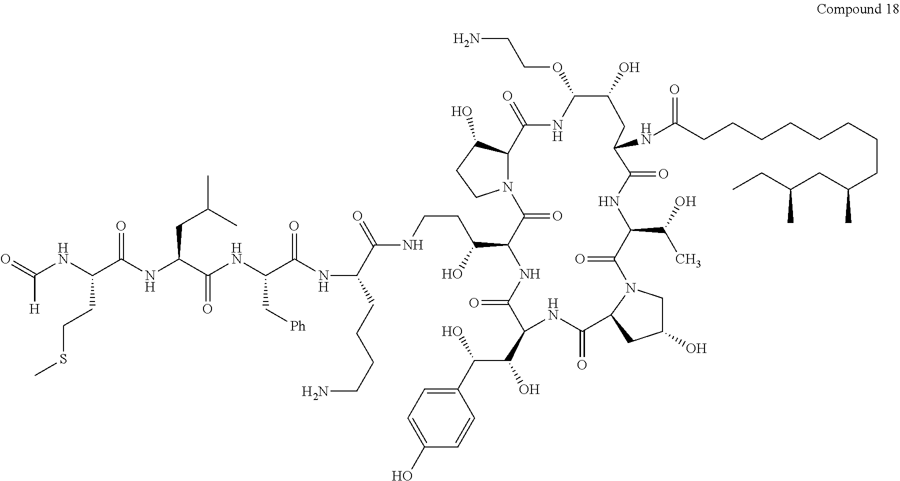 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)[C@H](CCCCN)NC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using Int-15 and fMLF-(Z)^{ε}—K-OSu as starting materials, Compound 18 is synthesized in an analogous manner as Compound 14. Compound 18 has a parent mass of 1640.91 amu.

### Example 20

### Synthesis of Compound 19

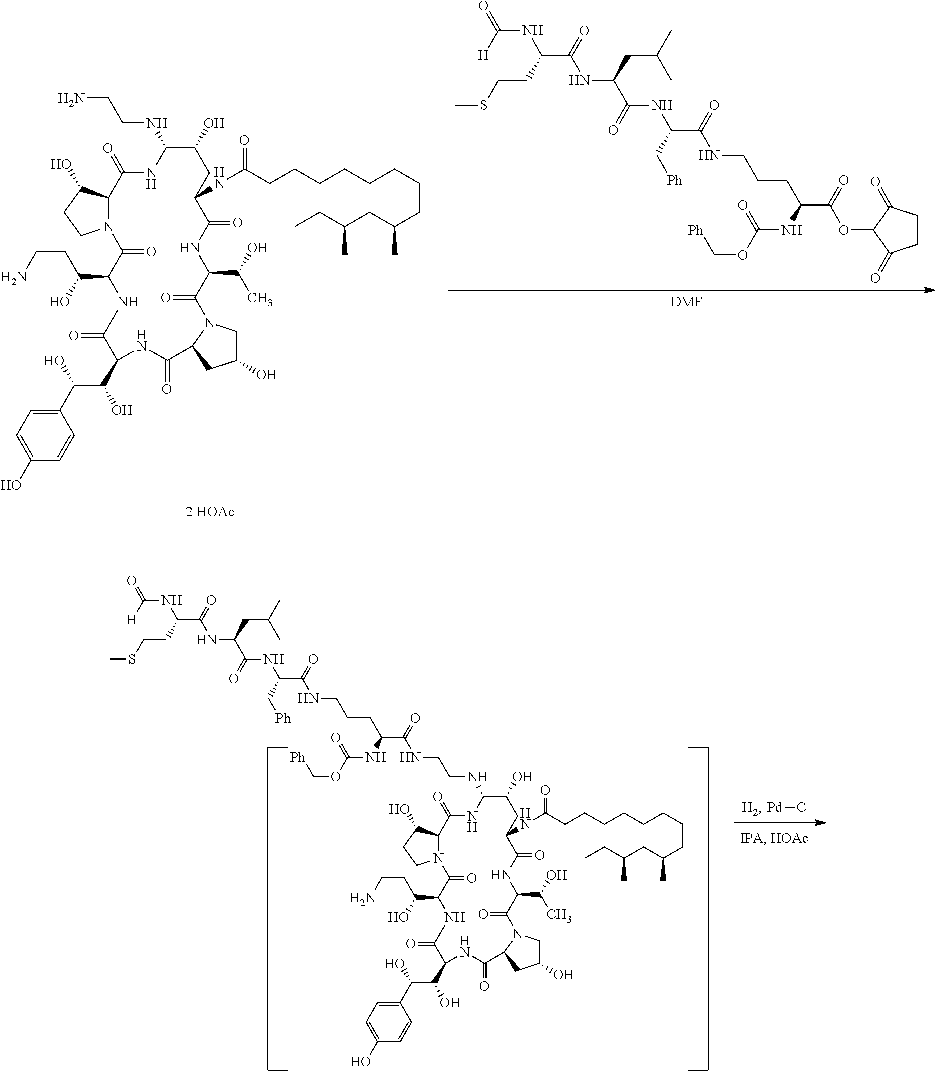

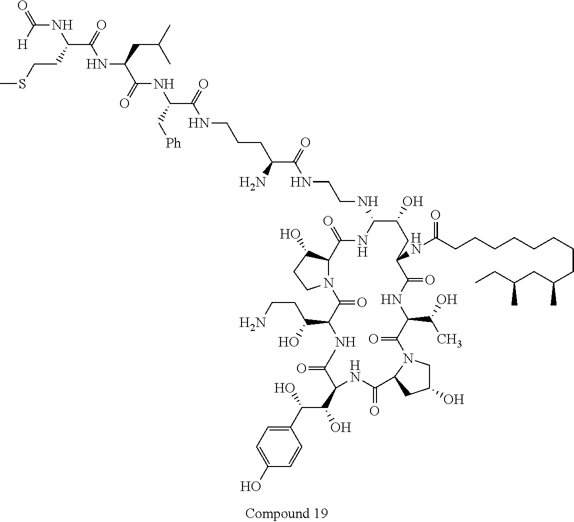 [SMILES: CC[C@H](C)C[C@H](C)CCCCCCCCC(=O)N[C@H]1C[C@@H](O)[C@@H](NCCNC(=O)[C@@H](N)CCCNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

To a solution of caspofungin acetate (150 mg, 0.124 mmol) in 8.0 mL dry DMF is added fMLF-(Z)^{α}-Orn-OSu (96.7 mg, 0.124 mmol) and the resulting mixture is stirred at 0 to 5° C. for 8 hours and then at room temperature overnight or until LC-MS analysis shows substantial reaction. Most of the DMF is removed via blowing a gentle stream of nitrogen at room temperature over the mixture and the crude product is purified via preparative HPLC to yield the desired product after solvent removal at reduced pressure at 30° C. and drying via lyophilization. To a solution of the above intermediate in a solvent mixture of iso-propanol (27 mL) and water (3 mL) containing acetic acid (1.35 mL) is added palladium on carbon (5 wt %, 0.23 g, 0.107 mmol) and the resulting mixture is stirred under hydrogen (balloon) at room temperature for 2 days. The catalyst is removed via filtration through a short path of celite and the majority of solvent is removed under reduced pressure. The crude product is purified via prep-HPLC to yield Compound 19 as a diacetate salt after solvent removal at reduced pressure at 30° C. and then lyophilization. Compound 19 has a parent mass of 1625.91 amu.

### Example 21

### Synthesis of Compound 20

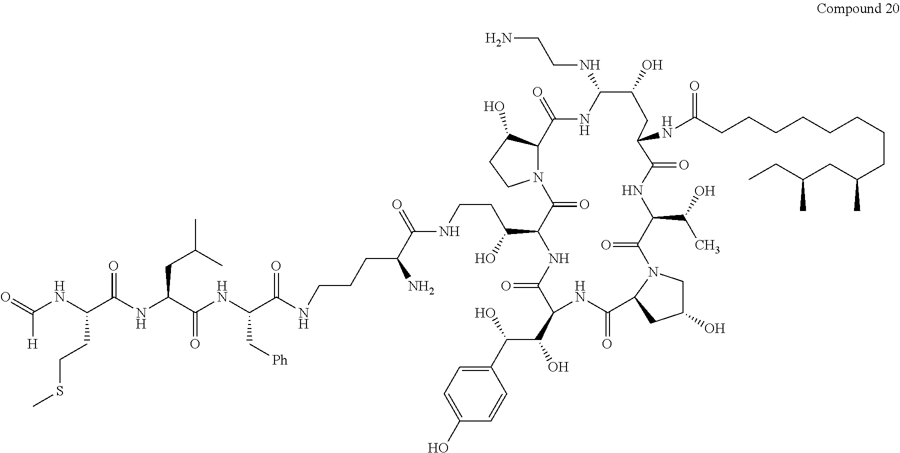 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@@H](N)CCCNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLF-(Z)^{α}-Orn-OSu as starting materials, Compound 20 is prepared in an analogous manner as Compound 15. Compound 20 has a parent mass of 1625.91 amu.

### Example 22

### Synthesis of Compound 21

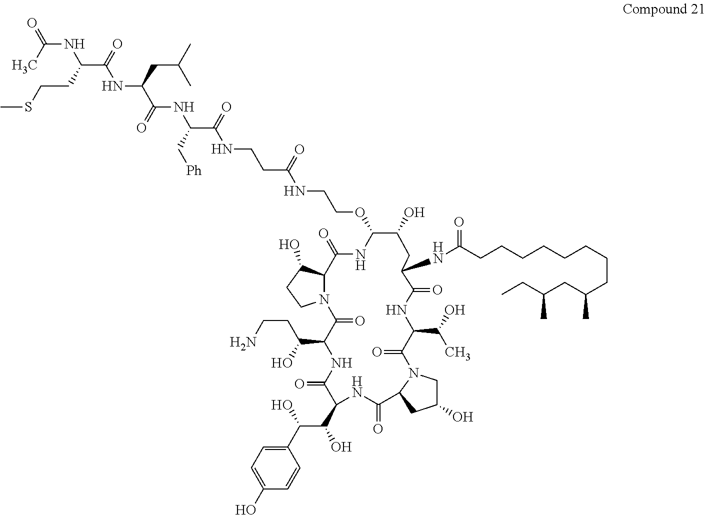 [SMILES: CC[C@H](C)C[C@H](C)CCCCCCCCC(=O)N[C@H]1C[C@@H](O)[C@@H](OCCNC(=O)CCNC(=O)[C@H](Cc2ccccc2)NC(=O)[C@H](CC(C)C)NC(=O)[C@H](CCSC)NC(C)=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Reacting Int-15 and a-MLF-β-Ala-OSu (Int-4) together in a manner analogous to the preparation of Compound 4, Compound 21 is synthesized. Compound 21 has a parent mass of 1597.87.

### Example 23

### Synthesis of Compound 22

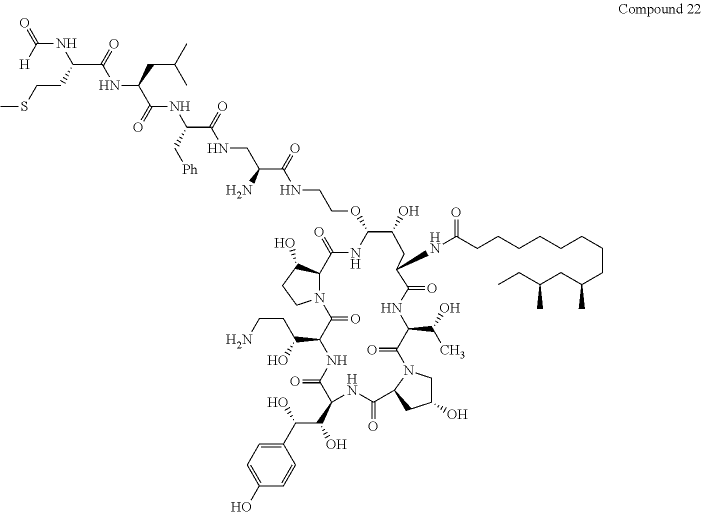 [SMILES: CC[C@H](C)C[C@H](C)CCCCCCCCC(=O)N[C@H]1C[C@@H](O)[C@@H](OCCNC(=O)[C@@H](N)CNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using Int-15 and fMLF-L-(Z)^{α}-Dap-OSu (Int-14) as starting materials, Compound 22 is synthesized in an analogous manner as Compound 19. Compound 22 has a parent mass of 1598.86 amu.

### Example 24

### Synthesis of Compound 23

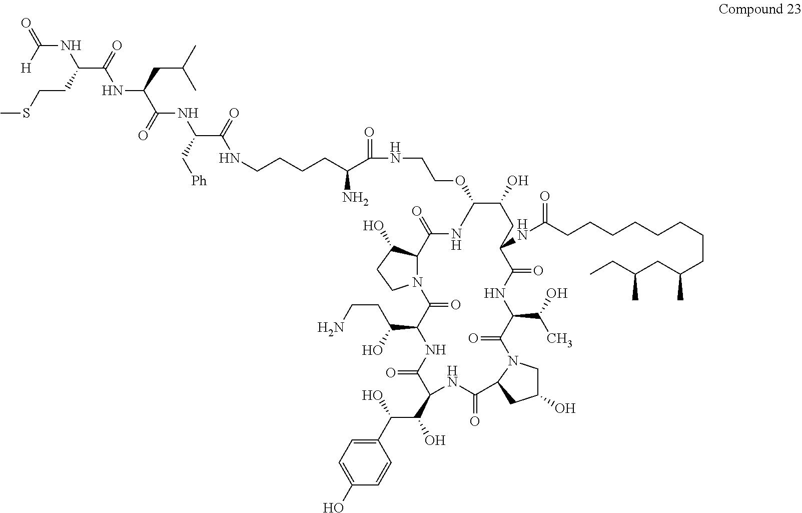 [SMILES: CC[C@H](C)C[C@H](C)CCCCCCCCC(=O)N[C@H]1C[C@@H](O)[C@@H](OCCNC(=O)[C@@H](N)CCCCNC(=O)[C@H](Cc2ccccc2)NC(=O)[C@H](CC(C)C)NC(=O)[C@H](CCSC)NC=O)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using Int-15 and fMLF-(Z)^{α}—K-OSu as starting materials, Compound 23 is prepared in an analogous manner as Compound 19. Compound 23 has a parent mass of 1640.91 amu.

### Example 25

### Synthesis of Compound 24

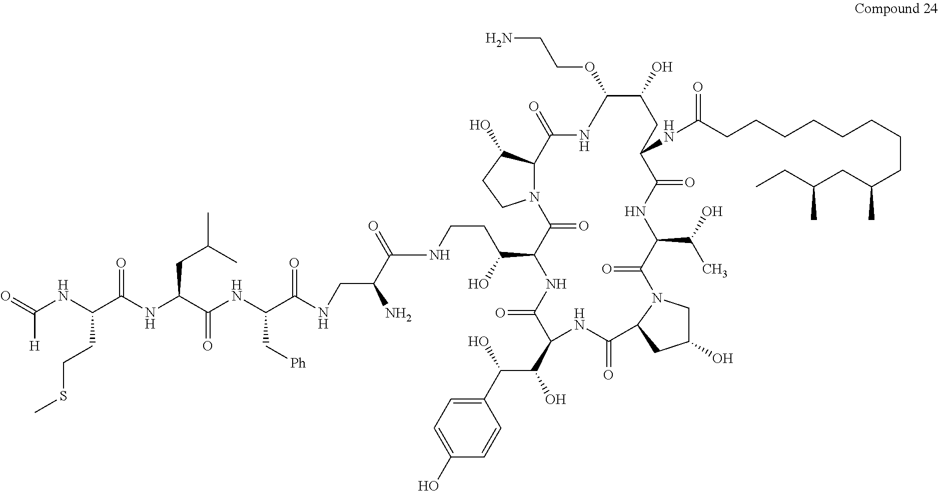 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)[C@@H](N)CNC(=O)[C@H](Cc2ccccc2)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using Int-15 and fMLF-L-(Z)^{α}-Dap-OSu (Int-14) as starting materials, Compound 24 is synthesized in an analogous manner as Compound 15. Compound 24 has a parent mass of 1598.86 amu.

### Example 26

### Synthesis of Compound 25

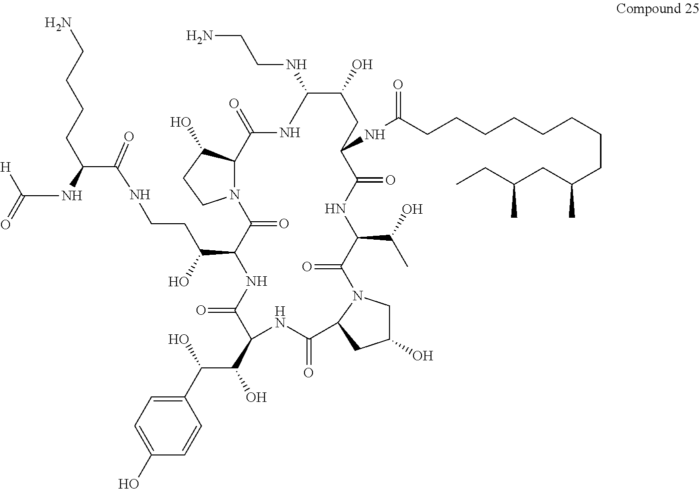 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCCCN)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and f^{α}-(Z)^{ε}—K-OSu as starting materials, Compound 25 was synthesized using using an analogous procedure as Compound 14. HPLC (ELSD), T_{R} 6.46 min (100%); H-NMR (400 MHz, Methanol-d₄) δ 8.11 (s, 1H), 7.13-7.15 (m, 2H), 6.74-6.77 (m, 2H), 5.00 (d, 1H, J=3.49 Hz), 4.94 (d, 1H, J=3.84 Hz), 1.95 (s, 6H), 0.85-0.96 (m, 10H); LC/MS, 1251.6 [M+H]⁺.

### Example 27

### Synthesis of Compound 26

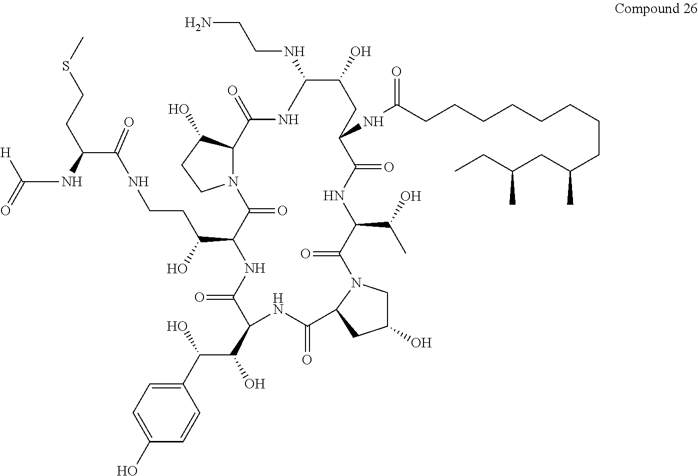 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

A mixture of formyl-L-methionine (0.0438 g, 0.2471 mmol), N-hydroxysuccinimide (0.030 g, 1.05 eq) in DMF (3 mL) was added DCC (0.051 g, 1.0 eq). The reaction mixture was stirred at RT overnight. The precipitate was filtered and the product as a solution in DMF was used directly for the next step. Using caspofungin acetate and fM-OSu prepared above as starting materials, Compound 26 was synthesized using the same procedure as Compound 1. HPLC (UV at 220 nm), T_{R} 7.75 min (93.8%); ¹H-NMR (400 MHz, Methanol-d₄) δ 8.09 (s, 1H), 7.13-7.15 (m, 2H), 6.74-6.76 (m, 2H), 5.02 (d, 1H, J=3.41 Hz), 4.94 (d, 1H, J=3.70 Hz), 4.53-4.59 (m, 6H), 4.24-4.36 (m, 4H), 4.18 (d, 1H, J=4.54 Hz), 3.89-4.01 (m, 3H), 3.77-3.83 (m, 3H), 3.37-3.44 (m, 1H), 3.18-3.25 (m, 1H), 3.08-3.15 (m, 1H), 2.80-3.01 (m, 3H), 2.41-2.54 (m, 3H), 2.24-2.31 (m, 3H), 2.09 (s, 3H), 1.91 (s, 3H), 1.80-2.21 (m, 6H), 1.57-1.71 (m, 3H), 1.20-1.52 (m, 19H), 1.05-1.13 (m, 2H), 0.85-0.96 (m, 10H); LC/MS, 1252.5 [M+H]⁺.

### Example 28

### Synthesis of Compound 27

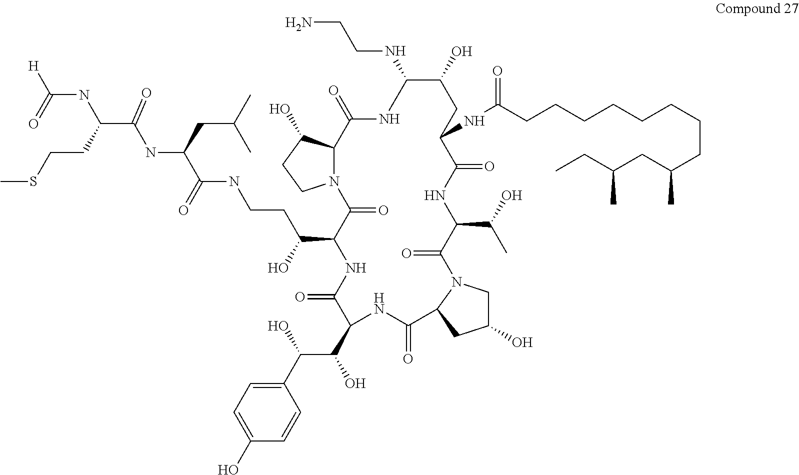 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fML-OSu as starting materials, Compound 27 was synthesized as a mixture of diastereomers using a similar procedure as for Compound 1. HPLC (ELSD), T_{R} 8.367 min (54.9%), 8.565 min (45.1%); LC/MS, 1366.6 [M+H]⁺.

### Example 29

### Synthesis of Compound 28

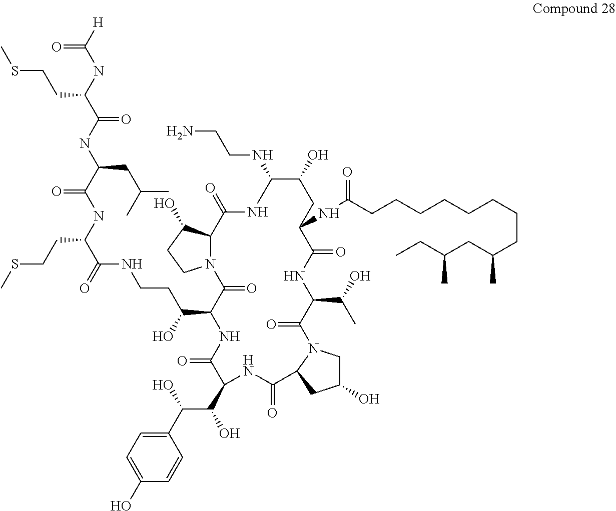 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCSC)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLM-OSu as starting materials, Compound 28 was synthesized as a mixture of diastereomers using the same procedure as Compound 1. HPLC (UV at 220 nm), T_{R} 7.66 min (100.0%); ¹H-NMR (400 MHz, Methanol-d₄) δ8.10 (s, 1H), 7.13-7.15 (m, 2H), 6.74-6.76 (m, 2H), 5.00 (dd, 2H, J₁=3.17 Hz, J₂=3.36 Hz), 1H), 4.88-4.89 (m, 1H), 4.50-4.60 (m, 5H), 4.26-4.42 (m, 6H), 4.20 (d, 1H, J=4.26 Hz), 3.95-4.05 (m, 2H), 3.78-3.90 (m, 4H), 3.09-3.15 (m, 1H), 2.78-3.01 (m, 3H), 2.42-2.62 (m, 5H), 1.98-2.32 (m, 13H), 2.10 (s, 3H), 2.09 (s, 3H), 1.91 (s, 3H), 1.55-1.86 (m, 8H), 1.21-1.51 (m, 20H), 1.04-1.14 (m, 2H), 0.85-0.99 (m, 19H); LC/MS, 1497.6 [M+H]⁺.

### Example 30

### Synthesis of Compound 29

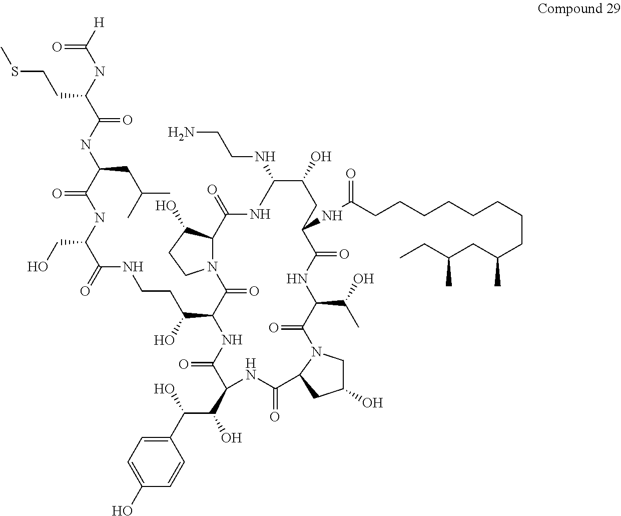 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CO)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Using caspofungin acetate and fMLS(OBn)-OSu as starting materials, Compound 29 was synthesized using the same procedure as Compound 14. HPLC (ELSD), T_{R} 7.353 min (90%); LC/MS, 1453.6 [M+H]⁺.

### Example 30a

### Synthesis of Compound 30

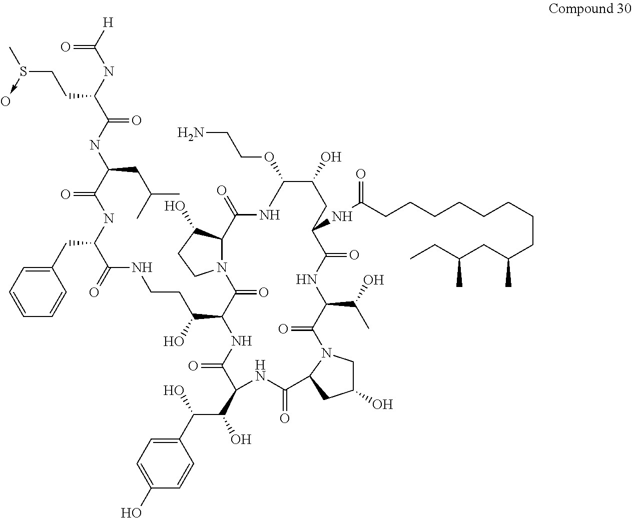 [SMILES: CC[C@H](C)C[C@H](C)CCCCCCCCC(=O)N[C@H]1C[C@@H](O)[C@@H](OCCN)NC(=O)[C@@H]2[C@@H](O)CCN2C(=O)[C@H]([C@H](O)CCNC(=O)[C@H](Cc2ccccc2)NC(=O)[C@H](CC(C)C)NC(=O)[C@H](CCS(C)=O)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Compound 11 (100 mg, 0.066 mmol) was dissolved in methanol (1.5 mL), and water (0.5 mL), at 25° C., then treated with Oxone (13 mg, 0.860 mmol) in one portion. LCMS after 2 h showed complete consumption of starting material. The product was isolated by reverse phase liquid chromatography (RPLC) using an Isco CombiFlash liquid chromatograph eluted with 10% to 100% acetonitrile and water, using 0.1% formic acid as the modifier. Removal of the solvent gave 58 mg of a white powder. ¹H-NMR (300 MHz, Methanol-d₄) δ 8.12 (d, 2H, J=14.4 Hz), 7.58 (d, 1H, J=6.7 Hz), 7.10-7.29 (m, 7H), 6.77 (d, 2H, J=8.7 Hz), 4.95-5.23 (m, 3H), 4.44-4.68 (m, 6H), 4.20-4.44 (m, 5H), 4.20-3.72 (m, 7H), 2.78-3.17 (m, 7H), 2.665 (s, 3H), 2.05-2.44 (m, 7H), 1.00-2.05 (m, 31H), 0.71-1.00 (m, 17H); LC/MS, 765.2 [M/2+H]+ calculated 764.9.

### Example 30b

### Synthesis of Compound 31

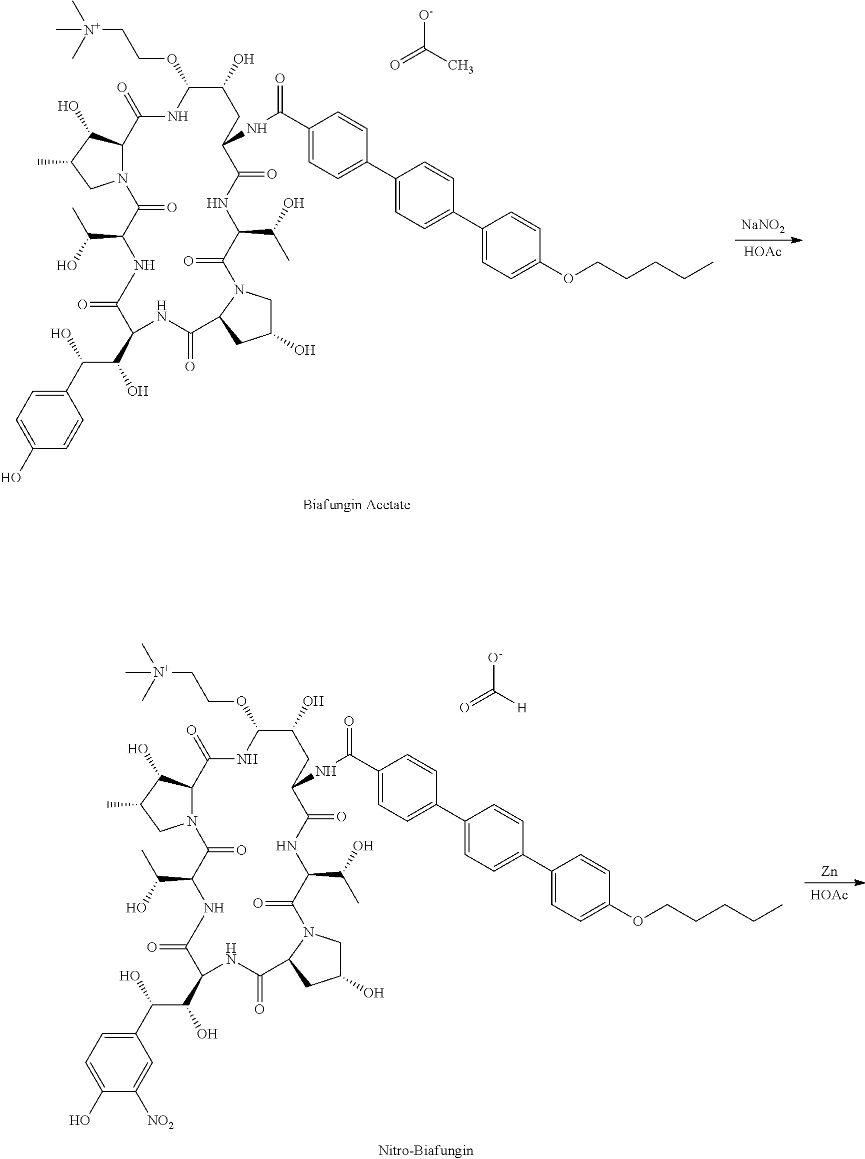

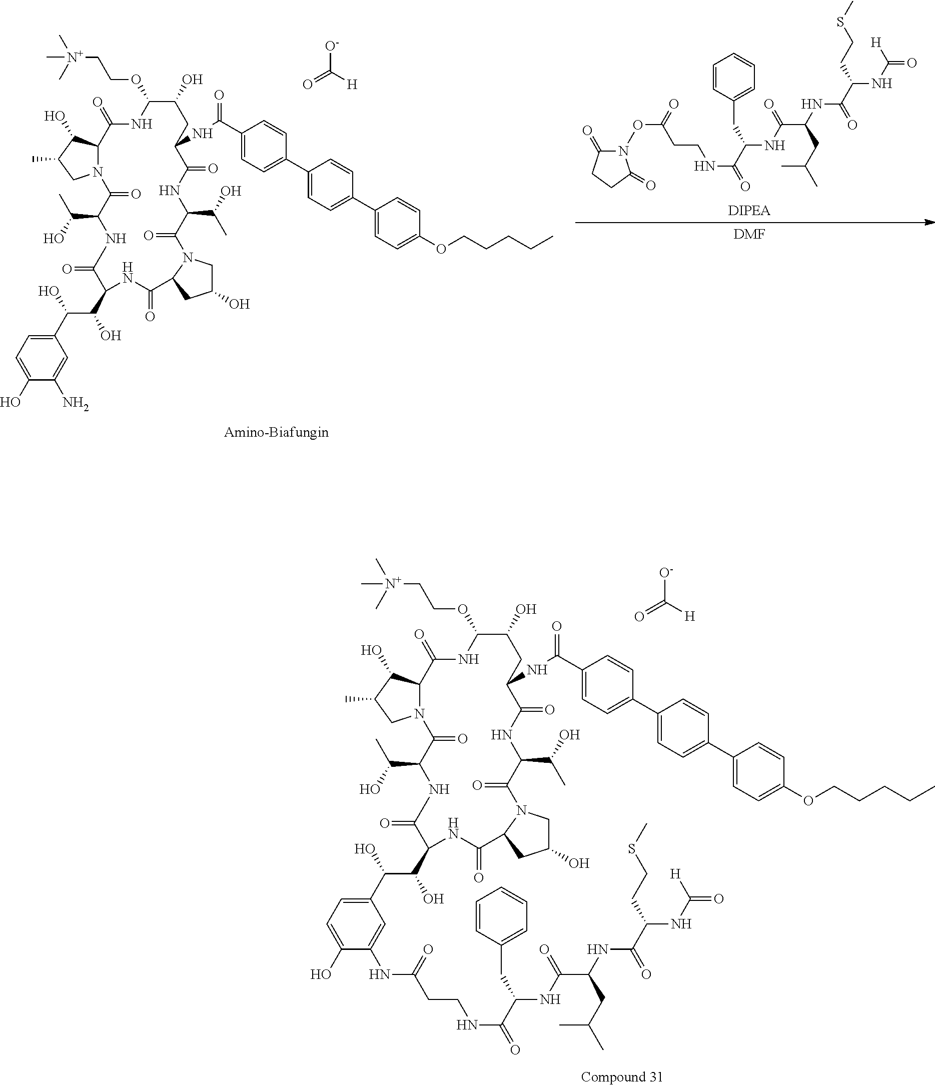

### Step a. Nitration of Biafungin Acetate

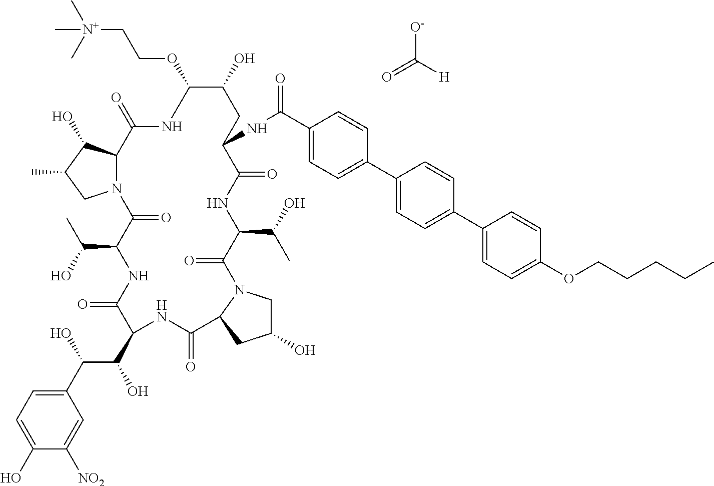

To a stirring solution of biafungin (100 mg, 0.078 mmol) in glacial acetic acid (1.5 mL) was added sodium nitrite (11 mg, 0.159 mmol) and the reaction was stirred at ambient temperature for 20 hours. The mixture was applied directly to reversed phase HPLC (Isco CombiFlash Rf; 50 g RediSep C18 column, 5 to 95% acetonitrile in DI water containing 0.1% formic acid: 15 minute gradient). The pure fractions were pooled and lyophilized to yield 85 mg of the desired product as a light yellow solid, formate salt. ¹H-NMR (300 MHz, Methanol-d₄) δ 8.58 (d, 1H, J=11.7 Hz), 8.47 (t, 2H, J=8.7 Hz), 8.05 (d, 1H, J=2.1 Hz), 7.99 (d, 2H, J=9.3 Hz), 7.82 (d, 2H, J=8.7 Hz), 7.79-7.60 (m, 12H), 7.17 (d, 1H, J=8.7 Hz), 7.03 (d, 2H, J=9 Hz), 5.48 (d, 1H, J=6 Hz), 5.08 (dd, 1H, J=1.2, 5.7 Hz), 4.95-4.73 (m, 5H), 4.68-4.56 (m, 2H), 4.53 (d, 1H, J=5.7 Hz), 4.48-4.39 (m, 2H), 4.31-3.79 (m, 6H), 4.04 (t, 2H, J=5.7 Hz), 3.72-3.44 (m, 3H), 3.18 (s, 9H), 2.60-1.99 (m, 5H), 1.83 (m, 2H, J=8.7 Hz), 1.56-1.35 (m, 5H), 1.28 (d, 6H, J=4.2 Hz), 1.09 (d, 3H, J=10.2 Hz), 0.99 (t, 3H, J=8.7 Hz); LC/MS, [M/2+H]+: 635.79, 635.80 calculated.

### Step b. Reduction of Nitro-Biafungin To Amino-Biafungin

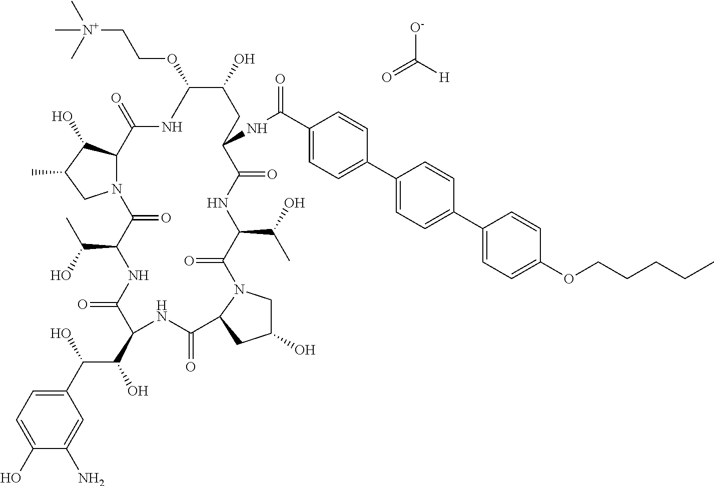

To a stirring solution of Nitro-Biafungin (100 mg, 0.075 mmol) in glacial acetic acid (1.5 mL) was added zinc powder (50 mg, 0.77 mmol) and the reaction was stirred at ambient temperature for 1 hour. The mixture was filtered and applied directly to reversed phase HPLC (Isco CombiFlash Rf, 50 g Redisep C18 column; 5 to 95% acetonitrile in DI water containing 0.1% formic acid: 15 minute gradient). The pure fractions were pooled and lyophilized to yield 55 mg of the desired product as a white solid, formate salt. ¹H-NMR (300 MHz, Methanol-d4) δ 8.47 (bs, 1H), 7.99 (d, 2H, J=10.8 Hz), 7.82 (d, 2H, J=7.5 Hz), 7.80-7.67 (m, 6H), 7.62 (d, 2H, J=8.7 Hz), 7.03 (d, 2H, J=7.5 Hz), 6.77 (d, 1H, J=1.9 Hz), 6.68 (d, 1H, J=8.2 Hz), 6.55 (dd, 2H, J=8.2, 1.9 Hz), 5.43 (d, 1H, J=2.5 Hz), 5.05 (d, 1H, J=3 Hz), 4.83-4.73 (m, 2H), 4.64-4.56 (m, 2H), 4.43-4.34 (m, 2H), 4.31-4.15 (m, 4H), 4.03-4.08 (m, 1H), 4.11-3.89 (m, 8H), 3.83 (d, 1H, J=10.8 Hz), 3.68-3.47 (m, 3H), 3.17 (s, 9H), 2.57-2.42 (m, 2H), 2.35-2.27 (m, 1H), 2.14-1.98 (m, 2H), 1.83 (m, 2H, J=6 Hz), 1.56-1.38 (m, 4H), 1.28 (dd, 6H, J=6.5, 2 Hz), 1.09 (d, 3H, J=7 Hz), 0.986 (t, 3H, J=7 Hz); High Res LC/MS: [M+H]+ 1241.6163; 1241.6136 calculated.

### Step c. Reaction of Amino-Biafungin with Int-2 to Produce Compound 31

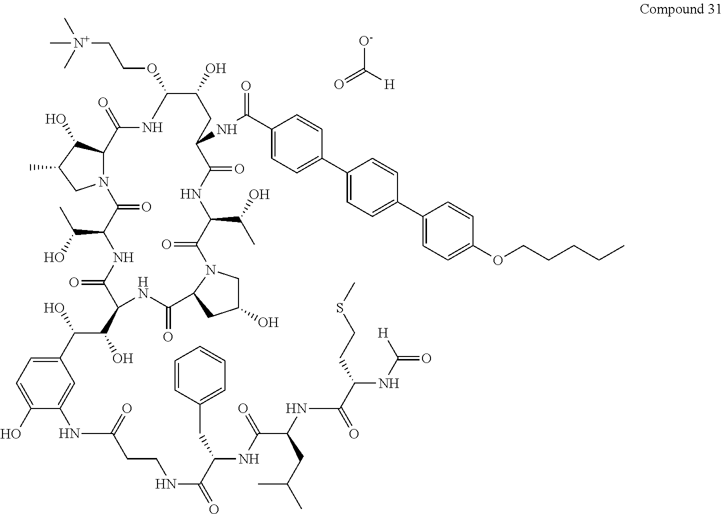

To a stirring solution of Amino-Biafungin (50 mg, 0.04 mmol) in DMF (1 mL) was added formyl-Met-Leu-Phe-β-Ala-OSu (Int-2) (36 mg, 0.06 mmol) and DIPEA (7 uL, 0.04 mmol). The reaction was stirred at ambient temperature for 18 hours. The mixture was applied directly to reversed phase HPLC (Isco CombiFlash Rf; 50 g Redisep C18 column; 5 to 95% acetonitrile in DI water containing 0.1% formic acid: 15 minute gradient). The pure fractions were pooled and lyophilized to yield 26 mg of a white solid as a formate salt. ¹H-NMR (300 MHz, Methanol-d4) δ 8.55 (bs, 1H), 8.44 (t, 1H, J=10 Hz), 8.18 (d, 1H, J=6 Hz), 8.11 (s, 1H), 7.99 (d, 2H, J=10 Hz), 7.84-7.70 (m, 6H), 7.63 (d, 2H, J=7.8 Hz), 7.32-7.19 (m, 6H), 7.03 (d, 4H, J=9 Hz), 6.87 (d, 1H, J=8.1 Hz), 5.44 (d, 1H, J=10.5 Hz), 5.05 (d, 1H, J=4.5 Hz), 4.83-4.74 (m, 2H), 4.66-4.50 (m, 6H), 4.45-4.29 (m, 10H), 4.19-3.82 (m, 10H), 3.67-3.57 (m, 6H), 3.17 (s, 9H), 2.64-2.46 (m, 6H), 2.14-1.92 (m, 6H), 1.84 (m, 4H, J=6 Hz), 1.62-1.40 (m, 8H), 1.32-1.22 (m, 6H), 1.09 (d, 3H, J=9 Hz), 0.99 (t, 3H, J=7.5 Hz), 0.88 (m, 6H, J=6.8 Hz); High Res LC/MS, [M/2+H]+865.4143, 865.4147 calculated.

### Example 30c

### Synthesis of Compound 32

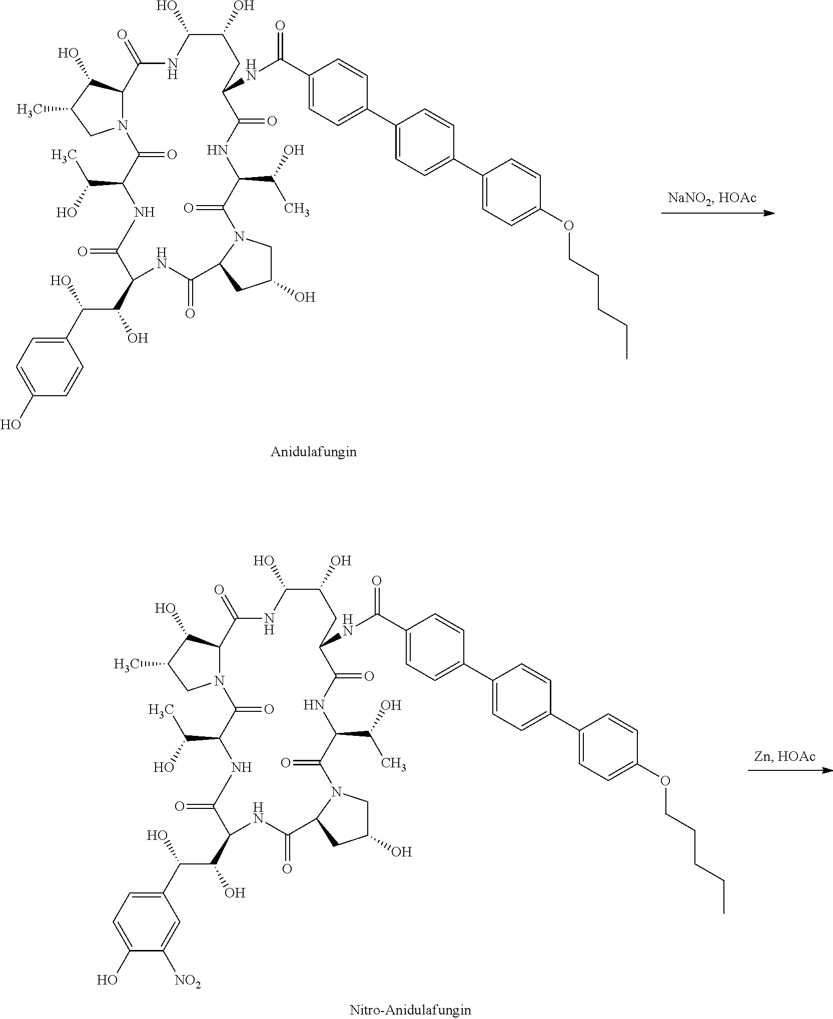

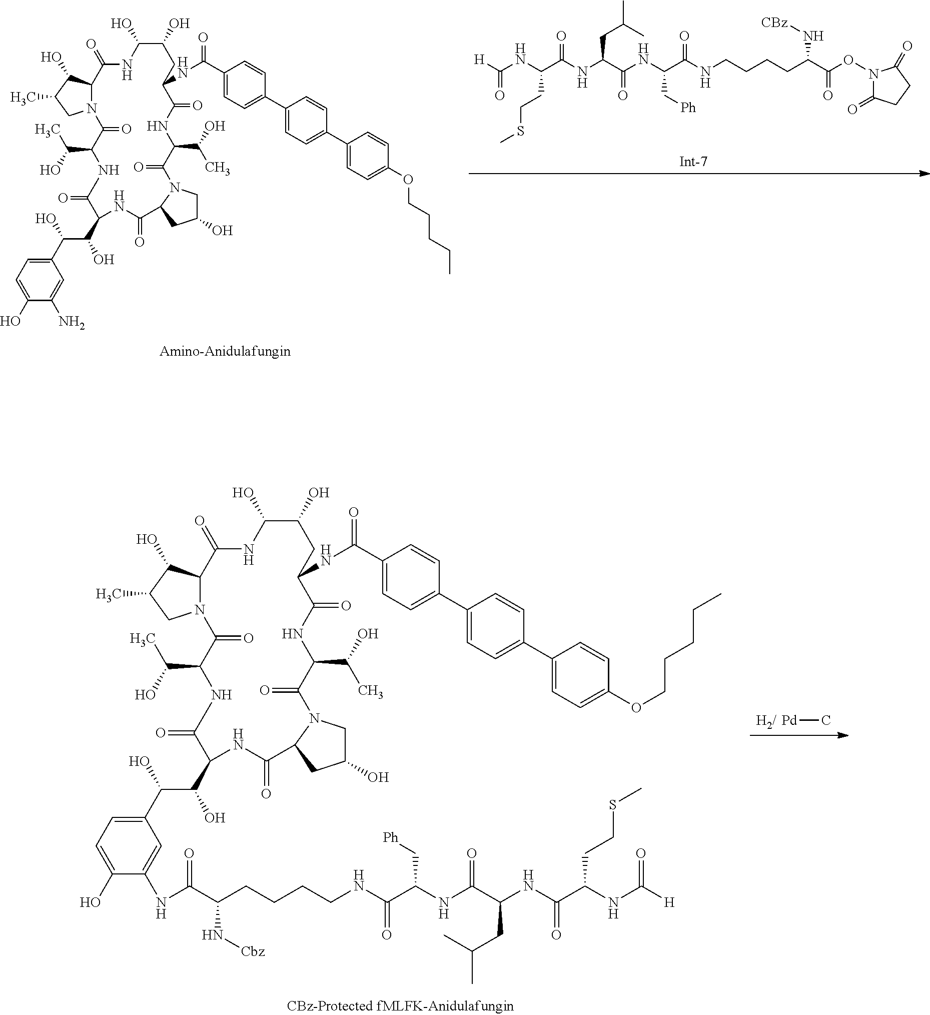

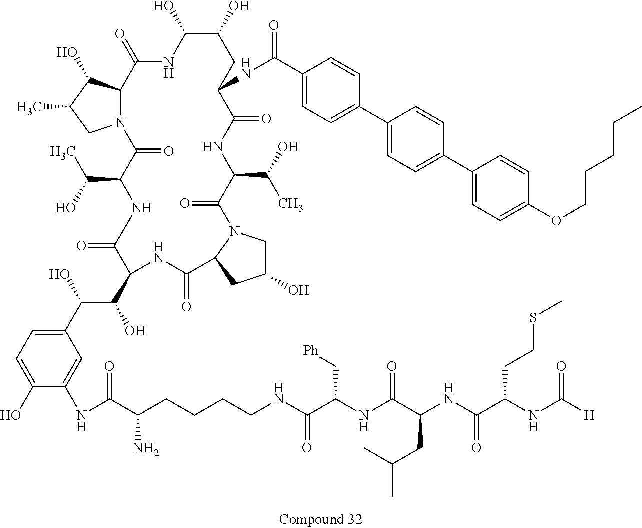 [SMILES: CCCCCOc1ccc(-c2ccc(-c3ccc(C(=O)N[C@H]4C[C@@H](O)[C@@H](O)NC(=O)[C@@H]5[C@@H](O)[C@@H](C)CN5C(=O)[C@H]([C@@H](C)O)NC(=O)[C@H]([C@H](O)[C@@H](O)c5ccc(O)c(NC(=O)[C@@H](N)CCCCNC(=O)[C@H](Cc6ccccc6)NC(=O)[C@H](CC(C)C)NC(=O)[C@H](CCSC)NC=O)c5)NC(=O)[C@@H]5C[C@@H](O)CN5C(=O)[C@H]([C@@H](C)O)NC4=O)cc3)cc2)cc1]

### Step a. Synthesis of Nitro-anidulafungin

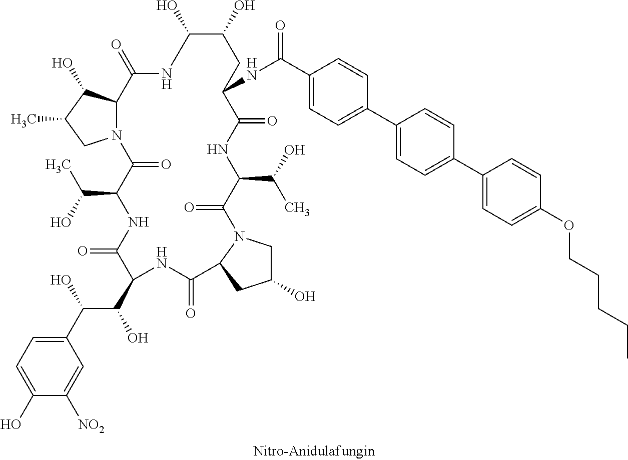

Anidulafugin (0.100 mmol) is added to acetic acid (1 mL) and is treated with NaNO₂ (0.200 mmol) at room temperature. The reaction is stirred until sufficient reaction has taken place. Nitro-anidulafungin is purified and isolated by RPLC using liquid chromatography eluting with 10% to 100% acetonitrile and water and 0.1% formic acid as the modifier. Product-containing fractions are pooled and removal of the volatiles in vacuo give nitro-anidulafungin. The product has an exact mass of 1184.49.

### Step b. Synthesis of Amino-Anidulafungin

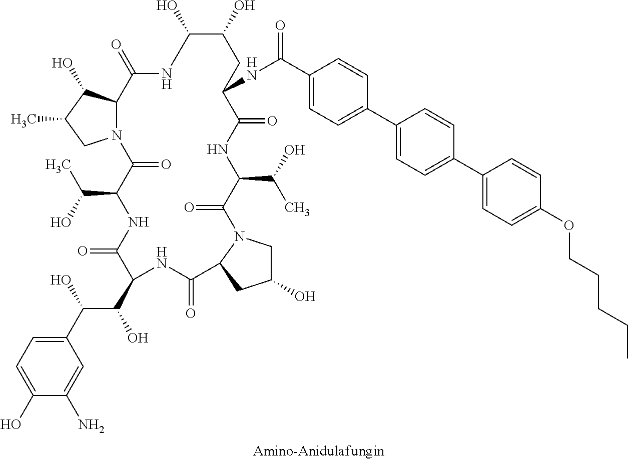

Nitro-anidulafungin (0.100 mmol) is added to acetic acid (1.0 ml) and is treated with Zn powder (0.500 mmol) portionwise over 15 min. After sufficient reaction as evidenced by LCMS the reaction is filtered and diluted with water. The mixture is purified by RPLC using liquid chromatography eluting with 10% to 100% acetonitrile and water and 0.1% formic acid. Product-containing fractions are pooled and removal of the volatiles in vacuo give amino-anidulafungin. The product has an exact mass of 1154.52.

### Step c. Synthesis of fMLFK(αCBz)-anidulafungin

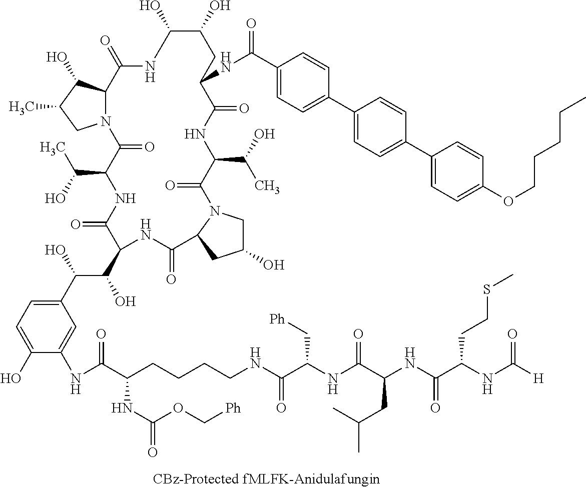

Amino-anidulafungin (0.100 mmol) in DMF (1.0 mL) is treated with fMLFK(αCBz)-OSu (Int-7) (0.150 mmol) at room temperature. After LCMS shows substantial conversion ov starting material, the product is isolated by RPLC using liquid chromatography eluting with 10% to 100% acetonitrile and water and 0.1% formic acid. Product-containing fractions are pooled and removal of the volatiles in vacuo give Fmoc-protected fMLFK-anidulafungin. The product has an exact mass of 1835.84.

### Step d. Synthesis of fMLFK-anidulafungin (Compound 32)

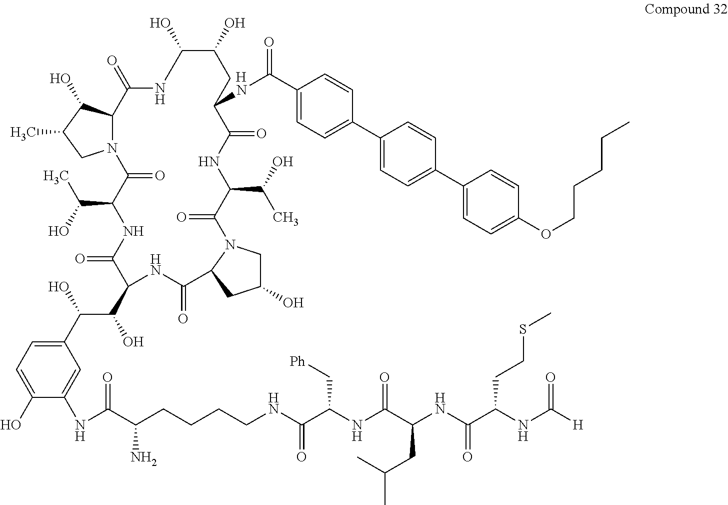

fMLFK(αCBz)-Anidulafungin (0.1 mmol) in ethanol (1.0 ml) is stirred vigorously with 10% palladium on carbon (10 mg) under one atmosphere of hydrogen gas at room temperature. After sufficient conversion of starting material is determined by LCMS the mixture is purified by RPLC liquid chromatography eluting with 10% to 100% acetonitrile and water and 0.1% formic acid. Product-containing fractions are pooled and removal of the volatiles in vacuo give Compound 33. Compound 33 has an exact mass of 1701.80.

### Example 30d

### Preparation of Compound 33

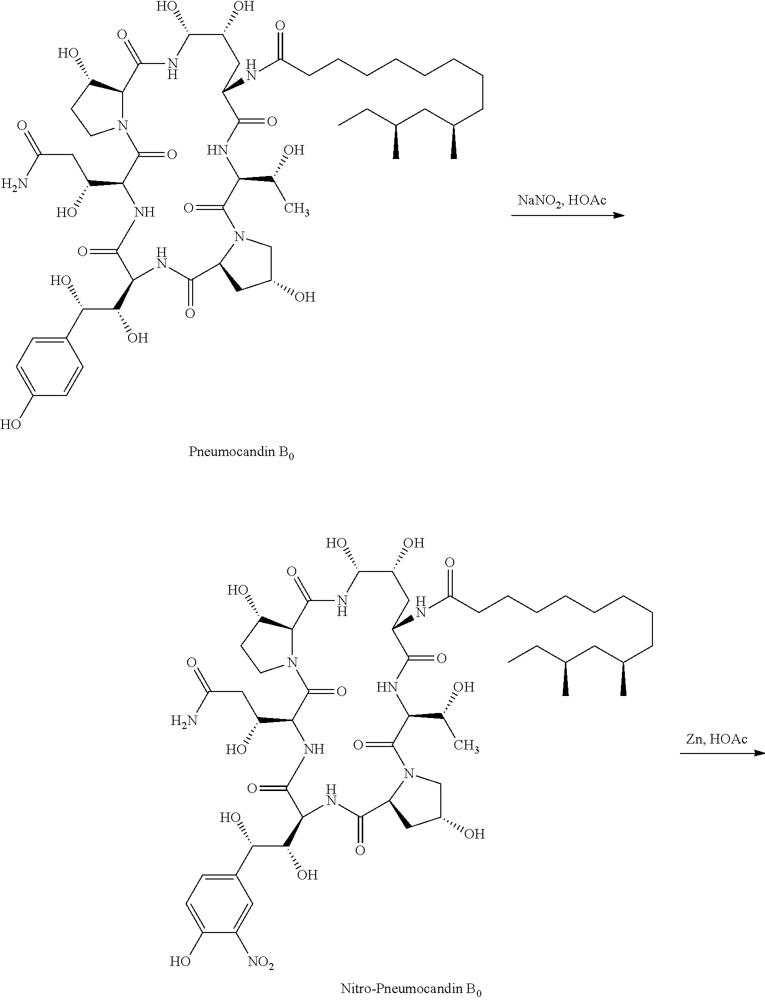

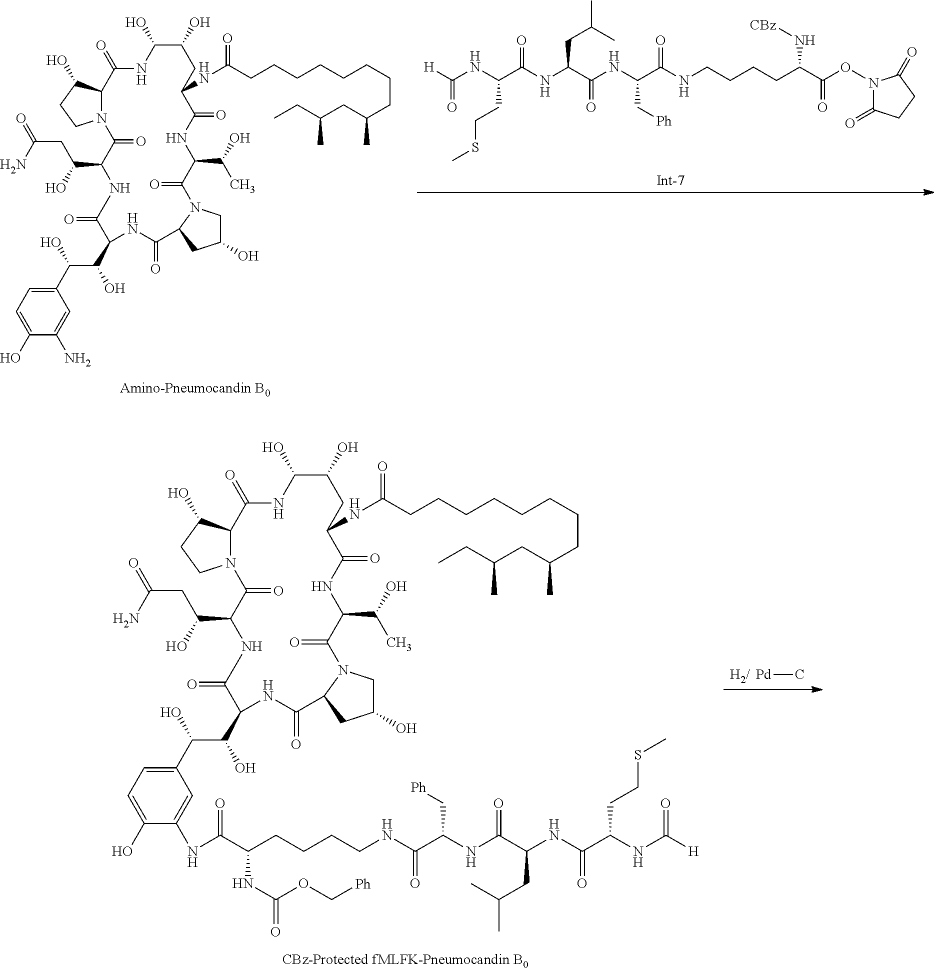

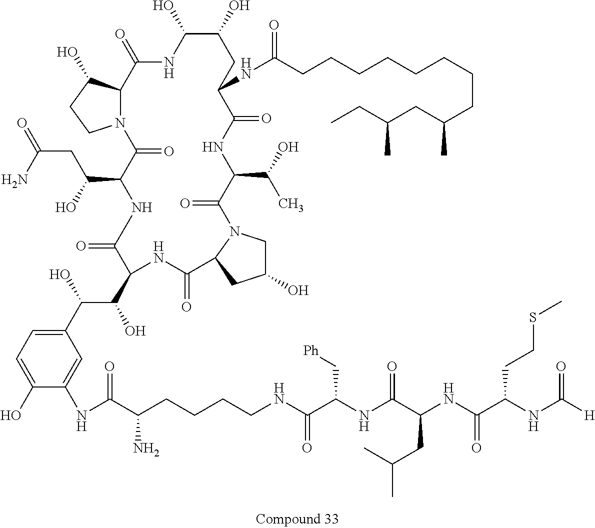 [SMILES: CC[C@H](C)C[C@H](C)CCCCCCCCC(=O)N[C@H]1C[C@@H](O)[C@@H](O)NC(=O)[C@@H]2[C@@H](O)CCN2C(=O)[C@H]([C@H](O)CC(N)=O)NC(=O)[C@H]([C@H](O)[C@@H](O)c2ccc(O)c(NC(=O)[C@@H](N)CCCCNC(=O)[C@H](Cc3ccccc3)NC(=O)[C@H](CC(C)C)NC(=O)[C@H](CCSC)NC=O)c2)NC(=O)[C@@H]2C[C@@H](O)CN2C(=O)[C@H]([C@@H](C)O)NC1=O]

In a manner analogous to the preparation of Compound 32, Compound 33 is prepared starting with pneumocandin B₀ and utilizing Int-7. Compound 33 has an exact mass of 1626.86.

### Example 30e

### Preparation of Compound 34

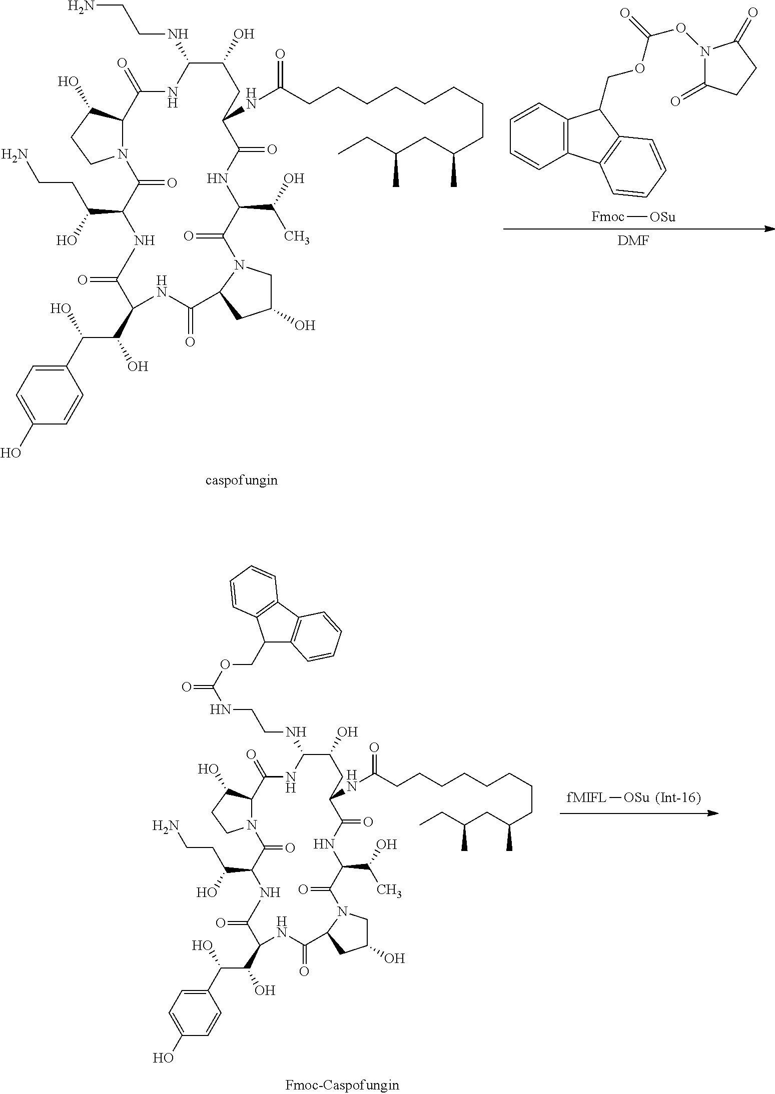

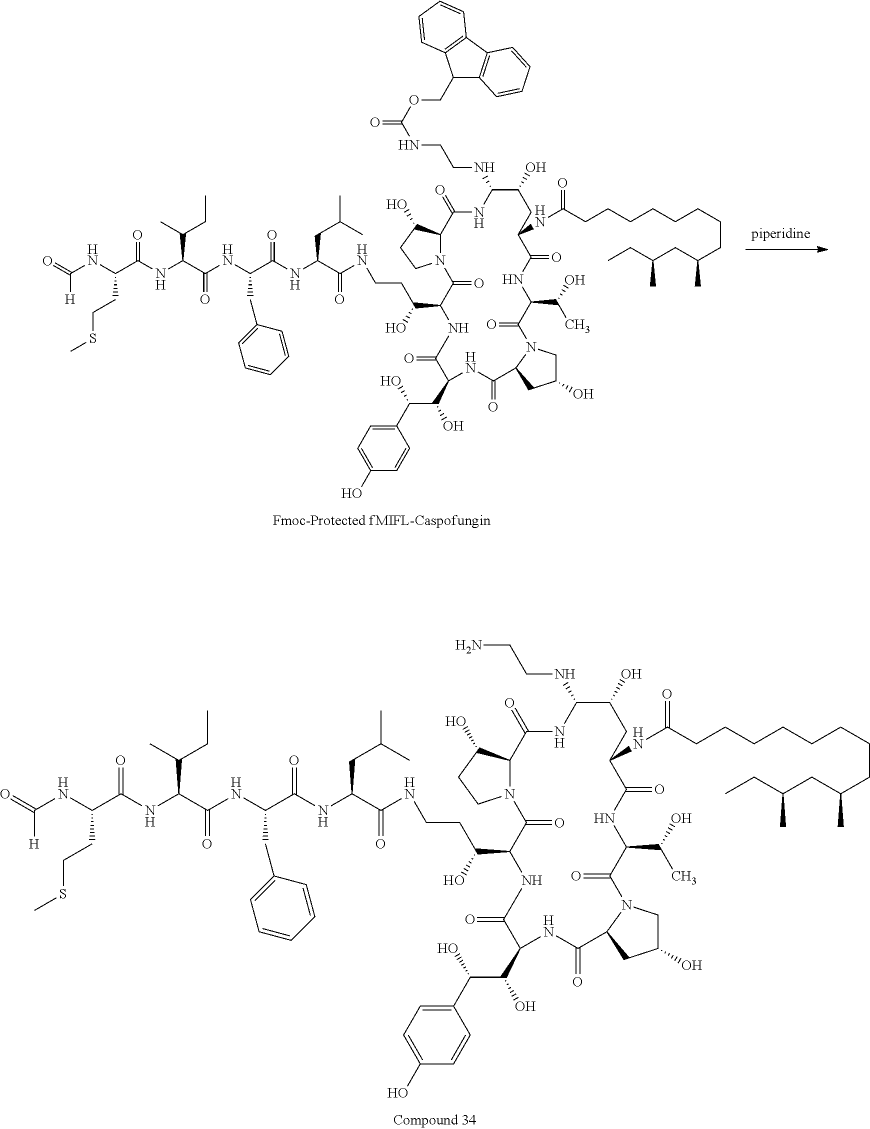

### Step a. Preparation of Fmoc-Caspofungin

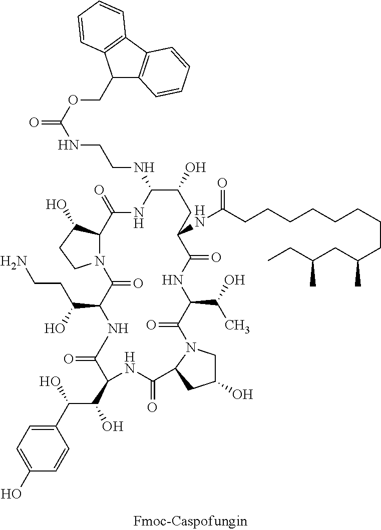 [SMILES: CC[C@H](C)C[C@H](C)CCCCCCCCC(=O)N[C@H]1C[C@@H](O)[C@@H](NCCNC(=O)OCC2c3ccccc3-c3ccccc32)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

FMOC-OSu (262 mg, 0.78 mmol, in 3 m of DMF) was added, dropwise, to a stirring solution of caspofungin acetate (860 mg, 0.71 mmol) in DMF (12 mL), cooled to −45° C. (acetonitrile dry ice bath) under an atmosphere of nitrogen. The reaction was stirred for 90 minutes while gradually rising to room temperature at which point both mono and bis FMOC adducts were observed by LC/MS. The solvent was reduced to approximately 6 mL on the rotovap and applied directly to reversed phase HPLC purification (Isco CombiFlash Rf; 50 g Redisep C18 column; 20 to 95% acetonitrile in DI water containing 0.1% trifluoroacetic acid: 15 minute gradient). The pure fractions were pooled and concentrated on the rotovap to afford 660 mg of the product as a white solid, trifluoracetate salt. LC/MS, [M/2+H]+ 658.4, 658.3 calculated.

### Step b. Conjugation of Fmoc-Caspofungin with fMIFL

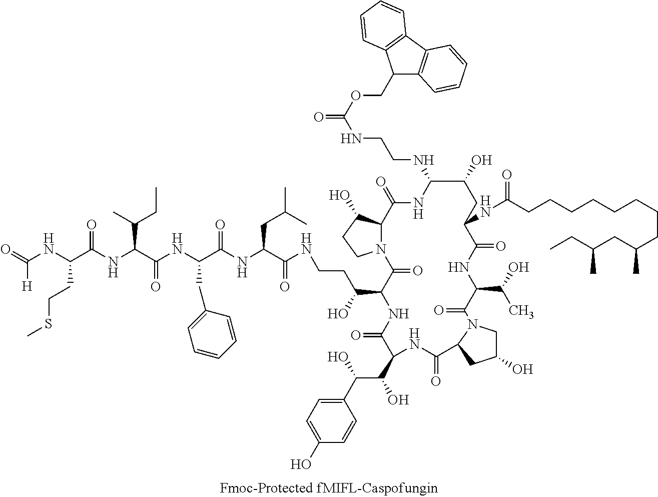 [SMILES: CCC(C)[C@H](NC(=O)[C@H](CCSC)NC=O)C(=O)N[C@@H](Cc1ccccc1)C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCNC(=O)OCC2c3ccccc3-c3ccccc32)NC(=O)[C@@H]2[C@@H](O)CCN2C1=O]

To a stirring solution of the mono-Fmoc-protected-caspofungin trifluoroacetate (0.87 mmol) in DMF (8 mL) is added N-methyl morpholine (3 mmol) and formyl-Met-Ile-Phe-Leu-OSu (Int-16) (1 mmol in 4 mL DMF). The reaction is stirred until analysis shows substantial reaction. The mixture is then applied directly to a reversed phase HPLC column and purified using gradient elution. The pure fractions are combined and concentrated in vacuo to afford the product with an exact mass of 1846.98.

### Step c. Deprotection of Fmoc-Protected fMIFL-Caspofungin to Yield Compound 34

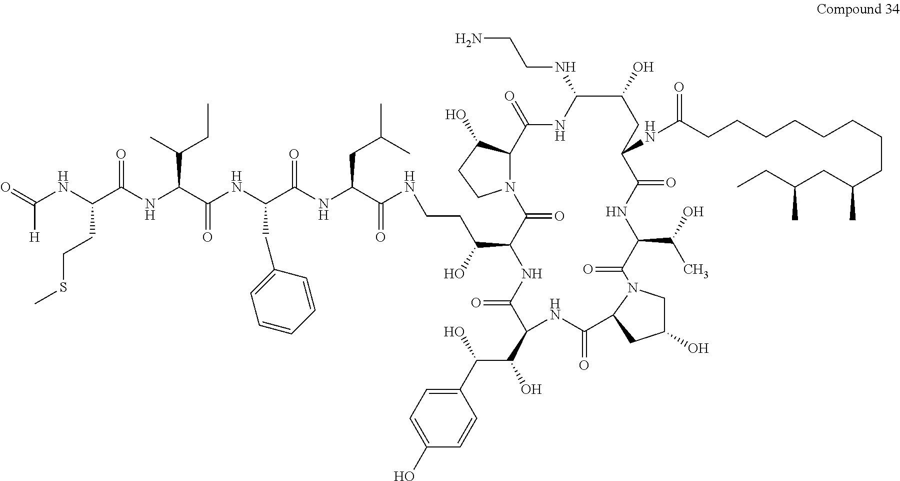 [SMILES: CCC(C)[C@H](NC(=O)[C@H](CCSC)NC=O)C(=O)N[C@@H](Cc1ccccc1)C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O]

To a stirring mixture of FMOC-protected-fMIFL-Caspofungin (0.56 mmol in 3 mL of DMF) is added 10% piperidine solution in DMF (3 m mL). The reaction is stirred at ambient temperature until substantial conversion of the starting material is seen by LC/MS analysis. The mixture is applied directly to reversed phase HPLC for purification and eluted using gradient elution. The pure fractions are pooled and most of the volatiles are removed in vacuo and the remaining mixture is lyophilized to afford the product with an exact mass of 1624.92.

### Example 30f

### Preparation of Compound 35

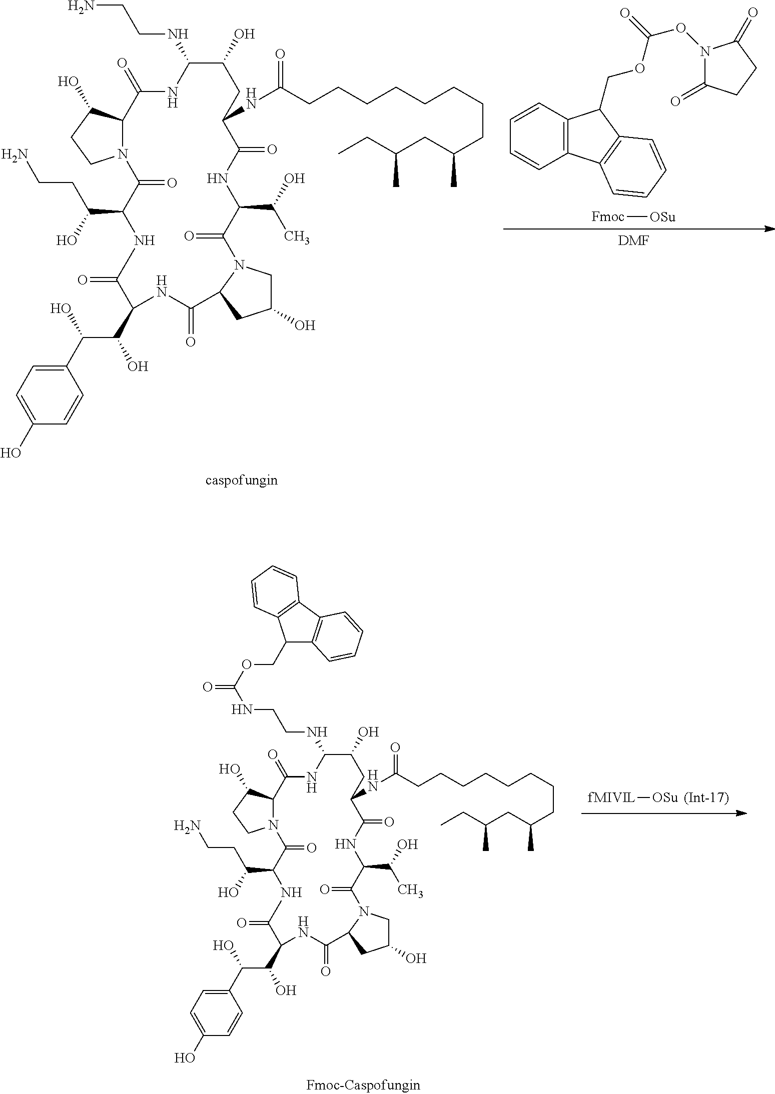

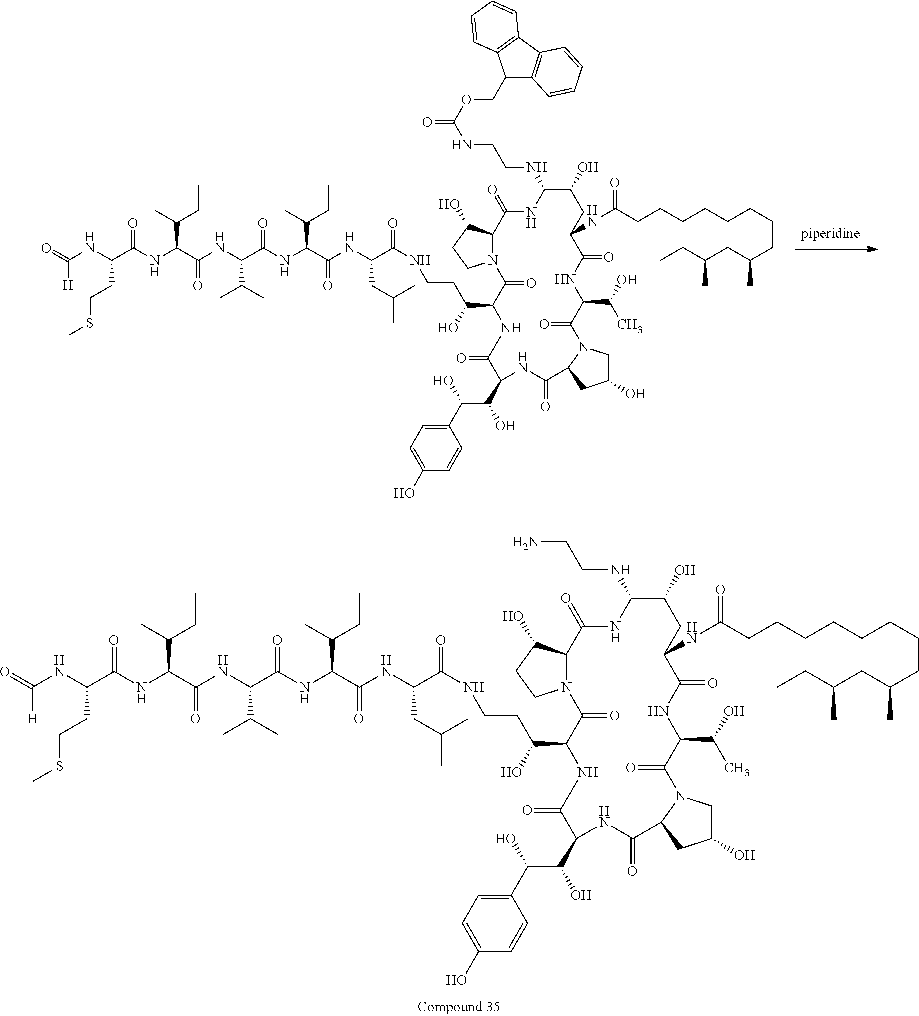

Step a. Preparation of Fmoc-Caspofungin.

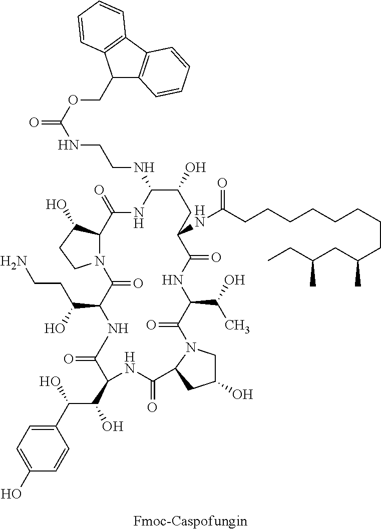 [SMILES: CC[C@H](C)C[C@H](C)CCCCCCCCC(=O)N[C@H]1C[C@@H](O)[C@@H](NCCNC(=O)OCC2c3ccccc3-c3ccccc32)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

FMOC-OSu (262 mg, 0.78 mmol, in 3 mL of DMF) was added, dropwise, to a stirring solution of caspofungin acetate (860 mg, 0.71 mmol) in DMF (12 mL), cooled to −45° C. (acetonitrile dry ice bath) under an atmosphere of nitrogen. The reaction was stirred for 90 minutes while gradually rising to room temperature at which point both mono and bis FMOC adducts were observed by LC/MS. The solvent was reduced to approximately 6 mL on the rotovap and applied directly to reversed phase HPLC purification (Isco CombiFlash Rf; 50 g Redisep C18 column; 20 to 95% acetonitrile in DI water containing 0.1% trifluoroacetic acid: 15 minute gradient). The pure fractions were pooled and concentrated on the rotovap to afford 660 mg of the product as a white solid, trifluoracetate salt. LC/MS, [M/2+H]+ 658.4, 658.3 calculated.

### Step b. Conjugation of Fmoc-Caspofungin with fMIVIL

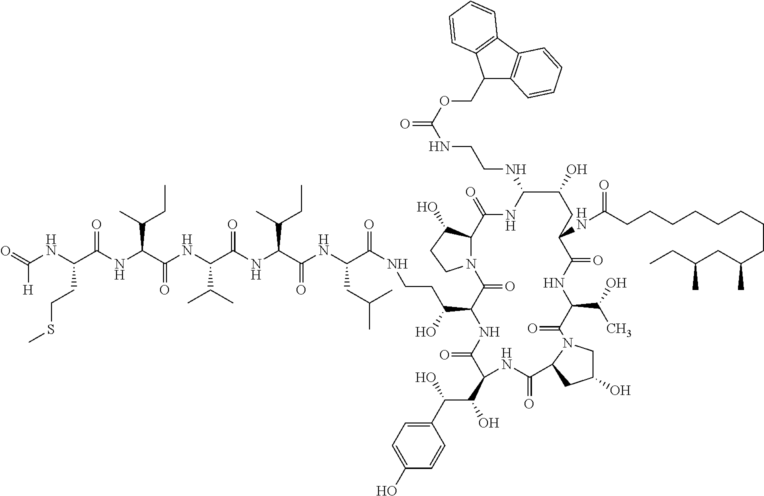 [SMILES: CCC(C)[C@H](NC(=O)[C@H](CCSC)NC=O)C(=O)N[C@H](C(=O)N[C@H](C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCNC(=O)OCC2c3ccccc3-c3ccccc32)NC(=O)[C@@H]2[C@@H](O)CCN2C1=O)C(C)CC)C(C)C]

To a stirring solution of the mono-FMOC-protected-caspofungin trifluoroacetate (0.90 mmol) in DMF (8 mL) is added N-methyl morpholine (3 mmol) and formyl-Met-Ile-Val-Ile-Leu-OSu (Int-17) (1 mmol in 4 mL DMF). The mixture is stirred until analysis shows substantial reaction. The mixture is then applied directly to a reversed phase HPLC column and purified using gradient elution. The pure fractions are combined and concentrated in vacuo to afford the product with an exact mass of 1912.07.

### Step c. Deprotection of FMOC-Protected fMIVIL-Caspofungin to Yield Compound 35

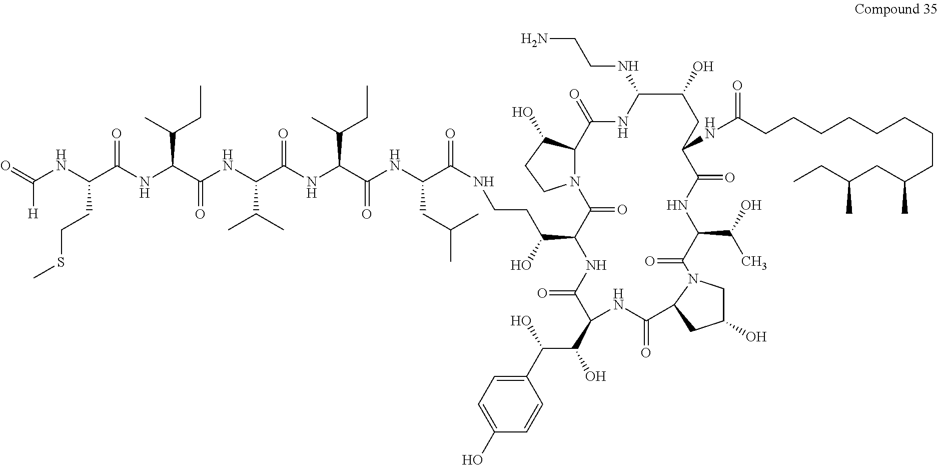 [SMILES: CCC(C)[C@H](NC(=O)[C@H](CCSC)NC=O)C(=O)N[C@H](C(=O)N[C@H](C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O)C(C)CC)C(C)C]

To a stirring solution of FMOC-protected-fMIFL-Caspofungin (0.5 mmol in 3 mL of DMF) is added 10% piperidine solution in DMF (3 mL). The reaction is stirred at ambient temperature until substantial conversion of the starting material is seen by LC/MS analysis. The mixture is applied directly to reversed phase HPLC for purification and eluted using gradient elution. The pure fractions are pooled and most of the volatiles are removed under reduced pressure and the mixture is then lyophilized to afford the product with an exact mass of 1690.00.

### Example 31

### Antifungal Activity of Compounds 1-29

Test Organisms

The test organisms consisted of strains from the Micromyx collection. Reference isolates were originally received from the American Type Culture Collection (ATCC; Manassas, Va.). Organisms received at Micromyx were initially streaked for isolation on Sabouraud dextrose or potato dextrose agar. Colonies were picked by swab from the medium and resuspended in the appropriate broth containing cryoprotectant. The suspensions were aliquoted into cryogenic vials and maintained at −80° C. Prior to testing, Candida isolates were streaked from the frozen vials on Sabouraud dextrose agar. The yeast isolates were incubated at overnight at 35° C. before use. The fungal isolates were incubated at least 7 days on Sabouraud dextrose agar slants at 35° C. before harvesting.

### Test Media

Isolates were tested in RPMI medium (Catalog No. SH30011.04; Lot No. AWA92121B; HyClone Labs, Logan, Utah) which was prepared according to CLSI guidelines. The pH of the medium was adjusted to 7.0 with 1 N NaOH. The medium was sterile filtered using a 0.2 μm PES filter and stored at 4° C. until used.

### Minimum Inhibitory Concentration (MIC) Assay Procedure

The MIC assay method employed automated liquid handlers to conduct serial dilutions and liquid transfers. Automated liquid handlers included the Multidrop 384 (Labsystems, Helsinki, Finland), Biomek 2000 and Biomek FX (Beckman Coulter, Fullerton Calif.). The wells in columns 2-12 in standard 96-well microdilution plates (Costar 3795) were filled with 150 μl of the correct diluent (50% DMSO for investigational compounds, 100% DMSO for comparator compounds). These would become the mother plates' from which ‘daughter’ or test plates would be prepared. Stocks were diluted to 40× the desired top concentration in the test plates in the indicated solvent, and 300 μL of the 40× stock was dispensed into the appropriate well in Column 1 of the mother plates. The Biomek 2000 was used to make serial serial 2-fold dilutions through Column 11 in the “mother plate”. The wells of Column 12 contained no drug and served as the organism growth control wells.

The daughter plates were loaded with 185 μL per well of RPMI described above using the Multidrop 384. The daughter plates were prepared using the Biomek FX which transferred 5 μL of drug solution from each well of a mother plate to the corresponding well of the daughter plate in a single step.

A standardized inoculum of each organism was prepared. For Candida, colonies were picked from the streak plate and a suspension was prepared in RPMI medium equal to a 0.5 McFarland standard, then diluted 1:100 in RPMI and transferred to compartments of sterile reservoirs divided by length (Beckman Coulter). For the Aspergillus isolates, previously prepared and quantitated suspensions were used to make dilutions in RPMI to reach 20× the final concentration. These dilutions were also transferred to compartments of sterile reservoirs divided by length (Beckman Coulter). The final concentration of the Aspergillus isolates was approximately 0.2-2.5×10⁴ CFU/mL.

The Biomek 2000 was used to inoculate the plates. Daughter plates were placed on the Biomek 2000 work surface reversed so that inoculation took place from low to high drug concentration. The Biomek 2000 delivered 10 μL of standardized inoculum into each well. Thus, the wells of the daughter plates ultimately contained 185 μL of RPMI, 5 μL of drug solution, and 10 μL of inoculum. The final concentration of DMSO in the test well was 2.5% for the evaluated comparators and 1.25% for the investigational agents.

Plates were stacked 3 high, covered with a lid on the top plate, placed into plastic bags, and incubated at 35° C. for approximately 24-48 hr prior to reading. Plates were read when inoculum was confluent in growth wells. Plates were viewed from the bottom using a plate viewer. An un-inoculated solubility control plate was observed for evidence of drug precipitation. MICs were read where visible growth of the organism was inhibited. MECs were read where the growth shifted to a small, rounded, compact hyphal form as compared to the hyphal growth seen in the growth control well.

TABLE 1
Antifungal Activity for Select Compounds

| | MIC (μg/mL) | | | | MEC (μg/mL) | | | | |
|---|---|---|---|---|---|---|---|---|---|
| | C. albicans | C. glabrata | C. krusei | C. parapsilosis | A. fumigatus | A. fumigatus | A. fumigatus | A. fumigatus | A. flavus |
| Compound | ATCC | ATCC | ATCC | ATCC | MYA- | MYA- | ATCC | MX | MX |
| Number | 90028 | 90030 | 14243 | 22019 | 3626 | 4609 | 13073 | 5944 | 5948 |
| 1 | 2 | 4 | 8 | 16 | 0.25-0.5 | 0.12-1 | 0.12-2 | 0.12-0.5 | 0.12-1 |
| 2 | 2 | 4 | 8-16 | 16 | 0.25-1 | 0.12-1 | 0.12-2 | 0.12-1 | 0.06-1 |
| 3 | 2 | 16 | 16 | >32 | 1 | 1 | 1 | 1 | 1 |
| 4 | 2 | 4 | 8 | 32 | 2 | 1 | 1 | 1 | 1 |
| 5 | 2 | 4 | >4 | >4 | 1 | 1 | 1 | 1 | 1 |
| 6 | 2 | 4 | 8 | >16 | 2 | 1 | 1 | 2 | 0.5 |
| 7 | 2 | 8 | 16 | 32 | 0.5 | 0.5 | 1 | 1 | 1 |
| 8 | 2 | 4 | 8 | 32 | 1 | 1 | 1 | 1 | 1 |
| 9 | 2 | 8 | 16->16 | >16-32 | 0.12 | 0.06-0.5 | 0.06-0.12 | 0.12 | 0.06-0.12 |
| 10 | 2 | 8 | 16 | 32 | 0.12 | 0.12 | 0.25 | 0.12 | 0.12 |
| 11 | 2 | 8 | 16 | 32 | 0.06-0.12 | 0.06 | ≦0.06-0.06 | 0.12 | 0.06 |
| 12 | 1-2 | 4-8 | 8-16 | 8-16 | 0.06-0.25 | 0.06-0.12 | ≦0.03-0.12 | 0.12-0.25 | ≦0.03-0.12 |
| 13a | 2 | 8 | 16 | 32 | 0.25 | 0.12 | 0.12 | 0.25 | 0.12 |
| 13b | 2 | 8 | 16 | 32 | 0.25 | 0.12 | 0.25 | 0.25 | 0.12 |
| 14 | 2 | 16 | 16 | 16 | 0.25 | 0.12 | 0.25 | 0.12 | 0.12 |
| 15 | 1 | 2 | 8 | 16 | 0.12 | 0.12 | 0.12 | 0.12 | 0.12 |
| 16 | 2 | 32 | >32 | >32 | 0.25 | 0.25 | 0.25 | 0.25 | 0.25 |
| 25 | 0.12 | 0.5 | 2 | 1 | ≦0.03 | ≦0.03 | ≦0.03 | ≦0.03 | ≦0.03 |
| 26 | 0.25 | 1 | 4 | 4 | ≦0.03 | ≦0.03 | ≦0.03 | ≦0.03 | ≦0.03 |
| 27 | 2 | 2 | 8 | 16 | 0.25 | 0.12 | 8 | 0.12 | 0.06 |
| 28 | 1 | 2 | 8 | 8 | 0.25 | 0.25 | 0.25 | 0.25 | 0.12 |
| 29 | 2 | 8 | 32 | >32 | 0.25 | 0.25 | 0.5 | 0.25 | 0.25 |
| caspofungin | 0.03-0.12 | 0.06-0.25 | 0.5-1 | 0.5-1 | ≦0.03-0.12 | ≦0.03-1 | ≦0.03-0.06 | 0.03-0.12 | ≦0.015-0.06 |
| acetate | | | | | | | | | |
| amphotericin | 0.12-1 | 0.25-1 | 0.25-2 | 0.5-2 | 0.5-2 | 0.5-4 | 0.5-4 | 0.5-2 | 0.5-2 |
| B | | | | | | | | | |
| voriconazole | 0.06-0.5 | 0.12-0.25 | 0.25-1 | 0.03-0.06 | 0.12-0.5 | 0.25-0.5 | 0.012-0.5 | 0.12-0.25 | 0.25-2 |

MIC = minimum inhibitory concentration; MEC = minimum effective concentration.

### Example 32

### Antifungal Activity of Compounds 30 and 31

MIC and MEC assays were run as described in Example 31 with the following exceptions. Starting solutions of all antifungal agents were prepared in 100% DMSO. Stock concentrations were made at 50× the highest final assay concentration and serially diluted 2-fold, 16 times in a 96-well PCR plate (VWR 83007-374). Candida and Aspergillus suspensions from Sabouraud dextrose agar plate cultures were prepared in 0.85% saline at 0.5 McFarland standard (˜0.1 OD₅₃₀ nm). Candida suspensions were diluted 1:500 in RPMI (MP Biomedicals, cat no. 1060124; buffered with MOPS and adjusted with NaOH to pH 7.0) to a concentration of ˜0.5-2.5×10³ CFU/mL and Aspergillus suspensions were diluted 1:50 in RPMI to 0.4-5×10⁴ CFU/mL final concentration. 98 μL of each cell suspension in RPMI were added to test wells in a 96-well assay plate (Costar cat. no. 3370). A Beckman Multimek 96 liquid handling robot was used to dispense 2 μL of each 50× stock compound into the plate containing 98 μL of each strain in RPMI (2% final solvent concentration). Plates were shaken then incubated at 35° C. for 24-48 h prior to reading. MIC values were read visually at 50% growth inhibition for echinocandins (24 h) and at 100% growth inhibition for amphotericin B (48 h). MEC values (24 h) were read with the aid of a microscope at the lowest echinocandin concentration where rounded/compact hyphal morphology was observed.

TABLE 2
Antifungal Activity for Select Compounds

| | | | | | | MEC (μg/mL) | | | | |
|---|---|---|---|---|---|---|---|---|---|---|
| | MIC (μg/mL) | | | | | | | | A. flavus | |
| | C. albicans | C. glabrata | C. tropicalis | C. krusei | C. parapsilosis | A. fumigatus | A. fumigatus | A. fumigatus | ATCC | A. niger |
| Compound | ATCC | ATCC | ATCC | ATCC | ATCC | MYA- | MYA- | ATCC | MYA- | ATCC |
| Number | 90028 | 90030 | 750 | 14243 | 22019 | 3626 | 4609 | 13073 | 3631 | 16404 |
| 30 | 2 | 8 | 2 | 32 | 32 | 1 | 0.5 | 0.25 | 0.5 | 0.5 |
| 31 | 0.5 | 2 | 0.25 | 0.5 | 0.5 | 0.06 | 0.03 | 0.015 | 0.06 | 0.25 |
| caspofungin | 0.125 | 0.25 | 0.125 | 0.25 | 0.5 | 0.06 | 0.06 | 0.03 | 0.06 | 0.06 |
| acetate | | | | | | | | | | |
| amphotericin | 0.5 | 1 | 1 | 1 | 1 | 0.25 | 1 | 0.25 | 1 | 0.5 |
| B | | | | | | | | | | |

### Example 33

### Chemotactic Activity of Compounds

### Human Neutrophil Transwell Migration Assay

Neutrophils were purified from human peripheral blood utilizing PolymorphPrep. Red blood cells were lysed with a lysis buffer and the cells were washed, counted and resuspended in assay buffer. Neutrophils (approximately 50,000 to 100,000 in number) were placed over 5-micon pore sized transwell plates in 24-well format with test compounds in the bottom chamber of each well. A negative control of media alone in the bottom chamber was set up in triplicate. A positive control where the neutrophils were added directly to the bottom well was set up in triplicate.

The plates were incubated at 37° C. for 45 minutes to allow for migration. After this time, migration was confirmed visually and then an aliquot (⅙^{th} of total cells) from each well was taken for ATP detection via luminescence with ATPlite (PerkinElmer) to quantify the number of migrated cells. The averaged reading for random migration detected in the negative control was subtracted from all readings to obtain the corrected migration number. These numbers were then divided by the averaged reading from the positive control to get the % Neutrophil Migration of the maximum migration possible.

TABLE 3
Chemotactic Activity for Select Compounds

| | % Neutrophil Migration @ conc in μg/mL | | | | |
|---|---|---|---|---|---|
| Compound | 10 | 1 | 0.1 | 0.01 | 0.001 |
| 1 | −11.2 | −10.6 | 70.7 | 67.9 | −4.7 |
| 2 | −11.0 | 30.6 | 97.5 | 28.5 | −5.3 |
| 3 | −11.0 | −9.7 | 66.2 | 109.3 | 1.5 |
| 4 | −11.2 | −9.8 | 15.1 | 88.0 | 3.8 |
| 11 | −10.9 | −8.0 | 28.3 | 80.3 | −4.7 |
| 13a | −7.6 | −7.3 | 8.5 | 37.1 | 9.1 |
| 13b | −7.6 | −8.5 | 11.5 | 26.1 | 6.2 |
| 14 | −9.0 | 21.4 | 14.8 | 1.9 | 0.9 |
| 15 | −7.0 | −6.3 | 13.0 | 47.3 | 9.6 |
| 25 | −6.8 | −6.4 | −4.4 | −3.8 | −0.9 |
| 26 | 15.7 | −4.1 | −5.1 | −3.4 | −3.4 |
| 27 | 37.0 | −1.6 | −4.2 | 3.4 | −1.6 |
| 28 | −7.4 | −0.2 | 43.4 | 2.2 | −0.4 |
| 29 | 8.4 | 11.0 | 2.2 | 0.1 | 1.4 |
| 30 | 19.7 | 48.2 | 234.3 | 133.6 | 105.2 |
| 31 | 64.0 | 134.3 | 146.0 | 115.7 | 126.4 |

### Example 34

### Migration and Killing of Aspergillus fumigatus by Human Neutrophils in the Presence or Absence of Compound or Control

### Methods

### Microfluidic Device Fabrication

Microfluidic devices used to measure leukocyte migration in response to Aspergillus fumigatus with or without drug (Compound 1 or Compound 11), antifungal control (caspofungin acetate) and/or chemoattractant (fMLP) were manufactured using known microfabrication techniques. Two layers of photoresist (SU8; Microchem), the first one 10 μm thin (corresponding to the migration channels) and the second one 70 μm thick, corresponding to the focal chemotactic chambers (FCCs) were patterned on one silicon wafer sequentially using two photolithographic masks and processing cycles according to the instructions from the manufacturer. The wafer with patterned photoresist was used as a mold to produce polydimethylsiloxane (PDMS) (Fisher Scientific) devices, which were then bonded to the base of glass-bottom 12- or 24-well plates, using an oxygen plasma machine (Nordson-March).

Aspergillus fumigatus Culture Preparation

Aspergillus fumigatus strain AF293 expressing either cytosolic red fluorescent protein (RFP) or green fluorescent protein (GFP) was grown on Sabouraud dextrose agar plates supplemented with 100 μg/mL ampicillin at 30° C. for 3-4 days. Conidia were harvested by gentle scraping followed by washing in ice-cold phosphate buffered saline (PBS) three times. Conidia were immediately used or stored at 4° C. for use within one week.

### Primary Human Neutrophil and White Blood Cell Isolation

De-identified, fresh human blood samples from healthy volunteers, aged 18 years and older, who were not receiving immunosuppressants, were purchased from Research Blood Components, LLC. Peripheral blood was drawn in tubes containing a final concentration of 5 mM EDTA (Vacutainer; Becton Dickinson) and processed within 2 h of blood collection. Using a sterile technique, neutrophils were isolated from whole blood using HetaSep followed by the EasySep Human Neutrophil Enrichment Kit (STEMCELL Technologies) following the manufacturer's protocol. The purity of neutrophils was assessed to be >98% using the Sysmex KX-21N Hematology Analyzer (Sysmex America, Inc.). White blood cells were isolated using Hetasep followed by a 5 min spin down and wash with 1×PBS. White blood cells were stained with Hoechst (32.4 μM) (Sigma) following the manufacture's protocols. The final aliquots of white blood cells were resuspended in RPMI plus 10% fetal bovine serum (FBS−stock 50 mL FBS/450 mL RPMI) (Sigma-Aldrich) at a concentration of 20,000 cells/2 μL and kept at 37° C. Cells were then immediately introduced into the microfluidic device for the chemotaxis and Aspergillus fumigatus killing assay. All experiments were repeated at least three times with neutrophils or white blood cells from three different healthy donors.

Microfluidic Neutrophil Chemotaxis and Aspergillus fumigatus Killing Assay Preparation

Immediately after bonding to the well plate, donut-shaped devices were filled with Aspergillus fumigatus conidia at a concentration of 10⁶ cells/mL with or without either Compound 1 or Compound 11 [10 nM], caspofungin acetate [10 nM] and/or a solution of the chemoattractant N-formyl-methionyl-leucyl-phenylalanine (fMLP) [100 nM](Sigma-Aldrich, St. Louis, Mo.). The device was then placed in a vacuum for 15 min. The solution filled all of the FCCs as the air was displaced. The devices were then vigorously washed five times with 1×PBS to remove any residual Aspergillus fumigatus conidia, drug or chemoattractant that was outside of the FCCs. The device was then submerged in 0.5 mL of cell media. Neutrophils or white blood cells (20,000 cells/2 μL) were then pipetted into the cell loading chamber (CLC) using a gel-loading pipette tip. Neutrophil migration into the migration channel toward the FCC started immediately and was recorded using time-lapse imaging on a fully automated Nikon TiE microscope (10× magnification) with biochamber heated to 37° C. with 5% carbon dioxide gas. Image analysis of cell migration counts and fungi growth was analyzed by hand using Image J software.

Neutrophil Chemotaxis and Aspergillus fumigatus Killing Analysis

Image analysis of cell migration counts and fungi growth was analyzed by hand using Image J software. Neutrophil counts per chamber were counted every 15 min for the first 2 hours of the experiment and then every hour for the remaining 16 hours. Percentage of conidia that converted to hyphal growth was measured by counting conidia loaded per chamber before neutrophils or WBCs were loaded into the chamber and counting numbers of these conidia that grew hyphae by 18 hours. Fungal growth velocity was calculated using Image J. The 16 chambers in each device (n=3) were analyzed for at least three different healthy donors.

### Results—Summary Data

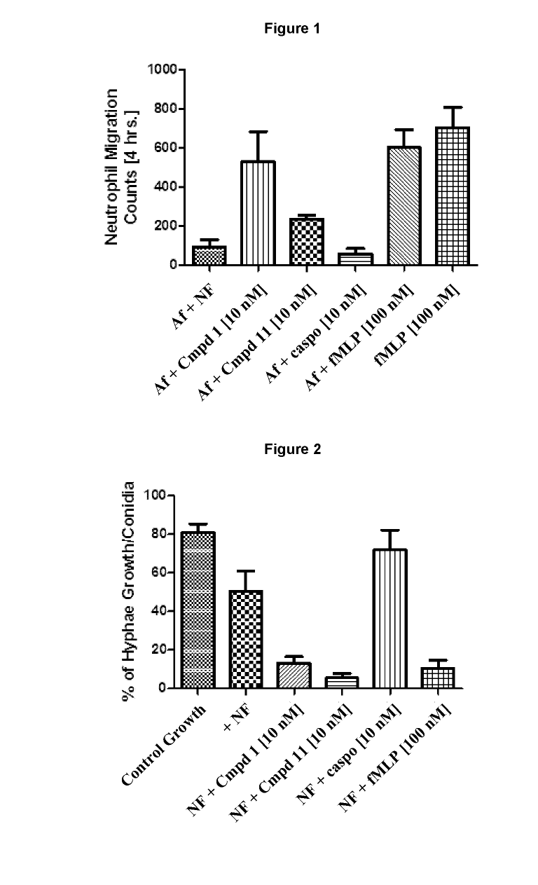

FIG. 1 shows data generated from movies of neutrophil (NF) migration counts at the 4 hour timepoint after addition of NFs to the cell loading chamber (CLC). Results demonstrate increased NF migration in the presence of Aspergillus fumigatus plus Compound 1, A. fumigatus plus Compound 11, A. fumigatus plus fMLP, and fMLP alone relative to A. fumigatus alone. A. fumigatus plus caspofungin did not give increased NF migration.

FIG. 2 shows the percent hyphal growth compared to remaining conidia. Results demonstrate inhibition of Aspergillus fumigatus hyphal growth from starting conidia for neutrophils (NF) alone, NFs plus Compound 1, NFs plus Compound 11, and NFs plus fMLP, but not for NFs plus caspofungin.

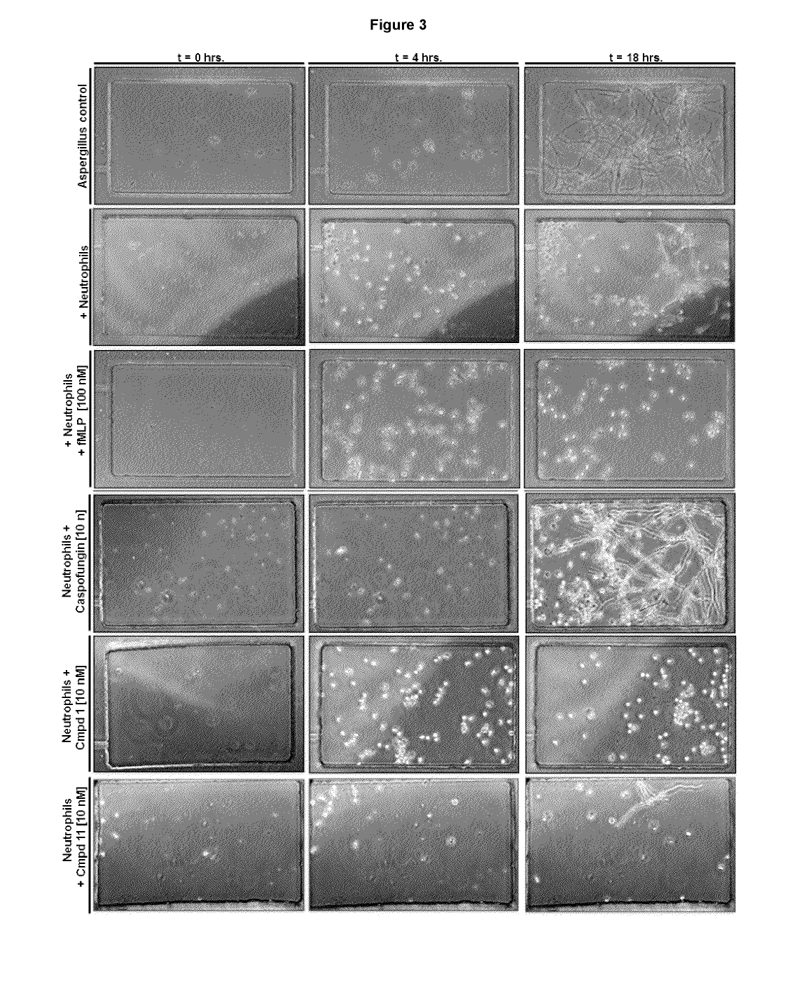

FIG. 3 shows representative single frames captured from movies used to generate data in FIGS. 1 and 2. Beginning of experiment (t=0 hrs.) with only Aspergillus fumigatus conidia in the well along with drug where indicated. Neutrophils (NFs) have been loaded to the CLC at this point. The central panels show wells at 4 hour timepoint after NFs have had the opportunity to migrate into the chamber. The far right panel shows the wells at the end of the experiment (18 hours from t=0 hrs). Red stained cells are A. fumigatus conidia and hyphae and indicate living cells. Blue stained cells are NFs.

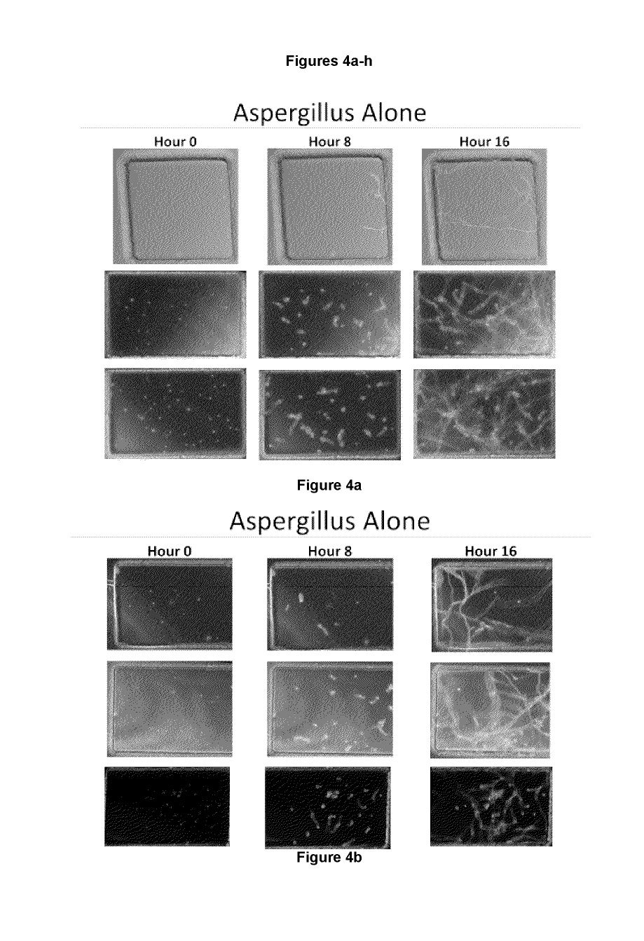

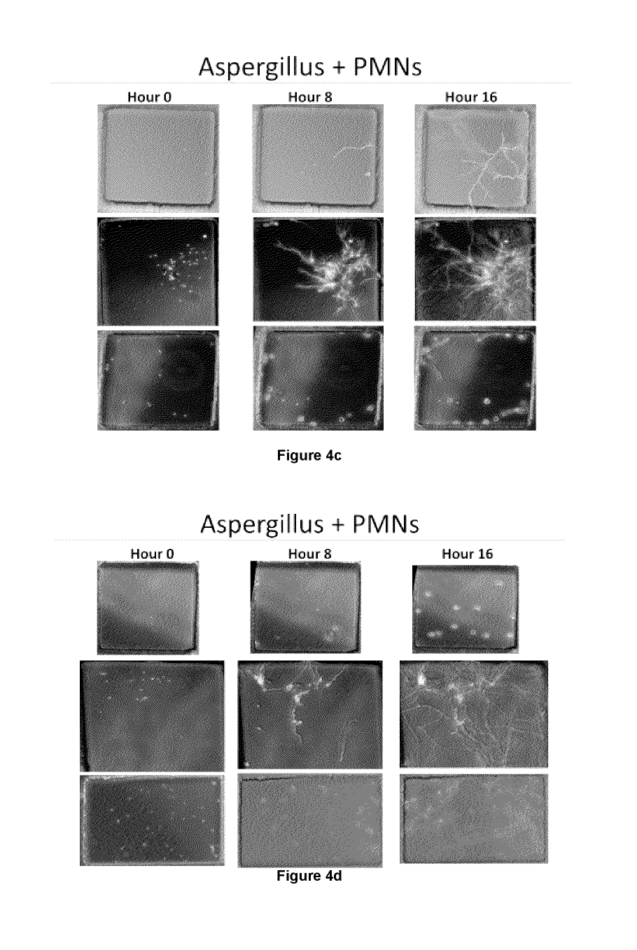

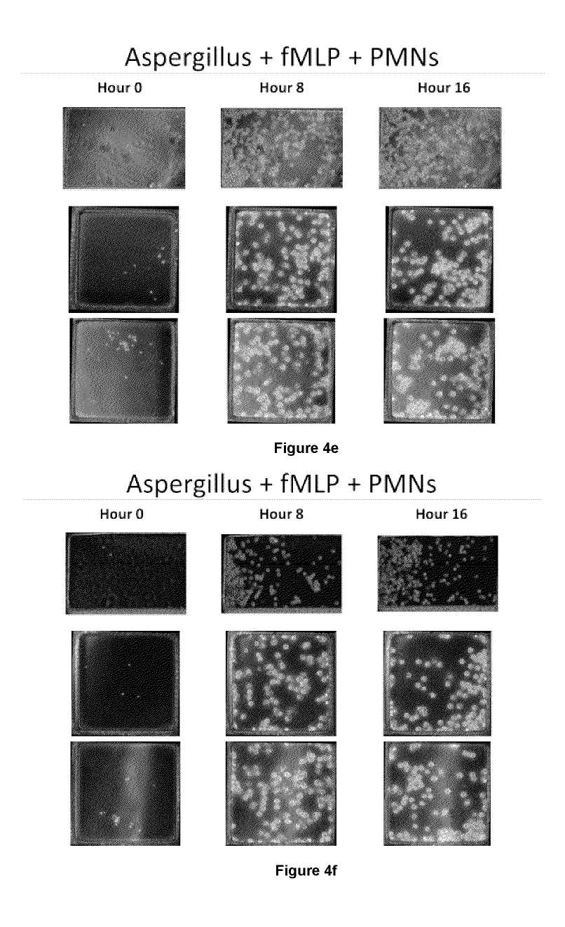

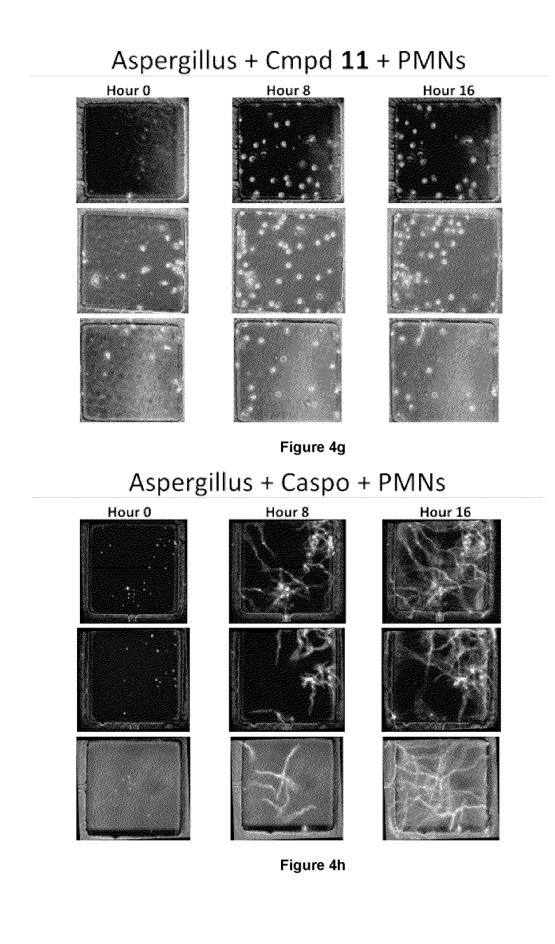

FIGS. 4a-4h show the timecourse of A. fumigatus (Af) growth and neutrophil (PMNs) migration in FCCs using three individual healthy blood donors at various timepoints (t=0, 8, and 16 hours) for the experiment indicated above. Rows show the timecourse of Af growth and PMNs migration and columns indicate three individual healthy blood donors. Elongated cells (i.e., filament-like) are Aspergillus fumigatus while circular cells are PMNs. FIGS. 4a and 4b show Af growth in the absence of PMNs or drug. FIGS. 4c and 4d show Af growth in the presence of PMNs alone. FIGS. 4e and 4f show inhibition of growth of Af and migration of PMNs in the presence of fMLP and PMNs. FIG. 4g shows inhibition of growth of Af and migration of PMNs in presence of Compound 11 and PMNs. FIG. 4h shows growth of Af in presence of caspofungin acetate (caspo) and PMNs.

Taken together, the data demonstrate that Compound 1, Compound 11, and fMLP, but not caspofungin are able to suppress hyphal growth of A. fumigatis in the presence of human neutrophils. Suppression of hyphal growth in the presence of neutrophils is greater for Compound 1, Compound 11, and fMLP than it is for neutrophils alone or neutrophils plus caspofungin.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

1. A compound or a pharmaceutically acceptable salt thereof comprising a pathogen pattern recognition receptor ligand conjugated to an inhibitor of β-1,3-glucan synthase.

2. The compound or a pharmaceutically acceptable salt thereof of claim 1, wherein said inhibitor of β-1,3-glucan synthase is cyclic lipopeptide.

3. The compound or a pharmaceutically acceptable salt thereof of claim 1 or 2, wherein said inhibitor of β-1,3-glucan synthase is a member of the aculeacin, echinocandin, pneumocandin, cyclopeptamine, mulundocandin, sporiofungin or WF11899A class of antifungal drugs.

4. The compound or a pharmaceutically acceptable salt thereof of any one of claims 1-3, wherein said inhibitor of β-1,3-glucan synthase is selected from the group consisting of caspofungin, echinocandin B, cilofungin, pneumocandin A₀, pneumocandin B₀, L-705589, L-731373, L-733560, A-174591, A-172013, A-175800, micafungin, anidulafungin, biafungin, AF-053, AF-033, amino-biafungin, amino-AF-053, and amino-AF-033.

5. The compound or a pharmaceutically acceptable salt thereof of any one of claims 1-4, wherein said inhibitor of β-1,3-glucan synthase is selected from the group consisting of caspofungin, echinocandin B, cilofungin, pneumocandin A₀, pneumocandin B₀, L-705589, L-731373, L-733560, A-174591, A-172013, A-175800, micafungin, and anidulafungin.

6. The compound or a pharmaceutically acceptable salt thereof of claim 4, wherein said inhibitor of β-1,3-glucan synthase is caspofungin.

7. The compound or pharmaceutically acceptable salt thereof of claim 4, wherein said inhibitor of β-1,3-glucan synthase is micafungin.

8. The compound or pharmaceutically acceptable salt thereof of claim 4, wherein said inhibitor of β-1,3-glucan synthase is anidulafungin.

9. The compound or a pharmaceutically acceptable salt thereof of claim 4, wherein said inhibitor of β-1,3-glucan synthase is L-733560.

10. The compound or a pharmaceutically acceptable salt thereof of claim 4, wherein said inhibitor of β-1,3-glucan synthase is amino-biafungin, amino-AF-053, or amino-AF-033.

11. The compound or a pharmaceutically acceptable salt thereof of claim 1, wherein said inhibitor of β-1,3-glucan synthase has the structure: 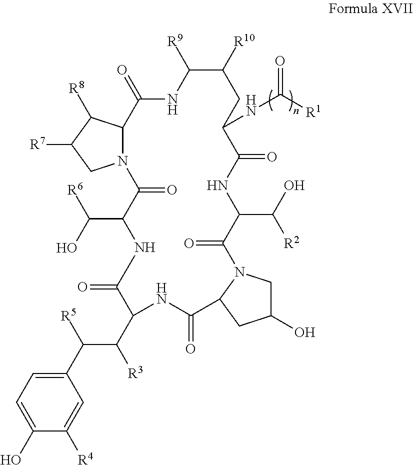
wherein R¹ is a C₁₀-C₃₆ moiety containing 0-8 heteroatoms selected from O, S, and N, optionally substituted with halo;
R² is hydrogen or methyl;
R³ and R⁵ are independently hydrogen or hydroxyl;
R⁴ is hydrogen, OR²⁰, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, hydroxyl, —NR¹¹R¹², or —NH(C═NH)NR¹¹R¹²;
R⁸ is hydrogen or hydroxyl;
R⁹ is -(Q₁(CH₂)_{b})_{c}Q₂, —NR¹¹R¹², —CH₂NR¹¹R¹², —CH₂NR¹¹R¹²R¹³, hydrogen, or hydroxyl;
R¹⁰ is hydrogen, hydroxyl, oxo, —NR¹¹R¹², NH(CH₂)ₐN¹¹R¹¹R¹², or —NH(CH₂)ₐN⁺R¹¹R¹¹R¹³;
R¹¹, R¹², and R¹³ are independently hydrogen or C₁-C₃ alkyl;
n is 0 or 1;
a is 2 to 4;
Q₁ is S, O, or NH;
b is 2-6;
c is 1-8;
Q₂ is NR¹¹R¹², N⁺R¹¹R¹²R¹³, or OR²⁰; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹².

12. The compound or a pharmaceutically acceptable salt thereof of claim 11, wherein said inhibitor of β-1,3-glucan synthase has the structure: 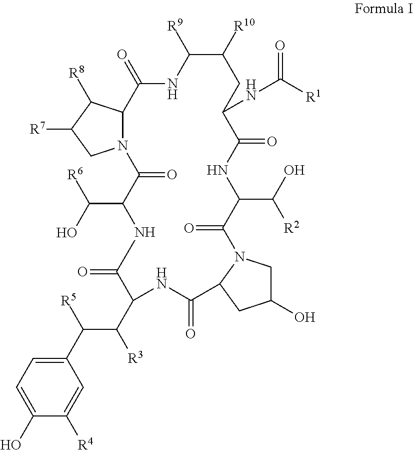
wherein R¹ is optionally substituted C₁₃-C₁₇ alkyl, optionally substituted C₁₃-C₁₇ alkenyl, optionally substituted aryl, or optionally substituted heteroaryl;
R² is hydrogen or methyl;
R³, R⁵, and R¹⁰ are independently hydrogen or hydroxyl;
R⁴ is hydrogen, hydroxyl, —OMe, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, or hydroxyl;
R⁸ is hydrogen or hydroxyl;
R⁹ is hydrogen, hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or C₁-C₃ alkyl; and
a is 2 to 4;
wherein at least one of R⁴, R⁶, or R⁹ contains —NR¹¹R¹².

13. The compound or a pharmaceutically acceptable salt thereof of claim 11, wherein said inhibitor of β-1,3-glucan synthase has the structure: 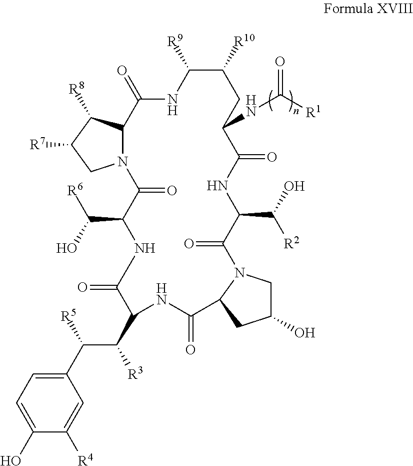
wherein R¹ is a C₁₀-C₃₆ moiety containing 0-8 heteroatoms selected from O, S, and N, optionally substituted with halo;
R² is hydrogen or methyl;
R³ and R⁵ are independently hydrogen or hydroxyl;
R⁴ is hydrogen, OR²⁰, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, hydroxyl, —NR¹¹R¹², or —NH(C═NH)NR¹¹R¹²;
R⁸ is hydrogen or hydroxyl;
R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl;
R¹⁰ is hydrogen, hydroxyl, oxo, —NR¹¹R¹², NH(CH₂)ₐNR¹¹R¹², or —NH(CH₂)ₐN⁺R¹¹R¹²R¹³;
R¹¹, R¹², and R¹³ are independently hydrogen or C₁-C₃ alkyl;
n is 0 or 1;
a is 2 to 4;
Q₁ is S, O or NH;
b is 2-6;
c is 1-8;
Q₂ is NR¹¹R¹², N⁺R¹¹R¹²R¹³, or OR²⁰; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹².

14. The compound or a pharmaceutically acceptable salt thereof of claim 13, wherein said inhibitor of β-1,3-glucan synthase has the structure: 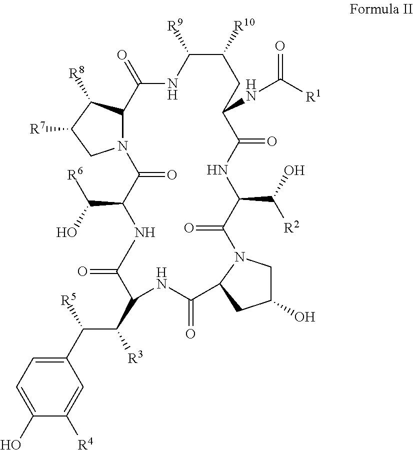
wherein R¹ is optionally substituted C₁₃-C₁₇ alkyl, optionally substituted C₁₃-C₁₇ alkenyl, optionally substituted aryl, or optionally substituted heteroaryl;
R² is hydrogen or methyl;
R³, R⁵, and R¹⁰ are independently hydrogen or hydroxyl;
R⁴ is hydrogen, hydroxyl, —OMe, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, or hydroxyl;
R⁸ is hydrogen or hydroxyl;
R⁹ is hydrogen, hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or C₁-C₃ alkyl; and
a is 2 to 4;
wherein at least one of R⁴, R⁶, or, R⁹ contains —NR¹¹R¹².

15. The compound or a pharmaceutically acceptable salt thereof of claim 1, wherein said inhibitor of β-1,3-glucan synthase has the structure: 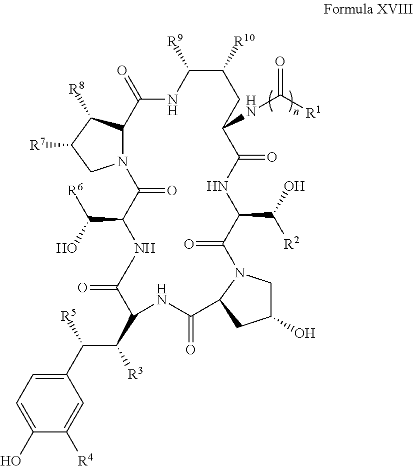
wherein R¹ is a moiety containing a C₁-C₁₇ alkyl or heteroalkyl, C₂-C₁₇ alkenyl or heteroalkenyl, aryl or heteroaryl, cyclic, polycyclic, heterocyclic or heteropolycyclic moiety, or a combination thereof to form a C₁₀-C₃₆ moiety;
R² is methyl;
R³ and R⁵ are hydroxyl;
R⁴ is hydrogen, hydroxyl, —OMe, —OSO₃H, or —NR¹¹R¹²;
R⁶ is methyl, —CH₂(C═O)NR¹¹R¹², —CH₂CH₂NR¹¹R¹², or —CH₂CH₂N⁺R¹¹R¹²R¹³;
R⁷ is hydrogen, methyl, —NR¹¹R¹², or —NH(C═NH)NR¹¹R¹²;
R⁸ is hydroxyl;
R⁹ is -(Q₁(CH₂)_{b})_{c}Q₂, hydrogen, or hydroxyl;
R¹⁰ is hydrogen, hydroxyl, oxo, —NR¹¹R¹², NH(CH₂)ₐNR¹¹R¹², or —NH(CH₂)ₐN⁺R¹¹R¹²R¹³;
R¹¹, R¹², and R¹³ are independently hydrogen or methyl;
n is 0 or 1; and
a is 2 to 4;
Q₁ is S, O or NH;
b is 2-6;
c is 1-8;
Q₂ is NR¹¹R¹², N⁺R¹¹R¹²R¹³, OR²⁰; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹².

16. The compound or a pharmaceutically acceptable salt thereof of claim 1, wherein said inhibitor of β-1,3-glucan synthase has the structure: 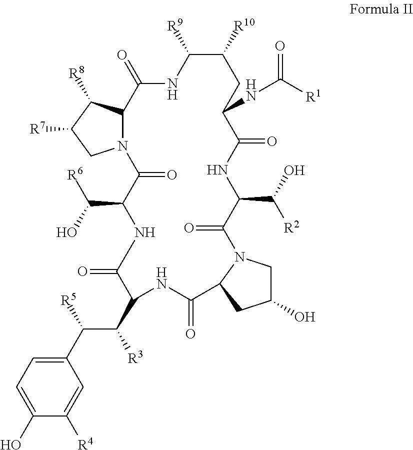
wherein R¹ is optionally substituted C₁₃-C₁₇ alkyl, optionally substituted C₁₃-C₁₇ alkenyl, optionally substituted aryl, or optionally substituted heteroaryl;
R² is methyl;
R³, R⁵, and R¹⁰ are hydroxyl;
R⁴ is hydrogen, hydroxyl, —OMe, —OSO₃H, or —NR¹¹R¹²;
R⁶ is methyl, —CH₂(C═O)NR¹¹R¹², —CH₂CH₂NR¹¹R¹², or —CH₂CH₂N⁺R¹¹R¹²R¹³;
R⁷ is hydrogen, or methyl;
R⁸ is hydroxyl;
R⁹ is hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or methyl; and
a is 2 to 4;
wherein at least one of R⁴, R⁶, or, R⁹ contains —NR¹¹R¹².

17. The compound of any one of claims 11-16, wherein R¹ is

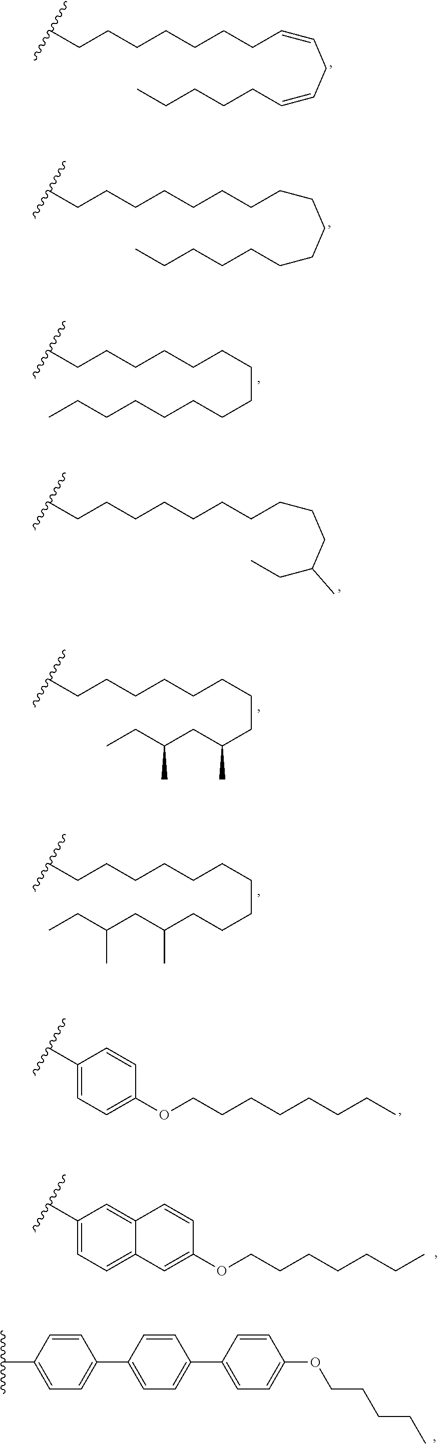

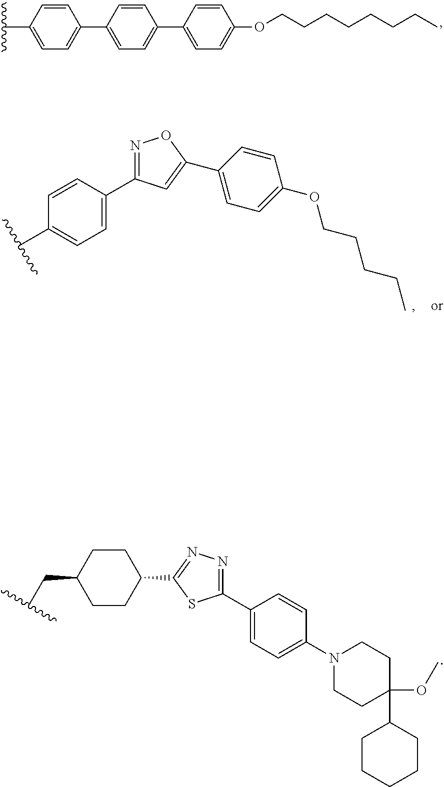 [SMILES: *c1ccc(-c2ccc(-c3ccc(OCCCCCCCC)cc3)cc2)cc1 ; *c1ccc(-c2cc(-c3ccc(OCCCCC)cc3)on2)cc1 ; *C[C@H]1CC[C@H](c2nnc(-c3ccc(N4CCC(OC)(C5CCCCC5)CC4)cc3)s2)CC1]

18. The compound of any one of claims 11-16, wherein R⁹ is hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³.

19. The compound of claim 18, wherein the R⁹ is hydrogen, hydroxyl, —NH₂, —O(CH₂)₂NH₂, —O(CH₂)₂N⁺(CH₃)₃, —O(CH₂)₂O(CH₂)₂NH₂, —NH(CH₂)₂NH₂, —NH(CH₂)₂N⁺(CH₃)₃, —S(CH₂)₂NH₂, or —CH₂NH₂.

20. The compound or a pharmaceutically acceptable salt thereof of any one of claims 1-19, wherein said pathogen pattern recognition receptor ligand is a ligand to a chemotaxis receptor.

21. The compound or a pharmaceutically acceptable salt thereof of claim 20, wherein said pathogen pattern recognition receptor ligand is a ligand to a formyl peptide receptor or to a member of the formyl peptide receptor family.

22. The compound or a pharmaceutically acceptable salt thereof of claim 21, wherein said pathogen pattern recognition receptor ligand is a ligand to FPR1, FPR2, FPR3, FPRL1, or FPRL2.

23. The compound or a pharmaceutically acceptable salt thereof of any one of claims 1-19, wherein said pathogen pattern recognition receptor ligand is a chemotactic peptide.

24. The compound or a pharmaceutically acceptable salt thereof of claim 23, wherein said chemotactic peptide comprises an amino acid residue having the formula:
R¹⁴—X1-X2-X3-X4-X5-X6-X7-X8-X9 Formula III
wherein X9 is any amino acid;
X1-X8 are any amino acid or absent;
R¹⁴ is hydrogen or 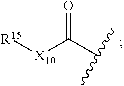
wherein X₁₀ is a bond, NH, or O; and
R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic;
wherein if R¹⁴ is hydrogen, X1 is not absent.

25. The compound or a pharmaceutically acceptable salt thereof of claim 24, wherein said chemotactic peptide comprises an amino acid residue having the formula:
R¹⁴—X1-X2-X9 Formula IV
wherein X1 is any amino acid;
X2 is leucine, isoleucine, or absent; and
X9 is any amino acid.

26. The compound or a pharmaceutically acceptable salt thereof of claim 25, wherein X1 is methionine, oxymethionine, or norleucine.

27. The compound or pharmaceutically acceptable salt of thereof of claim 25 or 26, wherein X2 is leucine, isoleucine, or absent.

28. The compound or pharmaceutically acceptable salt of any one of claims 25-27, wherein X9 is phenylalanine, 1-amino-2-phenylcyclopropane-1-carboxylic acid, methionine, serine, or absent.

29. The compound or pharmaceutically acceptable salt of any one of claims 25-28, wherein R¹⁴ is —C(O)H.

30. The compound or pharmaceutically acceptable salt of claim 29, wherein said pathogen pattern recognition receptor ligand has the structure:

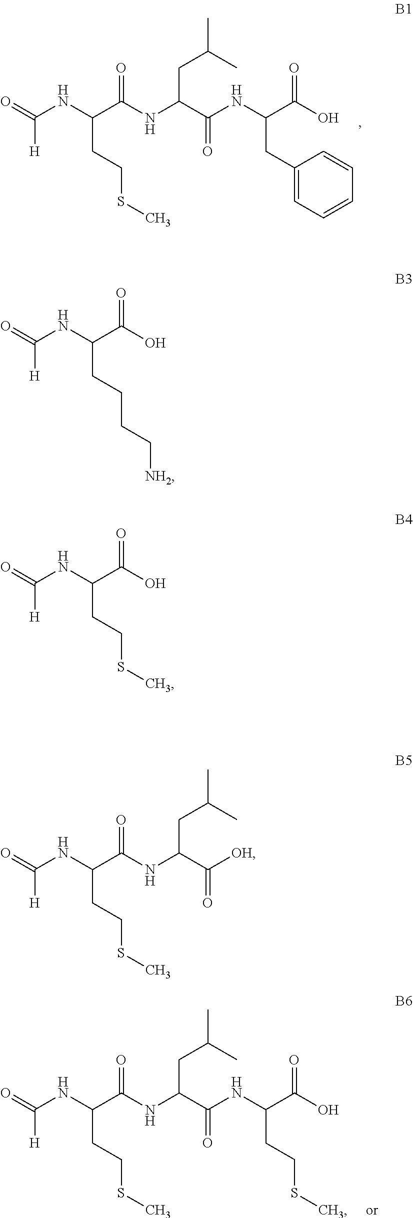 [SMILES: CSCCC(NC=O)C(=O)NC(CC(C)C)C(=O)NC(Cc1ccccc1)C(=O)O ; NCCCCC(NC=O)C(=O)O ; CSCCC(NC=O)C(=O)O ; CSCCC(NC=O)C(=O)NC(CC(C)C)C(=O)O ; CSCCC(NC(=O)C(CC(C)C)NC(=O)C(CCSC)NC=O)C(=O)O ; "or"]

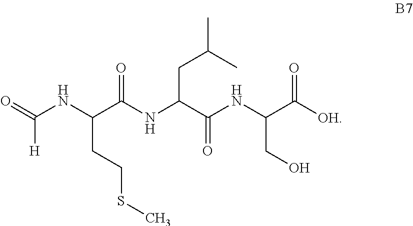 [SMILES: CSCCC(NC=O)C(=O)NC(CC(C)C)C(=O)NC(CO)C(=O)O]

31. The compound or pharmaceutically acceptable salt of any one of claims 25-28, wherein R¹⁴ is —C(O)CH₃.

32. The compound or pharmaceutically acceptable salt of claim 31, wherein said pathogen pattern recognition receptor ligand has the structure:

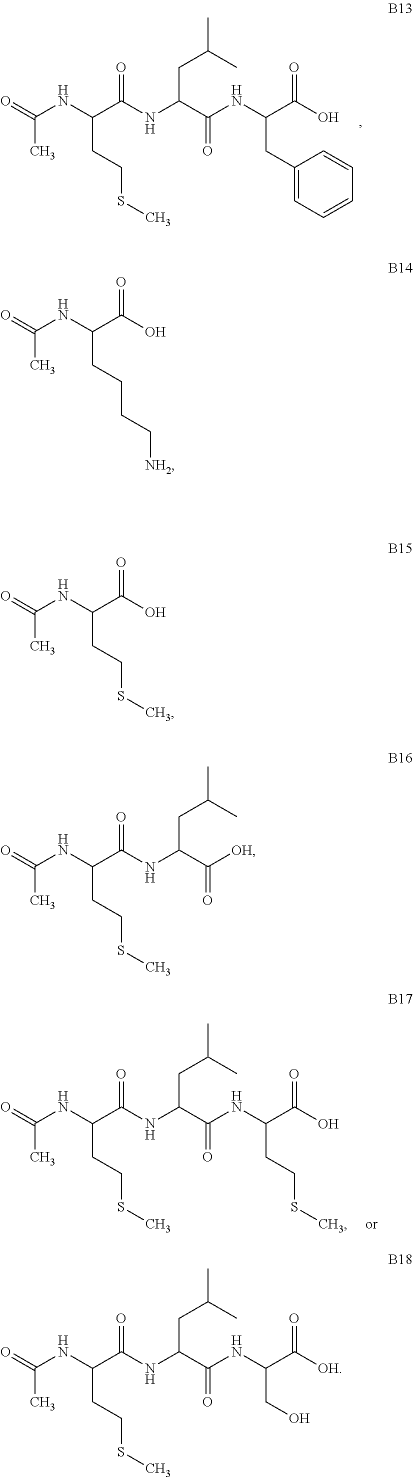

33. The compound or pharmaceutically acceptable salt of any one of claims 25-28, wherein R¹⁴ is —C(O)OCH₂CH(CH₃)₂.

34. The compound or pharmaceutically acceptable salt of claim 33, wherein said pathogen pattern recognition receptor ligand has the structure:

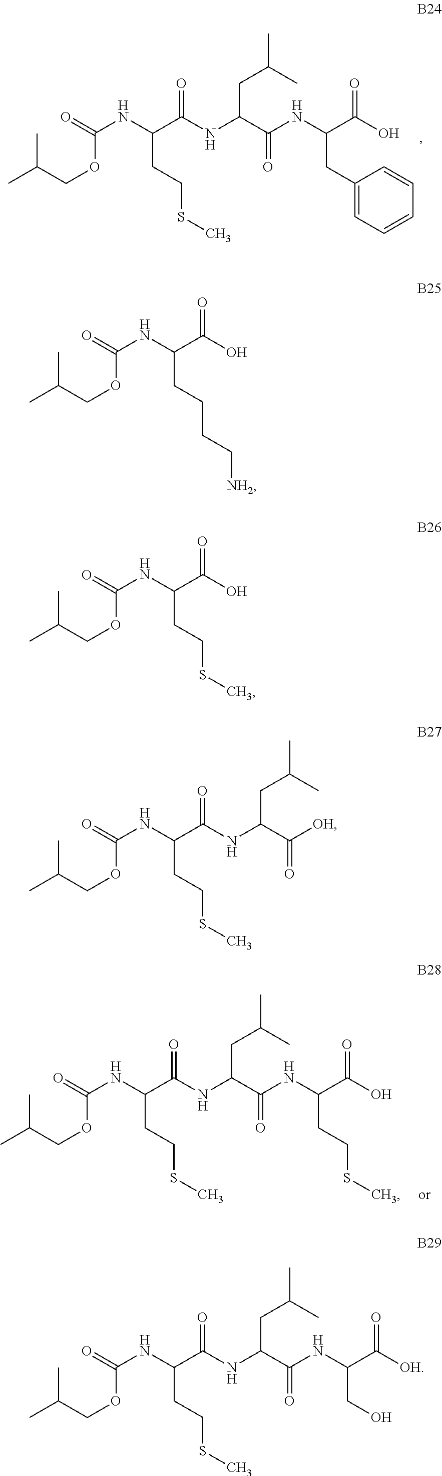

35. The compound or pharmaceutically acceptable salt of any one of claims 1-34, wherein said pathogen pattern recognition receptor ligand and said inhibitor of β-1,3-glucan synthase are conjugated by an amide bond.

36. The compound or pharmaceutically acceptable salt of any one of claims 1-34, wherein said pathogen pattern recognition receptor ligand and said inhibitor of β-1,3-glucan synthase are conjugated by a linker.

37. The compound or pharmaceutically acceptable salt of claim 36, wherein said linker comprises a non-reactive linking moiety of 1-100 atoms in length.

38. The compound or pharmaceutically acceptable salt of claim 37, wherein said linker has the structure:
G¹-(Z¹)_{b}(Y¹)_{c}—(Z²)_{d}—(R¹⁶)—(Z³)ₑ—(Y²)_{f}—(Z⁴)_{g}-G² Formula V
wherein G¹ is a bond between said linker and said inhibitor of β-1,3-glucan synthase;
G² is a bond between said pathogen pattern recognition receptor ligand and said linker;
Z¹, Z², Z³, and Z⁴ each, is independently, optionally substituted C₁-C₂ alkylene, optionally substituted C₁-C₃ heteroalkylene, O, S, or NR¹⁷;
R¹⁷ is hydrogen, optionally substituted C₁₋₄ alkyl, optionally substituted C₃₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
Y¹ and Y² each, is independently, carbonyl, thiocarbonyl, sulphonyl, or phosphoryl;
b, c, d, e, f, and g are, independently, 0 or 1; and
R¹⁶ is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₆₋₁₂ arylene, optionally substituted C₂-C₁₀₀ polyethylene glycolene, or optionally substituted C₁₋₁₀ heteroalkylene, or a chemical bond linking G¹-(Z¹)_{b}—(Y¹)_{c}—(Z²)_{d}— to —(Z³)ₑ—(Y²)_{f}—(Z⁴)_{g}-G².

39. The compound of claim 38, wherein Z⁴ is NH, g is 1, Y¹ is carbonyl, c is 1, and b is 0.

40. The compound or pharmaceutically acceptable salt of any one of claims 36-39, wherein said linker is: 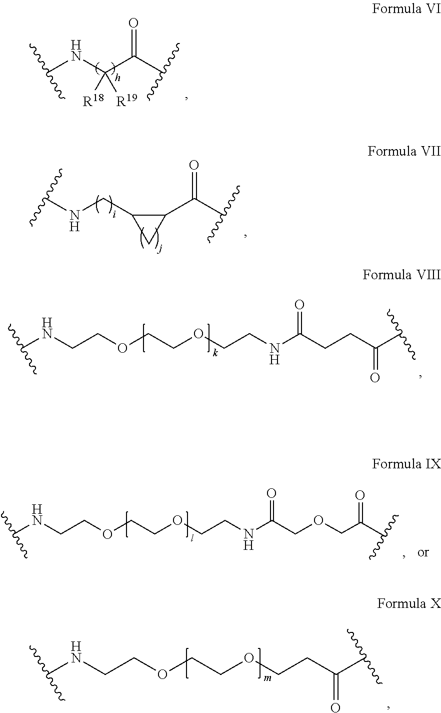
wherein h, i, j, k, l, and m are independently 1 to 12;
R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocycyl.

41. The compound or pharmaceutically acceptable salt of claim 40, wherein said linker is: 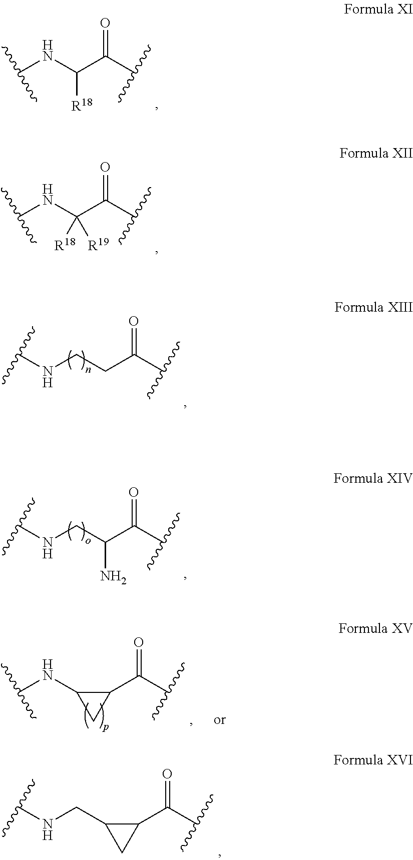
wherein n, o, and p are 1 to 4; and
R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocyclic.

42. The compound or a pharmaceutically acceptable salt thereof of any one of claims 36-41, wherein said linker is a polypeptide.

43. The compound or a pharmaceutically acceptable salt thereof of any one of claims 36-42, wherein said pathogen pattern recognition receptor ligand is attached to said linker via an amide bond and said inhibitor of β-1,3-glucan synthase is attached to said linker via an amide bond.

44. A compound is: 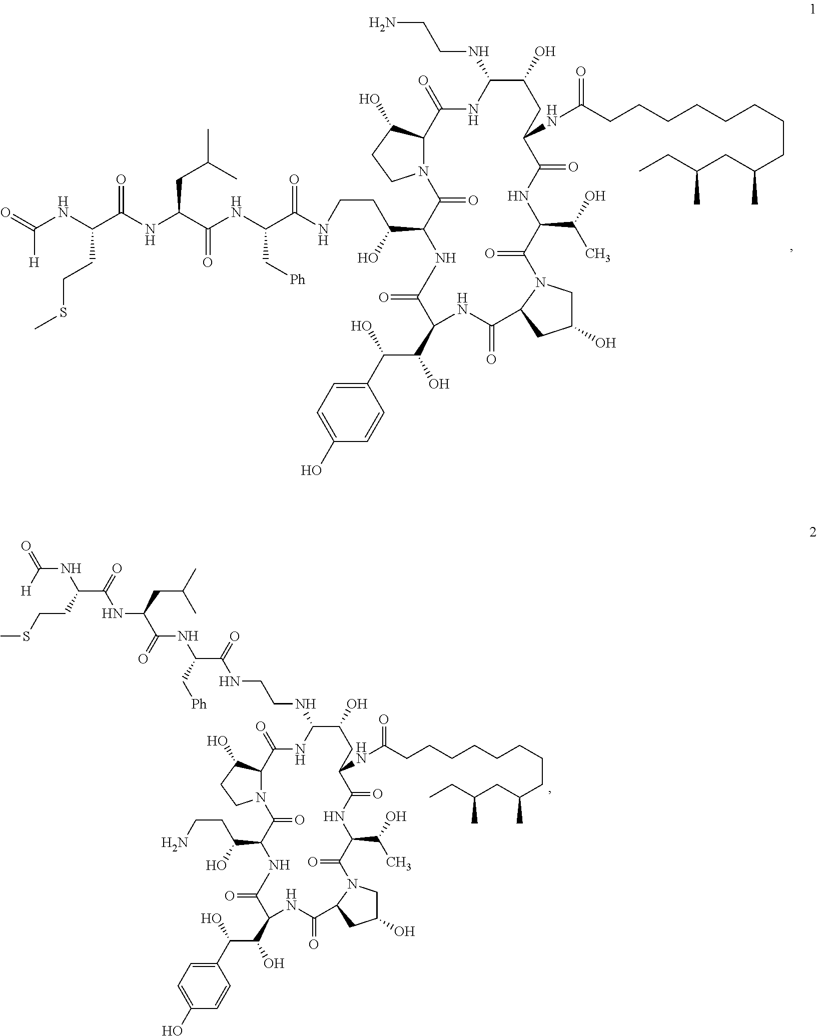 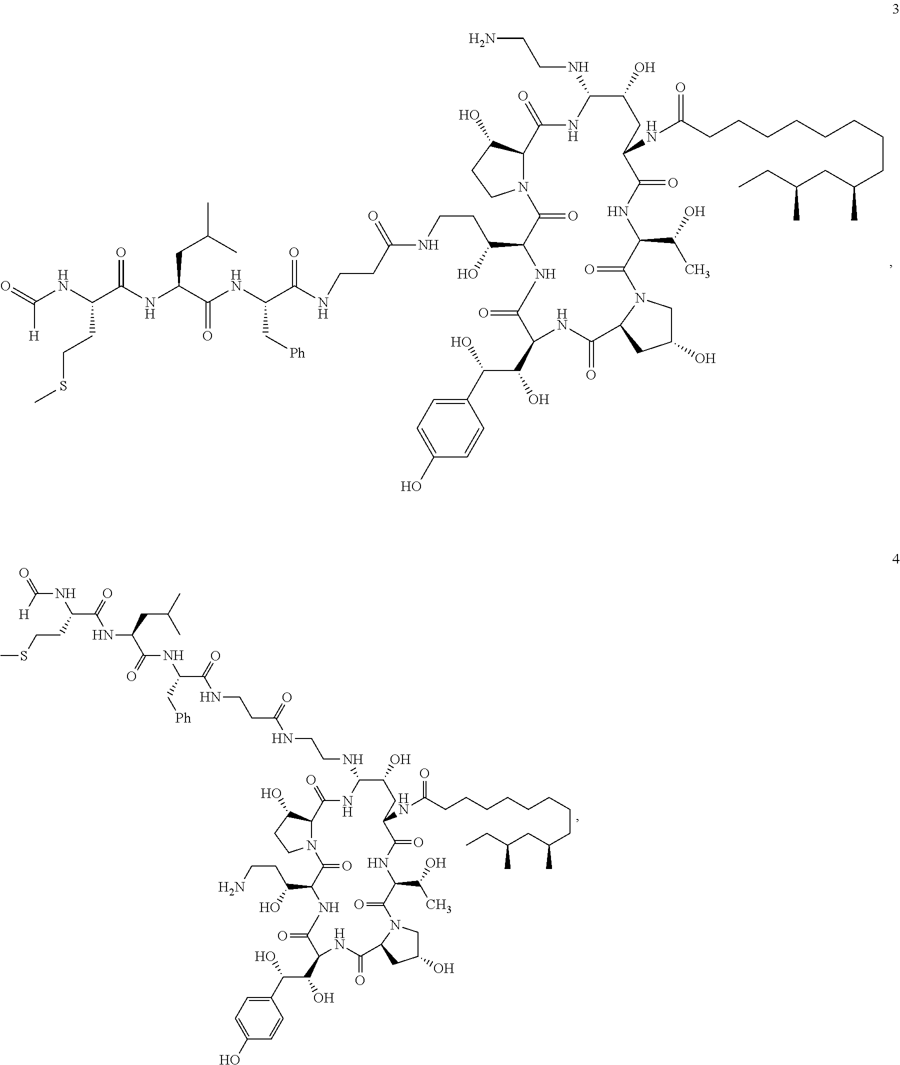 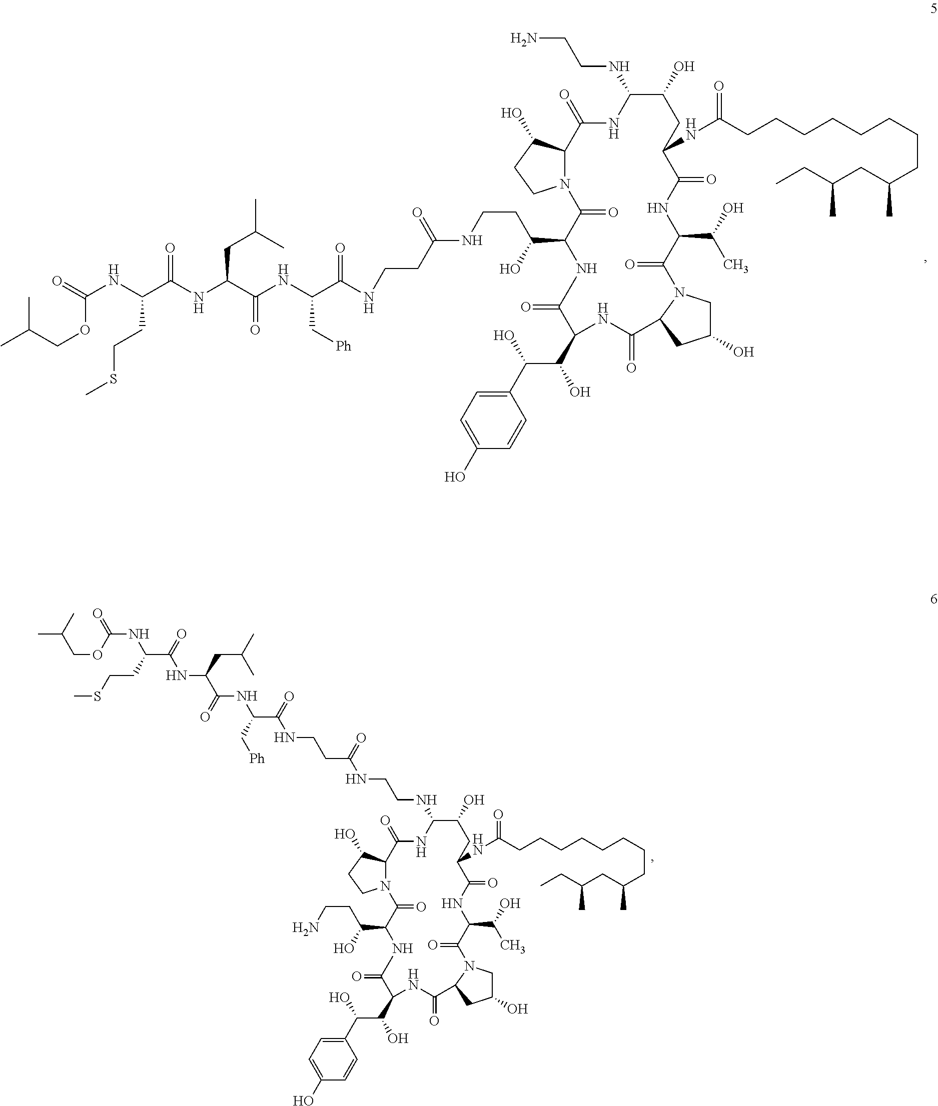 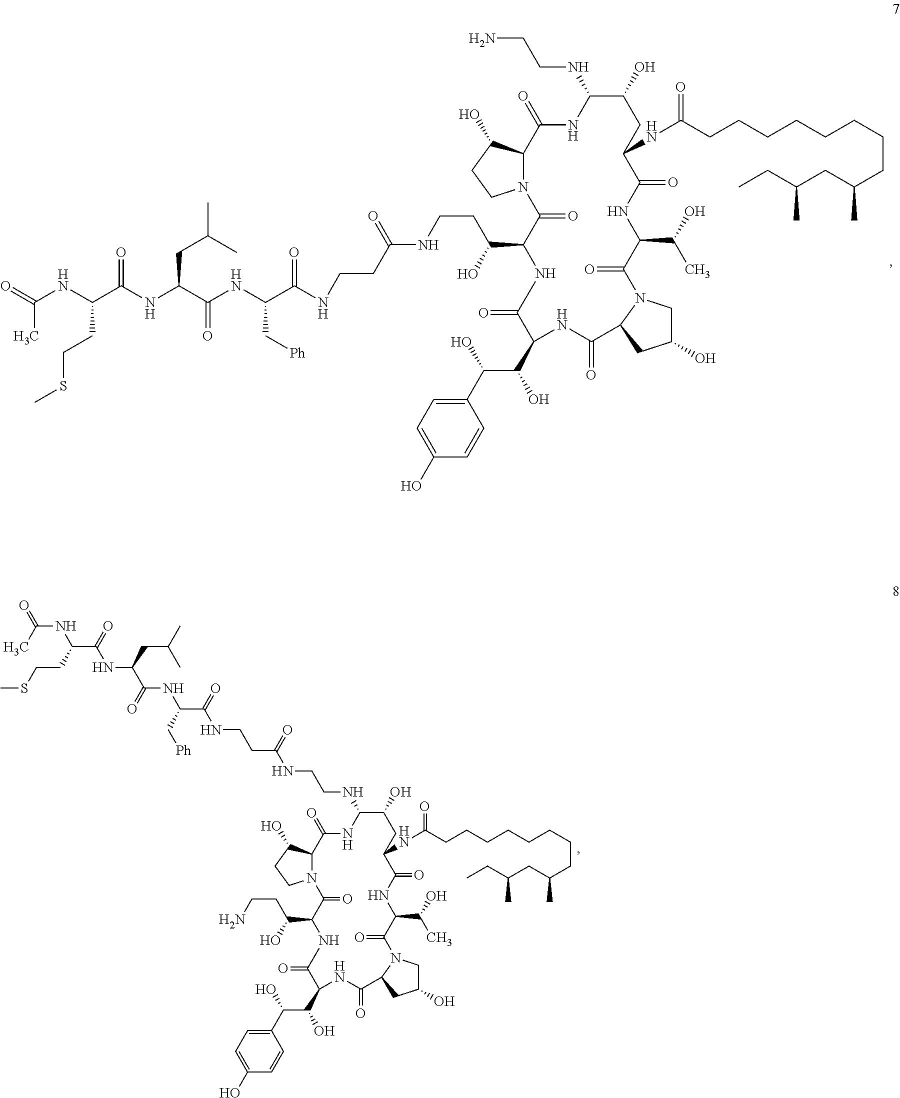 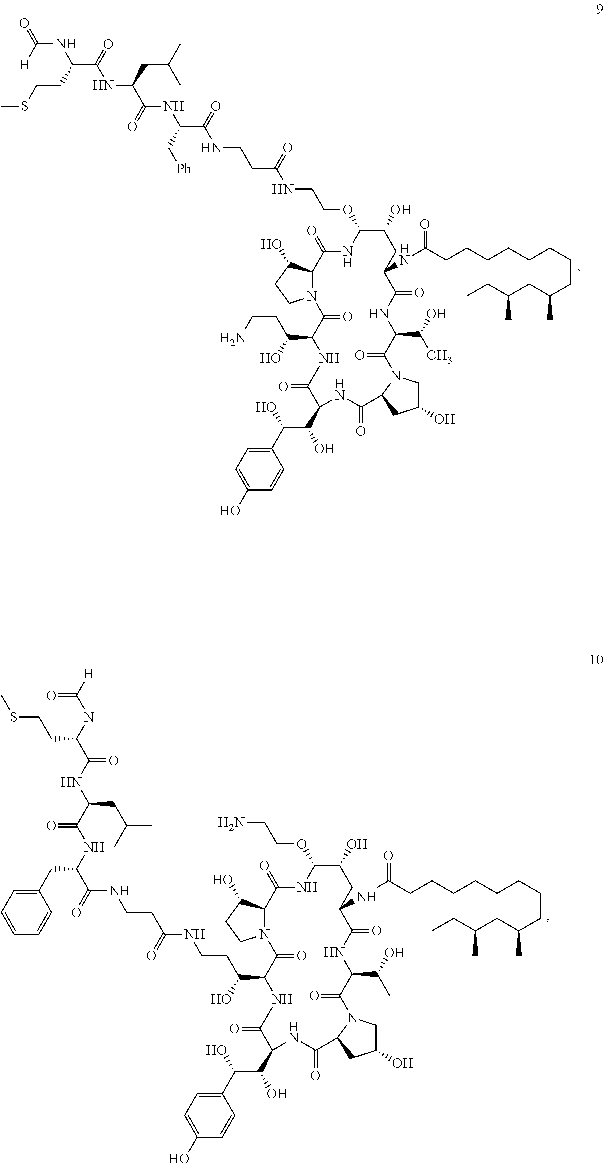 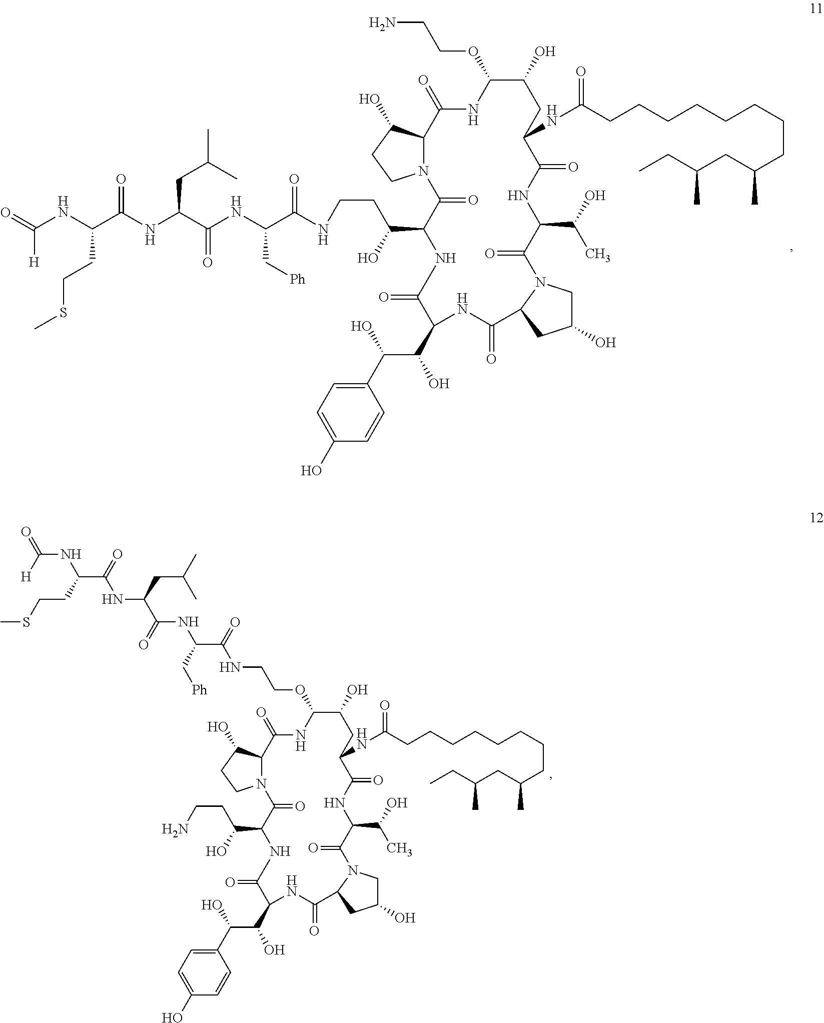 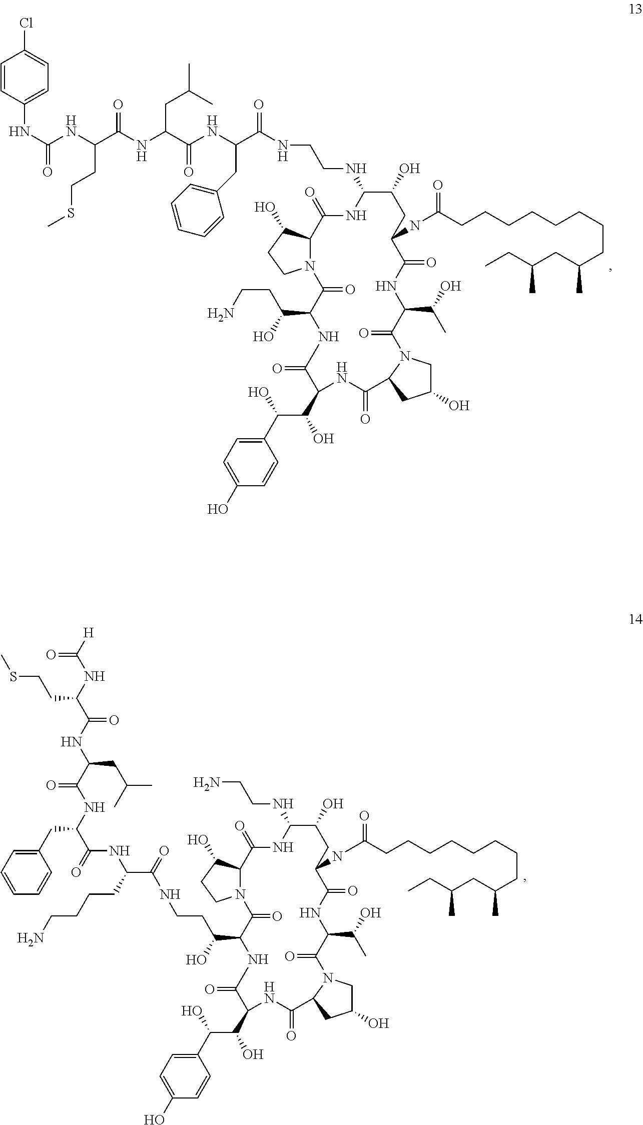 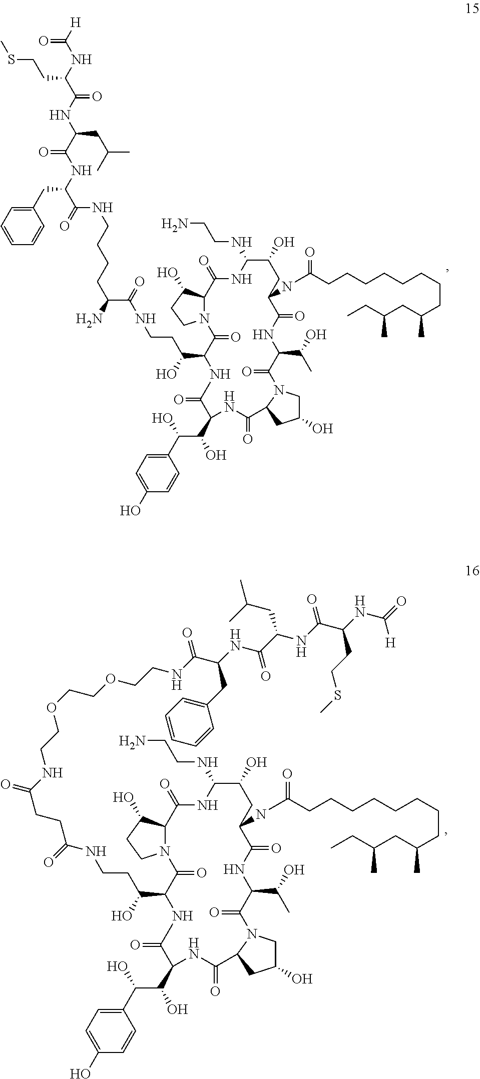 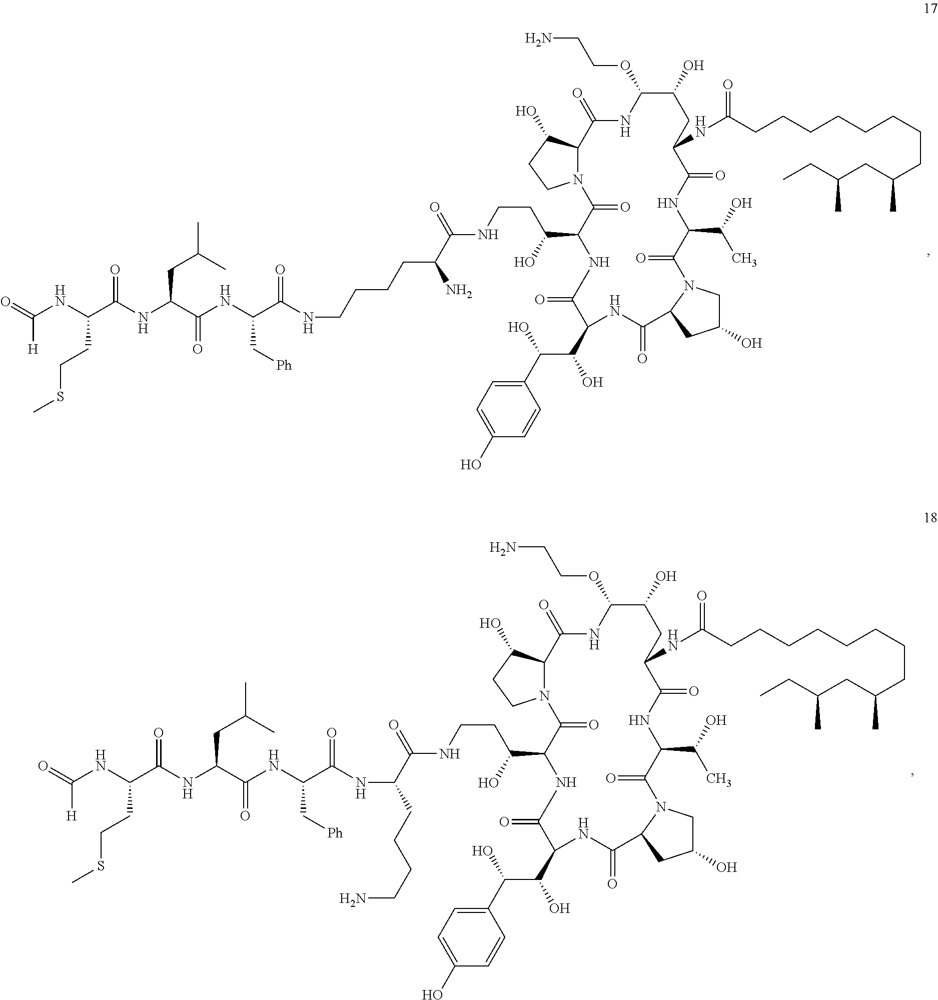 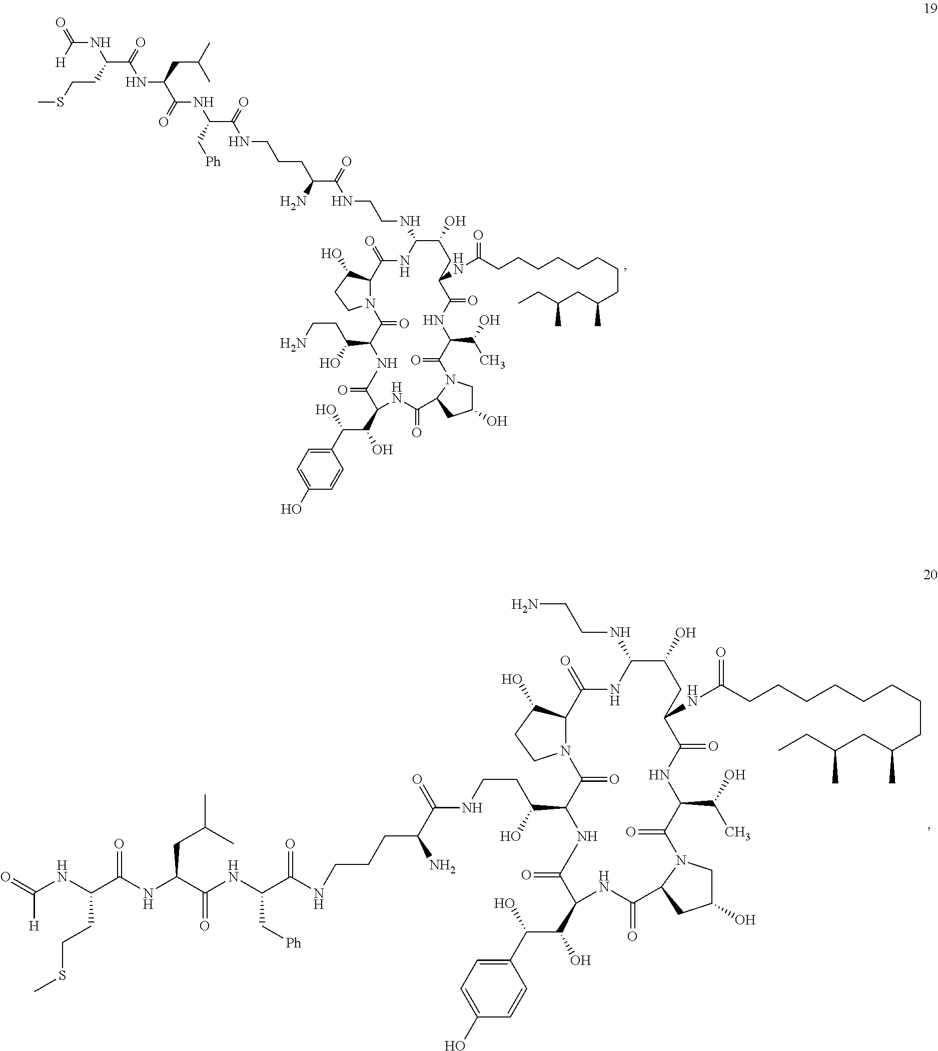 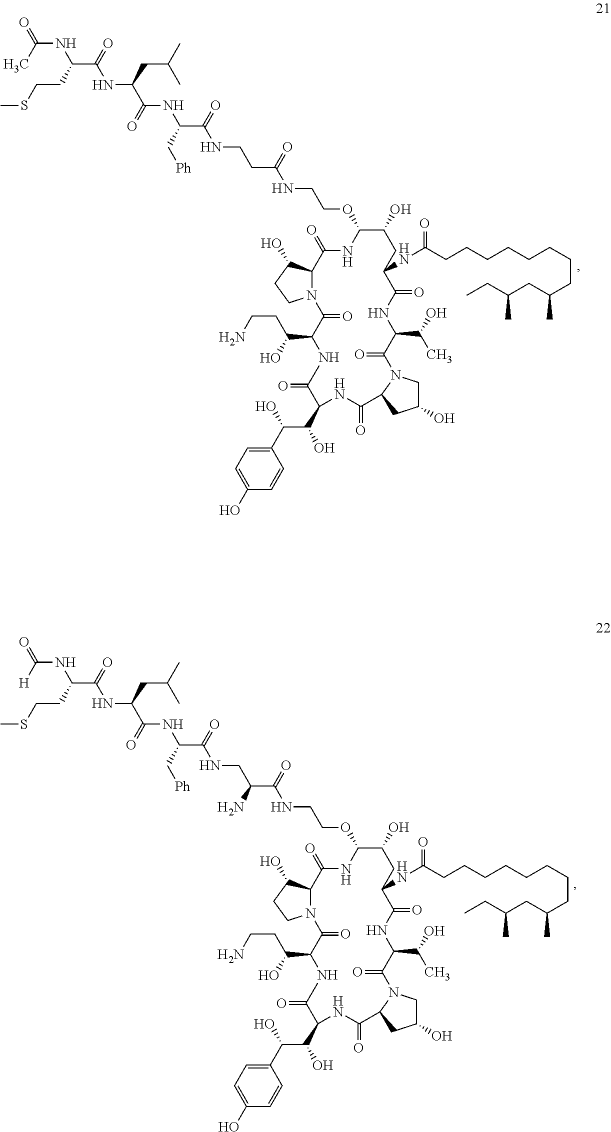 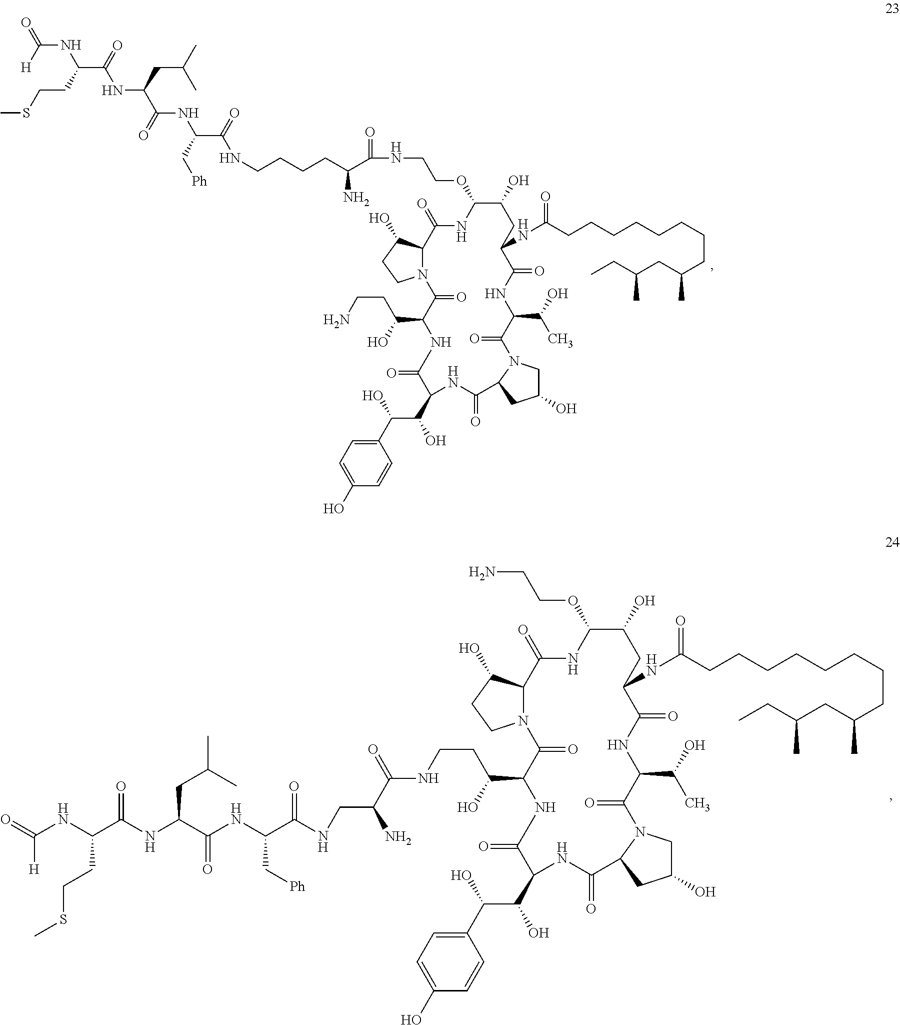 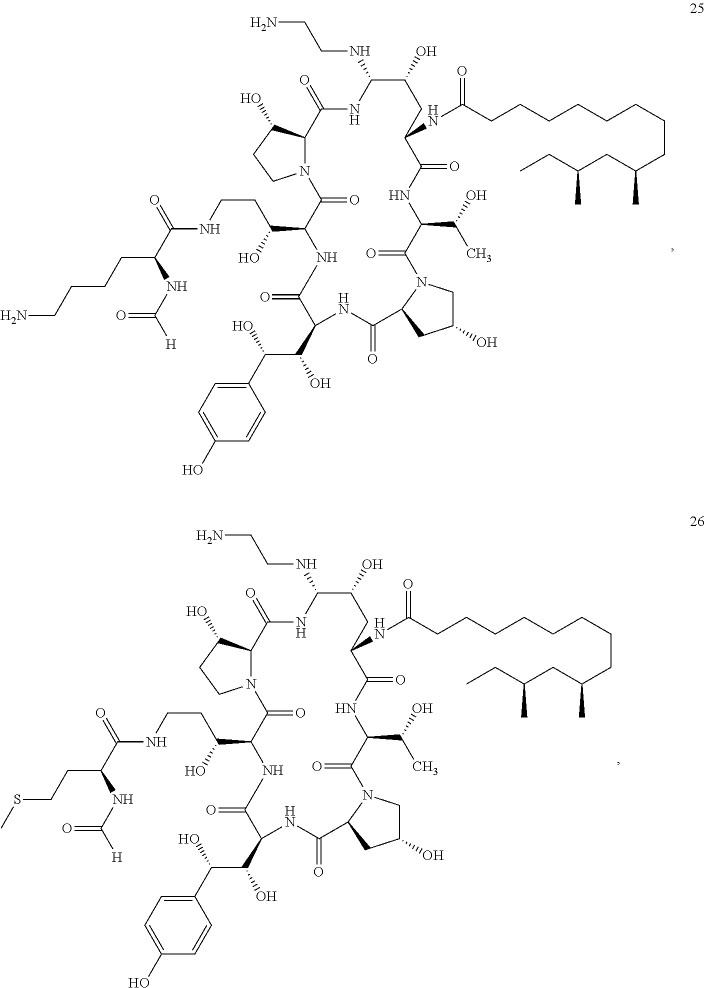 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCCCN)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O] 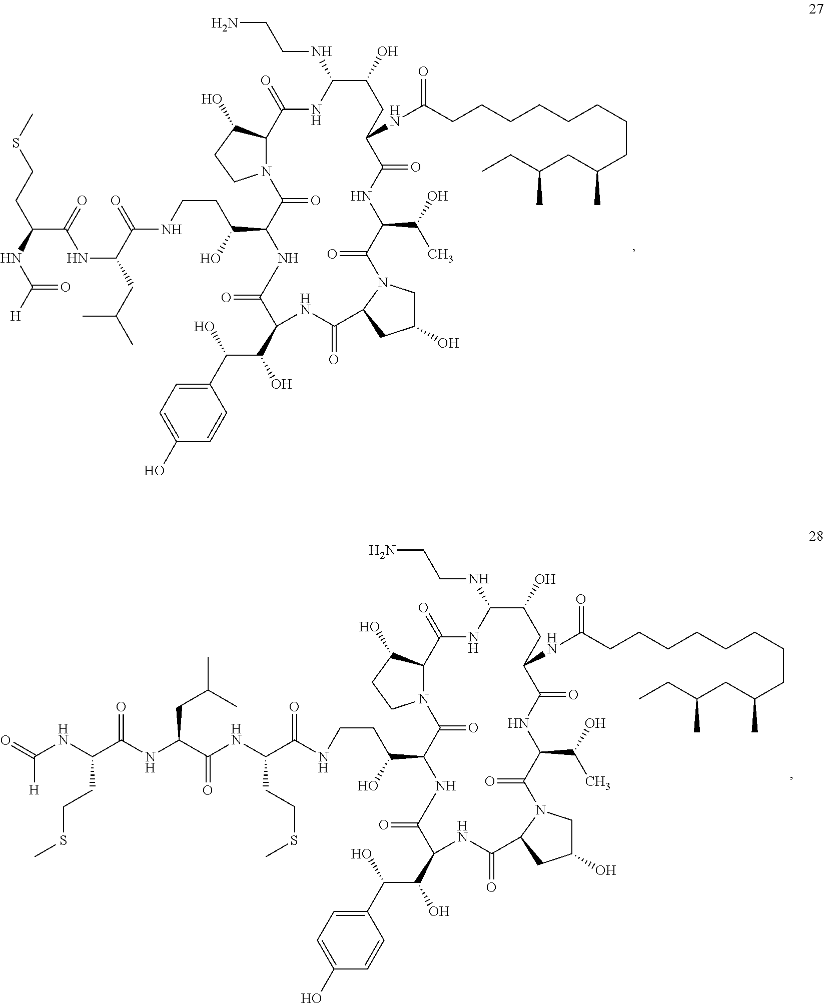 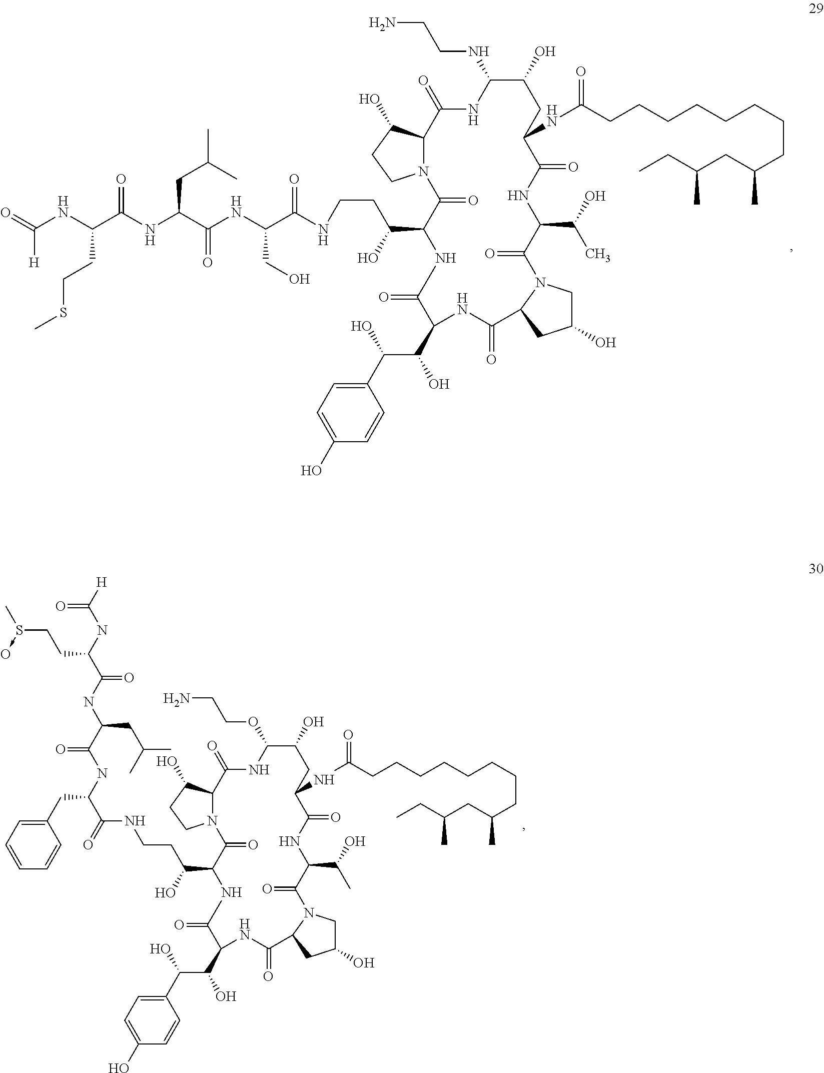 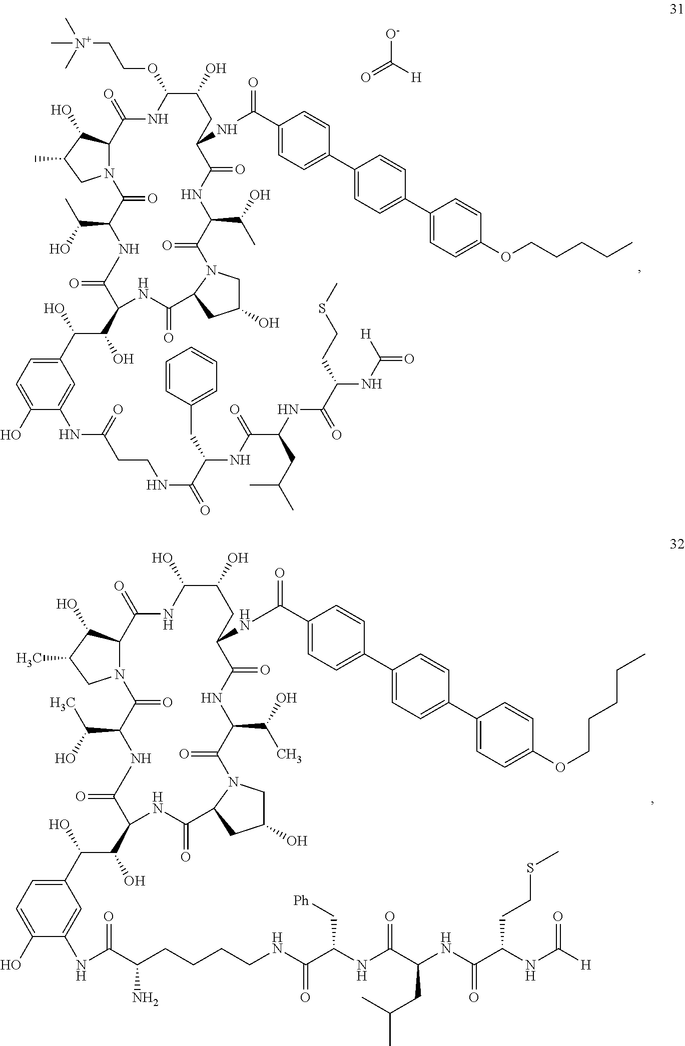 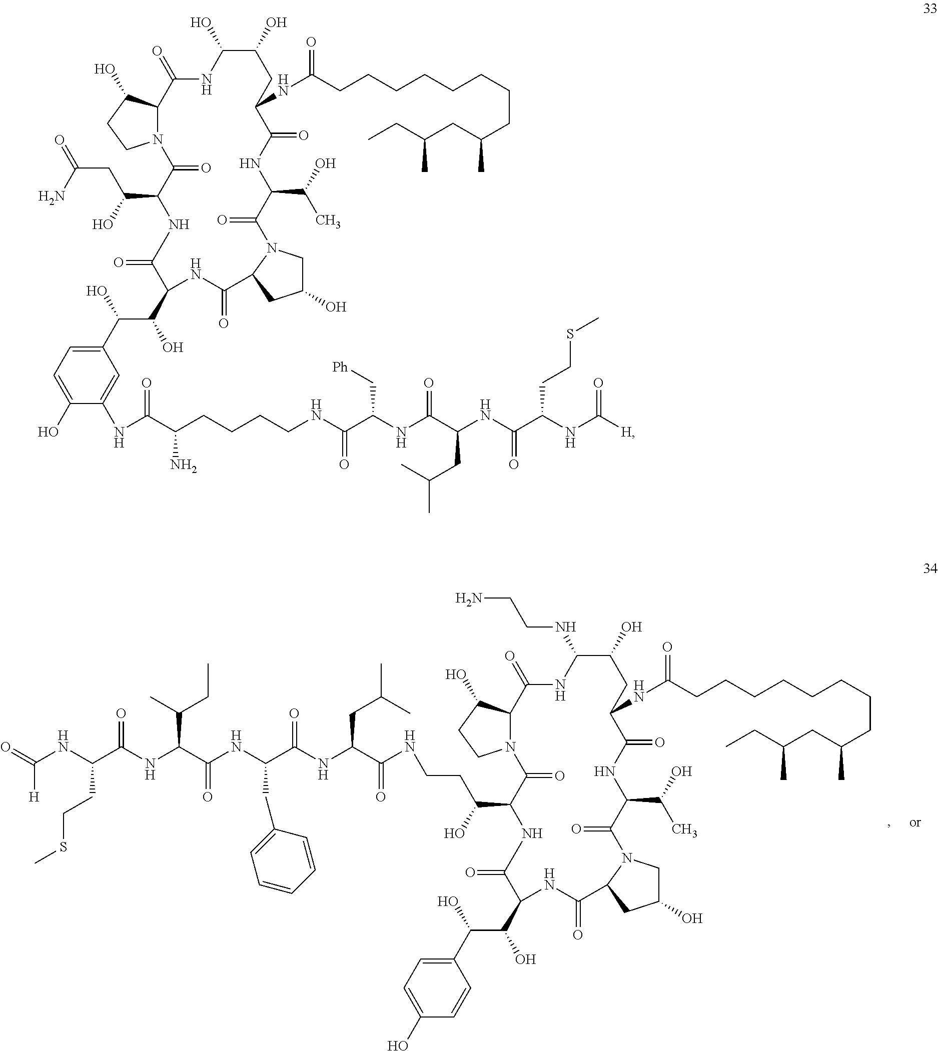 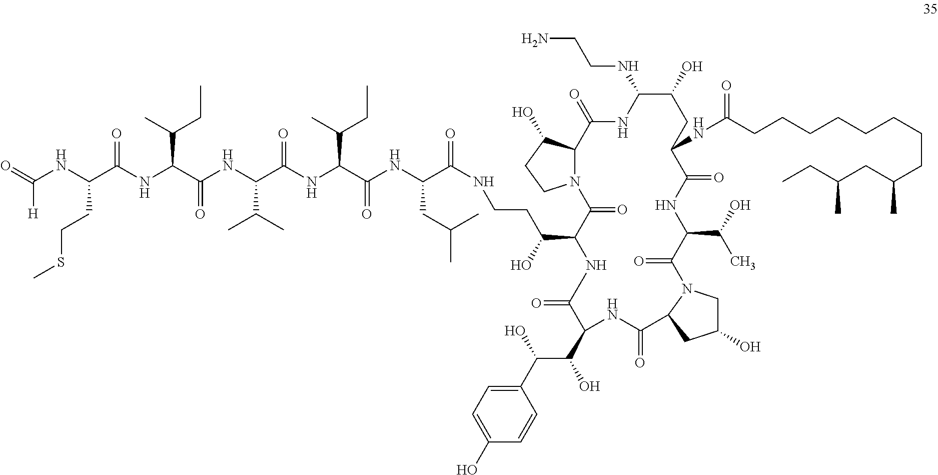 [SMILES: CCC(C)[C@H](NC(=O)[C@H](CCSC)NC=O)C(=O)N[C@H](C(=O)N[C@H](C(=O)N[C@@H](CC(C)C)C(=O)NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O)C(C)CC)C(C)C]
or a pharmaceutically acceptable salt thereof.

45. The compound of claim 44, wherein the compound is: 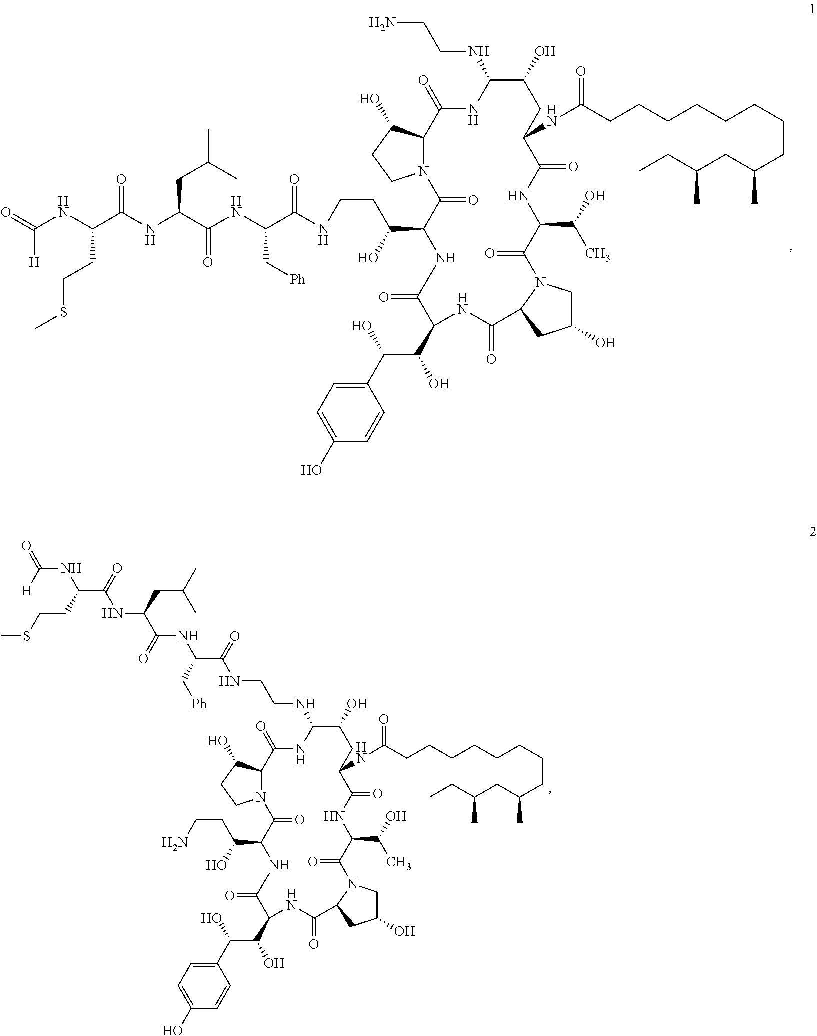 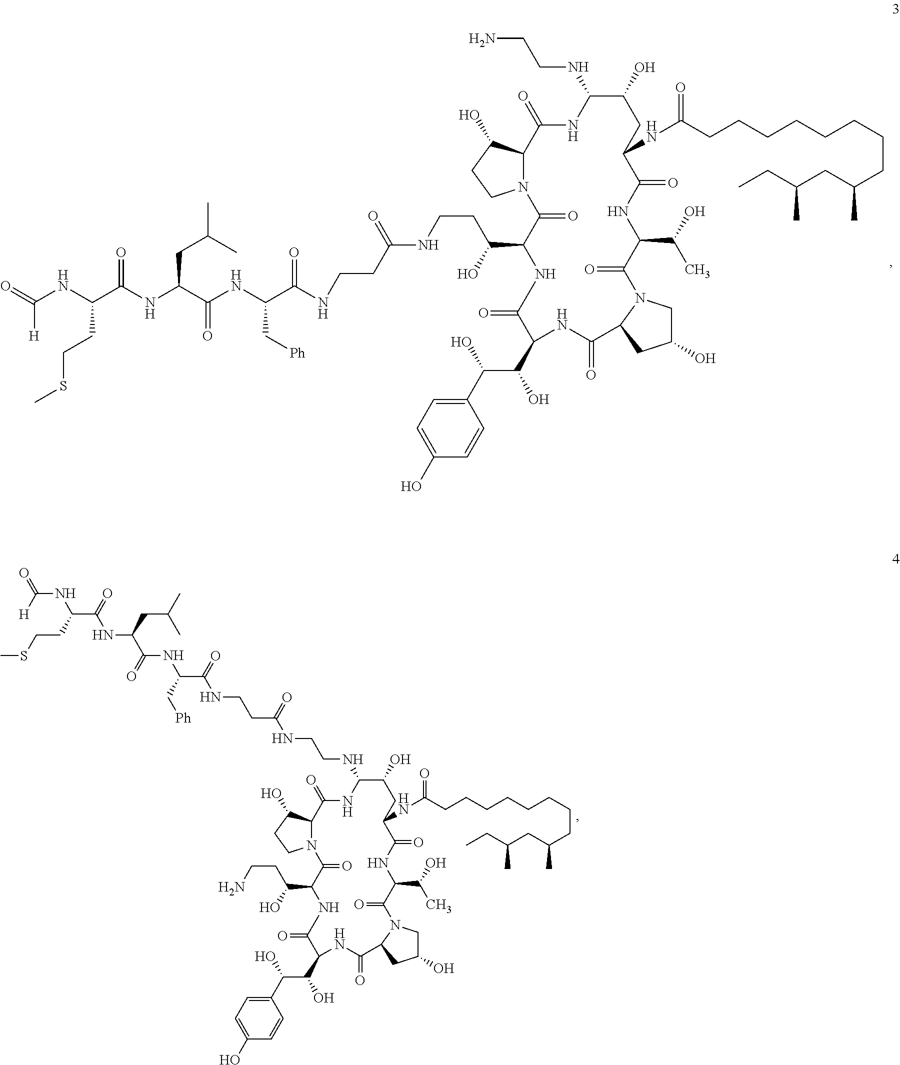 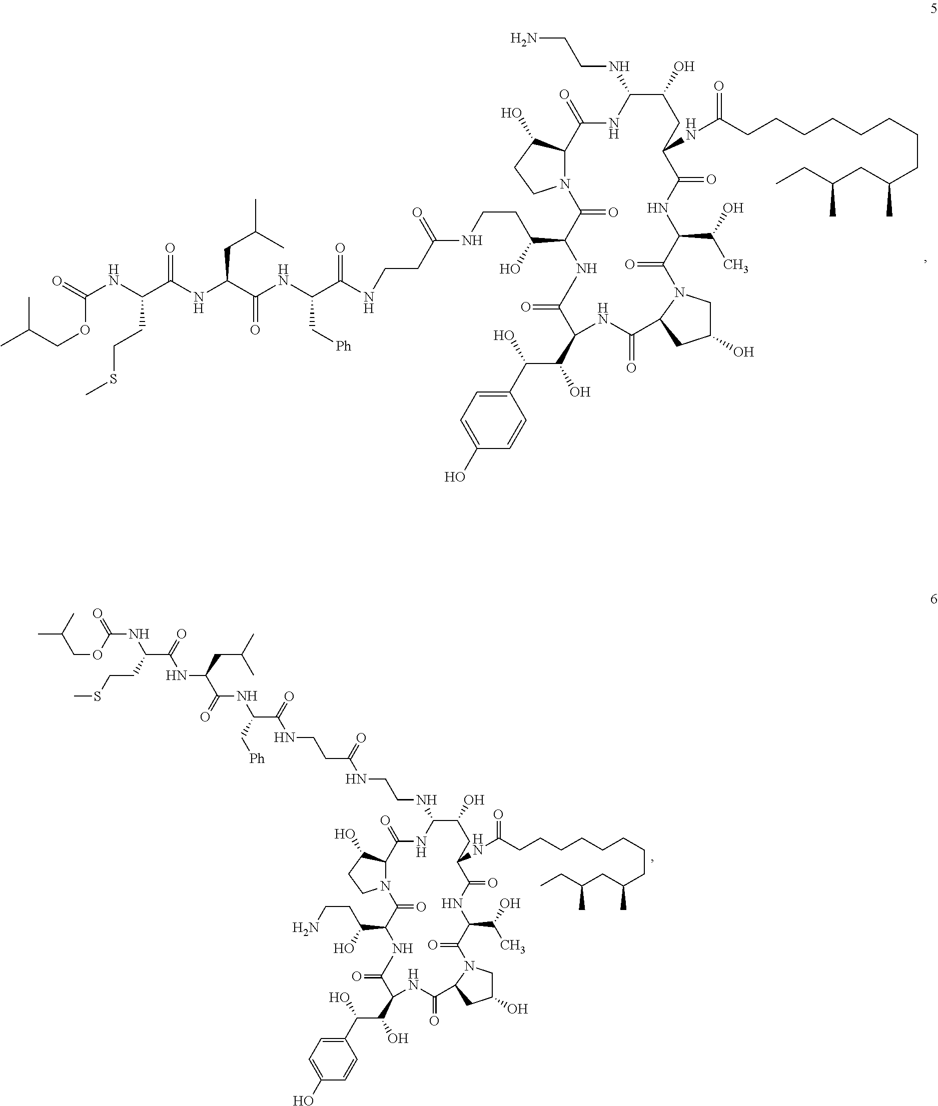 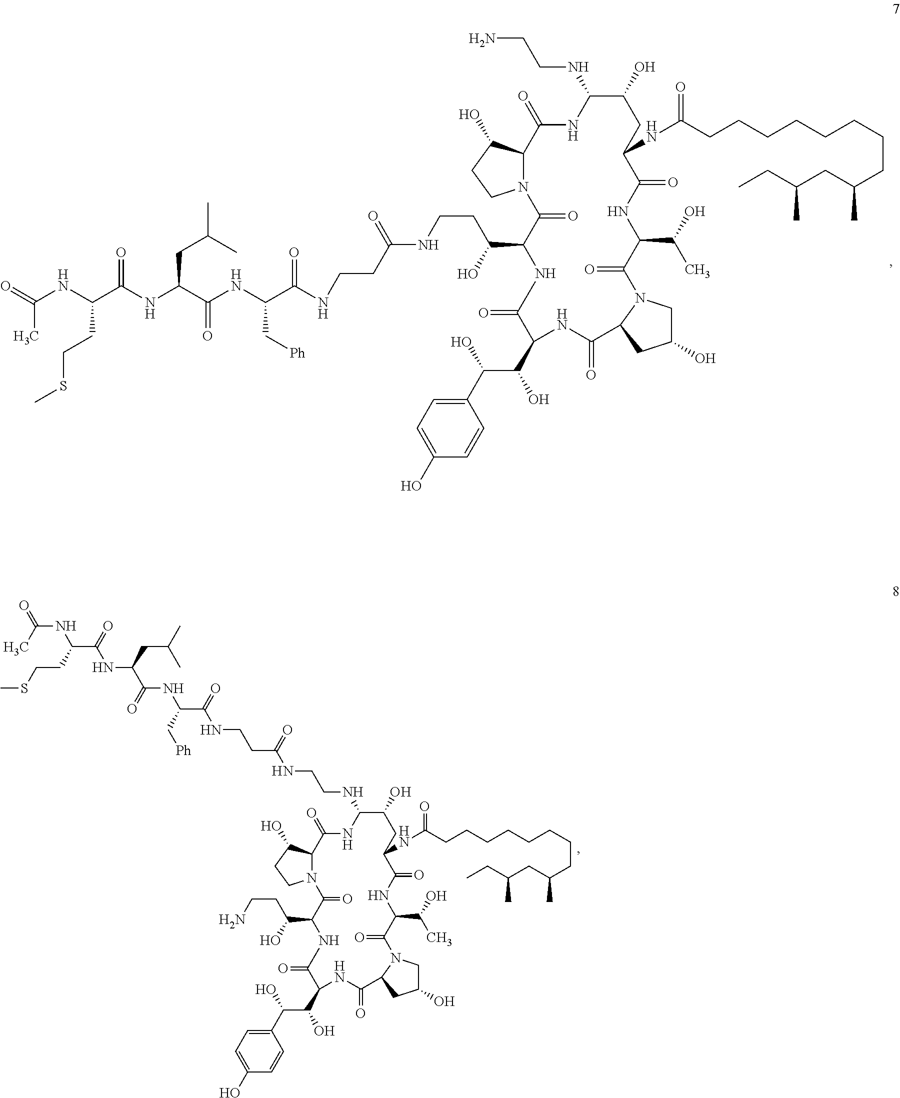 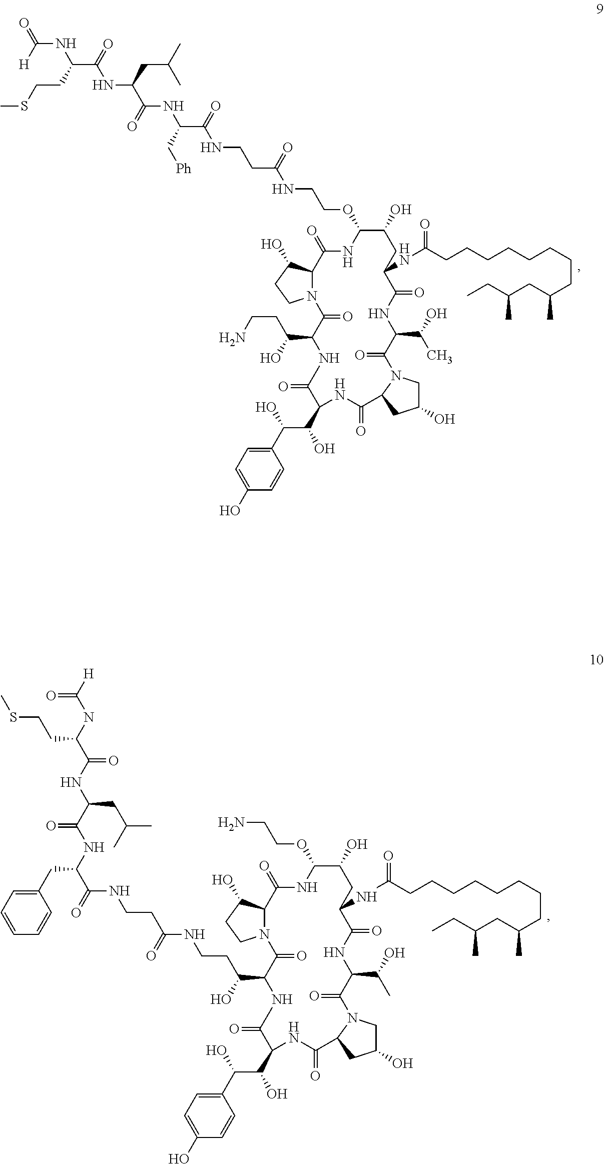 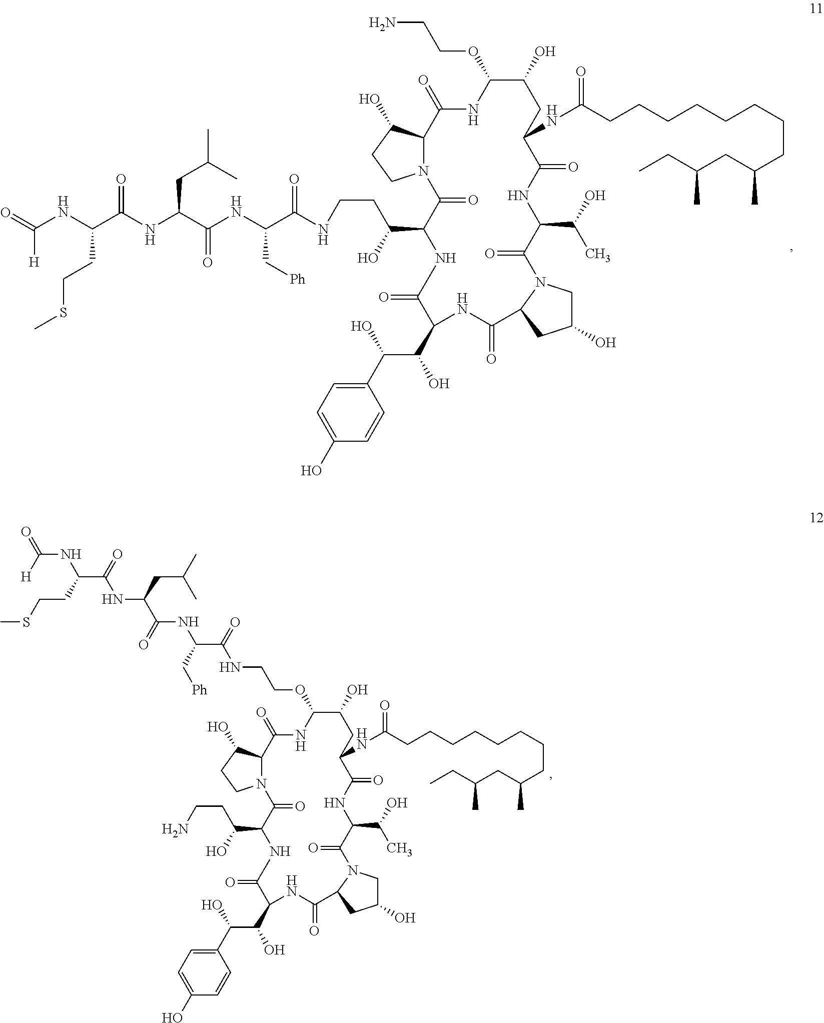 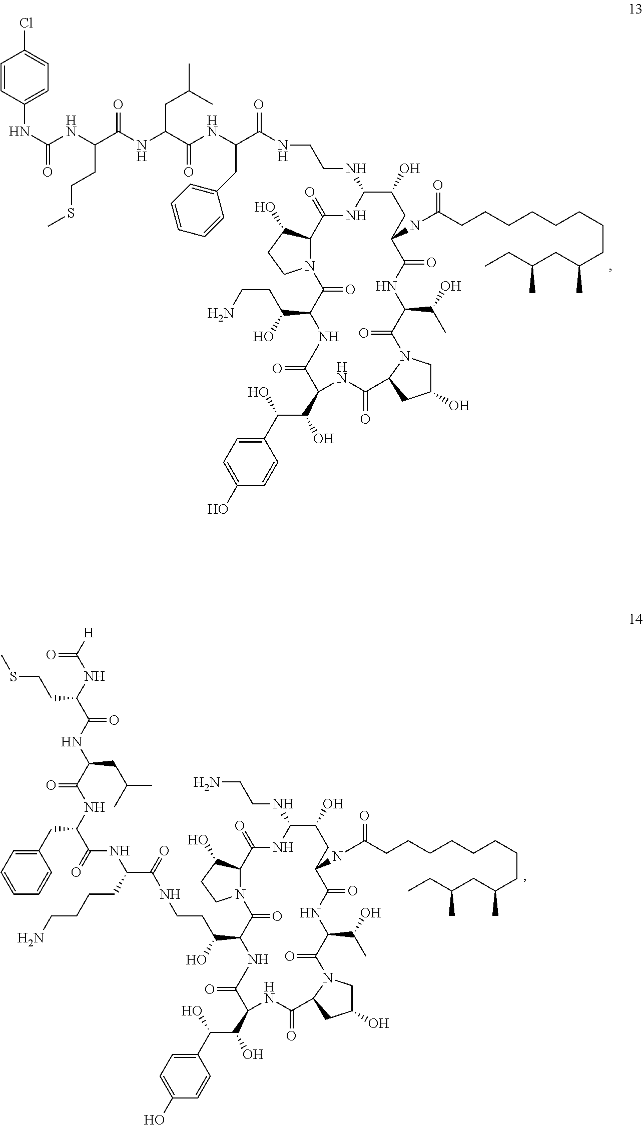 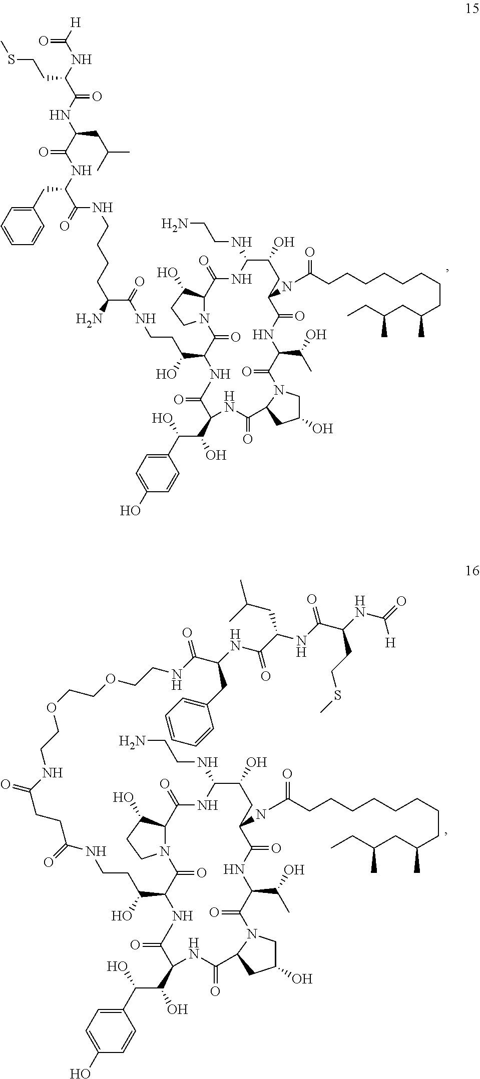 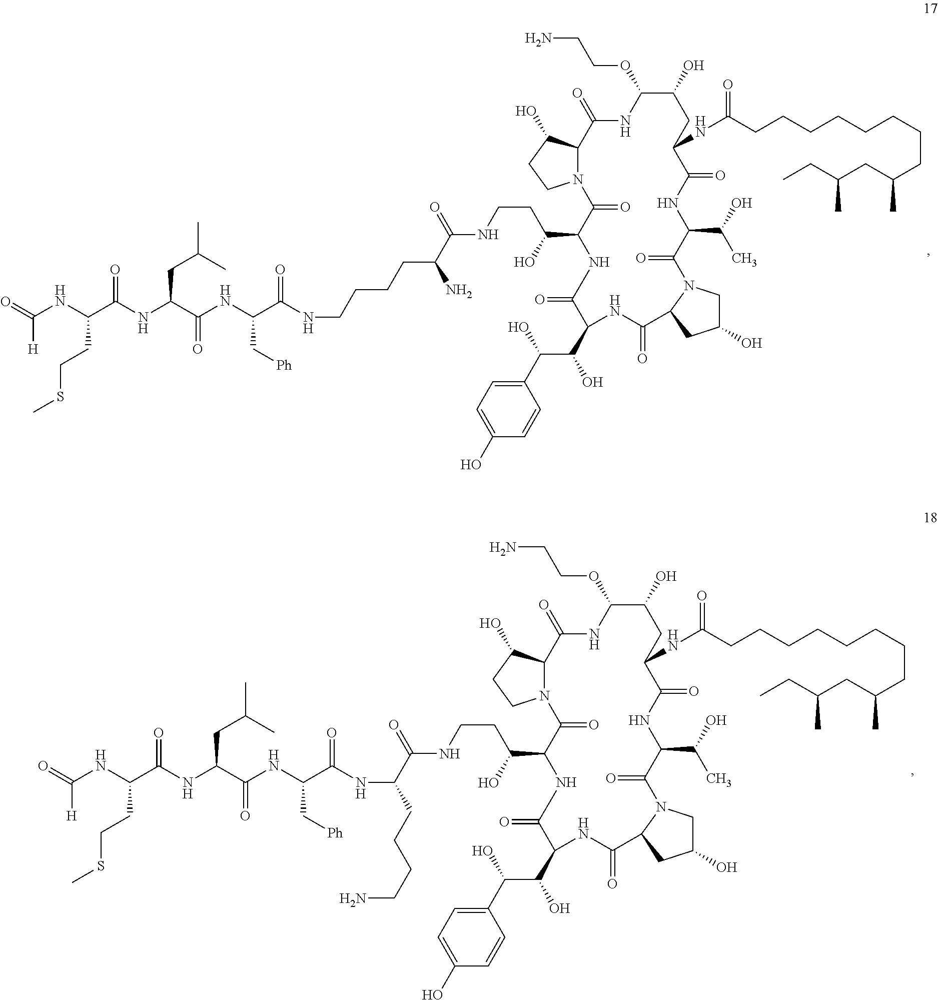 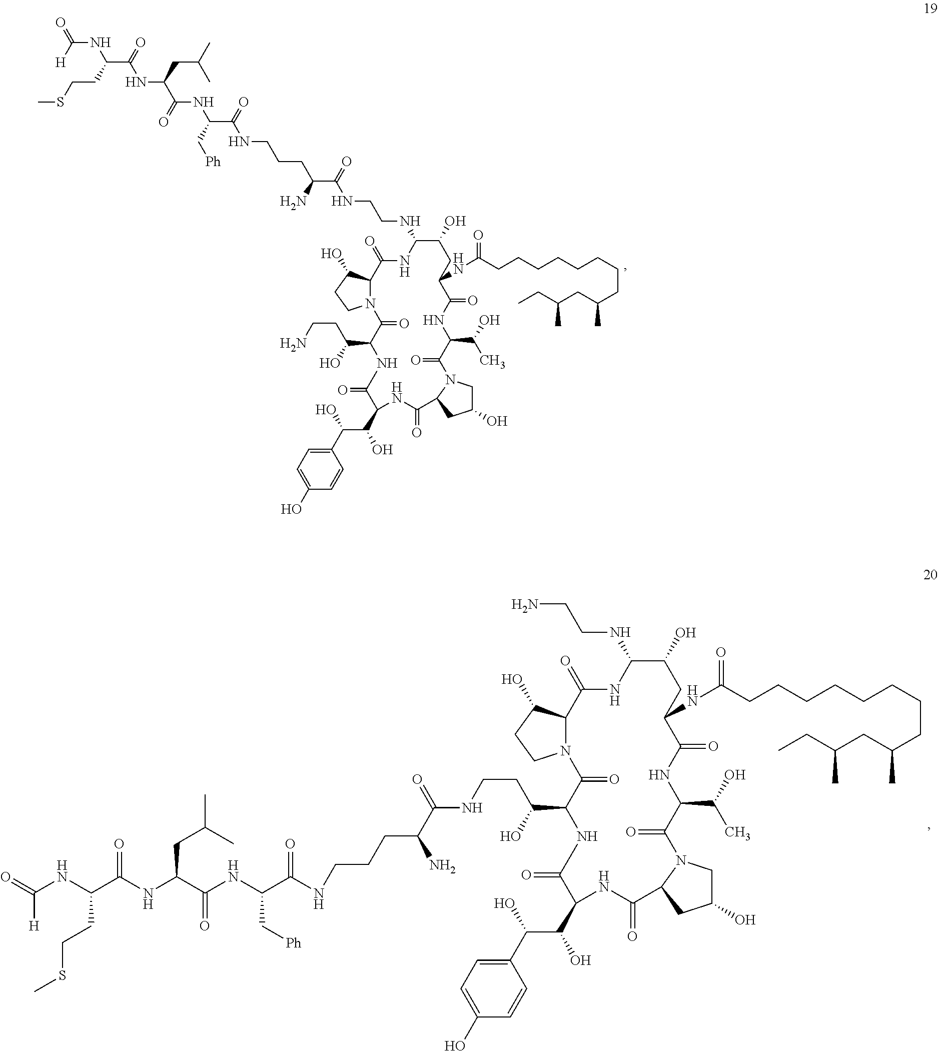 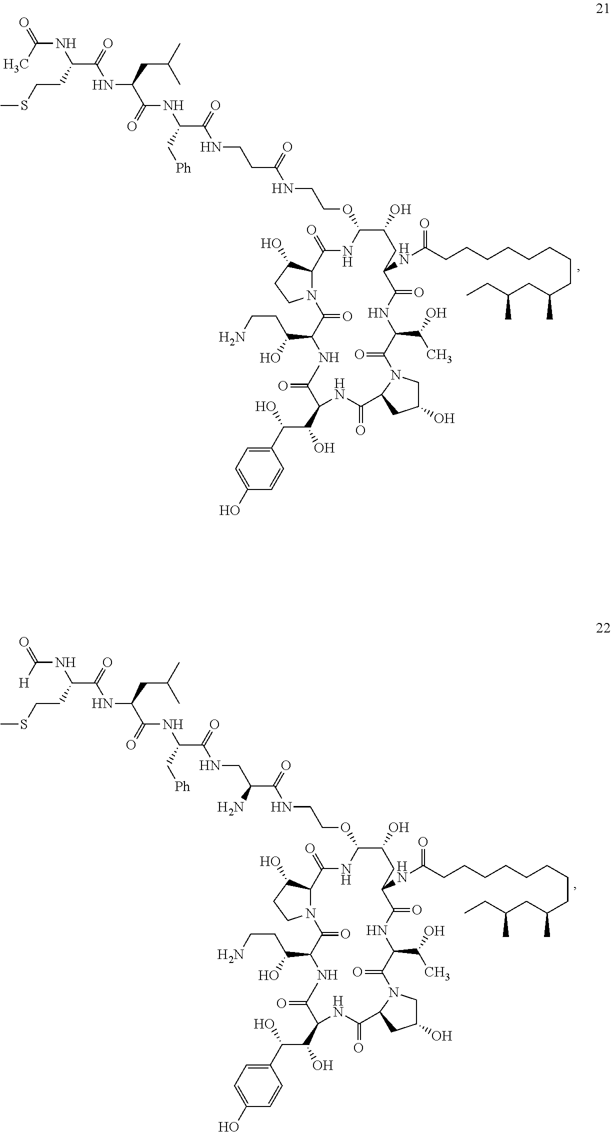 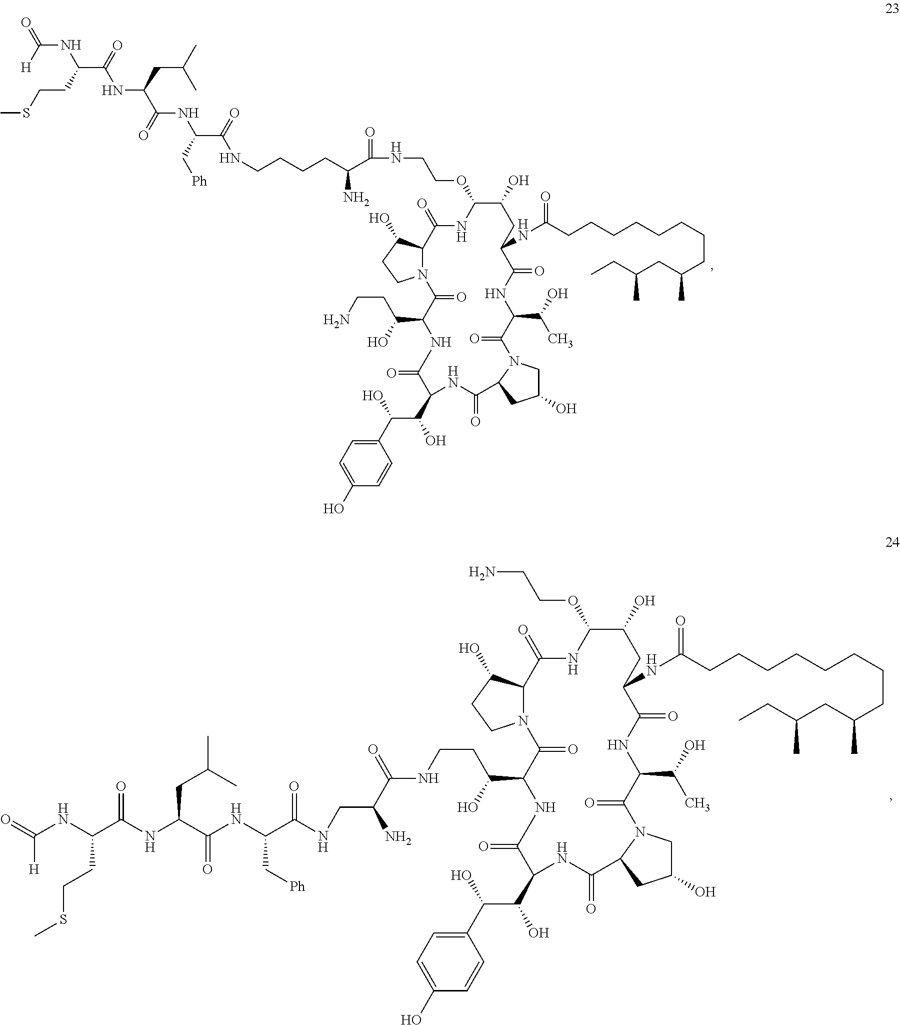 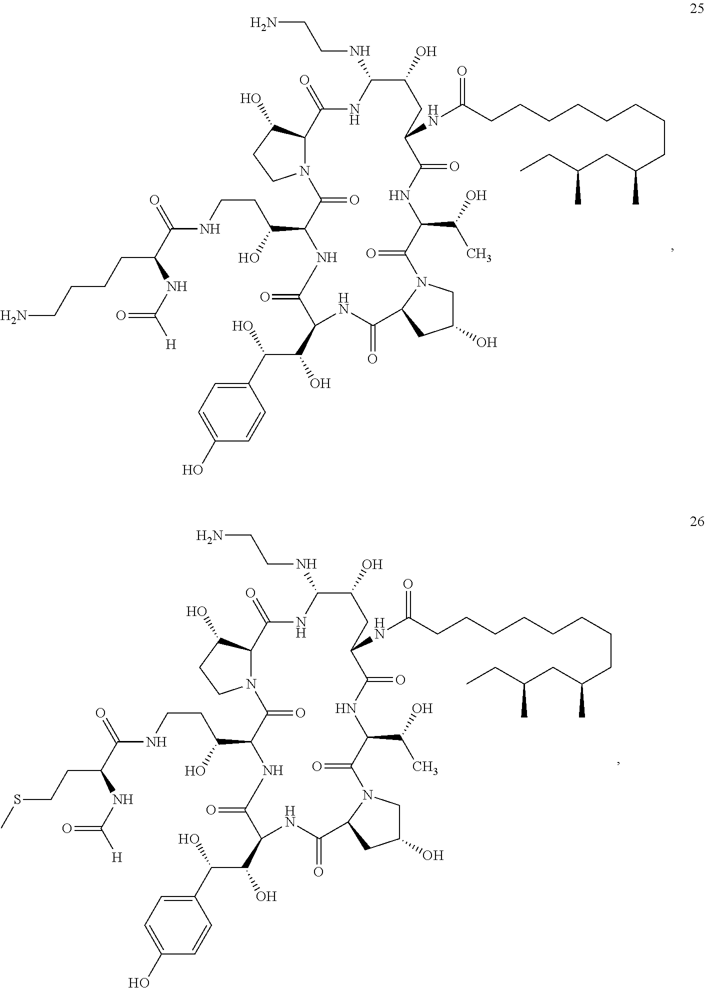 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCCCN)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O ; CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O] 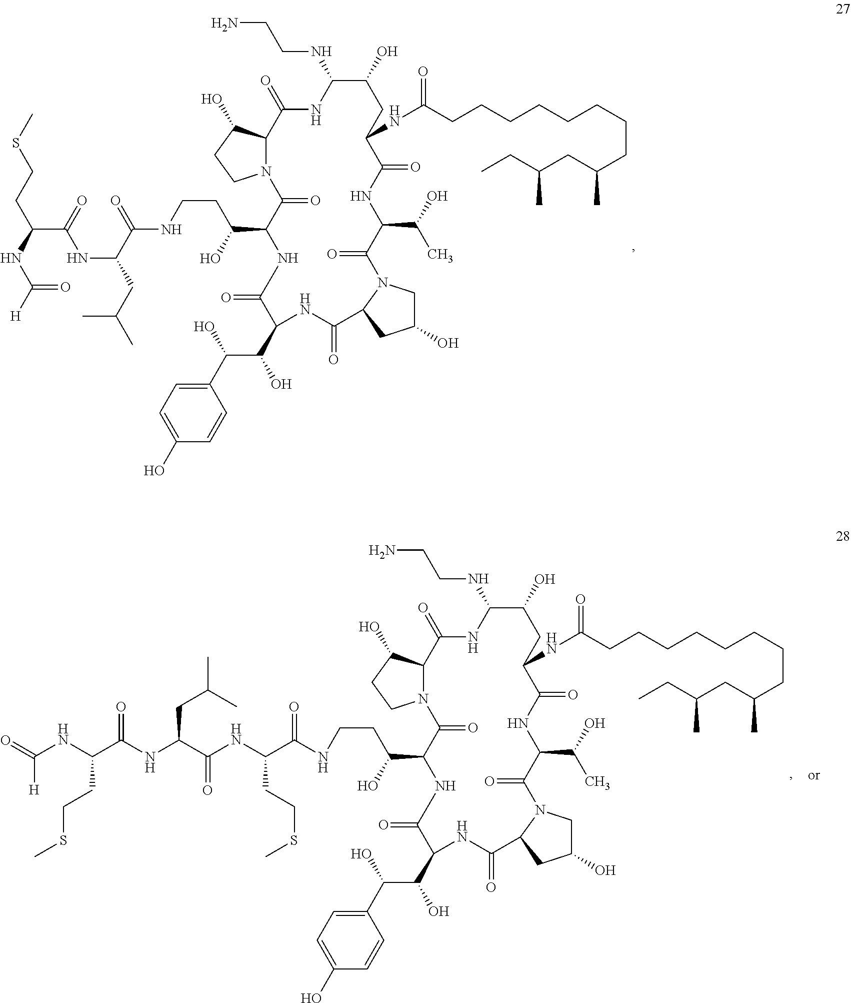 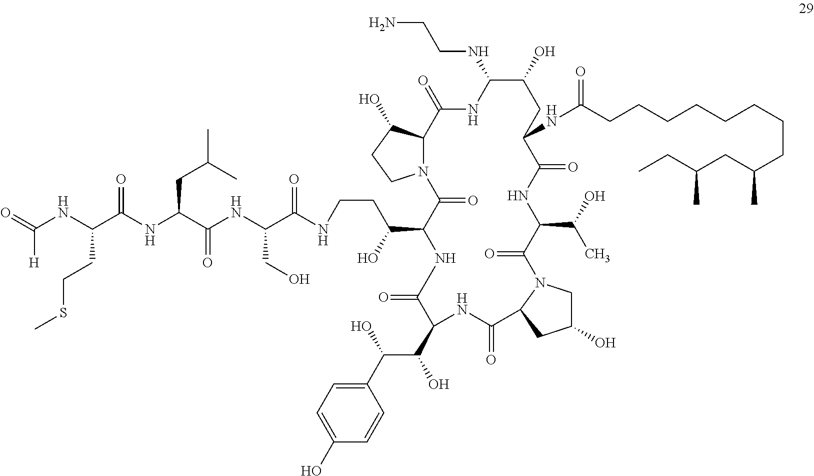 [SMILES: CC[C@H](C)C[C@H](C)CCCCCCCCC(=O)N[C@H]1C[C@@H](O)[C@@H](NCCN)NC(=O)[C@@H]2[C@@H](O)CCN2C(=O)[C@H]([C@H](O)CCNC(=O)[C@H](CO)NC(=O)[C@H](CC(C)C)NC(=O)[C@H](CCSC)NC=O)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]
or a pharmaceutically acceptable salt thereof.

46. A compound comprising:
(a) a moiety having the structure: 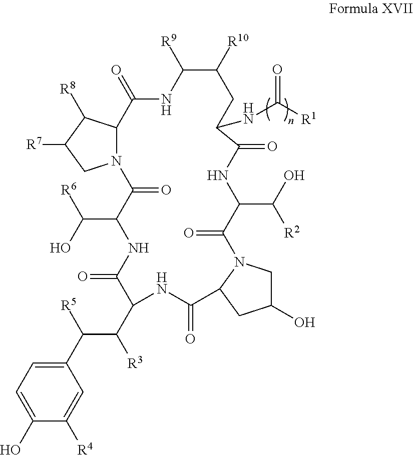
wherein R¹ is a C₁₀-C₃₆ moiety containing 0-8 heteroatoms selected from O, S, and N;
R² is hydrogen or methyl;
R³ and R⁵ are independently hydrogen or hydroxyl;
R⁴ is hydrogen, OR²⁰, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, hydroxyl, —NR¹¹R¹², or —NH(C═NH)NR¹¹R¹²;
R⁸ is hydrogen or hydroxyl;
R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl;
R¹⁰ is hydrogen, hydroxyl, oxo, —NR¹¹R¹², NH(CH₂)ₐNR¹¹R¹², or —NH(CH₂)ₐN⁺R¹¹R¹²R¹³;
R¹¹, R¹², and R¹³ are independently hydrogen or C₁-C₃ alkyl;
n is 0 or 1;
a is 2 to 4;
Q₁ is S, O or NH;
b is 2-6;
c is 1-8;
Q₂ is NR¹¹R¹², N⁺R¹¹R¹²R¹³, or OR²⁰; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹², wherein at least one R¹¹ is a bond conjugated to (b) a peptide comprising an amino acid residue having the formula:
R¹⁴—X1-X2-X3-X4-X5-X6-X7-X8-X9 Formula III
wherein X9 is any amino acid;
X1-X8 are any amino acid or absent;
R¹⁴ is hydrogen or 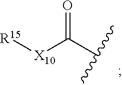
wherein X₁₀ is a bond, NH, or O; and
R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic;
wherein if R¹⁴ is hydrogen, X1 is not absent;
or a pharmaceutically acceptable salt thereof.

47. The compound of claim 46, wherein (a) has the structure: 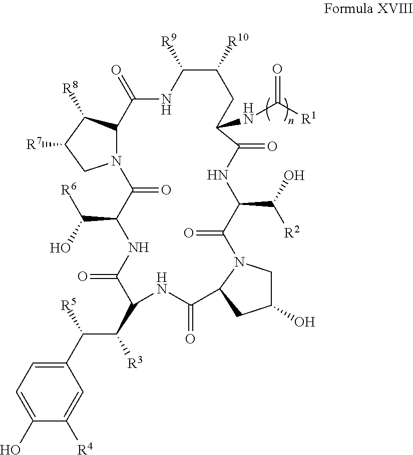
wherein R¹ is a C₁₀-C₃₆ moiety containing 0-8 heteroatoms selected from O, S, and N;
R² is hydrogen or methyl;
R³ and R⁵ are independently hydrogen or hydroxyl;
R⁴ is hydrogen, OR²⁰, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, hydroxyl, —NR¹¹R¹², or —NH(C═NH)NR¹¹R¹²;
R⁸ is hydrogen or hydroxyl;
R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl;
R¹⁰ is hydrogen, hydroxyl, oxo, —NR¹¹R¹², NH(CH₂)ₐNR¹¹R¹², or —NH(CH₂)ₐN⁺R¹¹R¹²R¹³;
R¹¹, R¹², and R¹³ are independently hydrogen or C₁-C₃ alkyl;
n is 0 or 1;
a is 2 to 4;
Q₁ is S, O or NH;
b is 2-6;
c is 1-8;
Q₂ is NR¹¹R¹², N⁺R¹¹R¹²R¹³, OR¹⁶; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹², wherein at least one R¹¹ is a bond.

48. The compound of claim 46, wherein (a) has the structure: 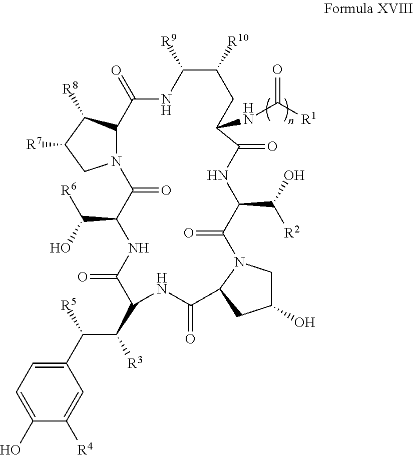
wherein R¹ is a moiety containing a C₁-C₁₇ alkyl or heteroalkyl, C₂-C₁₇ alkenyl or heteroalkenyl, aryl or heteroaryl, cyclic, polycyclic, heterocyclic or heteropolycyclic moiety, or a combination thereof to form a C₁₀-C₃₆ moiety;
R² is methyl;
R³ and R⁵ are hydroxyl;
R⁴ is hydrogen, OR²⁰, —OSO₃H, or —NR¹¹R¹²;
R⁶ is hydrogen, methyl, —CH₂(C═O)NR¹¹R¹², —CH₂CH₂NR¹¹R¹², or —CH₂CH₂N⁺R¹¹R¹²R¹³;
R⁷ is hydrogen, methyl, hydroxyl, —NR¹¹R¹², or —NH(C═NH)NR¹¹R¹²;
R⁸ is hydrogen or hydroxyl;
R⁹ is -(Q₁(CH₂)_{b})_{c}Q₂, —NR¹¹R¹², —CH₂NR¹¹R¹², —CH₂N⁺R¹¹R¹²R¹³, hydrogen, or hydroxyl;
R¹⁰ is hydrogen, hydroxyl, oxo, —NR¹¹R¹², NH(CH₂)ₐNR¹¹R¹², or —NH(CH₂)ₐN⁺R¹¹R¹²R¹³;
R¹¹, R¹², and R¹³ are independently hydrogen or methyl;
n is 0 or 1;
a is 2 to 4;
Q₁ is S, O or NH;
b is 2-6;
c is 1-8;
Q₂ is NR¹¹R¹², N⁺R¹¹R¹²R¹³, or OR²⁰; and
R²⁰ is CH₃, CH₂CH₃, CH₂CH₂CH₃, CH(CH₃)₂, or H;
wherein at least one of R⁴, R⁶, R⁷, R⁹, or R¹⁰ contains —NR¹¹R¹², wherein at least one R¹¹ is a bond.

49. The compound of any one of claim 46, wherein (a) has the structure: 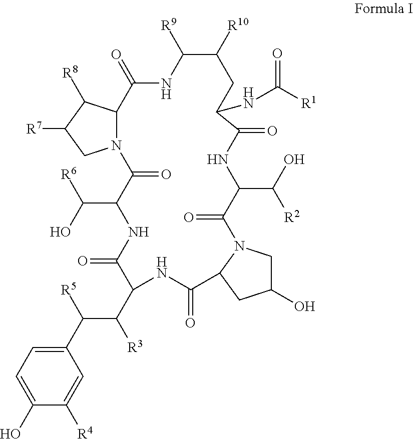
wherein R¹ is optionally substituted C₁₃-C₁₇ alkyl, optionally substituted C₁₃-C₁₇ alkenyl, optionally substituted aryl, or optionally substituted heteroaryl;
R² is methyl;
R³, R⁵, and R¹⁰ are hydroxyl;
R⁴ is hydrogen, hydroxyl, —OMe, —OSO₃H, or —NR¹¹R¹²;
R⁶ is methyl, —CH₂(C═O)NR¹¹R¹², —CH₂CH₂NR¹¹R¹², or —CH₂CH₂N⁺R¹¹R¹²R¹³;
R⁷ is hydrogen, or methyl;
R⁸ is hydroxyl;
R⁹ is hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or methyl; and
a is 2 to 4;
wherein at least one of R⁴, R⁶, or R⁹ contains —NR¹¹R¹², wherein at least one R¹¹ is a bond.

50. The compound of any one of claim 46, wherein (a) has the structure: 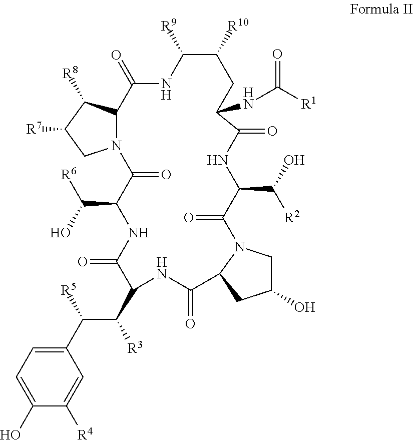
wherein R¹ is optionally substituted C₁₃-C₁₇ alkyl, optionally substituted C₁₃-C₁₇ alkenyl, optionally substituted aryl, or optionally substituted heteroaryl;
R² is methyl;
R³, R⁵, and R¹⁰ are hydroxyl;
R⁴ is hydrogen, hydroxyl, —OMe, —OSO₃H, or —NR¹¹R¹²;
R⁶ is methyl, —CH₂(C═O)NR¹¹R¹², —CH₂CH₂NR¹¹R¹², or —CH₂CH₂N⁺R¹¹R¹²R¹³;
R⁷ is hydrogen, or methyl;
R⁸ is hydroxyl;
R⁹ is hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or methyl; and
a is 2 to 4;
wherein at least one of R⁴, R⁶, or R⁹ contains —NR¹¹R¹², wherein at least one R¹¹ is a bond.

51. The compound of any one of claim 46, wherein (a) has the structure: 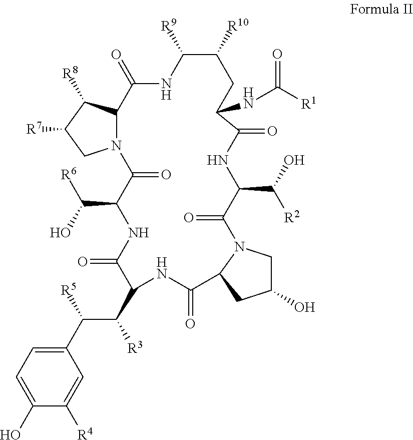
wherein R¹ is optionally substituted C₁₃-C₁₇ alkyl, optionally substituted aryl, or optionally substituted heteroaryl;
R² is methyl;
R³, R⁵, and R¹⁰ are hydroxyl;
R⁴ is hydrogen, hydroxyl, —OMe, —OSO₃H, or —NR¹¹R¹²;
R⁶ is methyl, —CH₂(C═O)NR¹¹R¹², —CH₂CH₂NR¹¹R¹², or —CH₂CH₂N⁺R¹¹R¹²R¹³;
R⁷ is hydrogen, or methyl;
R⁸ is hydroxyl;
R⁹ is hydroxyl, —NR¹¹R¹², —O(CH₂)ₐNR¹¹R¹², —O(CH₂)ₐN⁺R¹¹R¹²R¹³, —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³;
R¹¹, R¹², and R¹³ are independently hydrogen or methyl; and
a is 2 to 4;
wherein at least one of R⁴, R⁶, or R⁹ contains —NR¹¹R¹², wherein at least one R¹¹ is a bond.

52. The compound of any one of claims 46-51, wherein R¹ is

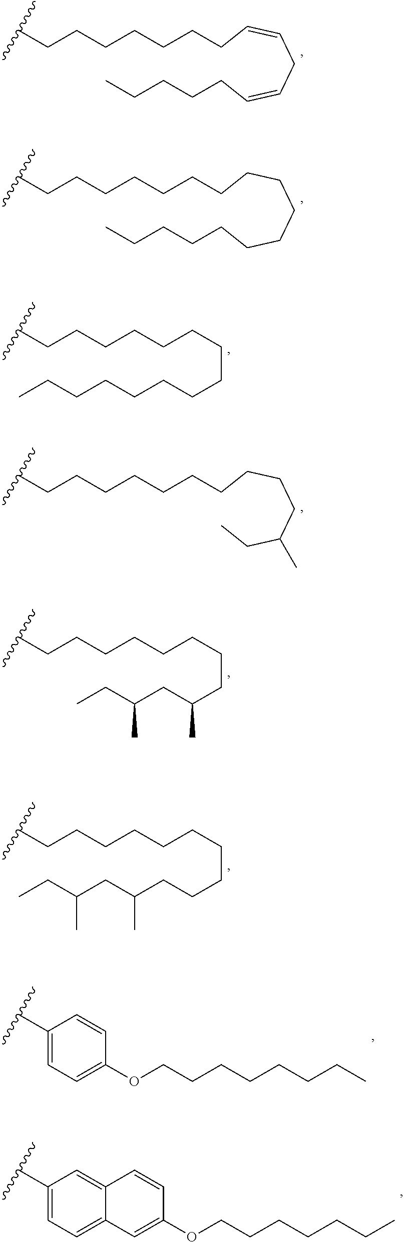

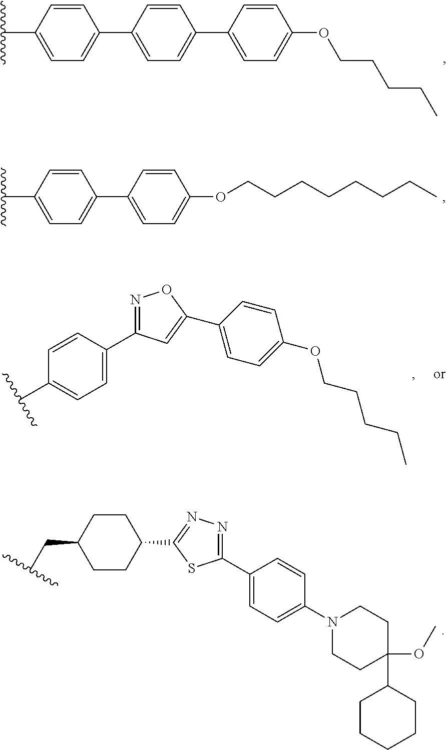 [SMILES: *c1ccc(-c2ccc(-c3ccc(OCCCCC)cc3)cc2)cc1 ; *c1ccc(-c2ccc(OCCCCCCCC)cc2)cc1 ; *c1ccc(-c2cc(-c3ccc(OCCCCC)cc3)on2)cc1 ; *C[C@H]1CC[C@H](c2nnc(-c3ccc(N4CCC(OC)(C5CCCCC5)CC4)cc3)s2)CC1]

53. The compound of any one of claim 52, wherein R⁹ is hydrogen, hydroxyl, —NR¹¹R¹², —O(CH₂)ₐNR¹¹R¹², —O(CH₂)ₐN⁺R¹¹R¹²R¹³, —O(CH₂)ₐO(CH₂)ₐNR¹¹R¹², —NH(CH₂)ₐNR¹¹R¹², —NH(CH₂)ₐN⁺R¹¹R¹²R¹³, —S(CH₂)ₐNR¹¹R¹², —S(CH₂)ₐN⁺R¹¹R¹²R¹³, —CH₂NR¹¹R¹², or —CH₂N⁺R¹¹R¹²R¹³.

54. The compound of claim 53, wherein the R⁹ is hydrogen, hydroxyl, —NH₂, —O(CH₂)₂NH₂, —O(CH₂)₂N⁺(CH₃)₃, —O(CH₂)₂O(CH₂)₂NH₂, —NH(CH₂)₂NH₂, —NH(CH₂)₂N⁺(CH₃)₃, —S(CH₂)₂NH₂, or —CH₂NH₂.

55. The compound of any one of claim 46, wherein (a) has the structure of:

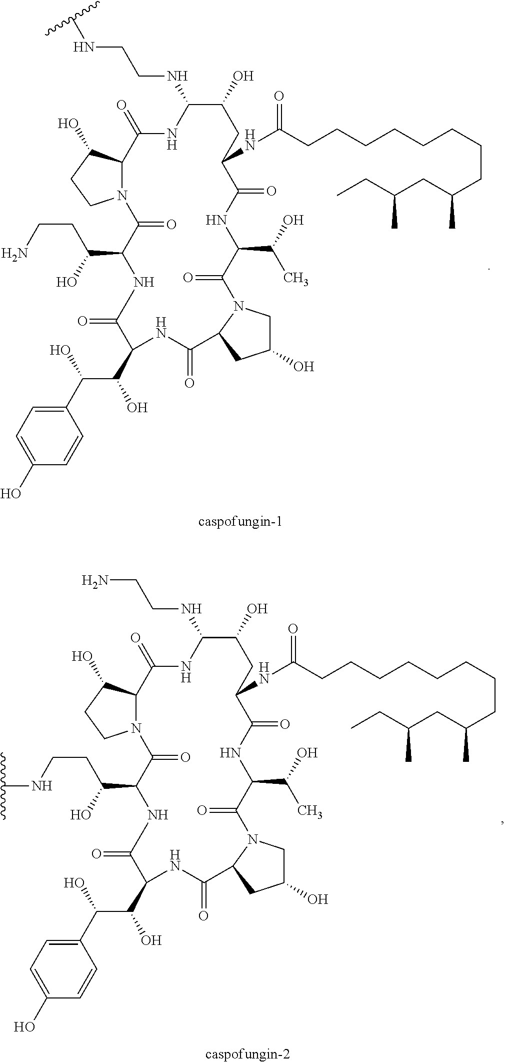 [SMILES: *NCCN[C@H]1NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@H]1O ; *NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](NCCN)NC(=O)[C@@H]2[C@@H](O)CCN2C1=O]

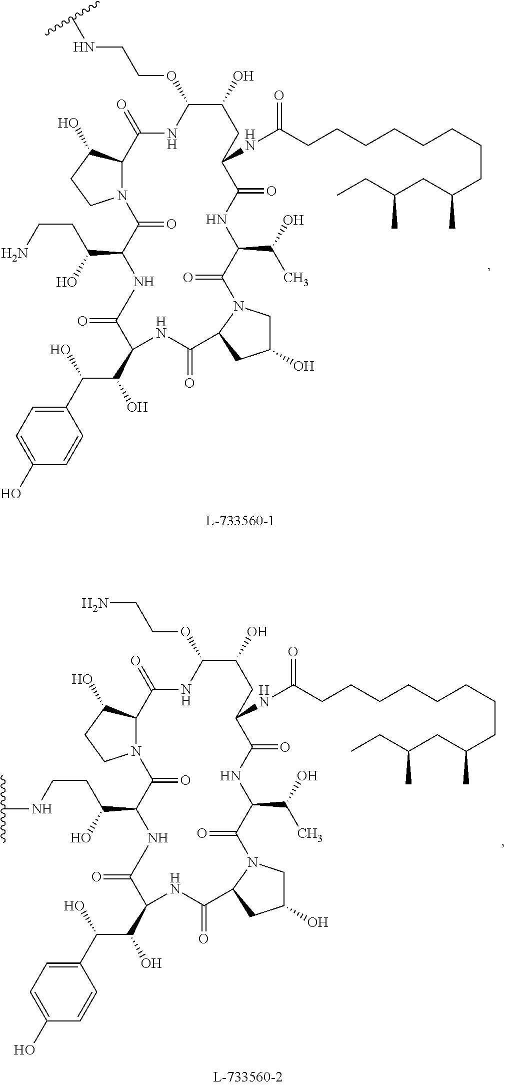 [SMILES: *NCCO[C@H]1NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@H]1O ; *NCC[C@@H](O)[C@@H]1NC(=O)[C@H]([C@H](O)[C@@H](O)c2ccc(O)cc2)NC(=O)[C@@H]2C[C@@H](O)CN2C(=O)[C@H]([C@@H](C)O)NC(=O)[C@@H](NC(=O)CCCCCCCC[C@@H](C)C[C@@H](C)CC)C[C@@H](O)[C@@H](OCCN)NC(=O)[C@@H]2[C@@H](O)CCN2C1=O]

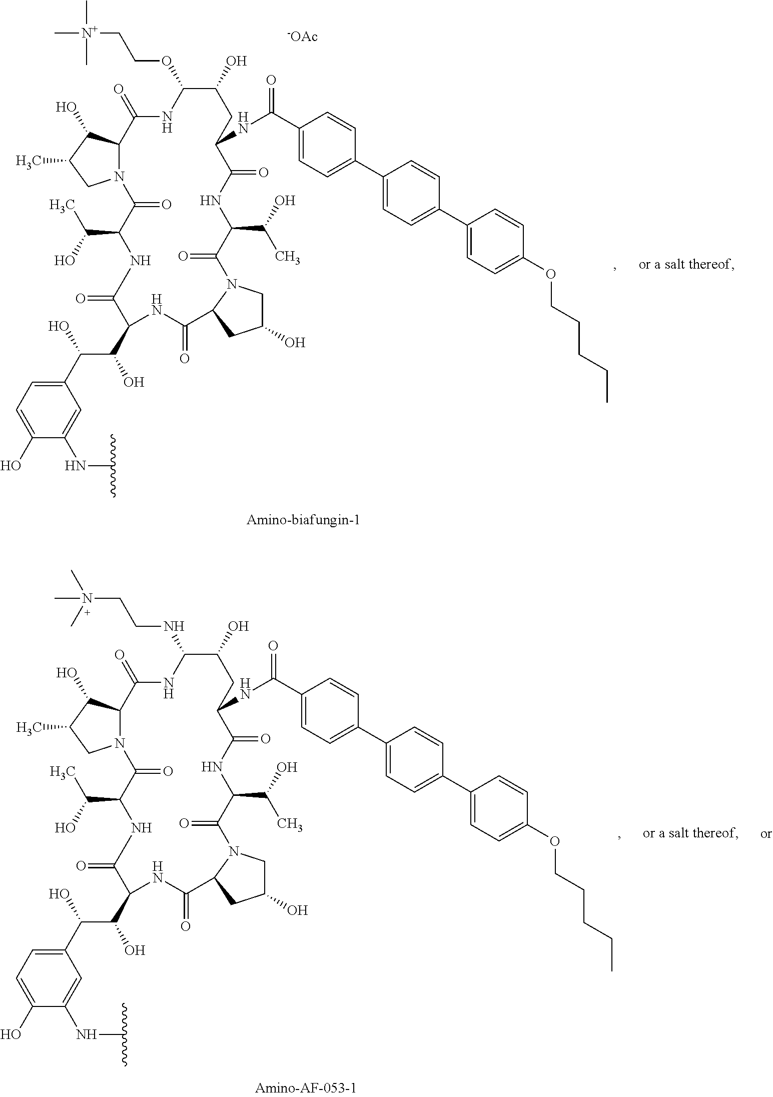

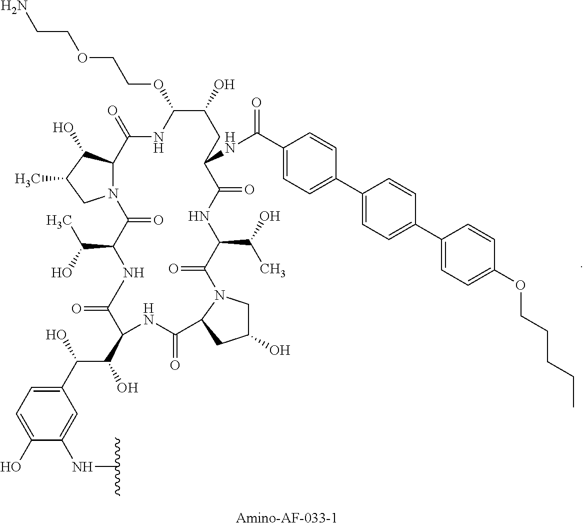 [SMILES: *Nc1cc([C@H](O)[C@@H](O)[C@@H]2NC(=O)[C@@H]3C[C@@H](O)CN3C(=O)[C@H]([C@@H](C)O)NC(=O)[C@@H](NC(=O)c3ccc(-c4ccc(-c5ccc(OCCCCC)cc5)cc4)cc3)C[C@@H](O)[C@@H](OCCOCCN)NC(=O)[C@@H]3[C@@H](O)[C@@H](C)CN3C(=O)[C@H]([C@@H](C)O)NC2=O)ccc1O]

56. The compound of any one of claims 46-55, wherein (b) comprises an amino acid residue having the formula:
R¹⁴—X1-X2-X9- Formula IV
wherein X1 is any amino acid;
X2 is leucine, isoleucine, or absent;
X9 is any amino acid or absent;
R¹⁴ is hydrogen or 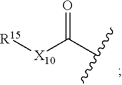
wherein X₁₀ is a bond, NH, or O; and
R¹⁵ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted oxime, optionally substituted hydrazone, optionally substituted aryl, or optionally substituted heterocyclic.

57. The compound of claim 56, wherein X1 is methionine, oxymethionine, or norleucine.

58. The compound of claim 56 or 57, wherein X2 is leucine or isoleucine, or is absent.

59. The compound of any one of claims 56-58, wherein X9 is phenylalanine, 1-amino-2-phenylcyclopropane-1-carboxylic acid, methionine, or serine, or is absent.

60. The compound of any one of claims 56-59, wherein R¹⁴ is —C(O)H.

61. The compound of claim 60, wherein (b) has the structure:

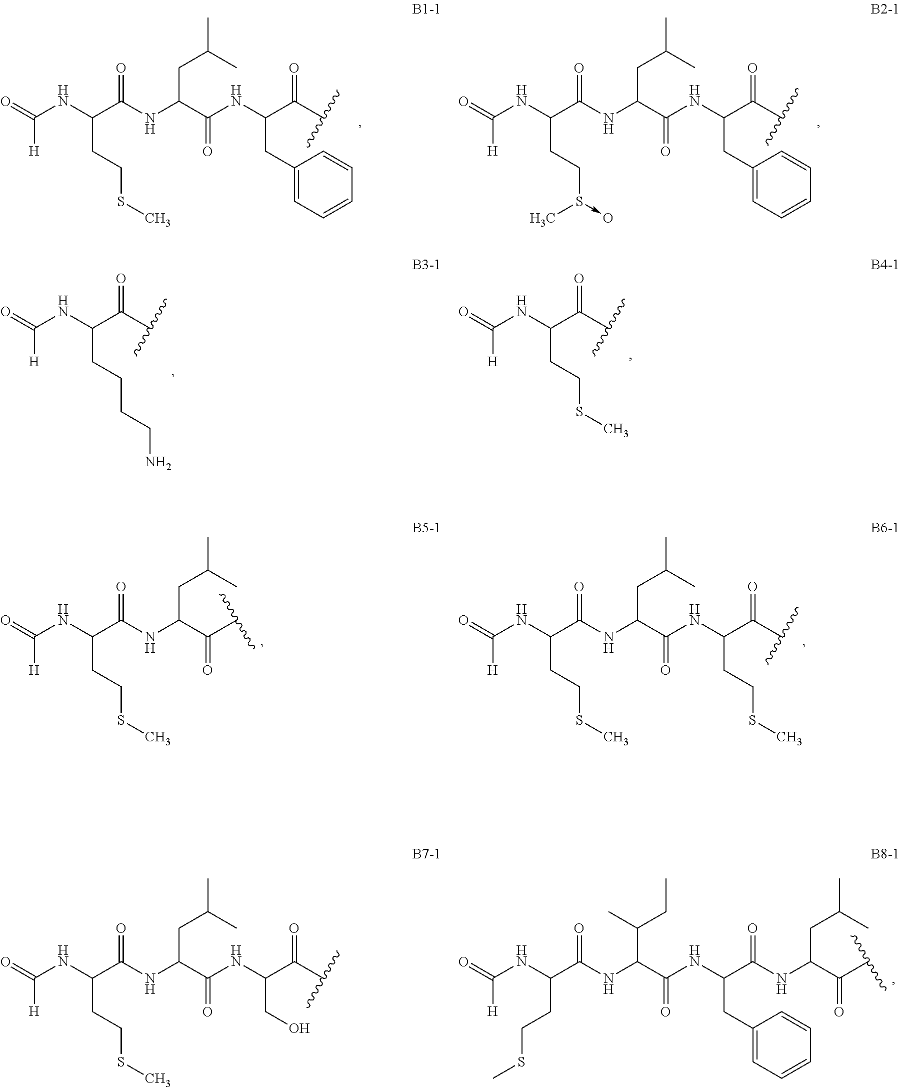

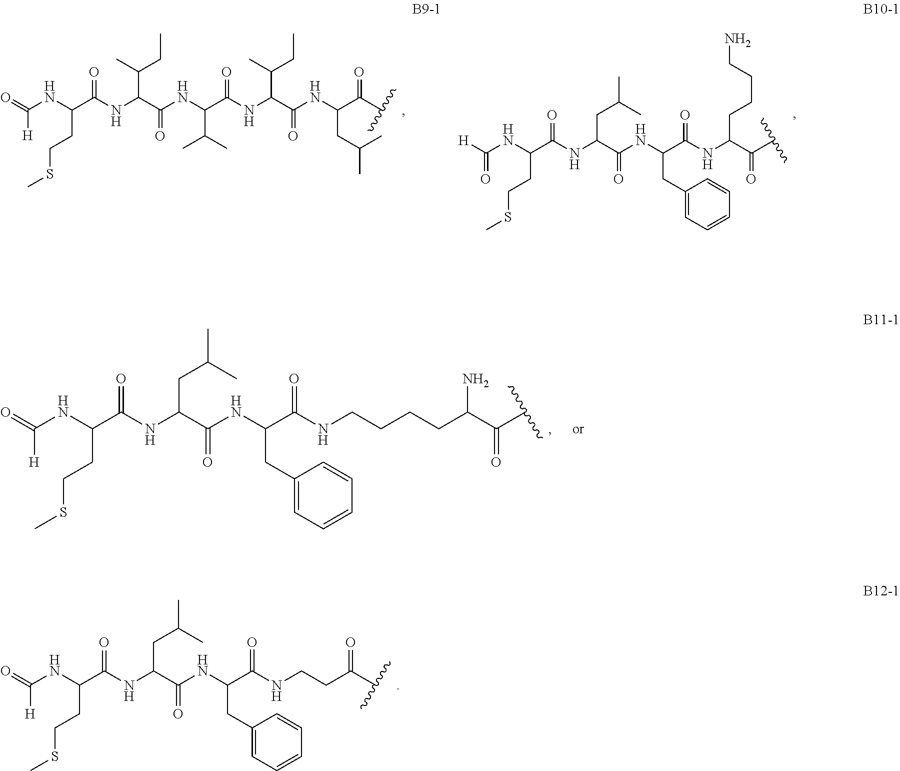 [SMILES: *C(=O)C(CC(C)C)NC(=O)C(NC(=O)C(NC(=O)C(NC(=O)C(CCSC)NC=O)C(C)CC)C(C)C)C(C)CC ; *C(=O)C(CCCCN)NC(=O)C(Cc1ccccc1)NC(=O)C(CC(C)C)NC(=O)C(CCSC)NC=O ; *C(=O)C(N)CCCCNC(=O)C(Cc1ccccc1)NC(=O)C(CC(C)C)NC(=O)C(CCSC)NC=O ; *C(=O)CCNC(=O)C(Cc1ccccc1)NC(=O)C(CC(C)C)NC(=O)C(CCSC)NC=O]

62. The compound of any one of claims 56-59, wherein R¹⁴ is —C(O)CH₃.

63. The compound of claim 62, wherein (b) has the structure:

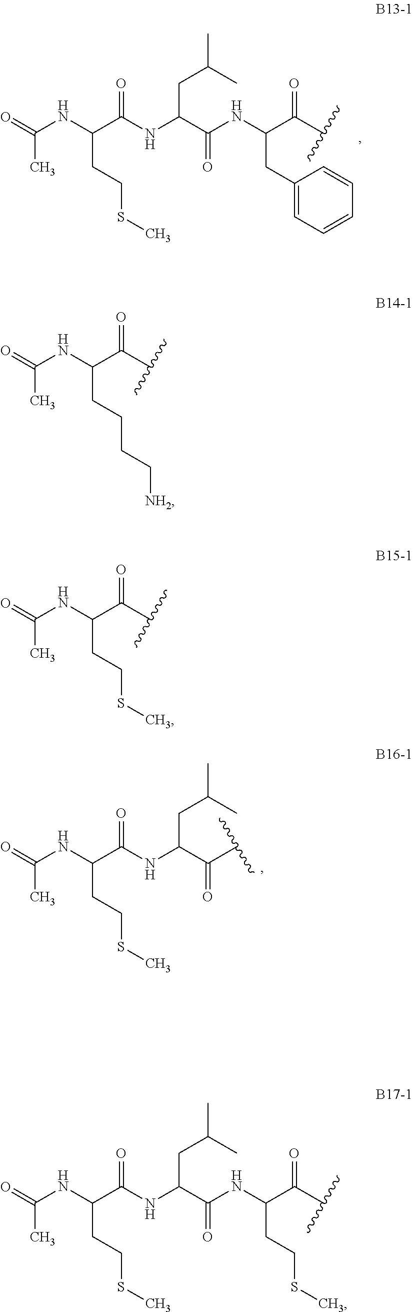 [SMILES: *C(=O)C(Cc1ccccc1)NC(=O)C(CC(C)C)NC(=O)C(CCSC)NC(C)=O ; *C(=O)C(CCCCN)NC(C)=O ; *C(=O)C(CCSC)NC(C)=O ; *C(=O)C(CC(C)C)NC(=O)C(CCSC)NC(C)=O ; *C(=O)C(CCSC)NC(=O)C(CC(C)C)NC(=O)C(CCSC)NC(C)=O]

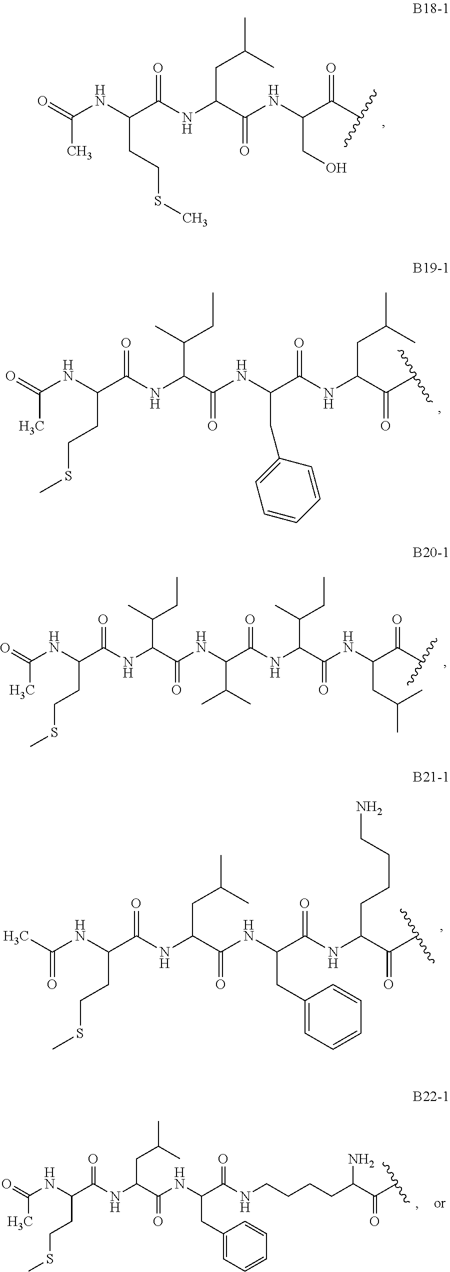

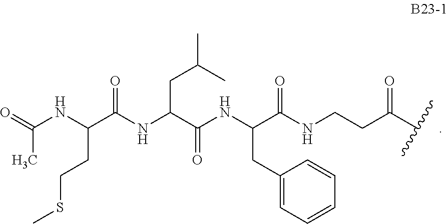 [SMILES: *C(=O)CCNC(=O)C(Cc1ccccc1)NC(=O)C(CC(C)C)NC(=O)C(CCSC)NC(C)=O]

64. The compound of any one of claims 56-59, wherein R¹⁴ is —C(O)OCH₂CH(CH₃)₂.

65. The compound of claim 64, wherein (b) has the structure:

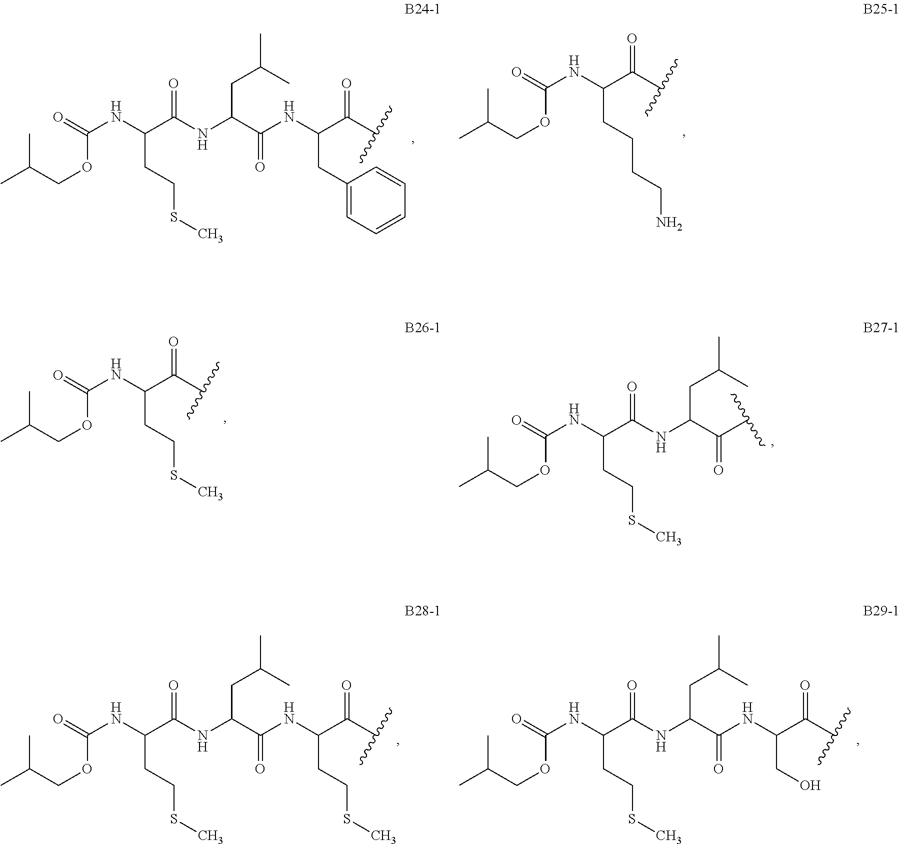 [SMILES: *C(=O)C(Cc1ccccc1)NC(=O)C(CC(C)C)NC(=O)C(CCSC)NC(=O)OCC(C)C ; *C(=O)C(CCCCN)NC(=O)OCC(C)C ; *C(=O)C(CCSC)NC(=O)OCC(C)C ; *C(=O)C(CC(C)C)NC(=O)C(CCSC)NC(=O)OCC(C)C ; *C(=O)C(CCSC)NC(=O)C(CC(C)C)NC(=O)C(CCSC)NC(=O)OCC(C)C ; *C(=O)C(CO)NC(=O)C(CC(C)C)NC(=O)C(CCSC)NC(=O)OCC(C)C]

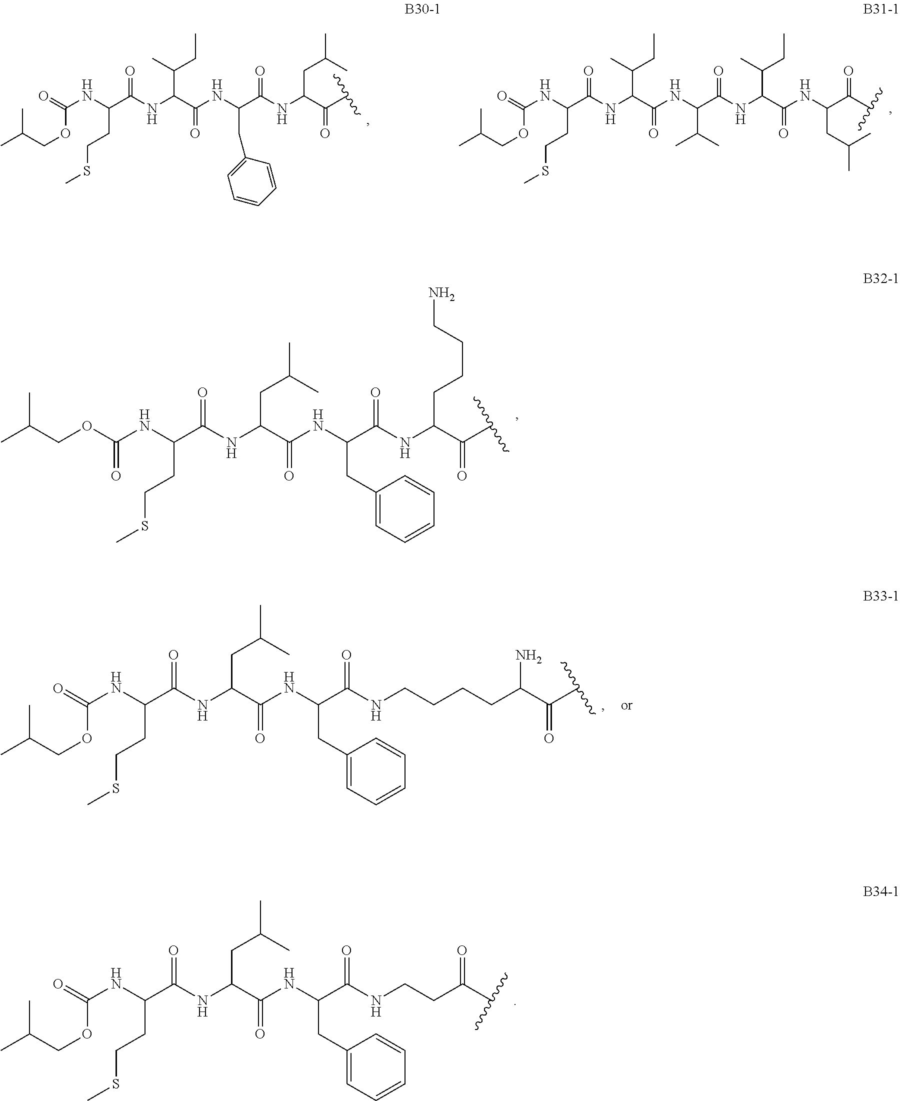 [SMILES: *C(=O)C(CC(C)C)NC(=O)C(Cc1ccccc1)NC(=O)C(NC(=O)C(CCSC)NC(=O)OCC(C)C)C(C)CC ; *C(=O)C(CC(C)C)NC(=O)C(NC(=O)C(NC(=O)C(NC(=O)C(CCSC)NC(=O)OCC(C)C)C(C)CC)C(C)C)C(C)CC ; *C(=O)C(CCCCN)NC(=O)C(Cc1ccccc1)NC(=O)C(CC(C)C)NC(=O)C(CCSC)NC(=O)OCC(C)C ; *C(=O)C(N)CCCCNC(=O)C(Cc1ccccc1)NC(=O)C(CC(C)C)NC(=O)C(CCSC)NC(=O)OCC(C)C ; *C(=O)CCNC(=O)C(Cc1ccccc1)NC(=O)C(CC(C)C)NC(=O)C(CCSC)NC(=O)OCC(C)C]

66. The compound of any one of claims 46-65, wherein (a) and (b) are conjugated via an amide bond.

67. The compound of any one of claims 46-65, wherein (a) and (b) are conjugated via a linker.

68. The compound or pharmaceutically acceptable salt of claim 67, wherein said linker comprises a non-reactive linking moiety of 1-100 atoms in length.

69. The compound of claim 68, wherein said linker has the structure:
G¹-(Z¹)_{b}—(Y¹)_{c}—(Z²)_{d}—(R¹⁶)—(Z³)ₑ—(Y²)_{f}—(Z⁴)_{g}-G² Formula V
wherein G¹ is a bond between said linker and said inhibitor of β-1,3-glucan synthase;
G² is a bond between said pathogen pattern recognition receptor ligand and said linker;
Z¹, Z², Z³, and Z⁴ each, is independently, optionally substituted C₁-C₂ alkylene, optionally substituted C₁-C₃ heteroalkylene, O, S, or NR¹⁷;
R¹⁷ is hydrogen, optionally substituted C₁₋₄ alkyl, optionally substituted C₃₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
Y¹ and Y² each, is independently, carbonyl, thiocarbonyl, sulphonyl, or phosphoryl;
b, c, d, e, f, and g are, independently, 0 or 1; and
R¹⁶ is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₆₋₁₂ arylene, optionally substituted C₂-C₁₀₀ polyethylene glycolene, or optionally substituted C₁₋₁₀ heteroalkylene, or a chemical bond linking G¹-(Z¹)_{b}—(Y¹)_{c}—(Z²)_{d}— to —(Z³)ₑ—(Y²)_{f}—(Z⁴)_{g}-G².

70. The compound of claim 78, wherein Z⁴ is NH, g is 1, Y¹ is carbonyl, c is 1, and b is 0.

71. The compound of any one of claims 67-70, wherein said linker is: 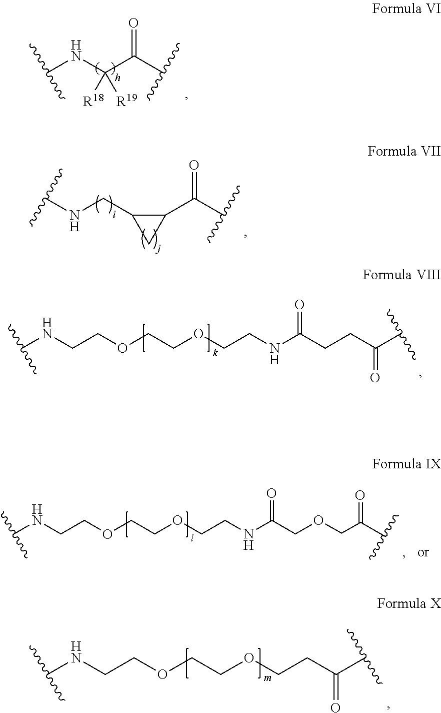
wherein h, i, j, k, l, and m are independently 1 to 12;
R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocycyl, or R¹⁸ and R¹⁹ taken together form a 3 to 6 membered cycloalkyl or heterocycle.

72. The compound of claim 71, wherein said linker is: 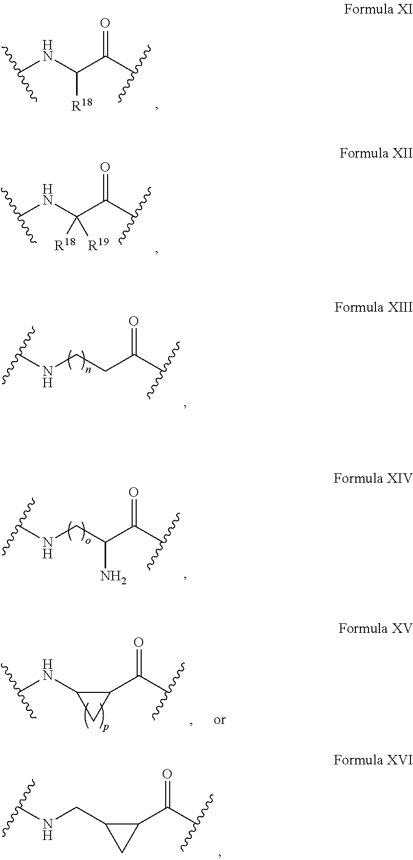
wherein n, o, and p are 1 to 4; and
R¹⁸ and R¹⁹ are independently hydrogen, amino, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted C₄-C₁₀ cycloalkenyl, optionally substituted C₄-C₁₀ cycloalkynyl, optionally substituted aryl, or optionally substituted heterocyclic, or R¹⁸ and R¹⁹ taken together form a 3 to 6 membered cycloalkyl or heterocycle.

73. The compound or pharmaceutically acceptable salt of any one of claims 67-72, wherein said linker is a polypeptide.

74. The compound of any one of claims 67-73, wherein (a) is attached to said linker via an amide bond and (b) is attached to said linker via an amide bond.

75. A pharmaceutical composition comprising a compound of any one of claims 1-74 or a pharmaceutically acceptable salt thereof and a pharmaceutically acceptable excipient.

76. A method for the treatment of a subject having a fungal infection or presumed to have a fungal infection, said method comprising administering to said subject an effective amount of a compound or composition of any one of claims 1-74.

77. A method for the prophylactic treatment of a fungal infection in a subject in need thereof, said method comprising administering to said subject an effective amount of a compound or composition of any one of claims 1-74.

78. The method of claim 76 or 77, wherein said fungal infection is caused by a fungus of the genus Aspergillus or Candida.

79. The method of any one of claims 76-78, wherein said fungal infection is aspergillosis.

80. The method of claim 79, wherein said aspergillosis is invasive aspergillosis.

81. The method of claim 79 or 80, wherein said aspergillosis is pulmonary aspergillosis.

82. The method of any one of claims 79-81, wherein said fungal infection is caused by Aspergillus fumigatus.

83. The method of any one of claims 76-78, wherein said fungal infection is candidiasis.

84. The method of claim 83, wherein said candidiasis is an intra-abdominal abscess, peritonitis, a pleural cavity infection, esophagitis, candidemia, or invasive candidiasis.

85. The method of claim 83 or 84, wherein said fungal infection is caused by Candida albicans.

86. The method of any one of claims 76-85, wherein said subject is immunocompromised.

87. The method of any one of claims 76-86, wherein said subject has been diagnosed with humoral immune deficiency, T cell deficiency, neutropenia, asplenia, or complement deficiency.

88. The method of any one of claims 76-87, wherein said subject is being treated or is about to be treated with immunosuppresive drugs.

89. The method of any one of claims 76-88, wherein said subject has been diagnosed with a disease which causes immunosuppression.

90. The method of claim 89, wherein said disease is cancer or acquired immunodeficiency syndrome.

91. The method of claim 90, wherein said cancer is leukemia, lymphoma, or multiple myeloma.

92. The method of any one of claims 76-91, wherein said subject has undergone or is about to undergo hematopoietic stem cell transplantation.

93. The method of any one of claims 76-91, wherein said subject has undergone or is about to undergo an organ transplant.

94. The method of any one of claims 76-93, wherein said administering comprises administering intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, locally, by inhalation, by injection, or by infusion.

95. A compound having the structure:

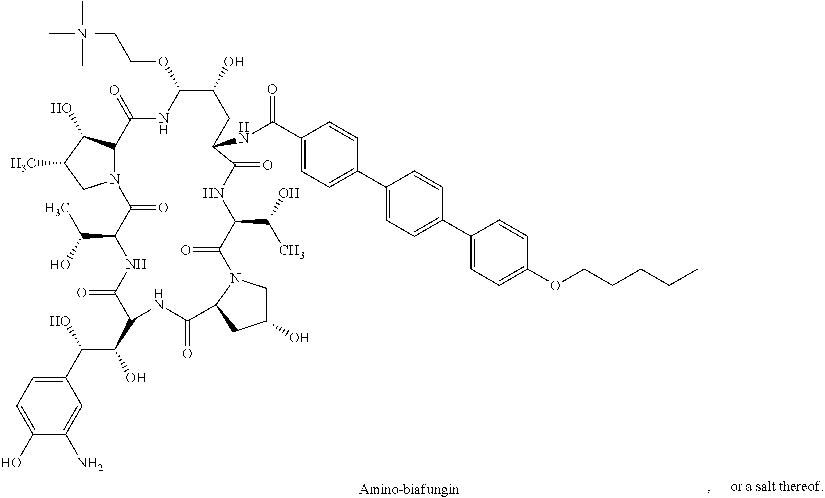

96. A compound having the structure:

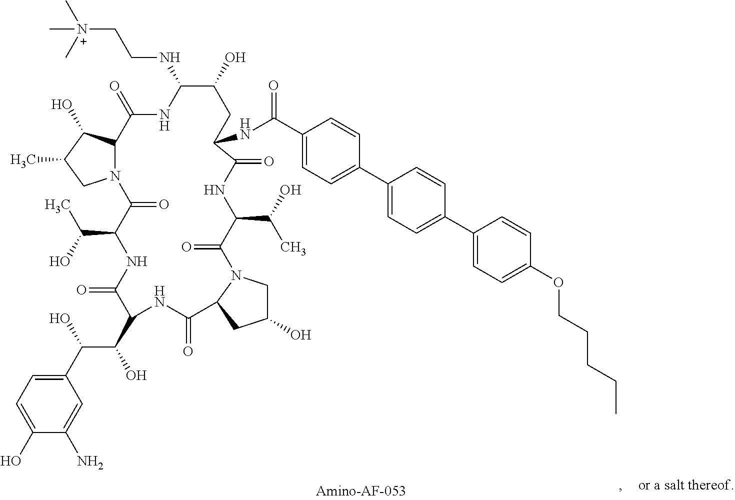

97. A compound having the structure: [chemical structure]
